(19) United States
(12) Patent Application Publication
(10) Pub. No.: US 2025/0360134 A1
(43) Pub. Date: Nov. 27, 2025

(54) 6-6 OR 5-6 FUSED BICYCLIC COMPOUNDS COMPRISING A PYRI(MI)DINE RING USEFUL IN THE|TREATMENT OF INFECTIOUS DISEASES

(71) Applicants: INSTITUT NATIONAL DE LA SANTE ET DE LA RECHERCHE MEDICALE (INSERM), Paris (FR); ECOLE POLYTECHNIQUE, Palaiseau (FR); UNIVERSITE DE BORDEAUX, Bordeaux (FR); INSTITUT PASTEUR, Paris (FR); CENTRE NATIONAL DE LA RECHERCHE SCIENTIFIQUE, Paris Cedex 16 (FR)

(72) Inventors: Jeanne CHIARAVALLI, PARIS (FR); Jean GUILLON, BORDEAUX (FR); Marc LAVIGNE, Paris (FR); Helene MUNIER-LEHMANN, Paris (FR); Jean-Louis MERGNY, Palaiseau (FR); Solene SAVRIMOUTOU, BORDEAUX (FR); Björn MEYER, PARIS (FR)

(21) Appl. No.: 18/258,070

(22) PCT Filed: Dec. 16, 2021
(86) PCT No.: PCT/EP2021/086262

§ 371 Date: Jun. 16, 2023

(30) Foreign Application Priority Data
Dec. 18, 2020 (EP) 20306606.3

(51) Int. Cl. A61K 31/519; A61K 31/437; A61K 31/52; A61P 33/02; C07D 471/04; C07D 473/00; C07D 473/40; C07D 487/04; C07D 495/04

(52) U.S. Cl. CPC A61K 31/519; A61K 31/437; A61K 31/52; A61P 33/02; C07D 471/04; C07D 473/00; C07D 473/40; C07D 487/04; C07D 495/04

(57) ABSTRACT

The present invention relates to new specific 6-6 or 5-6 fused bicyclic compounds comprising pyrimidine or pyridine useful in the prevention and/or treatment of infectious diseases. In particular, the present invention relates to a compound of formula (I) wherein: Aᵣ₁ is a (C₅-C₁₁)arylene or (C₅-C₁₁)heteroarylene group, X is —CH—, —S—, —NR⁵ or —N—, Y is —CH—, —NR⁵—S— or —NR⁶—CH₂—, T is —CH— and Q is —CH— or T is —N— and Q is —CR¹⁰— or —N—, provided that one or two of X, Q and Y comprise a heteroatom, and with the proviso that, at least one of R¹, R², and, if present, R⁵ or R⁶, contains a group —NH-Alk-NR³R⁴. The inventors showed that compounds of formula (I) present an activity against both W2 and 3D7 Plasmodium falciparum strains, an activity against T. brucei brucei but also an activity against SARS-CoV-2 virus, and that they are positive for G4 recognition. The invention also relates to the preparation process and to the therapeutic uses of the compounds of formula (I).

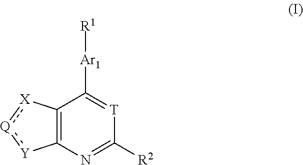

### SEQUENCE LISTING

This application includes as the Sequence Listing the complete contents of the .txt file “______”, created Dec. 22, 2023, containing 4,0096 bytes, hereby incorporated by reference.

### FIELD OF THE INVENTION

The present invention relates to new specific 6-6 or 5-6 fused bicyclic compounds comprising pyrimidine or pyridine useful as medicament.

Said new compounds are in particular useful in the prevention and/or treatment of infectious diseases, in particular parasitic and/or viral infectious diseases.

It further relates to the pharmaceutical compositions containing said new compounds and to the chemical synthesis processes for obtaining them.

### BACKGROUND

Infectious diseases, whether of bacterial, parasitic, viral, or other origin, present acute and chronic challenges to human health.

A first class of infectious diseases of particular interest to the World Health Organization (WHO) are caused by parasites.

On this basis, to the WHO, malaria remains a major public health problem, all the more worrying nowadays as epidemiological data show that no significant progress in reducing malaria cases was registered for the period 2015-2017, even though progress in reducing mortality from malaria occurs since 2017. The vast majority (99%) of cases are due to Plasmodium falciparum.

The increasing drug resistance of parasites worldwide, notably the resistance of Plasmodium falciparum to artemisinin, remains a major impediment to eradication of malaria, as it significantly reduces the potency of most used antimalarial compounds.

Thus, new antimalarial drugs with new potential mechanisms of action are now required to overcome this emerging resistance and also to control an ever-increasing number of epidemics due to the parasites.

Among other vector-borne parasitic diseases, those caused by parasites of the Trypanosomatidae family are also public health problems.

Indeed, leishmaniases, caused by parasites of the Leishmania genus which are transmitted by the bite of infected female phlebotomine sandflies, are among the most neglected parasitic diseases in the world.

Some 20 Leishmania species cause clinical manifestations of human leishmaniasis. Patients with leishmania have diverse symptoms grouped into three main clinical forms: cutaneous (the most common), mucocutaneous and visceral, also known as kala-azar and the most serious form of the disease since this is fatal if left untreated in over 95% of cases. Humans are the main reservoir for visceral leishmaniasis (VL) due to Leishmania donovani.

A limited number of drugs, all of which have high toxicities, resistances, and costs, can be used to treat leishmaniases, and although efforts have been made by WHO, non-governmental organizations, and manufacturers to improve access to medicines, leishmaniases persist as poverty-related diseases.

Furthermore, another neglected disease caused by Trypanosomatidae parasites of the Trypanosoma genus is Human African trypanosomiasis (HAT), also known as sleeping sickness, almost invariably fatal unless treated. There are two clinical forms: the slowly progressing form (gambiense HAT), caused by infection with Trypanosoma brucei gambiense (currently 98% of cases), and the faster progressing form (rhodesiense HAT), caused by infection with Trypanosoma brucei rhodesiense. Although the recent approval of a new medicine (fexinidazole) for the treatment of gambiense HAT has opened new possibilities for the management of cases, developing new drugs is of continuous interest.

Other parasites, either nematodes or platyhelminths, infect hundreds of millions of people, and cause pathologies such as Schistosomiasis, which causes over 200,000 deaths per year. The most effective treatment is praziquantel (PZ), which was developed 50 years ago. Unfortunately, this drug is ineffective against juvenile worms and resistance to PZ is suspected in the field.

Another class of infectious diseases of particular interest to the WHO are caused by viruses.

Indeed, viruses are one of the major causes of diseases around the world. Viruses are generally defined as small, non-living, infectious agents that replicate only within living cells, as they do not possess a completely autonomous replication mechanism. Although diverse in shape and size, they typically consist of a virus particle (known as a “virion”), made from a protein coat which comprises at least one nucleic acid molecule and optionally, depending on the type of virus, one or more proteins or nucleoproteins.

SARS-CoV-2, previously known as 2019-nCoV, has emerged in China in 2019 and rapidly propagated in numerous countries. It belongs to the Coronaviridae family which is part of the group IV of the Baltimore classification and causes an acute respiratory disease, named the coronavirus disease 2019, or COVID-19. This coronavirus shows sustained human-to-human transmission, along with many exported cases across the globe. As of Nov. 18, 2021, there have been 255,848,790 confirmed cases and 5,14,709 deaths with WHO having declared COVID-19 a “global pandemic” because of the unusually fast rate in which the virus is spreading.

Antiviral drugs for managing infections with human coronaviruses are not yet approved, posing a serious challenge to current global efforts aimed at containing the outbreak of COVID-19.

For obvious reasons, there is a need to find medicines that can counter the evolution of this emerging disease.

G-quadruplexes (G4s) are non-canonical structures that form in guanine-rich nucleic acids. These structures are stabilized by the stacking of G-quartets, planar arrays of four guanines that are held together by Hoogsteen base pairing in the presence of cations, generally monovalent cations. G4s can adopt intramolecular and intermolecular structures formed by one or more DNA or RNA strands, respectively, in parallel, hybrid or antiparallel configurations.

Quadruplexes have been ascribed roles in fundamental biological processes, including gene expression regulation, replication and transcription, RNA translation and processing, DNA recombination and telomere maintenance.

Telomeres are specialized nucleoprotein complexes that cap and protect the extremities of eukaryote chromosomes. In most eukaryotes, the telomeric DNA strand running from 5′ to 3′ consists of tandem repeats of a short motif that bears consecutive guanines. This strand extends beyond the complementary strand and results in a single-stranded, G-rich 3′ overhang. The inability of DNA polymerases to replicate linear chromosomes completely results in telomere shortening at each DNA replication. Telomere erosion can be compensated by a telomerase, a specialized reverse transcriptase, first identified in ciliates, capable of adding telomeric repeats to the 3′ ends of telomeres.

In the human genome, G4s are mainly associated with telomeric motifs (5′GGGTTA3′ repeats) and regulatory regions, such as transcription start sites (TSS), promoters, replication origins and nucleosome-depleted regions.

G4s stabilization was shown to inhibit telomerase elongation in vitro, and G-quadruplex-stabilizing ligands were therefore initially considered as potential telomerase inhibitors. Besides affecting telomere length, G4 ligands were also shown to induce short-term responses in human cells as a result of their ability to disrupt telomere structure.

So far, G4 ligands have been investigated as potential inhibitors of cancer cell proliferation.

Occurrence of G4s in other organisms including yeast, bacteria, plants, viruses and parasites has been studied. High proportion of non-canonical G4 structures that bear long loops or bulges, in several eukaryotic and prokaryotic genomes has been revealed.

Interestingly, DNA G4s can be detected in the nuclei of the malaria parasite Plasmodium falciparum, which has one of the most A/T-biased genomes sequenced and therefore possesses few guanine-rich sequences with the potential to form G-quadruplexes. Several G4-stabilizing molecules that were initially developed as potential anticancer agents displayed antimalarial activity. In particular, P. falciparum parasites are sensitive to several G-quadruplex-stabilizing drugs, including quarfloxin (Harris et al., Antimicrobial Agents and Chemotherapy, March 2018, Volume 62, Issue 3)

A recent study has highlighted a significant enrichment of G4 DNA motifs in var genes (family of genes encoding virulence factors, and subjected to numerous recombination) and in nucleosome-depleted regions of the P. falciparum genome and showed that the highly selective G4 molecule pyridostatin causes genome wide gene deregulation (Gazanion et al., PLoS Genet., 2020, 16(7): e1008917).

Parasitic G4s are also observed in L. spp and T. brucei (J. Guillon et al., Journal of Enzyme Inhibition and Medicinal Chemistry, 2020, 35(1), pp. 432-459).

The G4s of the different protozoa could constitute attractive drug targets.

In particular, G4 ligands, by binding specifically to RNA or DNA G-quadruplexes, may interfere with essential functions of the pathogen, for example by preventing opening of this structure during replication or transcription and inhibiting these processes.

In other words, compounds targeting specifically G4 can be potential candidates as antiparasitic agents.

In several viruses including human immunodeficiency virus (HIV), Herpes viruses, human papillomavirus (HPV), Epstein-Barr (EBV) and Hepatitis C virus (HCV), viral G4s present in promoters have been shown to modulate promoter activity.

Thus compounds specifically targeting G4 can further be potential candidates as antiviral agents.

Very recently, several PQSs in SARS-CoV-2 have been predicted by bioinformatic analysis and they are considered as potential binding motifs for SARS-CoV-2 protein. It has been recently proven that PQSs in SARS-CoV-2 can fold into stable unimolecular RNA G4 structures in live cells. The RNA G4 structures can be stabilized by G4 specific targeting compounds, such as PDP (pyridostatin derivative), accessing the regulation of G4 biofunctions. Actually, the protein levels of SARS-CoV-2 are reduced both in vitro and in vivo by PDP targeting RG-1 G4 structure (Chuanqi Zhao et al, Angew. Chem. Int. Ed. 2020, 59, 2-9). In addition, quadruplex binding proteins of viral origin may also bind to mRNA G-quadruplexes from the host cell, interfering with its antiviral response.

These results indicate that RNA G4 structures may be a novel target for developing antiviral drugs against viruses, including SARS-CoV-2.

EP 2 947 084 discloses five-and-six membered heterocyclic compounds having an activity as Jause kinase (JAK) inhibitors and their use for treating diseases such as cancers.

However, this document does not concern the treatment of infectious diseases and is silent regarding any specific 6-6 or 5-6 fused bicyclic compounds comprising pyrimidine or pyridine according to the present invention.

J. Guillon et al., Journal of Enzyme Inhibition and Medicinal Chemistry, 2020, 35(1), pp. 432-459 concerns the design, synthesis, and antiprotozoal evaluation of new 2,4-bis[(substituted aminomethyl) phenyl]quinoline, 1,3-bis[(substituted-aminomethyl)phenyl]isoquinoline and 2,4-bis[(substituted-aminomethyl)phenyl]quinazoline derivatives.

However, there is still a need to identify new compounds for treating and/or preventing infectious diseases in general, and particularly viral infectious diseases, such as Severe acute respiratory syndrome (SARS) induced by SARS-CoV-2 and/or parasitic infectious diseases caused by P. falciparum, L. donovani, S. mansoni or T. brucei.

In particular, there is still a need to identify new G4s specific targeting compounds, that could bind specifically to RNA or DNA G4s, thus interfering with essential functions of the parasites and/or viruses of which replication cycle key processes involve DNA or RNA G-quadruplexes from the parasite and/or virus genome or transcriptome itself or from host cell genome or transcriptome.

### SUMMARY OF THE INVENTION

It has now been found that the compounds as defined in formula (I) hereinafter are useful in the treatment and/or prevention of infectious diseases, in particular of parasitic infectious diseases and/or viral infectious diseases.

Indeed, as demonstrated in the examples below, compounds of formula (I) in accordance with the present invention exhibit antimalarial and antitrypasonomal activity along with antiviral activity against SARS-CoV-2 virus.

In particular, as demonstrated in the experimental part below, most of compounds of formula (I) exhibits in vitro antiplasmodial activity against the chloroquine-sensitive (3D7) and/or the chloroquine-resistant (W2) strains of the malaria parasite P. falciparum. Some of them also show in vitro efficacy against medically important protozoan T. brucei brucei.

Moreover, the in vitro cytotoxicity of compounds of formula (I) was assessed in human HepG2 cells, as an index of selectivity, and confirm the low toxicity of the claimed compounds.

Biological results also show antiviral activity against SARS Cov-2 of some compounds of formula (I).

Finally, most compounds of formula (I) have proven to be efficient ligands to bind G4s present into oligonucleotides mimicking the Plasmodium telomeric, the Trypanosoma 9 and 11 chromosomic and human telomeric sequences.

The present invention therefore relates to a compound of formula (I), as defined below.

The present invention further relates to a pharmaceutical composition comprising it, to a process for manufacturing it and to intermediate compounds involved in such process.

The present invention further relates to a compound of formula (I) as defined below for use as a medicament.

The present invention further relates to a compound of formula (I) as defined below for use in the treatment and/or prevention of infectious diseases, in particular of parasitic and/or viral infectious diseases.

The present invention further relates to the use of a compound of formula (I) as defined below for the manufacture of a medicament, in particular a medicament for the treatment and/or prevention of infectious diseases, more particularly of parasitic and/or viral infectious diseases.

The present invention further relates to the use of a compound of formula (I) as defined below for the treatment and/or prevention of infectious diseases, in particular of parasitic and/or viral infectious diseases.

### Definitions

As used herein, the term “patient” refers to either an animal, such as a valuable animal for breeding, company or preservation purposes, or preferably a human or a human child, which is afflicted with, or has the potential to be afflicted with, one or more diseases and conditions described herein.

In particular, as used in the present application, the term “patient” refers to a mammal such as a rodent, cat, dog, primate or human, and also extends to birds, preferably said subject is a human.

The identification of those patients who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those patients who are in need of such treatment.

In the context of the invention, the term “treating” or “treatment”, as used herein, means preventing, reversing, alleviating, inhibiting the progress of, or preventing the disease and its resulting cognitive, motor or metabolic changes.

Therefore, the term “treating” or “treatment” encompasses within the framework of the present invention the improvement of medical conditions of patients suffering from the diseases as described herein, in particular in the paragraph “PATHOLOGIES”.

As used herein, an “effective amount” refers to an amount of a compound of the present invention which is effective in preventing, reducing, eliminating, treating or controlling the symptoms of the herein-described diseases and conditions.

The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

The term “effective amount” includes “prophylaxis-effective amount” as well as “treatment-effective amount”.

The term “preventing”, as used herein, means reducing the risk of onset or slowing the occurrence of a given phenomenon, namely in the present invention, a disease as described herein.

As used herein, “preventing” also encompasses “reducing the likelihood of occurrence” or “reducing the likelihood of reoccurrence”.

The term “prophylaxis-effective amount” refers to a concentration of compound of this invention that is effective in inhibiting, preventing, decreasing the likelihood of occurrence of anyone of the herein described diseases.

Likewise, the term “treatment-effective amount” refers to a concentration of compound that is effective in treating the herein described diseases.

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

### DETAILED DESCRIPTION OF THE INVENTION

The inventors have surprisingly found that the compounds of formula (I) as disclosed herein after are useful for preventing and/or treating various infectious diseases, in particular parasitic and/or viral infectious diseases. This assertion is based on data as illustrated in the following examples and more detailed herein after.

According to a first aspect, a subject-matter of the present invention relates to a compound of formula (I), or a pharmaceutically acceptable salt thereof:

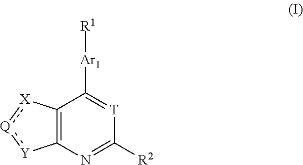

wherein:
Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom, a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group optionally substituted by a halogen atom or —(CH₂)ₙ—NH-Alk-NR³R⁴,
X is —CH—, —S—, —NR⁵—, or —N—,
Y is —CH—, —NR⁵—, —S— or —NR⁶—CH₂—,
T is —CH— and Q is —CH— or T is —N— and Q is —CR¹⁰— or —N—,
Each [symbol] is independently a simple or double bond, and at most one of [symbol] is a double bond,
Alk is a (C₁-C₆)alkanediyl group, optionally substituted by one or more group chosen from —COOR⁷ or —CONR³R⁴,
R⁵, if present, is a hydrogen atom, a (C₁-C₆)alkyl group optionally substituted by one or more (C₅-C₁₁)aryl or (C₅-C₁₁)heteroaryl group, a —(CH₂)ₙ—NH-Alk-NR³R⁴ or a (C₅-C₁₁)aryl group or (C₅-C₁₁)heteroaryl group optionally substituted by one or more halogen atoms, (C₁-C₆)alkoxy or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl group or (C₅-C₁₁)heteroaryl group optionally substituted by one or more halogen atoms, (C₁-C₆)alkoxy or —(CH₂)ₙ—NH-Alk-NR³R⁴,
Each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more, preferably one, (C₁-C₅)alkyl groups, preferably ethyl, propyl, butyl or pentyl,
n represent an integer chosen from 1, 2, 3, 4 and 5,
Each R⁷, R⁸ and R⁹ independently represents a hydrogen atom or a (C₁-C₆)alkyl group,
R¹⁰ is a hydrogen atom or a (C₅-C₁₁)aryl group or (C₅-C₁₁)heteroaryl,
provided that one or two of X, Q and Y comprise a heteroatom, and
with the proviso that, at least one of R¹, R², and, if present, R⁵ or R⁶, contains a group —NH-Alk-NR³R⁴.

Preferably, the compound may be of formula (I), or a pharmaceutically acceptable salt thereof, wherein:
Ar₁ is a (C₅-C₁₁)arylene, in particular (C₅-C₉)arylene, preferably a phenylene and R¹ is a hydrogen atom, a halogen atom, or a —(CH₂)ₙ—NH-Alk-NR³R⁴; or Ar₁ is a (C₅-C₁₁)heteroarylene group, in particular (C₅-C₉)heteroarylene group, preferably chosen among thiophenylene and triazolylene groups and R¹ is a halogen atom or a —(CH₂)ₙ—NH-Alk-NR³R⁴, and
X is —CH—, —S—, —NR⁵—, or —N—,
Y is —CH—, —NR⁵—, —S— or —NR⁶—CH₂—, and
T is —N— and Q is —CH—,
or
Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom, a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group optionally substituted by a halogen atom or —(CH₂)ₙ—NH-Alk-NR³R⁴,
X is —CH—, —S—, —NR⁵—, or —N—,
Y is —CH—, —NR⁵—, —S— or —NR⁶—CH₂—, and
T is —CH— and Q is —CH— or T is —N— and Q is —N—,
and
Each [symbol] is independently a simple or double bond, and at most one of [symbol] is a double bond,
Alk is a (C₁-C₆)alkanediyl group, optionally substituted by one or more group chosen from —COOR⁷ or —CONR³R⁴,
R⁵, if present, is a hydrogen atom, a (C₁-C₆)alkyl group optionally substituted by one or more (C₅-C₁₁)aryl or (C₅-C₁₁)heteroaryl group, a —(CH₂)ₙ—NH-Alk-NR³R⁴ or a (C₅-C₁₁)aryl group or (C₅-C₁₁)heteroaryl optionally substituted by one or more halogen atoms, (C₁-C₆)alkoxy or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl group or (C₅-C₁₁)heteroaryl optionally substituted by one or more halogen atoms, (C₁-C₆)alkoxy or —(CH₂)ₙ—NH-Alk-NR³R⁴,
Each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more, preferably one, (C₁-C₅)alkyl groups, preferably ethyl, propyl, butyl or pentyl,
n represent an integer chosen from 1, 2, 3, 4 and 5.
Each R⁷, R⁸ and R⁹ independently represents a hydrogen atom or a (C₁-C₆)alkyl group, provided that one or two of X, Q and Y comprise a heteroatom, and
with the proviso that, at least one of R¹, R², and, if present, R⁵ or R⁶, contains a group —NH-Alk-NR³R⁴.
   In particular, the compounds may be of formula (I), or a pharmaceutically acceptable salt thereof, wherein:
Ar₁ is a (C₅-C₁₁)arylene, in particular (C₅-C₉)arylene, preferably a phenylene and R¹ is a hydrogen atom, a halogen atom, or a —(CH₂)ₙ—NH-Alk-NR³R⁴; or Ar₁ is a (C₅-C₁₁)heteroarylene group, in particular (C₅-C₉)heteroarylene group, preferably chosen among thiophenylene and triazolylene groups and R¹ is a halogen atom, or a —(CH₂)ₙ—NH-Alk-NR³R⁴, and
Q, X, Y, T and R² are as defined here-above.

According to a particular embodiment, the present invention relates to a subgroup of compounds of formula (I), having following formula (I′), or a pharmaceutically acceptable salt thereof:

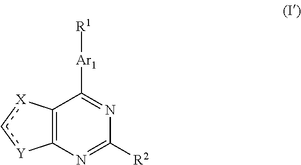

wherein:
Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom, a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group, optionally substituted by a halogen atom or —(CH₂)ₙ—NH-Alk-NR³R⁴,
X is —CH—, —S— or —NR⁵—,
Y is —CH—, —NR⁵—, —S— or —NR⁶—CH₂—,
Each [symbol] is independently a simple or double bond, and at most one of [symbol] is a double bond,
Alk is a (C₁-C₆)alkanediyl group, optionally substituted by one or more group chosen from —COOR⁷ or —CONR³R⁴,
R⁵, if present, is a hydrogen atom, a (C₁-C₆)alkyl group, a —(CH₂)ₙ—NH-Alk-NR³R⁴ or a (C₅-C₁₁)aryl group or (C₅-C₁₁)heteroaryl, optionally substituted by one or more halogen atoms or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl group or (C₅-C₁₁)heteroaryl, optionally substituted by one or more halogen atoms or —(CH₂)ₙ—NH-Alk-NR³R⁴,
Each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more, preferably one, (C₁-C₅)alkyl groups, preferably ethyl, propyl, butyl or pentyl,
n represent an integer chosen from 1, 2, 3, 4 and 5.
Each R⁷, R⁸ and R⁹ independently represents a hydrogen atom or a (C₁-C₆)alkyl group, provided that one of X and Y comprises a heteroatom, and
with the proviso that, at least one of R¹, R², and, if present, R⁵ or R⁶, contains a group —NH-Alk-NR³R⁴.

Preferably, the compound may be of formula (I′), or a pharmaceutically acceptable salt thereof, wherein:
Ar₁ is a (C₅-C₁₁)arylene, in particular (C₅-C₉)arylene, preferably a phenylene and R¹ is a hydrogen atom, a halogen atom, or a —(CH₂)ₙ—NH-Alk-NR³R⁴; or
Ar₁ is a (C₅-C₁₁)heteroarylene group, in particular (C₅-C₉)heteroarylene group, preferably chosen among thiophenylene and triazolylene groups, and R¹ is a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group, optionally substituted by a halogen atom or —(CH₂)ₙ—NH-Alk-NR³R⁴,
X is —CH—, —S— or —NR⁵—,
Y is —CH—, —NR⁵—, —S— or —NR⁶—CH₂—,
Each [symbol] is independently a simple or double bond, and at most one of [symbol] is a double bond,
Alk is a (C₁-C₆)alkanediyl group, optionally substituted by one or more group chosen from —COOR⁷ or —CONR³R⁴,
R⁵, if present, is a hydrogen atom, a (C₁-C₆)alkyl group, a —(CH₂)ₙ—NH-Alk-NR³R⁴ or a (C₅-C₁₁)aryl or (C₅-C₁₁)heteroaryl group, optionally substituted by one or more halogen atoms or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl or (C₅-C₁₁)heteroaryl group, optionally substituted by one or more halogen atoms or —(CH₂)ₙ—NH-Alk-NR³R⁴,
Each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more, preferably one, (C₁-C₅)alkyl groups, preferably ethyl, propyl, butyl or pentyl,
n represent an integer chosen from 1, 2, 3, 4 and 5.
Each R⁷, R⁸ and R⁹ independently represents a hydrogen atom or a (C₁-C₆)alkyl group, provided that one of X and Y comprises a heteroatom, and
with the proviso that, at least one of R¹, R², and, if present, R⁵ or R⁶, contains a group —NH-Alk-NR³R⁴.

According to a particular embodiment, the present invention relates to a first subgroup of compounds of formula (I) or (I′), of following formula (Ia), or a pharmaceutically acceptable salt thereof:

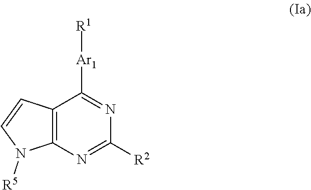

wherein Ar₁, R¹, R² and R⁵ are as defined here-above for formula (I) or (I′).

Preferably, Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom or a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group or a (C₅-C₁₁)aryl group or (C₅-C₁₁)heteroaryl, optionally substituted by one or more (C₁-C₆)alkoxy or —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   More preferably, Ar₁ is a (C₅-C₁₁)arylene, in particular (C₅-C₉)arylene, preferably a phenylene, and R¹ is a hydrogen atom or a —(CH₂)ₙ—NH-Alk-NR³R⁴, or
Ar₁ is a (C₅-C₁₁)heteroarylene group, in particular (C₅-C₉)heteroarylene group, preferably chosen among thiophenylene and triazolylene groups, and R¹ is a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group or a (C₅-C₁₁)aryl or (C₅-C₁₁)heteroaryl group, optionally substituted by one or more —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   These compounds may more precisely be designated as pyrrolopyrimidine derivatives, or pharmaceutically acceptable salts thereof.

According to this embodiment, compounds may be of following formula (Ia-a), or a pharmaceutically acceptable salt thereof:

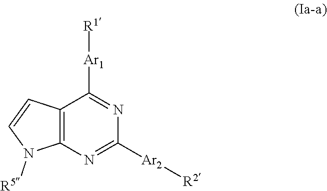

wherein each R^{1′} and R^{2′} is a —(CH₂)ₙ—NH-Alk-NR³R⁴,
R^{5″} is a (C₁-C₆)alkyl group or a phenyl group,
Each Ar₁ and Ar₂ is independently a phenylene or a thiophenylene, and
each Alk, R³, R⁴ and n is as defined here-above for formula (I), (I′) or (Ia).

Preferably R^{5″} is a phenyl, each R^{5′} and R^{2′} is a —(CH₂)—NH-Alk-NR³R⁴, each Ar is phenylene, Alk is an unsubstituted (C₂-C₅)alkanediyl group, preferably linear propylene, linear butylene or linear pentylene, each R³ and R⁴ independently represents a (C₁-C₆)alkyl group, preferably a methyl, an ethyl or an isopropyl, or form together with the nitrogen atom bearing them a (C₃-C₈)heterocycloalkyl group optionally substituted by one or more, preferably one, (C₁-C₅)alkyl groups, preferably a N-methylpiperazinyl.

Still according to this embodiment, compounds may be of following formula (Ia-b), or a pharmaceutically acceptable salt thereof:

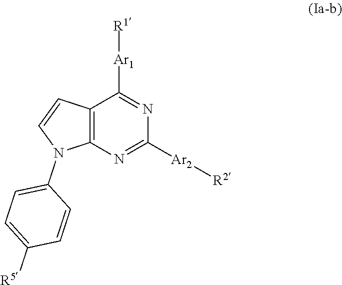

wherein each R^{1′} and R^{2′} is a hydrogen atom or a —(CH₂)ₙ—NH-Alk-NR³R⁴,
R^{5′} is a (C₁-C₆)alkoxy or —(CH₂)ₙ—NH-Alk-NR³R⁴,
Ar₁ and Ar₂ are independently a phenylene or a thiophenylene, and
each Alk, R³, R⁴ and n is as defined here-above for formula (I), (I′) or (Ia).
   Preferably, Ar₁ is a phenylene and R^{1′} is a hydrogen atom or a —(CH₂)ₙ—NH-Alk-NR³R⁴, or
Ar₁ is a thiophenylene and R^{1′} is a —(CH₂)ₙ—NH-Alk-NR³R⁴,
R^{2′} is a hydrogen atom or a —(CH₂)ₙ—NH-Alk-NR³R⁴,
R^{5′} is (C₁-C₆)alkoxy or —(CH₂)ₙ—NH-Alk-NR³R⁴,
Ar₂ is a phenylene or a thiophenylene, and
each Alk, R³, R⁴ and n is as defined here-above for formula (I), (I′) or (Ia).

More preferably, Each R^{5′}, R^{1′} and R^{2′} is a —(CH₂)—NH-Alk-NR³R⁴, Ar₁ and Ar₂ are phenylene, Alk is an unsubstituted (C₂-C₄)alkanediyl group, preferably linear propylene, linear butylene or linear pentylene, and each R³ and R⁴ independently represent a (C₁-C₆)alkyl group, preferably a methyl, or form together with the nitrogen atom bearing them a (C₃-C₈)heterocycloalkyl group optionally substituted by one or more, preferably one, (C₁-C₅)alkyl groups, preferably a N-methylpiperazinyl.

Still according to this embodiment, compounds may be of following formula (Ia-c), or a pharmaceutically acceptable salt thereof:

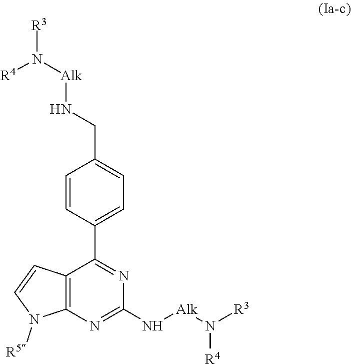

wherein:
R^{5″} is a hydrogen atom or a phenyl group, and
each Alk, R³, R⁴ and n is as defined here-above for formula (I), (I′) or (Ia).

Preferably, R^{5′} is a phenyl group, Alk is an unsubstituted (C₂-C₄)alkanediyl group, preferably a linear propylene, linear butylene or linear pentylene, and each R³ and R⁴ is a methyl or form together with the nitrogen atom bearing them a N-methylpiperazinyl.

Still according to this embodiment, compounds may be of following formula (Ia-d), or a pharmaceutically acceptable salt thereof:

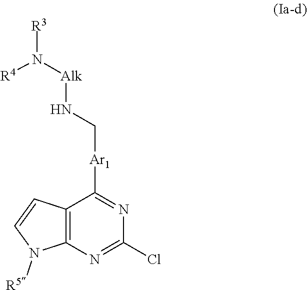

wherein Ar₁, Alk, R³, R⁴ are as defined here-above for formula (I), (I′) or (Ia) and R^{5″} is as defined here-above for formula (Ia-a).

Preferably, R^{5″} is a hydrogen atom or a phenyl group,
Ar₁ is chosen from phenylene and thiophenylene,
Alk is an unsubstituted (C₂-C₄)alkanediyl group, preferably a linear propylene, linear butylene or linear pentylene,
each R³ and R⁴ independently represents a (C₁-C₆)alkyl group, preferably a methyl, or form together with the nitrogen atom bearing them a (C₃-C₈)heterocycloalkyl group optionally substituted by one or more (C₁-C₅)alkyl groups, preferably a N-methylpiperazinyl.

Still according to this embodiment, compounds may be of following formula (Ia-e), or a pharmaceutically acceptable salt thereof:

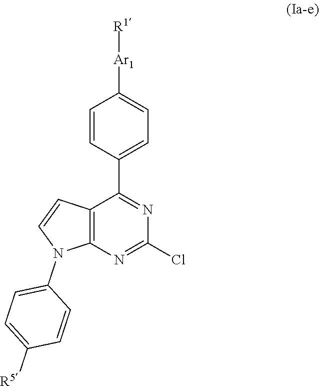

wherein Ar₁ is as defined here-above for formula (I), (I′) or (Ia) and R^{1′} and R^{5′} are as defined here-above for formula (Ia-b).

According to a particular embodiment, the present invention relates to a second subgroup of compounds of formula (I) or (I′), having following formula (Ib), or a pharmaceutically acceptable salt thereof:

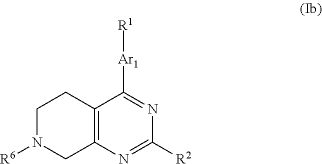

wherein Ar₁, R¹, R² and R⁶ are as defined here-above for formula (I) or (I′).

Preferably, Ar₁ is a (C₅-C₁₁)arylene, in particular (C₅-C₉)arylene, preferably a phenylene,
R¹ is —(CH₂)ₙ—NH-Alk-NR³R⁴,
R² is a (C₅-C₁₁)aryl, optionally substituted by a —(CH₂)ₙ—NH-Alk-NR³R⁴,
Alk is a (C₁-C₆)alkanediyl group, preferably a (C₂-C₄)alkanediyl group,
R⁶ is a hydrogen atom or a —COOR⁸,
each R³ and R⁴ independently represents a (C₁-C₆)alkyl group, or form together with the nitrogen atom bearing them a (C₃-C₈)heterocycloalkyl group, optionally substituted by one or more (C₁-C₅)alkyl, preferably a N-methylpiperazinyl,
each n independently represents an integer chosen from 1 and 2, preferably is 1,
R⁸, if present, represents a hydrogen atom or a (C₁-C₆)alkyl group.

These compounds may more precisely be designated as dihydropyridopyrimidine derivatives, or pharmaceutically acceptable salts thereof.

According to this embodiment, compounds may preferably be of following formula (Ib-a), or a pharmaceutically acceptable salt thereof:

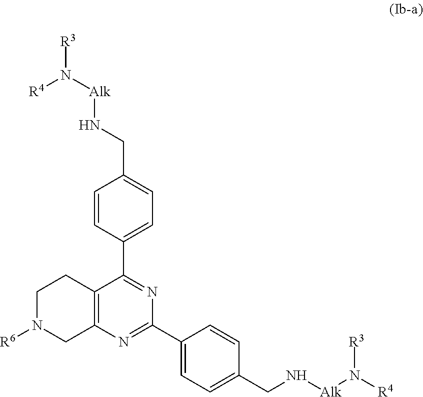

wherein R⁶, Alk, R³ and R⁴ are as defined here-above for formula (I), (I′) or (Ib).

Preferably, each R³ and R⁴ is a methyl or form together with the nitrogen atom bearing them a N-methylpiperazinyl, Alk is a propylene and R⁶ is a hydrogen atom or —COOR⁸ with R⁸ being a (C₁-C₆)alkyl group, preferably a (C₁-C₄)alkyl group, more preferably a tert-butyl.

According to a particular embodiment, the present invention relates to a third subgroup of compounds of formula (I) or (I′), having following formula (Ic) or a pharmaceutically acceptable salt thereof:

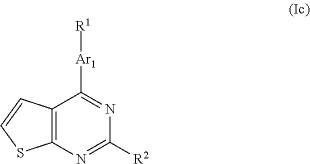

wherein Ar₁, R¹ and R² are as defined here-above for formula (I) or (I′).

Preferably, Ar₁ is a (C₅-C₁₁)arylene, in particular (C₅-C₉)arylene, preferably a phenylene,
R¹ is —(CH₂)ₙ—NH-Alk-NR³R⁴,
R² is a halogen atom, —NH-Alk-NR³R⁴ or (C₅-C₁₁)aryl or (C₅-C₁₁)heteroaryl group substituted by a —(CH₂)ₙ—NH-Alk-NR³R⁴,
Alk is a (C₁-C₆)alkanediyl group, preferably a (C₂-C₄)alkanediyl group,
Each R³ and R⁴ independently represent a (C₁-C₆)alkyl group or form together with the nitrogen atom bearing them a (C₃-C₈)heterocycloalkyl group, substituted by one or more, preferably one, (C₁-C₅)alkyl groups, preferably methyl, and
n represents an integer chosen from 1, 2, 3, 4 and 5, preferably is 1.

These compounds may more precisely be designated as thienopyrimidine derivatives, or pharmaceutically acceptable salts thereof.

According to this embodiment, compounds may more particularly be of following formula (Ic-a), or a pharmaceutically acceptable salt thereof:

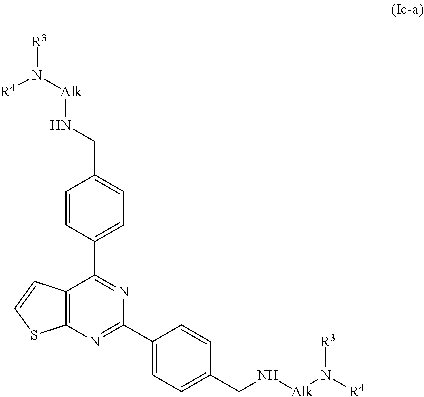

wherein Alk, R³ and R⁴ are as defined here-above for formula (I), (I′) or (Ic).

Preferably, Alk is an unsubstituted (C₂-C₄)alkanediyl group, preferably a linear propylene, and each R³ and R⁴ is a methyl or form, together with the nitrogen atom bearing them, a N-methylpiperazinyl.

Still according to this embodiment, compounds may be of following formula (Ic-b), or a pharmaceutically acceptable salt thereof:

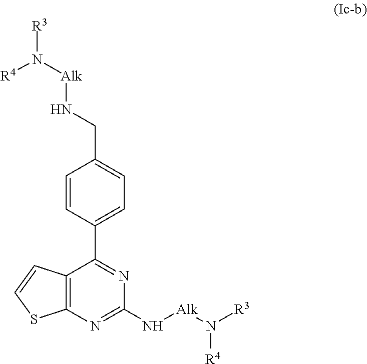

wherein each Alk, R³ and R⁴ is as defined here-above for formula (I), (I′) or (Ic).

Preferably Alk is an unsubstituted (C₂-C₄)alkanediyl group, preferably a linear propylene, and each R³ and R⁴ is a methyl or form together with the nitrogen atom bearing them a N-methylpiperazinyl.

Still according to this embodiment, compounds may be of following formula (Ic-c), or a pharmaceutically acceptable salt thereof:

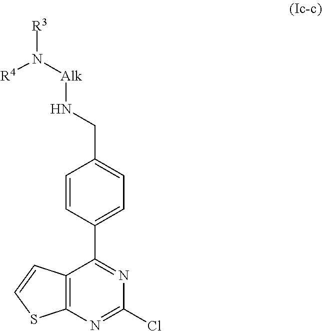

wherein Alk, R³ and R⁴ are as defined here-above for formula (I), (I′) or (Ic).

Preferably, Alk is propylene and each R³ and R⁴ is a methyl or R³ and R⁴ form, together with the nitrogen atom bearing them a N-methyl-piperazinyl group.

According to a particular embodiment, the present invention relates to a fourth subgroup of compounds of formula (I) or (I′), having following formula (Id), or a pharmaceutically acceptable salt thereof:

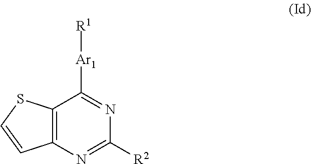

wherein Ar₁, R¹ and R² are as defined here-above for formula (I) or (I′).

Preferably Ar₁ is a (C₅-C₁₁)arylene, in particular (C₅-C₉)arylene, preferably a phenylene,
R¹ is —(CH₂)ₙ—NH-Alk-NR³R⁴,
R² is a halogen atom, —NH-Alk-NR³R⁴ or a (C₅-C₁₁)aryl substituted by a —(CH₂)ₙ—NH-Alk-NR³R⁴,
Alk is a (C₁-C₆)alkanediyl group, preferably a (C₂-C₄)alkanediyl group,
each R³ and R⁴ independently represents a (C₁-C₆)alkyl group, or form together with the nitrogen atom bearing them a (C₃-C₈)heterocycloalkyl group, optionally substituted by one or more, preferably one, (C₁-C₅)alkyl groups, preferably methyl, and
n represents an integer chosen from 1, 2, 3, 4 and 5, preferably is 1.

These compounds may more precisely be designated as thienopyrimidine derivatives, or pharmaceutically acceptable salts thereof.

According to this embodiment, compounds may be of following formula (Id-a), or a pharmaceutically acceptable salt thereof:

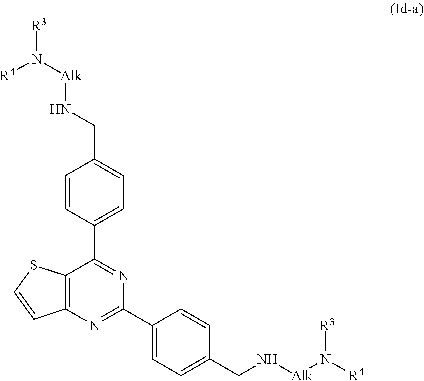

wherein Alk, R³ and R⁴ are as defined here-above for formula (I), (I′) or (Id).

Preferably, Alk is propylene and each R³ and R⁴ is a methyl or R³ and R⁴ form together with the nitrogen atom bearing them a (C₃-C₈)heterocycloalkyl group, optionally substituted by one or more, preferably one, (C₁-C₅)alkyl groups, preferably methyl.

Still according to this embodiment, compounds may be of following formula (Id-b), or a pharmaceutically acceptable salt thereof:

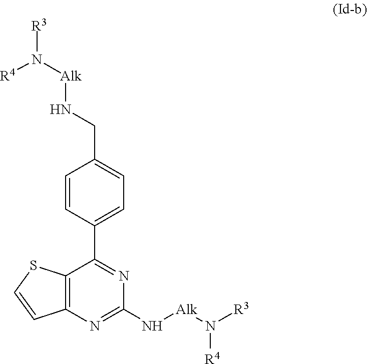

wherein Alk, R³ and R⁴ are as defined here-above for formula (I), (I′) or (Id).

Preferably, Alk is propylene and each R³ and R⁴ is a methyl or form together with the nitrogen atom bearing them a N-methylpiperazinyl.

Still according to this embodiment, compounds may be of following formula (Id-c), or a pharmaceutically acceptable salt thereof:

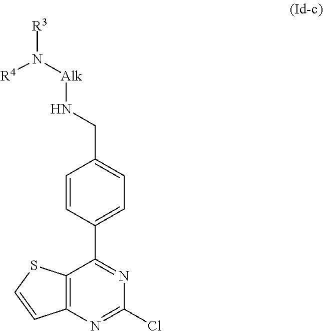

wherein Alk, R³ and R⁴ are as defined here above for formula (I), (I′) or (Id).

Preferably, Alk is propylene and each R³ and R⁴ is a methyl or R³ and R⁴ form, together with the nitrogen atom bearing them a N-methyl-piperazinyl group.

According to a particular embodiment, the present invention relates to a fifth subgroup of compounds of formula (I), having following formula (Ie), or a pharmaceutically acceptable salt thereof:

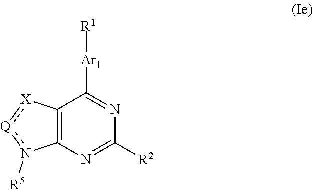

wherein Ar₁, R¹, R² and R⁵ are as defined hereabove for formula (I), and
X is —CH— and Q is —N— or
X is —N— and Q is —CH—.

According to this embodiment, compounds may be of following formula (Ie-a), or a pharmaceutically acceptable salt thereof:

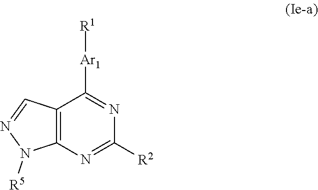

wherein Ar₁, R¹, R² and R are as defined here-above for formula (I) or (Ie).

Preferably Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom or a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group or a (C₅-C₁₁)aryl or (C₅-C₁₁)heteroaryl group, optionally substituted by one or more —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   These compounds may more precisely be designated as pyrazolopyrimidine derivatives, or pharmaceutically acceptable salts thereof.
   Even more preferably, compounds according to this embodiment are of following formula (Ie-a′):

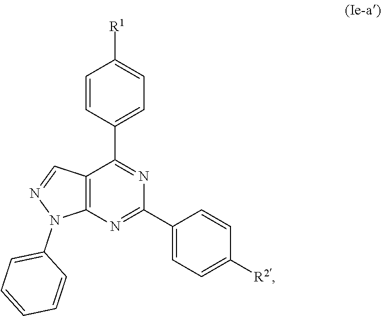

wherein R^{2′} is a hydrogen atom or a —(CH₂)ₙ—NH-Alk-NR³R⁴, and
R¹, Alk, R³, R⁴ and n are as defined hereabove for formula (I), (Ie) or (Ie-a)
with the proviso that at least one of R¹ and R^{2′} contains a —NH-Alk-NR³R⁴,
preferably wherein R¹ and R^{2′} are —(CH₂)—NH-Alk-NR³R⁴,
Alk is an unsubstituted (C₁-C₆)alkanediyl group, preferably a (C₃-C₅)alkanediyl group, in particular linear propylene, linear butylene, or linear pentylene,
Each R³ and R⁴ independently represent a (C₁-C₆)alkyl group, preferably a methyl, or alternatively R³ and R⁴ form together with the nitrogen atom bearing them a (C₃-C₈)heterocycloalkyl group, preferably a piperazinyl group, optionally substituted by one or more, preferably one, (C₁-C₅)alkyl groups, preferably methyl.

Still according to this embodiment, compounds may be of following formula (Ie-b), or a pharmaceutically acceptable salt thereof:

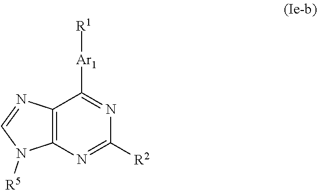

wherein Ar₁, R¹, R² and R⁵ are as defined here-above for formula (I) or (Ie).
Preferably Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom or a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group or a (C₅-C₁₁)aryl or (C₅-C₁₁)heteroaryl group, optionally substituted by one or more —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   These compounds may more precisely be designated as imidazopyrimidine derivatives, or pharmaceutically acceptable salts thereof.
   Even more preferably, compounds according to this embodiment are of following formula (Ie-b′):

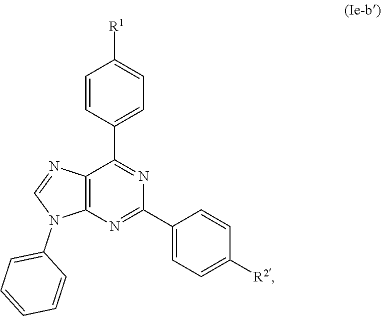

wherein R^{2′} is a hydrogen atom or a —(CH₂)ₙ—NH-Alk-NR³R⁴, and
R¹, Alk, R³, R⁴ and n are as defined hereabove for formula (I), (Ie) or (Ie-b)
with the proviso that at least one of R¹ and R^{2′} contains a —NH-Alk-NR³R⁴,
preferably wherein R¹ and R² are —(CH₂)—NH-Alk-NR³R⁴,
Alk is an unsubstituted (C₁-C₆)alkanediyl group, preferably a (C₃-C₅)alkanediyl group, in particular a linear butylene or linear pentylene group,
Each R³ and R⁴ independently represent a (C₁-C₆)alkyl group, preferably a methyl, or alternatively R³ and R⁴ form together with the nitrogen atom bearing them a (C₃-C₈)heterocycloalkyl group, preferably a piperazinyl group, optionally substituted by one or more, preferably one, (C₁-C₅)alkyl groups, preferably methyl.
   Alternatively, compounds according to this embodiment may be of following formula (Ie-b″):

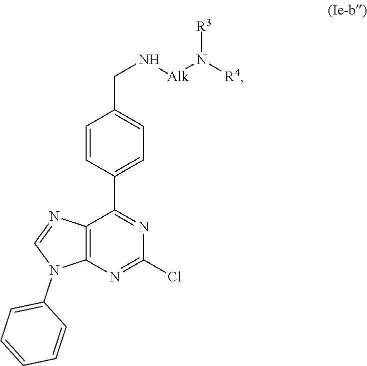

wherein Alk, R³, R⁴ and n are as defined hereabove for formula (I), (Ie) or (Ie-b)
preferably wherein Alk is an unsubstituted (C₁-C₆)alkanediyl group, preferably a (C₃-C₅)alkanediyl group, in particular a linear propylene, and
Each R³ and R⁴ independently represent a (C₁-C₆)alkyl group, preferably a methyl.

According to a particular embodiment, the present invention relates to a sixth subgroup of compounds of formula (I), having following formula (If), or a pharmaceutically acceptable salt thereof:

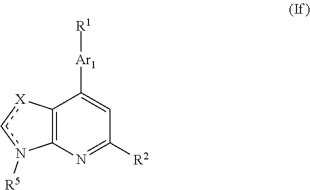

wherein Ar₁, R¹, R² and R⁵ are as defined hereabove for formula (I), and
X is —CH— or —N—.

According to this embodiment, compounds may be of following formula (If-a), or a pharmaceutically acceptable salt thereof:

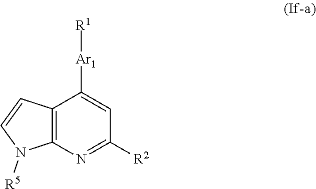

wherein Ar₁, R¹, R² and R⁵ are as defined here-above for formula (I) or (If).
   Preferably Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom or a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group, optionally substituted by one or more —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   These compounds may more precisely be designated as pyrrolopyridine derivatives, or pharmaceutically acceptable salts thereof.
   Advantageously, compounds according to this embodiment may be of following formula (If-a′):

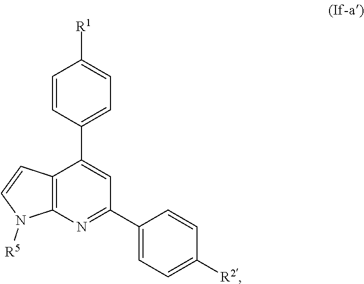

wherein R^{2′} is a hydrogen atom or a —(CH₂)ₙ—NH-Alk-NR³R⁴, and
R¹, Alk, R³, R⁴, R⁵ and n are as defined hereabove for formula (I), (If) or (If-a)
with the proviso that at least one of R¹ and R^{2′} contains a —NH-Alk-NR³R⁴,
preferably wherein R¹ and R^{2′} are —(CH₂)—NH-Alk-NR³R⁴,
Alk is an unsubstituted (C₁-C₆)alkanediyl group, preferably a (C₃-C₅)alkanediyl group, in particular a linear butylene.
Each R³ and R⁴ independently represent a (C₁-C₆)alkyl group, preferably a methyl, or alternatively R³ and R⁴ form together with the nitrogen atom bearing them a (C₃-C₈)heterocycloalkyl group, preferably a piperazinyl group, optionally substituted by one or more, preferably one, (C₁-C₅)alkyl groups, preferably methyl,
R⁵ is a hydrogen atom or a (C₁-C₆)alkyl group substituted by one or more, preferably one, phenyl group, preferably is a benzyl.
   Alternatively, compounds according to this embodiment may be of following formula (If-a″):

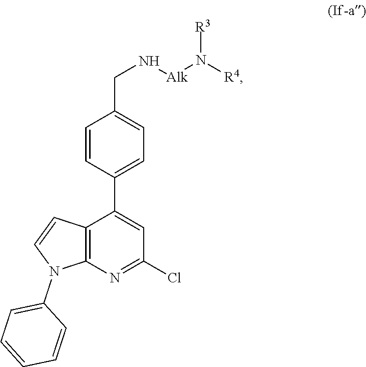

wherein Alk, R³, R⁴ and n are as defined hereabove for formula (I), (If) or (If-a),
preferably wherein Alk is an unsubstituted (C₁-C₆)alkanediyl group, preferably a (C₃-C₅)alkanediyl group, in particular a linear propylene, and
Each R³ and R⁴ independently represent a (C₁-C₆)alkyl group, preferably a methyl.

Still according to this embodiment, compounds may be of following formula (If-b), or a pharmaceutically acceptable salt thereof:

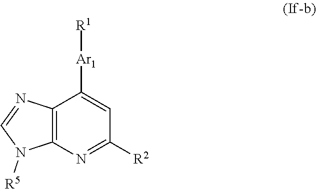

wherein Ar₁, R¹, R² and R⁵ are as defined here-above for formula (I) or (If).
   Preferably Ar₁ is a (C₅-C₁₁)arylene or (C₅-C₁₁)heteroarylene group, in particular (C₅-C₉)arylene or (C₅-C₉)heteroarylene group, preferably chosen among phenylene, thiophenylene and triazolylene groups,
R¹ is a hydrogen atom or a —(CH₂)ₙ—NH-Alk-NR³R⁴,
R² is a halogen atom, a —NH-Alk-NR³R⁴ or a (C₅-C₁₁)aryl or (C₅-C₁₁)heteroaryl group, optionally substituted by a —(CH₂)ₙ—NH-Alk-NR³R⁴,
Alk is a (C₁-C₆)alkanediyl group, optionally substituted by one or more —COOR⁷,
R⁵ is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group, optionally substituted by one or more —(CH₂)ₙ—NH-Alk-NR³R⁴,
Each R³ and R⁴ independently represent a hydrogen atom, a (C₁-C₆)alkyl group, or a —COOR⁹ group, or alternatively R³ and R⁴ form together with the nitrogen atom bearing them a (C₃-C₈)heterocycloalkyl group optionally substituted by one or more, preferably one, (C₁-C₅)alkyl groups, preferably methyl,
n is 1 and
each R⁷, when present, independently represents a (C₁-C₆)alkyl group, preferably a methyl group,
each R⁹, when present, independently represents a (C₁-C₆)alkyl group, preferably a (C₄)alkyl group.
with the proviso that, at least one of R¹, R² and R⁵, contains a group —NH-Alk-NR³R⁴.
   These compounds may more precisely be designated as imidazopyridine derivatives, or pharmaceutically acceptable salts thereof.

According to a particular embodiment, the present invention relates to a seventh subgroup of compounds of formula (I), having following formula (Ig), or a pharmaceutically acceptable salt thereof:

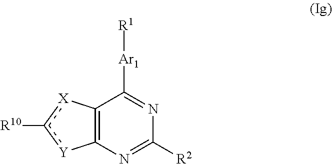

wherein X, Y, Ar₁, R¹, R² and R¹⁰ are as defined hereabove for formula (I),
preferably X is —CH—, —S— or —N—, Y is —CH— or —S—, Ar₁, R¹, R² and R¹⁰ are as defined hereabove for formula (I).

According to this embodiment, compounds may be of following formula (Ig-a), or a pharmaceutically acceptable salt thereof:

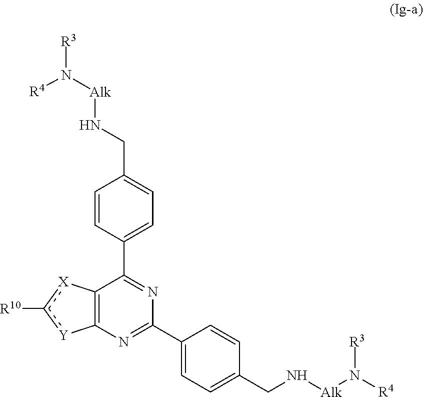

wherein Alk, R³ and R⁴ are as defined here-above for formula (I), (I′) or (Ig) and R¹⁰ is a (C₅-C₁₁)aryl group or (C₅-C₁₁)heteroaryl.

Preferably, Alk is butylene, each R³ and R⁴ is a methyl and R¹⁰ is a phenyl.

In the context of the present invention, the term:
“halogen” is understood to mean chlorine, fluorine, bromine, or iodine, and in particular denotes chlorine, fluorine or bromine, preferably chlorine;
“(C₁-Cₓ)alkyl”, as used herein, respectively refers to a C₁-Cₓ normal, secondary or tertiary monovalent saturated, linear or branched, hydrocarbon radical, for example (C₁-C₆)alkyl. Examples are, but are not limited to, methyl, ethyl, propyl, n-propyl, isopropyl, butyl, isobutyl, sec-butyl, tert-butyl, pentyl, isopentyl, hexyl and isohexyl groups, and the like;
“(C₁-Cₓ)alkanediyl”, as used herein, refers to a divalent saturated hydrocarbon radical which is branched or linear, comprising from 1 to X carbon atoms, and more particularly a methylene, ethylene, propylene, butylene, pentylene, such as linear ethylene, propylene butylene and pentylene, said alkanediyl may be substituted as it is apparent from the following description;
“(C₃-C₈)cycloalkyl”, as used herein, refers to a cyclic saturated hydrocarbon radical, comprising from 3 to 8 carbon atoms, saturated or partially unsaturated and unsubstituted or substituted. Examples are, but are not limited to, cyclopropyl, cyclobutyl, cyclopentyl, cyclohexyl, cycloheptyl and cyclooctyle;
“(C₃-C₈)heterocycloalkyl group”, as used herein, refers to a (C₃-C₈)cycloalkyl group wherein one or two of the carbon atoms are replaced with a heteroatom such as oxygen, nitrogen or sulphur, and more particularly such as an oxygen or a nitrogen atom. Such heterocycloalkyl group may be saturated or partially saturated and unsubstituted or substituted. Examples are, but are not limited to, morpholinyl, piperazinyl, piperidinyl, pyrrolidinyl, aziridinyl, oxanyl, oxetanyl, tetrahydropyranyl, tetrahydrofuranyl, oxepanyl, diazepanyl and dioxanyl, and more particularly morpholinyl, pyrrolidinyl, piperidinyl, piperazinyl and N-methylpiperazinyl;
“(C₅-C₁₁)aryl group”, as used herein, refers to a monocyclic aromatic group or to a bicyclic aromatic group where at least one of the ring is aromatic. By way of examples of aryl groups, mention may be made of, but not limited to phenyl and naphthalenyl;
“(C₅-C₁₁)arylene group”, as used herein, refers to a bivalent monocyclic aromatic group or bicyclic aromatic group where at least one of the ring is aromatic. These (C₅-C₁₁)arylene groups are formed from (C₅-C₁₁)aryl group as defined hereabove by removing a hydrogen atom. Example of such arylene groups is phenylene;
“(C₅-C₁₁)heteroaryl group”, as used herein, refers to a monocyclic aromatic group or to a bicyclic aromatic group where at least one of the ring is aromatic and wherein one to three ring carbon atom is replaced by a heteroatom, such as nitrogen, oxygen or sulphur. By way of examples of heteroaryl groups, mention may be made of, but not limited to: pyrrolyl, thiophenyl, oxazolyl, isoxazolyl, pyridinyl, pyrimidinyl, pyridazinyl, triazinyl, pyrazinyl, oxadiazolyl, furanyl, pyrazolyl, thiazolyl, isothiazolyl, thiadiazolyl, imidazolyl, triazolyl and the like. In the framework of the present invention, the heteroaryl is advantageously pyridinyl, imidazolyl, pyrazinyl, furanyl, thiazolyl, pyrazolyl, thiadiazolyl, pyridazinyl and pyrimidinyl;
“(C₅-C₁₁)heteroarylene group”, also sometimes referred as “(C₅-C₁₂)heteroarenediyl group”, as used herein, refers to a bivalent monocyclic aromatic group or bicyclic aromatic group where at least one of the ring is aromatic, one to three ring carbon atoms being replaced by heteroatoms, such as nitrogen, oxygen or sulphur. These (C₅-C₁₁)heteroarylene groups are formed from (C₅-C₁₁)heteroaryl group as defined hereabove by removing a hydrogen atom. Example of such heteroarylene groups is thiophenylene;
an aromatic ring means, according to Hückel's rule, that a molecule has 4n+2 π-electrons; and
“(C₁-C₆)alkoxy as used therein respectively refers to O—(C₁-C₆)alkyl moiety, wherein (C₁-C₆)alkyl is as defined above. Examples are methoxy and ethoxy.

In the context of the present invention, the terms “aromatic ring”, and “heteroaryl” include all the positional isomers.

“Pharmaceutically acceptable salt” refers to salts which are formed from acid addition salts formed with inorganic acids (e.g. hydrochloric acid, hydrobromic acid), as well as salts formed with organic acids such as oxalic acid, acetic acid, tartaric acid, fumaric acid, succinic acid.

Suitable pharmaceutically acceptable acid addition salts of compounds of formula (I) particularly include oxalate.

The compounds of formula (I) or any of their pharmaceutically acceptable salts may form solvates or hydrates and the invention includes all such solvates and hydrates.

The terms “hydrates″ and “solvates” simply mean that the compounds (I) according to the invention can be in the form of a hydrate or solvate, i.e. combined or associated with one or more water or solvent molecules. This is only a chemical characteristic of such compounds, which can be applied for all organic compounds of this type.

The compounds of formula (I), (I′), (Ia), (Ia-a), (Ia-b), (Ia-c), (Ia-d), (Ia-e), (Ib), (Ib-a), (Ic), (Ic-a), (Ic-b), (Ic-c), (Id), (Id-a), (Id-b), (Id-c), (Ie), (Ie-a), (Ie-a′), (Ie-b), (Ie-b′), (Ie-b″), (If), (If-a), (If-a′), (If-a″) (If-b), (Ig) and (Ig-a) can comprise one or more asymmetric carbon atoms. They can thus exist in the form of enantiomers or of diastereoisomers. These enantiomers, diastereoisomers and their mixtures, including the racemic mixtures, are encompassed within the scope of the present invention.

The nomenclature of the following compounds (1) to (49) was generated according to the principles of the International Union of Pure and Applied Chemistry, using IUPAC rules for organic compounds.

In the context of the present invention, compounds (X) gather all compounds encompassed within formula (I), including their free forms and pharmaceutically acceptable salts. For example, there below, (XA) is the free form of 6-6 or 5-6 fused bicyclic compound and (XB) is the corresponding oxalate salt.

According to a preferred embodiment of the present invention, the compound of formula (I) is chosen from the following compounds:
(1A) 2,4-bis{4-[(3-dimethylaminopropyl)aminomethyl]phenyl}-7-phenyl-7H-pyrrolo[2,3-d]pyrimidine,
(1B) 2,4-bis{4-[(3-dimethylaminopropyl)aminomethyl]phenyl}-7-phenyl-7H-pyrrolo[2,3-d]pyrimidine oxalate,
(2A) 2,4-bis{4-[(3-(4-methylpiperazin-1-yl)propyl)aminomethyl]phenyl}-7-phenyl-7H-pyrrolo[2,3-d]pyrimidine,
(2B) 2,4-bis{4-[(3-(4-methylpiperazin-1-yl)propyl)aminomethyl]phenyl}-7-phenyl-7H-pyrrolo[2,3-d]pyrimidine oxalate,
(3A) 2,4-bis{4-[(3-dimethylaminobutyl)aminomethyl]phenyl}-7-phenyl-7H-pyrrolo[2,3-d]pyrimidine,
(3B) 2,4-bis{4-[(3-dimethylaminobutyl)aminomethyl]phenyl}-7-phenyl-7H-pyrrolo[2,3-d]pyrimidine oxalate,
(4A) 2,4-bis{4-[(3-(4-methylpiperazin-1-yl)butyl)aminomethyl]phenyl}-7-phenyl-7H-pyrrolo[2,3-d]pyrimidine,
(4B) 2,4-bis{4-[(3-(4-methylpiperazin-1-yl)butyl)aminomethyl]phenyl}-7-phenyl-7H-pyrrolo[2,3-d]pyrimidine oxalate,
(5A) 2,4-bis{4-[(3-dimethylaminopentyl)aminomethyl]phenyl}-7-phenyl-7H-pyrrolo[2,3-d]pyrimidine,
(5B) 2,4-bis{4-[(3-dimethylaminopentyl)aminomethyl]phenyl}-7-phenyl-7H-pyrrolo[2,3-d]pyrimidine oxalate,
(6A) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine,
(6B) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine oxalate,
(7A) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine,
(7B) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine oxalate,
(8A) 2,4-bis{4-[(3-dimethylaminopropyl)aminomethyl]phenyl}-7-methyl-7H-pyrrolo[2,3-d]pyrimidine,
(8B) 2,4-bis{4-[(3-dimethylaminopropyl)aminomethyl]phenyl}-7-methyl-7H-pyrrolo[2,3-d]pyrimidine oxalate,
(9A) 2,4-bis{4-[(3-(4-methylpiperazin-1-yl)propyl)aminomethyl]phenyl}-7-methyl-7H-pyrrolo[2,3-d]pyrimidine,
(9B) 2,4-bis{4-[(3-(4-methylpiperazin-1-yl)propyl)aminomethyl]phenyl}-7-methyl-7H-pyrrolo[2,3-d]pyrimidine oxalate,
(10A) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine,
(10B) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine oxalate,
(11A) 2,4-Bis{4-[(5-amino-5-(methoxycarbonyl)pentyl)aminomethyl]phenyl}-7-methyl-7H-pyrrolo[2,3-d]pyrimidine,
(11B) 2,4-Bis{4-[(5-amino-5-(methoxycarbonyl)pentyl)aminomethyl]phenyl}-7-methyl-7H-pyrrolo[2,3-d]pyrimidine oxalate,
(12A) 7-(4-[(3-Dimethylaminopropyl)aminomethyl]phenyl)-2,4-diphenyl-7H-pyrrolo[2,3-d]pyrimidine,
(12B) 7-(4-[(3-Dimethylaminopropyl)aminomethyl]phenyl)-2,4-diphenyl-7H-pyrrolo[2,3-d]pyrimidine oxalate,
(13A) 2,4,7-Tri{4-[(3-dimethylaminopropyl)aminomethyl]phenyl}-7H-pyrrolo[2,3-d]pyrimidine,
(13B) 2,4,7-Tri{4-[(3-dimethylaminopropyl)aminomethyl]phenyl}-7H-pyrrolo[2,3-d]pyrimidine oxalate,
(14A) 2-(3-Dimethylaminopropylamino)-4-{4-[(4-dimethylaminopropyl)aminomethyl]phenyl}-7-phenyl-7H-pyrrolo[2,3-d]pyrimidine,
(14B) 2-(3-Dimethylaminopropylamino)-4-{4-[(4-dimethylaminopropyl)aminomethyl]phenyl}-7-phenyl-7H-pyrrolo[2,3-d]pyrimidine oxalate,
(15A) 2-Chloro-4-{4-[(3-dimethylaminopropyl)aminomethyl]phenyl}-7H-pyrrolo[2,3-d]pyrimidine,
(15B) 2-Chloro-4-{4-[(3-dimethylaminopropyl)aminomethyl]phenyl}-7H-pyrrolo[2,3-d]pyrimidine oxalate,
(16A) 2-Chloro-4-{4-[(3-(4-methylpiperazin-1-yl)propyl)aminomethyl]phenyl}-7H-pyrrolo[2,3-d]pyrimidine,
(16B) 2-Chloro-4-{4-[(3-(4-methylpiperazin-1-yl)propyl)aminomethyl]phenyl}-7H-pyrrolo[2,3-d]pyrimidine oxalate,
(17A) 2-Chloro-4-{4-[(3-dimethylaminopropyl)aminomethyl]phenyl}-7-phenyl-7H-pyrrolo[2,3-d]pyrimidine,
(17B) 2-Chloro-4-{4-[(3-dimethylaminopropyl)aminomethyl]phenyl}-7-phenyl-7H-pyrrolo[2,3-d]pyrimidine oxalate,
(18A) 2-chloro-4-{4-[(3-(4-methylpiperazin-1-yl)propyl)aminomethyl]phenyl}-7-phenyl-7H-pyrrolo[2,3-d]pyrimidine,
(18B) 2-chloro-4-{4-[(3-(4-methylpiperazin-1-yl)propyl)aminomethyl]phenyl}-7-phenyl-7H-pyrrolo[2,3-d]pyrimidine oxalate,
(19A) 2-Chloro-4-{5-[(3-dimethylaminopropyl)aminomethyl]thien-2-yl}-7-phenyl-7H-pyrrolo[2,3-d]pyrimidine,
(19B) 2-Chloro-4-{5-[(3-dimethylaminopropyl)aminomethyl]thien-2-yl}-7-phenyl-7H-pyrrolo[2,3-d]pyrimidine oxalate,
(20A) 2-Chloro-4-{2-[(3-dimethylaminopropyl)aminomethyl]thien-4-yl}-7-phenyl-7H-pyrrolo[2,3-d]pyrimidine,
(20B) 2-Chloro-4-{2-[(3-dimethylaminopropyl)aminomethyl]thien-4-yl}-7-phenyl-7H-pyrrolo[2,3-d]pyrimidine oxalate,
(21A) 2-Chloro-4-{2-[(3-dimethylaminopropyl)aminomethyl]thien-3-yl}-7-phenyl-7H-pyrrolo[2,3-d]pyrimidine,
(21B) 2-Chloro-4-{2-[(3-dimethylaminopropyl)aminomethyl]thien-3-yl}-7-phenyl-7H-pyrrolo[2,3-d]pyrimidine oxalate,
(22A) 2-Chloro-7-(4-[(3-dimethylaminopropyl)aminomethyl]phenyl)-4-phenyl-7H-pyrrolo[2,3-d]pyrimidine,
(22B) 2-Chloro-7-(4-[(3-dimethylaminopropyl)aminomethyl]phenyl)-4-phenyl-7H-pyrrolo[2,3-d]pyrimidine oxalate,
(23A) 2-Chloro-7-(4-[(3-(4-methylpiperazin-1-yl)propyl)aminomethyl]phenyl)-4-phenyl-7H-pyrrolo[2,3-d]pyrimidine,
(23B) 2-Chloro-7-(4-[(3-(4-methylpiperazin-1-yl)propyl)aminomethyl]phenyl)-4-phenyl-7H-pyrrolo[2,3-d]pyrimidine oxalate,
(24A) Tert-butyl 2,4-bis{4-[(3-dimethylaminopropyl)aminomethyl]phenyl})-5,6-dihydropyrido[3,4-d]pyrimidine-7(8H)-carboxylate,
(24B) Tert-butyl 2,4-bis{4-[(3-dimethylaminopropyl)aminomethyl]phenyl})-5,6-dihydropyrido[3,4-d]pyrimidine-7(8H)-carboxylate oxalate,
(25A) 2,4-Bis{4-[(3-dimethylaminopropyl)aminomethyl]phenyl})-5,6,7,8-dihydropyrido[3,4-d]pyrimidine,
(25B) 2,4-Bis{4-[(3-dimethylaminopropyl)aminomethyl]phenyl})-5,6,7,8-dihydropyrido[3,4-d]pyrimidine oxalate,
(26A) 2,4-Bis{4-[(4-dimethylaminopropyl)aminomethyl]phenyl}thieno[2,3-d]pyrimidine,
(26B) 2,4-Bis{4-[(4-dimethylaminopropyl)aminomethyl]phenyl}thieno[2,3-d]pyrimidine oxalate,
(27A) 2,4-Bis{4-[(3-(4-methylpiperazin-1-yl)propyl)aminomethyl]phenyl}thieno[2,3-d]pyrimidine,
(27B) 2,4-Bis{4-[(3-(4-methylpiperazin-1-yl)propyl)aminomethyl]phenyl}thieno[2,3-d]pyrimidine oxalate,
(28A) 2-(3-Dimethylaminopropylamino)-4-{4-[(4-dimethylaminopropyl)aminomethyl]phenyl}thieno[2,3-d]pyrimidine,
(28B) 2-(3-Dimethylaminopropylamino)-4-{4-[(4-dimethylaminopropyl)aminomethyl]phenyl}thieno[2,3-d]pyrimidine oxalate,
(29A) 2,4-Bis{4-[(4-dimethylaminopropyl)aminomethyl]phenyl}thieno[3,2-d]pyrimidine,
(29B) 2,4-Bis{4-[(4-dimethylaminopropyl)aminomethyl]phenyl}thieno[3,2-d]pyrimidine oxalate,
(30A) 2,4-Bis{4-[(3-(4-methylpiperazin-1-yl)propyl)aminomethyl]phenyl}thieno[3,2-d]pyrimidine,
(30B) 2,4-Bis{4-[(3-(4-methylpiperazin-1-yl)propyl)aminomethyl]phenyl}thieno[3,2-d]pyrimidine oxalate,
(31A) 2-(3-Dimethylaminopropylamino)-4-{4-[(4-dimethylaminopropyl)aminomethyl]phenyl}thieno[3,2-d]pyrimidine,
(31B) 2-(3-Dimethylaminopropylamino)-4-{4-[(4-dimethylaminopropyl)aminomethyl]phenyl}thieno[3,2-d]pyrimidine oxalate,
(32A) 2,7-bis{4-[(3-dimethylaminobutyl)aminomethyl]phenyl}-4-phenyl-7H-pyrrolo[2,3-d]pyrimidine,
(32B) 2,7-bis{4-[(3-dimethylaminobutyl)aminomethyl]phenyl}-4-phenyl-7H-pyrrolo[2,3-d]pyrimidine oxalate,
(33A) 2,4-bis{4-[(3-diethylaminobutyl)aminomethyl]phenyl}-7-phenyl-7H-pyrrolo[2,3-d]pyrimidine,
(33B) 2,4-bis{4-[(3-diethylaminobutyl)aminomethyl]phenyl}-7-phenyl-7H-pyrrolo[2,3-d]pyrimidine oxalate,
(34A) 2,4-bis{4-[(3-diisopropylaminopentyl)aminomethyl]phenyl}-7-phenyl-7H-pyrrolo[2,3-d]pyrimidine,
(34B) 2,4-bis{4-[(3-diisopropylaminopentyl)aminomethyl]phenyl}-7-phenyl-7H-pyrrolo[2,3-d]pyrimidine oxalate,
(35A) 2,7-bis{4-[(3-dimethylaminopentyl)aminomethyl]phenyl}-4-phenyl-7H-pyrrolo[2,3-d]pyrimidine,
(35B) 2,7-bis{4-[(3-dimethylaminopentyl)aminomethyl]phenyl}-4-phenyl-7H-pyrrolo[2,3-d]pyrimidine oxalate,
(36A) 4,6-Bis{4-[(3-dimethylaminopropyl)aminomethyl]phenyl}-1-phenyl-1H-pyrazolo[3,4-d]pyrimidine,
(36B) 4,6-Bis{4-[(3-dimethylaminopropyl)aminomethyl]phenyl}-1-phenyl-1H-pyrazolo[3,4-d]pyrimidine oxalate,
(37A) 4,6-Bis{4-[(4-dimethylaminobutyl)aminomethyl]phenyl}-1-phenyl-1H-pyrazolo[3,4-d]pyrimidine,
(37B) 4,6-Bis{4-[(4-dimethylaminobutyl)aminomethyl]phenyl}-1-phenyl-1H-pyrazolo[3,4-d]pyrimidine,
(38A) 4,6-Bis{4-[(5-dimethylaminopentyl)aminomethyl]phenyl}-1-phenyl-1H-pyrazolo[3,4-d]pyrimidine,
(38B) 4,6-Bis{4-[(5-dimethylaminopentyl)aminomethyl]phenyl}-1-phenyl-1H-pyrazolo[3,4-d]pyrimidine oxalate,
(39A) 4,6-Bis{4-[(4-(4-methylpiperazin-1-yl)butyl)aminomethyl]phenyl}-1-phenyl-1H-pyrazolo[3,4-d]pyrimidine,
(39B) 4,6-Bis{4-[(4-(4-methylpiperazin-1-yl)butyl)aminomethyl]phenyl}-1-phenyl-1H-pyrazolo[3,4-d]pyrimidine oxalate,
(40A) 2,4-Bis{4-[(4-dimethylaminobutyl)aminomethyl]phenyl}-7-phenyl-7H-imidazo[4,5-d]pyrimidine,
(40B) 2,4-Bis{4-[(4-dimethylaminobutyl)aminomethyl]phenyl}-7-phenyl-7H-imidazo[4,5-d]pyrimidine oxalate,
(41A) 2,4-Bis{4-[(5-dimethylaminopentyl)aminomethyl]phenyl}-7-phenyl-7H-imidazo[4,5-d]pyrimidine,
(41B) 2,4-Bis{4-[(5-dimethylaminopentyl)aminomethyl]phenyl}-7-phenyl-7H-imidazo[4,5-d]pyrimidine oxalate,
(42A) 2,4-Bis{4-[(4-(4-methylpiperazin-1-yl)butyl)aminomethyl]phenyl}-7-phenyl-7H-imidazo[4,5-d]pyrimidine,
(42B) 2,4-Bis{4-[(4-(4-methylpiperazin-1-yl)butyl)aminomethyl]phenyl}-7-phenyl-7H-imidazo[4,5-d]pyrimidine oxalate,
(43A) 2-Chloro-4-{4-[(3-dimethylaminopropyl)aminomethyl]phenyl}-7-phenyl-7H-imidazo[4,5-d]pyrimidine,
(43B) 2-Chloro-4-{4-[(3-dimethylaminopropyl)aminomethyl]phenyl}-7-phenyl-7H-imidazo[4,5-d]pyrimidine oxalate,
(44A) 4,6-Bis{4-[(4-dimethylaminobutyl)aminomethyl]phenyl}-1H-pyrrolo[2,3-b]pyridine,
(44B) 4,6-Bis{4-[(4-dimethylaminobutyl)aminomethyl]phenyl}-1H-pyrrolo[2,3-b]pyridine oxalate,
(45A) 4,6-Bis{4-[(4-(4-methylpiperazin-1-yl)butyl)aminomethyl]phenyl}-1H-pyrrolo[2,3-b]pyridine,
(45B) 4,6-Bis{4-[(4-(4-methylpiperazin-1-yl)butyl)aminomethyl]phenyl}-1H-pyrrolo[2,3-b]pyridine oxalate,
(46A) 4,6-Bis{4-[(4-dimethylaminobutyl)aminomethyl]phenyl}-1-benzyl-1H-pyrrolo[2,3-b]pyridine,
(46B) 4,6-Bis{4-[(4-dimethylaminobutyl)aminomethyl]phenyl}-1-benzyl-1H-pyrrolo[2,3-b]pyridine oxalate,
(47A) 6-Chloro-4-{4-[(3-dimethylaminopropyl)aminomethyl]phenyl}-1-phenyl-1H-pyrrolo[2,3-b]pyridine,
(47B) 6-Chloro-4-{4-[(3-dimethylaminopropyl)aminomethyl]phenyl}-1-phenyl-1H-pyrrolo[2,3-b]pyridine oxalate,
(48A) 2,4-bis{4-[(3-dimethylaminobutyl)aminomethyl]phenyl}-7-(4-methoxyphenyl)-7H-pyrrolo[2,3-d]pyrimidine,
(48B) 2,4-bis{4-[(3-dimethylaminobutyl)aminomethyl]phenyl}-7-(4-methoxyphenyl)-7H-pyrrolo[2,3-d]pyrimidine oxalate,
(49A) 2,4-Bis{4-[(4-dimethylaminobutyl)aminomethyl]phenyl}-6-phenyl-thieno[3,2-d]pyrimidine, and
(49B) 2,4-Bis{4-[(4-dimethylaminobutyl)aminomethyl]phenyl}-6-phenyl-thieno[3,2-d]pyrimidine oxalate.

According to an even more preferred embodiment of the present invention, the compound of formula (I) is chosen from the group consisting of compounds (1), (2), (3), (4), (5), (6), (7), (8), (9), (13), (15), (16), (19), (20), (21), (22), (23), (26), (27), (28), (29), (30), (33), (34) and (35), in particular from the group consisting of compounds (1B), (2B), (3B), (4B), (5B), (6B), (7B), (8B), (9B), (13B), (15B), (16B), (19B), (20B), (21B), (22B), (23B), (26B), (27B), (28B), (29B), (30B), (33B), (34B) and (35B), even more particularly compounds (3B), (5B), (13B), (15B), (16B), (17B), (18B), (19B), (20B), (21B), (22B), (23B), (27B), (28B) and (34B), and their pharmaceutically acceptable salts.

According to another aspect, a subject-matter of the present invention relates to a pharmaceutical composition comprising at least one compound of formula (I) as defined above or any of its pharmaceutically acceptable salts, in particular at least one of compounds (1) to (49), in particular (1) to (32).

According to another aspect, a subject-matter of the present invention relates to a compound of formula (I) as defined above or any of its pharmaceutically acceptable salts, or at least any of compounds (1) to (49), in particular (1) to (32), for use as a medicament.

The compounds of the present invention can be prepared by conventional methods of organic synthesis practiced by those skilled in the art. The general reaction sequences outlined below represent a general method useful for preparing the compounds of the present invention and are not meant to be limiting in scope or utility.

TABLE 1
List of abbreviations:

| Abbreviation/ | |
|---|---|
| acronym | name |
| Alk | Alkanediyl |
| Ar | Aryl |
| C | Concentration |
| CC₅₀ | Half maximal cytotoxic concentration |
| CHO/OHC | Formyl |
| Cpd No | Compound number |
| CQ | Chloroquine |
| DME | 1,2-dimethoxyethane |
| DMSO | Dimethylsulfoxide |
| EDTA | Ethylenediaminetetraacetic acid |
| Equiv. | Equivalent |
| Et | Ethyl |
| FCS | Fetal Calf Serum |
| GP | General protocol |
| Hal | Halogen |
| HPLC | High performance liquid chromatography |
| IC₅₀ | Half maximal inhibitory concentration |
| IR | Infrared |
| M | Molarity |
| MALDI | Matrix Assisted Laser Desorption Ionization |
| MTT | 3-(4,5-dimethylthiazol-2-yl)-2,5-diphenyltetrazolium |
| | bromide |
| Me | Methyl |
| Mp | Melting point |
| MQ | Mefloquine |
| MS | Mass spectroscopy |
| MW | Molecular weight |
| NMR | Nuclear magnetic resonance |
| N/A | Not applicable |
| n.t. | Not tested |
| Rac | Racemic |
| RPMI | Roswell Park Memorial Institute medium |
| RT | Room temperature |
| TEA | Triethylamine |
| TOF | Time of Flight |
| UV | Ultraviolet |
| v/v | Volume per volume |
| w/v | Weight per volume |
| δ_{H} | Hydrogen chemical shift |

The compounds of the present invention can be prepared by conventional methods of organic synthesis practiced by those skilled in the art. The general reaction sequences outlined below represent general methods useful for preparing the compounds of the present invention and are not meant to be limiting in scope or utility.

In all the schemes that follow, the below definitions apply:

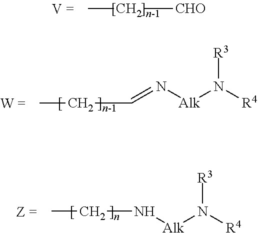

wherein n, Alk, R³ and R⁴ are as defined here-above.

According to one embodiment, the compounds of general formula (I) wherein:
Ar₁, R¹, R², X, Y, T, Q, Alk, R³ and R⁴ are as defined here-above;
R⁵, when present, is a hydrogen atom, a C₁-C₆ alkyl optionally substituted by one or more (C₅-C₁₁)aryl or (C₅-C₁₁)heteroaryl group or a (C₅-C₁₁)aryl group or (C₅-C₁₁)heteroaryl optionally substituted by one or more (C₁-C₆)alkoxy; and
R⁶, if present, is a (C₁-C₆)alkyl group, a (C₅-C₁₁)aryl group or (C₅-C₁₁)heteroaryl optionally substituted by one or more (C₁-C₆)alkoxy or a group —COOR⁸,
can be prepared according to schemes 1 to 4 below.

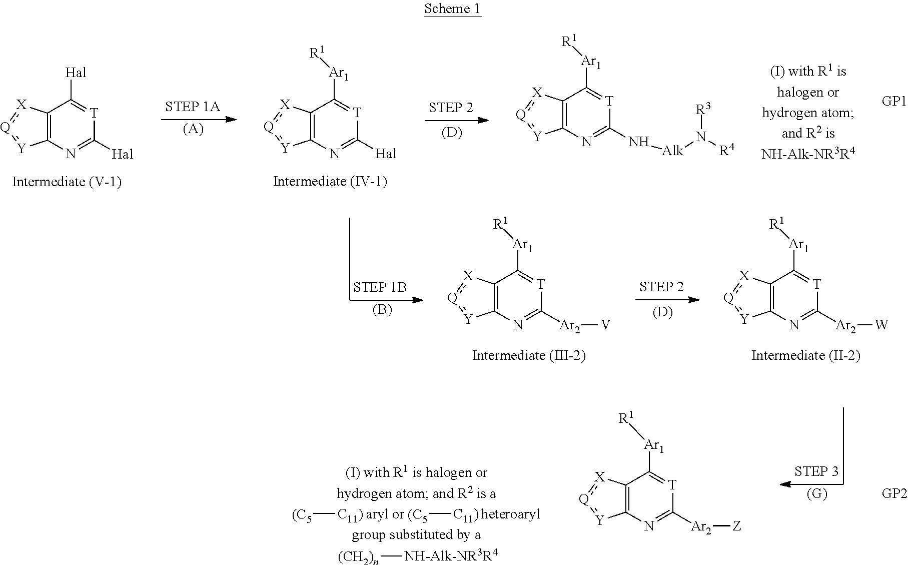

According to General protocol 1 (GP1), compounds of formula (I) are obtained wherein:
Ar₁, [symbol] X, Y, Q, T, Alk, R³ and R⁴, n and R⁸, if present, are as defined for formula (I) here-above.
R¹ is a halogen atom or hydrogen atom,
R² is —NH-Alk-NR³R⁴,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— with a protecting group, such as a tert-butyloxycarbonyl (Boc) protecting group, using known method in the art. By way of example, it can be performed under the following conditions. Intermediate (V-1) may be placed in solution into an appropriate solvent, such as CH₂Cl₂, in presence of N,N-Diisopropylethylamine (DIPEA) in a molar ratio ranging from 1 to 1.5, of 4-Dimethylaminopyridine (DMAP) in a molar ratio ranging from 0.1 to 0.5 and of di-tert-butyl decarbonate (Boc₂O) in a molar ratio ranging from 1.2 to 1.8. Then, the reaction mixture can be stirred under reflux for a duration ranging from 5 to 20 minutes and the solvent be evaporated under reduced pressure. The obtained residue can be purified, for example by silica gel column chromatography using a proper eluent, such as CHCl₃, so that to obtained protected Intermediate (V-1).

In STEP 1A, reaction conditions (A) are implemented as follows.

Optionally protected Intermediate (V-1) may be placed in solution into a proper solvent, such as 1,2-dimethoxyethane, dioxane, toluene, benzene, tetrahydrofuran or dimethylformamide, preferably 1,2-dimethoxyethane, in presence of a (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, optionally substituted by a halogen atom, in a molar ratio ranging from 1 to 1.5 with respect to intermediate (V-1) in presence of a solution of palladium catalyst, such as tetrakis(triphenylphosphine) palladium, for example in a molar ratio ranging from 0.04 to 0.15 with respect to intermediate (V-1). A previously degassed for between 5 and 20 minutes with an inert gas, such as nitrogen, aqueous solution of an inorganic base such as Na₂CO₃, K₂CO₃, NaHCO₃, NaOH or KOH, preferably Na₂CO₃, for example in a concentration of 2M, may then be added at room temperature.

Then, the mixture can be warmed to reflux and stirred, for example for between 12 hours and 36 hours, under an inert gas, for example nitrogen, and positive pressure. The reaction mixture can be cooled down to room temperature and the solvent be evaporated under vacuum. The organic layer can be extracted with an appropriate extracting solvent, such as CH₂Cl₂ and the organic phase can be filtered, for example on filter paper, then washed with water, for example 3 times with 20 mL of water, dried, for example over anhydrous sodium sulfate and activated charcoal, filtered and evaporated under vacuum. The residue can then be cooled and triturated with a minimum of proper solvent(s), for example EtOH and EtO₂ and filtered, for example on sintered glassware, to give the crude product. The residue can then be purified, for example by silica gel column chromatography (CH₂Cl₂/CH₃OH 95:5), then cooled and triturated again in EtOH, filtered, for example on sintered glassware, washed with a minimum of proper solvent(s), for example EtOH, then EtO₂ and petroleum ether and dried under pressure to give intermediate (IV-1), optionally protected.

In case where Intermediate (V-1) has been subjected, prior to step 1A, to step of protecting the function —NR⁵— with a protecting group, the protected intermediate (IV-1) obtained from STEP 1A is subjected, prior to STEP 2, to a step of deprotecting the function —NR⁵— using methods conventionally known in the art. By way of example, it can be performed under the following conditions.

Protected Intermediate (IV-1) may be placed in solution into an appropriate solvent, such as CH₂Cl₂, to which is added trifluoroacetic acid (TFA), for example in a content of between 5 and 20 mL. The reaction mixture can be stirred at room temperature for between 5 and 15 minutes. Then, the solvent and TFA can be evaporated under reduced pressure to dryness and the residue cooled down. The residue can then be triturated with a minimum of proper solvent(s), for example EtOH and EtO₂ and filtered, for example on sintered glassware, washed with proper solvent(s), for example EtOH, EtO₂ and/or petroleum ether, and dried under reduced pressure to obtain Intermediate (IV-1).

In STEP 2, reaction conditions (D) are implemented as follows.

Intermediate (IV-1) may be added to a solution of the appropriate diamine in a proper solvent, such as ethanol, methanol, tetrahydrofuran, toluene or benzene, preferably ethanol, for example in a molar ratio ranging from 1 to 1.5 with respect to intermediate (IV-1). The reaction mixture may then be heated under reflux, for example for between 2 hours and 24 hours, and then evaporated to dryness under reduced pressure. After cooling, the residue can be extracted with an appropriate extracting solvent, for example dichloromethane, in an amount such as between 15 and 30 mL. The organic layer can then be dried, for example over sodium sulfate and activated charcoal and evaporated to dryness to obtain compound (I) as defined here-above.

According to General protocol 2 (GP2), compounds of formula (I) are obtained wherein:
Ar₁, [symbol] X, Y, Q, T, Alk, R³ and R⁴, n and R⁸, if present, are as defined for formula (I) here-above.
R¹ is a halogen or hydrogen atom
R² is a (C₅-C₁₁)aryl or (C₅-C₁₁)heteroaryl group substituted by a —(CH₂)ₙ—NH-Alk-NR³R⁴
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— such as detailed here-above for GP1.

STEP 1A is performed as disclosed here-above for GP1.

In STEP 1B reaction conditions (B) are implemented as follows.

Optionally protected Intermediate (IV-1) may be placed in solution for example into an appropriate solvent, such as 1,2-dimethoxyethane dioxane, toluene, benzene, tetrahydrofuran or dimethylformamide, preferably 1,2-dimethoxyethane, in presence of a (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted with a —(CH₂)ₙ₋₁—CHO group, in a molar ratio ranging from 1 to 1.5 with respect to Intermediate (IV-1) in presence of a solution of palladium catalyst, such as tetrakis(triphenylphosphine) palladium, for example in a molar ratio ranging from 0.04 to 0.15 with respect to Intermediate (IV-1). A previously degassed for between 5 and 20 minutes with an inert gas, such as nitrogen, aqueous solution of an inorganic base, such as Na₂CO₃, K₂CO₃, NaHCO₃, NaOH or KOH, preferably Na₂CO₃, for example in a concentration of 2M, may then be added at room temperature.

Then, the mixture can be warmed to reflux and stirred, for example for between 12 hours and 36 hours, under an inert gas, for example nitrogen, and positive pressure. The reaction mixture can be cooled down to room temperature and the solvent be evaporated under vacuum. The organic layer can be extracted with an appropriate extracting solvent, such as CH₂Cl₂ and the organic phase can be filtered, for example on filter paper, then washed with water, for example 3 times with between 10 mL and 20 mL of water, dried, for example over anhydrous sodium sulfate and activated charcoal, filtered and evaporated under vacuum. The residue can then be cooled and triturated with a minimum of proper solvent(s), for example EtOH and EtO₂ and filtered, for example on sintered glassware, to give the crude product. The residue can then be purified, for example by silica gel column chromatography (for example CH₂Cl₂/CH₃OH 95:5), then optionally further cooled and triturated again in EtOH, filtered, for example on sintered glassware. The residue, eventually purified, can be washed with a minimum of proper solvent(s), for example EtOH, EtO₂ and/or petroleum ether and dried under pressure to give intermediate (III-2), optionally protected.

In case where Intermediate (V-1) has been subjected, prior to step 1A, to step of protecting the function —NR⁵— with a protecting group, the intermediate (III-2) obtained from STEP 1B is subjected, prior to STEP 2, to a step of deprotecting the function —NR⁵— such as detailed here-above for GP1.

In STEP 2, reaction conditions (D) are implemented as in STEP 2 of GP1, but starting from intermediate (III-2) and resulting to intermediate (II-2).

In STEP 3, reaction conditions (G) are implemented as follows, so that imine functions may be reduced to amine functions.

Intermediate (II-2) may be placed in solution into a proper alcoholic solvent, such as methanol. Then, a reducing agent, such a sodium borohydride can be added portion-wise at a temperature of between −5° C. to 5° C., for example in a molar ratio ranging from 2.5 to 8.5 with respect to intermediate (II-2). The reaction mixture can then be stirred at a temperature ranging from 20° C. and 80° C., for example for between 1 hours and 3 hours. Then it can be evaporated to dryness under reduced pressure. After cooling, the residue can be triturated in water and extracted with an extracting solvent, such as dichloromethane, for example between 15 and 40 mL. The organic layer can be separated, dried, for example over sodium sulfate and activated charcoal and evaporated to dryness to give compounds (I) as defined here-above.

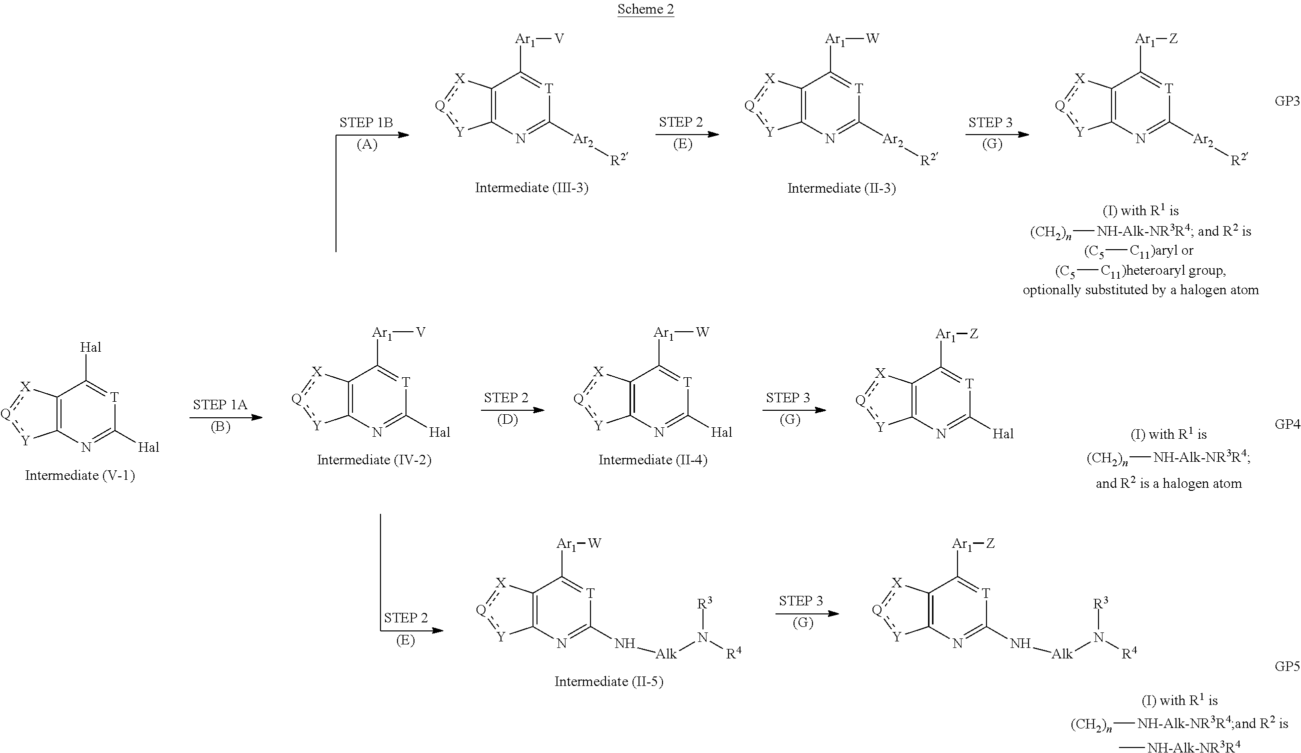

According to General protocol 3 (GP3), compounds of formula (I) are obtained wherein:
Ar₁, [symbol] X, Y, Q, T, Alk, R³ and R⁴, n and R⁸, if present, are as defined for formula (I) here-above.
R¹ is (CH₂)ₙ—NH-Alk-NR³R⁴,
R² is (C₅-C₁₁)aryl or (C₅-C₁₁)heteroaryl group, optionally substituted by a halogen atom,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— with a protecting group such as detailed here-above for GP1.

In STEP 1A reaction conditions (B) are implemented as in STEP 1B of GP2, but starting from intermediate (V-1) and resulting in intermediate (IV-2).

In STEP 1B, reaction conditions (A) are implemented as in STEP 1A of GP1, but starting from intermediate (IV-2) and resulting in intermediate (III-3).

In case where Intermediate (V-1) has been subjected, prior to step 1A, to step of protecting the function —NR⁵— with a protecting group, the intermediate (III-3) obtained from STEP 1B is subjected, prior to STEP 2, to a step of deprotecting the function —NR⁵— such as detailed here-above for GP1.

In STEP 2, reaction conditions (E) are implemented as follows.

Intermediate (III-3) may be added to a solution of the appropriate diamine in a proper solvent, such as ethanol, methanol, tetrahydrofuran, toluene or benzene, preferably ethanol, for example in a molar ratio ranging from 2 to 2.5 with respect to intermediate (III-3). The reaction mixture may then be heated under reflux, for example for between 2 hours and 24 hours, and then evaporated to dryness under reduced pressure. After cooling, the residue can be extracted with an appropriate extracting solvent, for example dichloromethane, in an amount such as between 15 and 50 mL. The organic layer can then be dried, for example over sodium sulfate and activated charcoal and evaporated to dryness to obtain intermediate (II-3).

In STEP 3, reaction conditions (G) are implemented as in STEP 3 of GP2, so that imine functions of intermediate (II-3) may be reduced to amine functions, thus obtaining compounds of formula (I) as defined here-above.

According to General protocol 4 (GP4), compounds of formula (I) are obtained wherein:
Ar₁, [symbol] X, Y, Q, T, Alk, R³ and R⁴, n and R⁸, if present, are as defined for formula (I) here-above.
R¹ is (CH₂)ₙ—NH-Alk-NR³R⁴,
R² is a halogen atom,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— with a protecting group such as detailed here-above for GP1.

STEP 1A is performed as in GP3 here-above.

In case where Intermediate (V-1) has been subjected, prior to step 1A, to step of protecting the function —NR⁵— with a protecting group, the intermediate (IV-2) obtained from STEP 1A is subjected, prior to STEP 2, to a step of deprotecting the function —NR⁵— such as detailed here-above for GP1.

In STEP 2 reaction conditions (A) are implemented as in STEP 1A of GP1, but starting from intermediate (IV-2) and resulting to intermediate (II-4).

In STEP 3, reaction conditions (G) are implemented as in STEP 3 of GP2, so that imine functions of intermediate (II-4) may be reduced to amine functions, thus obtaining compounds of formula (I) as defined here-above.

According to General protocol 5 (GP5), compounds of formula (I) are obtained wherein:
Ar₁, [symbol] X, Y, Q, T, Alk, R³ and R⁴, n and R⁸, if present, are as defined for formula (I) here-above.

R¹ is (CH₂)ₙ—NH-Alk-NR³R⁴,
R² is —NH-Alk-NR³R⁴,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— with a protecting group such as detailed here-above for GP1.

STEP 1A is performed as in GP3 here-above.

In case where Intermediate (V-1) has been subjected, prior to step 1A, to step of protecting the function —NR⁵— with a protecting group, the intermediate (IV-2) obtained from STEP 1A is subjected, prior to STEP 2, to a step of deprotecting the function —NR⁵— such as detailed here-above for GP1.

In STEP 2 reaction conditions (E) are implemented as in STEP 2 of GP3 but starting from intermediate (IV-2) and resulting to intermediate (II-5).

In STEP 3, reaction conditions (G) are implemented as in STEP 3 of GP2, so that imine functions of intermediate (II-5) may be reduced to amine functions, thus obtaining compounds of formula (I) as defined here-above.

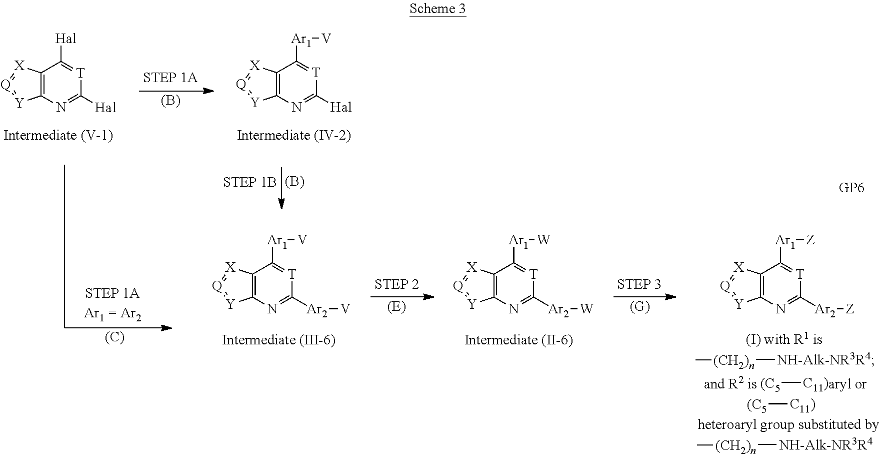

According to General protocol 6 (GP6), compounds of formula (I) are obtained wherein:
Ar₁, [symbol] X, Y, Q, T, Alk, R³ and R⁴, n and R⁸, if present, are as defined for formula (I) here-above.
R¹ is —(CH₂)ₙ—NH-Alk-NR³R⁴,
R² is (C₅-C₁₁)aryl or (C₅-C₁₁)heteroaryl group substituted by a —(CH₂)ₙ—NH-Alk-NR³R⁴,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1A, intermediate (V-1) wherein Y is a group —NR⁵— with R⁵ being a hydrogen atom, may be subjected to an optional step of protecting the function —NR⁵— with a protecting group such as detailed here-above for GP1.

In STEP 1A:

When Ar₁≠Ar₂, STEP 1A is performed as in GP3 here-above so that to obtain intermediate (IV-2).

Then, in STEP 1B, reaction conditions (B) are implemented as in as STEP 1B of GP2, but starting from intermediate (IV-3) and resulting in intermediate (III-6).

When Ar₁=Ar₂, in STEP 1A reaction conditions (C) are implemented as follows.

Optionally protected Intermediate (V-1) may be placed in solution into an appropriate solvent, such as 1,2-dimethoxyethane, dioxane, toluene, benzene, tetrahydrofuran or dimethylformamide, preferably 1,2-dimethoxyethane, in presence of a (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted with a —(CH₂)ₙ₋₁—CHO group, in a molar ratio ranging from 2 to 2.5 with respect to Intermediate (V-1) in presence of a solution of palladium catalyst, such as tetrakis(triphenylphosphine) palladium, for example in a molar ratio ranging from 0.04 to 0.15 with respect to Intermediate (V-1). A previously degassed for between 5 and 20 minutes with an inert gas, such as nitrogen, aqueous solution of an inorganic base, such as Na₂CO₃, K₂CO₃, NaHCO₃, NaOH or KOH, for example in a concentration of 2M, may then be added at room temperature.

Then, the mixture can be warmed to reflux and stirred, for example for between 12 hours and 36 hours, under an inert gas, for example nitrogen, and positive pressure. The reaction mixture can be cooled down to room temperature, the solvent be evaporated under vacuum, the organic layer can be extracted with an appropriate extracting solvent, such as CH₂Cl₂ and the organic phase can be filtered, for example on filter paper, then washed with water, for example 2 or 3 times with between 10 mL and 20 mL of water, dried, for example over anhydrous sodium sulfate and activated charcoal, filtered and evaporated under vacuum.

Otherwise, the reaction mixture can be cooled down to room temperature, filtered, for example on filter paper, and washed successively with one or more appropriate solvents, such as 1,2-dimethoxyethane, water, ethanol and/or diethyl ether. The residue can then be cooled and triturated with a minimum of proper solvent(s), for example EtOH and EtO₂ and filtered, for example on sintered glassware, to give the crude product. The residue can then be purified, for example by silica gel column chromatography (CH₂Cl₂ or CHCl₃/CH₃OH 95:5 or 90:10).
The optionally purified residue can then be cooled and triturated again in a proper solvent, such as EtOH, filtered, for example on sintered glassware, washed with a minimum of proper solvent(s), for example EtOH, EtO₂ and petroleum ether, and dried under pressure to give intermediate (III-6), optionally protected.

In case where Intermediate (V-1) has been subjected, prior to step 1A, to step of protecting the function —NR⁵— with a protecting group, the intermediate (III-6) obtained from STEP 1A or 1B is subjected, prior to STEP 2, to a step of deprotecting the function —NR⁵— such as detailed here-above for GP1.

In STEP 2 reaction conditions (E) are implemented as in STEP 2 of GP3 but starting from intermediate (III-6) and resulting to intermediate (II-6).

In STEP 3, reaction conditions (G) are implemented as in STEP 3 of GP2, so that imine functions of intermediate (II-6) may be reduced to amine functions, thus obtaining compounds of formula (I) as defined here-above.

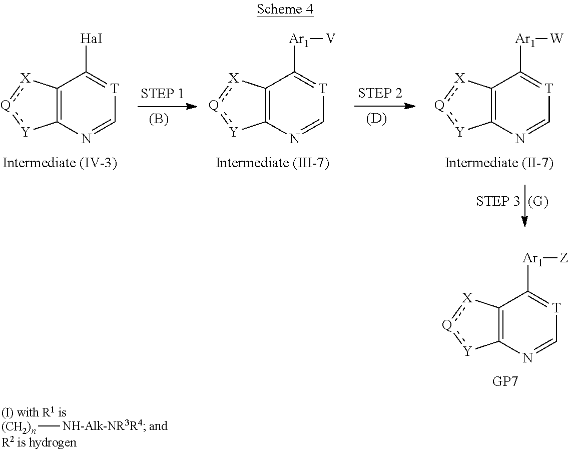

According to General protocol 7 (GP7), compounds of formula (I) are obtained wherein:
Ar₁, [symbol] X, Y, Q, T, Alk, R³ and R⁴, n and R⁸, if present, are as defined for formula (I) here-above.
R¹ is —(CH₂)ₙ—NH-Alk-NR³R⁴
R² is a hydrogen atom,
R⁵, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more (C₁-C₆)alkoxy,
R⁶, if present, is a (C₁-C₆)alkyl group, a group —COOR⁸ or a (C₅-C₁₁)aryl or (C₅-C₁₁)heteroaryl group optionally substituted by one or more (C₁-C₆)alkoxy.

Prior to STEP 1, intermediate (V-1) wherein Y is a group —NR⁵— with R⁵ being a hydrogen atom, may be subjected to an optional step of protecting the function —NR⁵— with a protecting group such as detailed here-above for GP1

In STEP 1 reaction conditions (B) are implemented as in STEP 1B of GP2 but starting from optionally protected intermediate (IV-3) and resulting to optionally protected intermediate (III-7).

In case where Intermediate (V-1) has been subjected, prior to step 1A, to step of protecting the function —NR⁵— with a protecting group, the intermediate (III-7) obtained from STEP 1 is subjected, prior to STEP 2, to a step of deprotecting the function —NR⁵— such as detailed here-above for GP1.

In STEP 2, reaction conditions (D) are implemented as in STEP 2 of GP1, but starting from intermediate (III-7) and resulting to intermediate (11-7).

In STEP 3, reaction conditions (G) are implemented as in STEP 3 of GP2, so that imine functions of intermediate (II-7) may be reduced to amine functions, and obtaining compounds of formula (I) as defined here-above.

According to one embodiment, the compounds of general formula (I) wherein:
T, Q, Ar₁, R¹, R², Alk, R³ and R⁴ are as defined here-above;
X=—CH— or —NR⁵—,
Y=—CH— or —NR⁵—,
with the proviso that at least one of X and Y is —NR⁵—, and
R⁵, when present, is a (C₅-C₁₁)aryl group or (C₅-C₁₁)heteroaryl substituted with —(CH₂)ₙ—NH-AlkNR³R⁴,
can be prepared according to schemes 5 to 9 below.

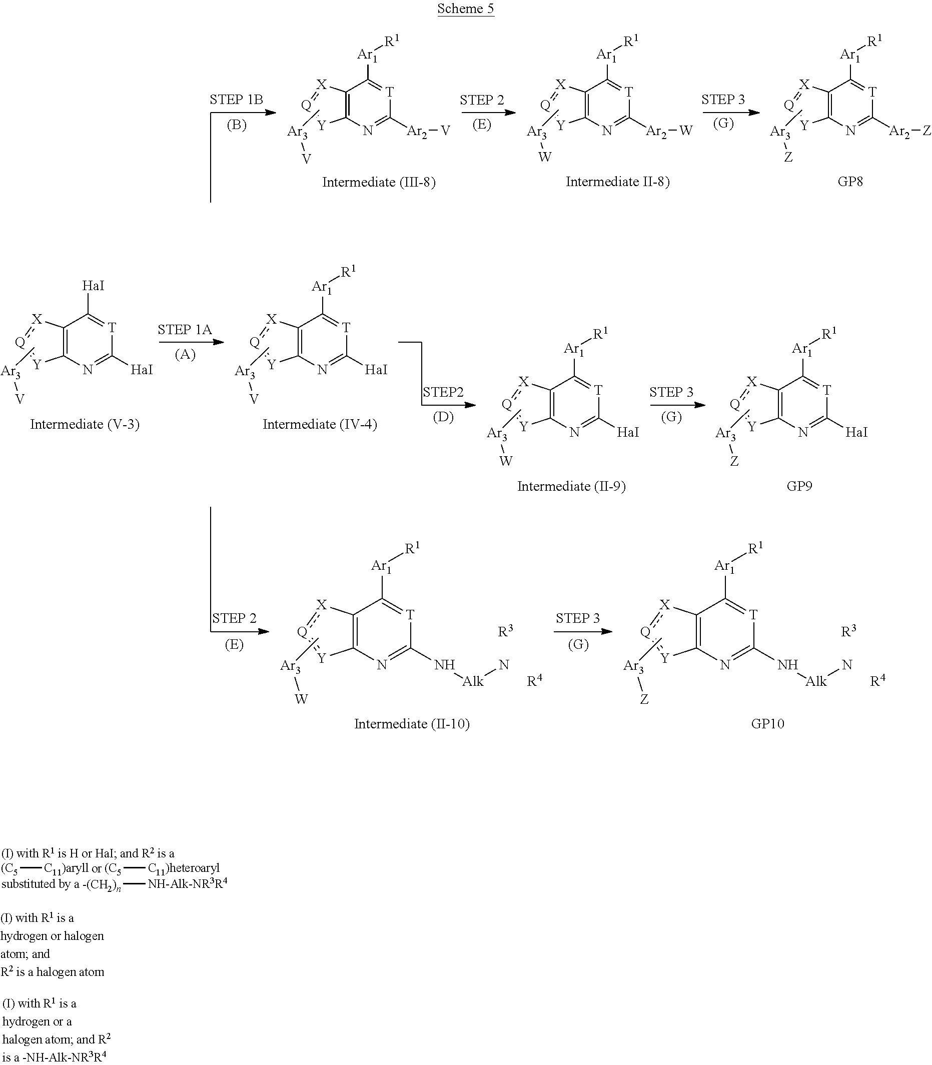

According to General protocol 8 (GP8), compounds of formula (I) are obtained wherein:
Ar₁, [symbol] , Q, T, Alk, R³ and R⁴, n and R⁸, if present, are as defined for formula (I) here-above.
each X and Y is independently —CH— or —NR⁵—, with the proviso that at least one of X and Y is —NR⁵—,
R¹ is a hydrogen atom or halogen atom,
R² is a (C₅-C₁₁)aryl or (C₅-C₁₁)heteroaryl, substituted by a —(CH₂)ₙ—NH-Alk-NR³R⁴, and
R⁵ is a (C₅-C₁₁)aryl group or (C₅-C₁₁)heteroaryl, substituted with —(CH₂)ₙ—NH-AlkNR³R⁴.

In STEP 1A, reaction conditions (A) are implemented as in STEP 1A of GP1, but starting from intermediate (V-3) and resulting to intermediate (IV-4).

In STEP 1B reaction conditions (B) are implemented as in STEP 1B of GP2 but starting from intermediate (IV-4) and resulting to intermediate (III-8).

In STEP 2 reaction conditions (E) are implemented as in STEP 2 of GP3 but starting from intermediate (III-8) and resulting to intermediate (II-8).

In STEP 3, reaction conditions (G) are implemented as in STEP 3 of GP2, so that imine functions of intermediate (II-8) may be reduced to amine functions, and obtaining compounds of formula (I) as defined here-above.

According to General protocol 9 (GP9), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is a hydrogen atom or halogen atom,
R² is a halogen atom, and
R⁵ is a (C₅-C₁₁)aryl group or (C₅-C₁₁)heteroaryl, substituted with —(CH₂)ₙ—NH-AlkNR³R⁴.

STEP 1A is performed as disclosed here-above for GP8.

In STEP 2, reaction conditions (D) are implemented as in STEP 2 of GP1, but starting from intermediate (IV-4) and resulting to intermediate (II-9).

In STEP 3, reaction conditions (G) are implemented as in STEP 3 of GP2, so that imine functions of intermediate (II-9) may be reduced to amine functions, and obtaining compounds of formula (I) as defined here-above.

According to General protocol 10 (GP10), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is a hydrogen atom or halogen atom,
R² is —NH-Alk-NR³R⁴, and
R⁵ is a (C₅-C₁₁)aryl group or (C₅-C₁₁)heteroaryl, substituted with —(CH₂)ₙ—NH-AlkNR³R⁴.

STEP 1A is performed as disclosed here-above for GP8.

In STEP 2 reaction conditions (E) are implemented as in STEP 2 of GP3 but starting from intermediate (IV-4) and resulting to intermediate (II-10).

In STEP 3, reaction conditions (G) are implemented as in STEP 3 of GP2, so that imine functions of intermediate (II-10) may be reduced to amine functions, and obtaining compounds of formula (I) as defined here-above.

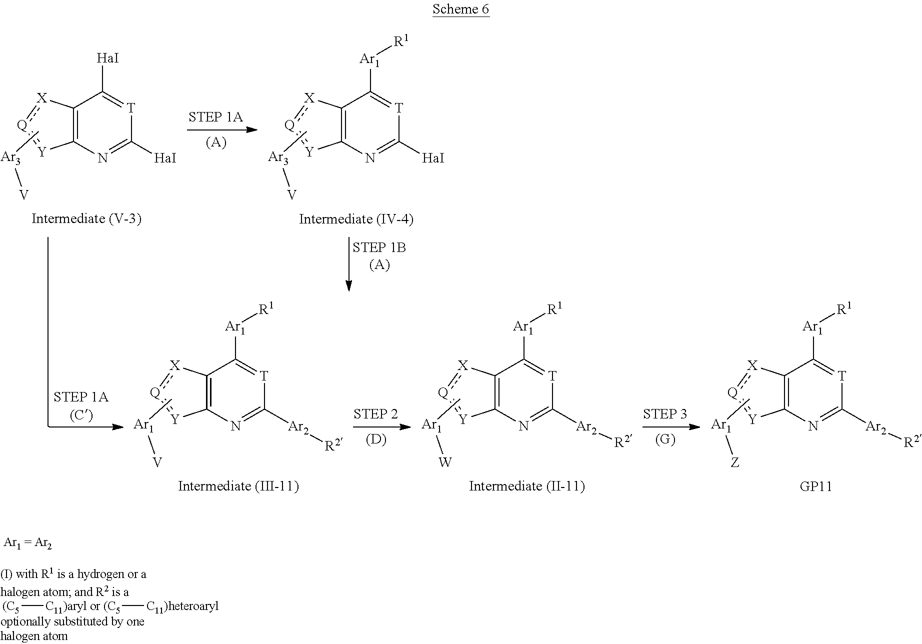

According to General protocol 11 (GP11), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is a hydrogen atom or halogen atom,
R² is a (C₅-C₁₁)aryl or (C₅-C₁₁)heteroaryl, optionally substituted by a halogen atom, and
R⁵ is a (C₅-C₁₁)aryl group or (C₅-C₁₁)heteroaryl, substituted with —(CH₂)ₙ—NH-AlkNR³R⁴.

When Ar₁≠Ar₂, in STEP 1A, reaction conditions (C′) may be performed as follows.

Intermediate (V-3) may be placed in solution into a proper solvent, such as 1,2-dimethoxyethane, dioxane, toluene, benzene, tetrahydrofuran or dimethylformamide, preferably 1,2-dimethoxyethane, in presence of a (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, optionally substituted by a halogen atom, in a molar ratio ranging from 2 to 2.5 with respect to intermediate (V-3) in presence of a solution of palladium catalyst, such as tetrakis(triphenylphosphine) palladium, for example in a molar ratio ranging from 0.04 to 0.15 with respect to intermediate (V-3). A previously degassed for between 5 and 20 minutes with an inert gas, such as nitrogen, aqueous solution of an inorganic base such as Na₂CO₃, K₂CO₃, NaHCO₃, NaOH or KOH, preferably Na₂CO₃, for example in a concentration of 2M, may then be added at room temperature.

Then, the mixture can be warmed to reflux and stirred, for example for between 12 hours and 36 hours, under an inert gas, for example nitrogen, and positive pressure. The reaction mixture can be cooled down to room temperature and the solvent be evaporated under vacuum. The organic layer can be extracted with an appropriate extracting solvent, such as CH₂Cl₂ and the organic phase can be filtered, for example on filter paper, then washed with water, for example 3 times with 20 mL of water, dried, for example over anhydrous sodium sulfate and activated charcoal, filtered and evaporated under vacuum. The residue can then be cooled and triturated with a minimum of proper solvent(s), for example EtOH and EtO₂ and filtered, for example on sintered glassware, to give the crude product. The residue can then be purified, for example by silica gel column chromatography (CH₂Cl₂/CH₃OH 95:5), then cooled and triturated again in EtOH, filtered, for example on sintered glassware, washed with a minimum of proper solvent(s), for example EtOH, then EtO₂ and petroleum ether and dried under pressure to give intermediate (III-11).

When Ar₁≠Ar₂, STEP 1A is performed under reaction conditions (A) as in STEP 1A of GP1 and then STEP 1B is performed as STEP 1B of GP2 but starting from intermediate (IV-4) as disclosed here-above and resulting in intermediate (III-11).

In STEP 2, reaction conditions (D) are implemented as in STEP 2 of GP1, but starting from intermediate (III-11) and resulting to intermediate (II-11).

In STEP 3, reaction conditions (G) are implemented as in STEP 3 of GP2, so that imine functions of intermediate (II-11) may be reduced to amine functions, and obtaining compounds of formula (I) as defined here-above.

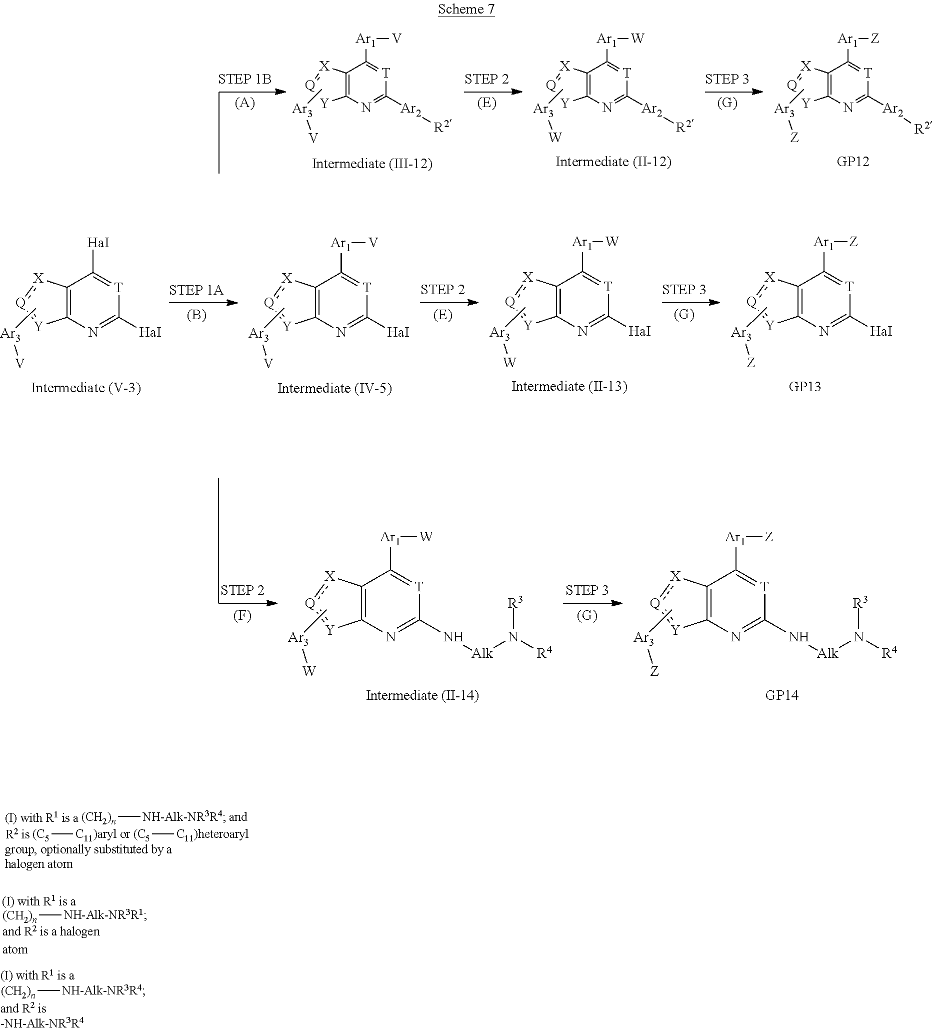

According to General protocol 12 (GP12), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is —(CH₂)ₙ—NH-Alk-NR³R⁴
R² is a (C₅-C₁₁)aryl or (C₅-C₁₁)heteroaryl, optionally substituted by a halogen atom,
R⁵ is a (C₅-C₁₁)aryl group or (C₅-C₁₁)heteroaryl, substituted with —(CH₂)ₙ—NH-AlkNR³R⁴.

In STEP 1A reaction conditions (B) are implemented as in STEP 1B of GP2, but starting from intermediate (V-3) and resulting to intermediate (IV-5).

In STEP 1B reaction conditions (A) are implemented as in STEP 1A of GP1, but starting from intermediate (IV-5) and resulting to intermediate (11-12).

In STEP 2 reaction conditions (E) are implemented as in STEP 2 of GP3 but starting from intermediate (III-12) and resulting to intermediate (II-12).

In STEP 3, reaction conditions (G) are implemented as in STEP 3 of GP2, so that imine functions of intermediate (II-12) may be reduced to amine functions, and obtaining compounds of formula (I) as defined here-above.

According to General protocol 13 (GP13), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is —(CH₂)ₙ—NH-Alk-NR³R⁴
R² is a halogen atom, and
R⁵ is a (C₅-C₁₁)aryl group or (C₅-C₁₁)heteroaryl, substituted with —(CH₂)ₙ—NH-AlkNR³R⁴.

STEP 1A is performed as described here-above for GP12.

In STEP 2 reaction conditions (E) are implemented as in STEP 2 of GP3 but starting from intermediate (IV-5) and resulting to intermediate (II-13).

In STEP 3, reaction conditions (G) are implemented as in STEP 3 of GP2, so that imine functions of intermediate (II-13) may be reduced to amine functions, and obtaining compounds of formula (I) as defined here-above.

According to General protocol 14 (GP14), compounds of formula (I) are obtained wherein:
Ar₁, [symbol] , R³ and R⁴, n and R⁸, if present, are as defined for formula (I) here-above.
each X and Y is independently —CH— or NR⁵, with the proviso that at least one of X and Y is —NR⁵,
R¹ is —(CH₂)ₙ—NH-Alk-NR³R⁴
R² is —NH-Alk-NR³R⁴, and
R⁵ is a (C₅-C₁₁)aryl group or (C₅-C₁₁)heteroaryl, substituted with —(CH₂)ₙ—NH-AlkNR³R⁴.

STEP 1A is performed as described here-above for GP12.

In STEP 2 reaction conditions (F) are implemented as follows.

Intermediate (IV-5) may be added to a solution of the appropriate diamine in a proper solvent, such as ethanol, methanol, tetrahydrofuran, toluene or benzene, preferably ethanol, for example in a molar ratio ranging from 3 to 3.5 with respect to intermediate (IV-6). The reaction mixture may then be heated under reflux, for example for between 2 hours and 24 hours, and then evaporated to dryness under reduced pressure. After cooling, the residue can be extracted with an appropriate extracting solvent, for example dichloromethane, in an amount such as between 15 and 30 mL. The organic layer can then be dried, for example over sodium sulfate and activated charcoal and evaporated to dryness to obtain intermediate (II-14).

In STEP 3, reaction conditions (G) are implemented as in STEP 3 of GP2, so that imine functions of intermediate (II-14) may be reduced to amine functions, and obtaining compounds of formula (I) as defined here-above.

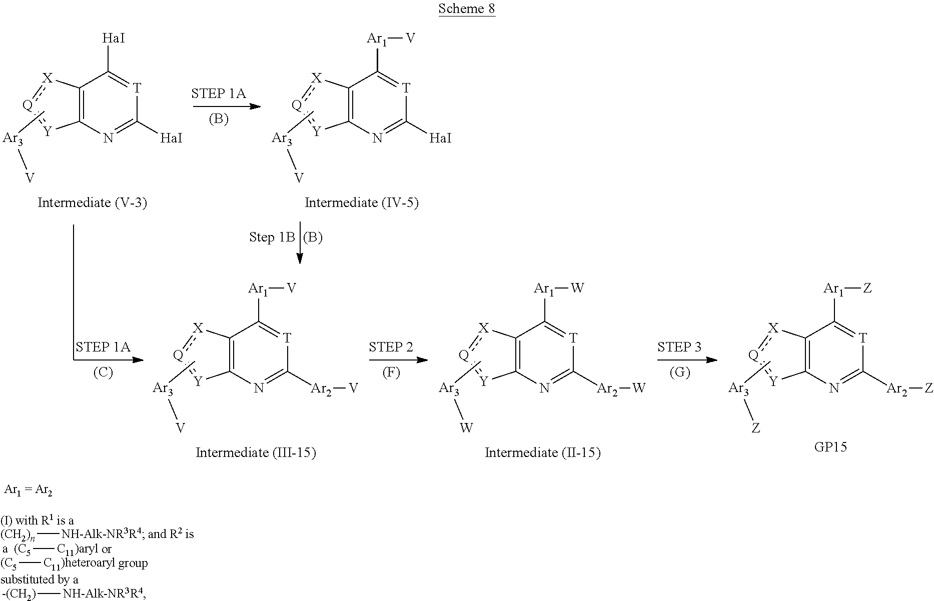

According to General protocol 15 (GP15), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is —(CH₂)ₙ—NH-Alk-NR³R⁴
R² is a (C₅-C₁₁)aryl or (C₅-C₁₁)heteroaryl substituted by a —(CH₂)ₙ—NH-Alk-NR³R⁴, and
R⁵ is a (C₅-C₁₁)aryl group or (C₅-C₁₁)heteroaryl group substituted with —(CH₂)ₙ—NH-AlkNR³R⁴.

When Ar₁=Ar₂, in STEP 1A reaction conditions (C) are implemented as in STEP 1A of GP6 for Ar₁=Ar₂, but starting from intermediate (V-3) and resulting in intermediate (11-15).

When Ar₁≠Ar₂, STEP 1A is performed as described here-above for GP12 to obtain intermediate (IV-5) and then, in STEP 1B reaction conditions (B) are implemented as in STEP 1B of GP2 but starting from intermediate (IV-5) and resulting to intermediate (III-15).

In STEP 2 reaction conditions (F) are implemented as in STEP 2 of GP14 but starting from intermediate (III-15) and resulting to intermediate (II-15).

In STEP 3, reaction conditions (G) are implemented as in STEP 3 of GP2, so that imine functions of intermediate (II-15) may be reduced to amine functions, and obtaining compounds of formula (I) as defined here-above.

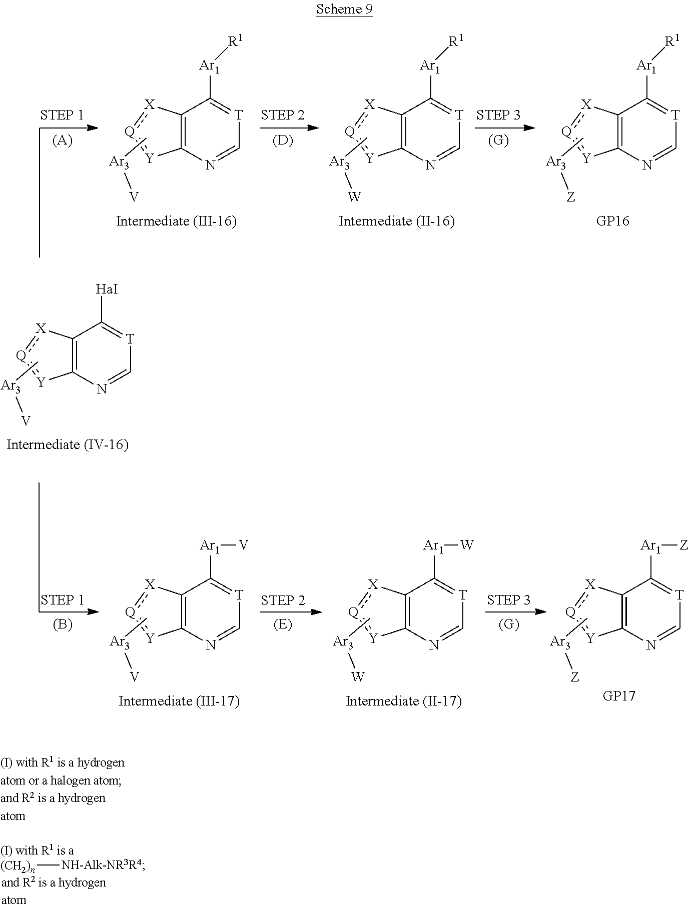

According to General protocol 16 (GP16), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is a halogen or hydrogen atom
R² is a hydrogen atom, and
R⁵ is a (C₅-C₁₁)aryl group or (C₅-C₁₁)heteroaryl group substituted with —(CH₂)ₙ—NH-AlkNR³R⁴.

In STEP 1 reaction conditions (A) are implemented as in STEP 1A of GP1, but starting from intermediate (IV-6) and resulting to intermediate (III-16).

In STEP 2, reaction conditions (D) are implemented as in STEP 2 of GP1, but starting from intermediate (III-16) and resulting to intermediate (II-16).

In STEP 3, reaction conditions (G) are implemented as in STEP 3 of GP2, so that imine functions of intermediate (II-16) may be reduced to amine functions, and obtaining compounds of formula (I) as defined here-above.

According to General protocol 17 (GP17), compounds of formula (I) are obtained wherein:
Ar₁, [symbol] , Q, T, Alk, R³ and R⁴, n and R⁸, if present, are as defined for formula (I) here-above.
each X and Y is independently —CH— or NR⁵, with the proviso that at least one of X and Y is —NR⁵,
R¹ is —(CH₂)ₙ—NH-Alk-NR³R⁴
R² is a hydrogen atom, and
R⁵ is a (C₅-C₁₁)aryl group or (C₅-C₁₁)heteroaryl group substituted with —(CH₂)ₙ—NH-AlkNR³R⁴.

In STEP 1 reaction conditions (B) are implemented as in STEP 1B of GP2 but starting from intermediate (IV-6) and resulting to intermediate (III-17).

In STEP 2 reaction conditions (E) are implemented as in STEP 2 of GP3 but starting from intermediate (III-17) and resulting to intermediate (II-17).

In STEP 3, reaction conditions (G) are implemented as in STEP 3 of GP2, so that imine functions of intermediate (II-17) may be reduced to amine functions, and obtaining compounds of formula (I) as defined here-above.

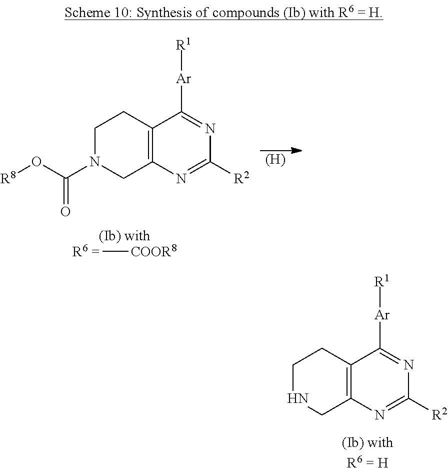

Compounds of formula (Ib) with R⁶=H may be obtained from compounds of formula (Ib) with R⁶=—COOR⁸, and R⁸ is as defined here-above for compound (Ib), under condition (H) as follows.

To a solution of compound (Ib) with R⁶=—COOR⁸ in an appropriate solvent, such as dichloromethane, can be added an acid selected from the group consisting of acetic acid, hydrochloric acid, nitric acid, trifluoroacetic acid, trichloroacetic acid, and a combination thereof, in excess. The mixture can be stirred at room temperature, for example for between 12 hours and 36 hours, then neutralized with a saturated aqueous solution of a buffering agent, for example potassium carbonate, for example at a volume ranging from 50 mL and 80 mL, and extracted with a proper extracting solvent, such as dichloromethane, for example at a volume ranging from 10 mL to 20 mL. The organic layer can be washed with water, then brine and dried, for example with anhydrous sodium sulphate. The solvent can be removed under reduced pressure to give the compound (Ib) with R⁶=H.

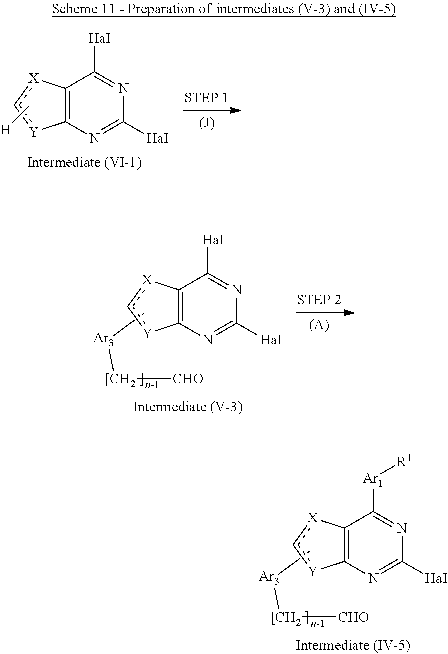

STEP 1: Intermediate (VI-1) may be placed in solution into a proper alcoholic solvent, such as methanol, in presence of a (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted with at least one —(CH₂)ₙ₋₁—CHO, for example in a molar ratio ranging from 1 to 1.5 with respect to intermediate (VI-1) and in the presence of a proper oxidating agent, such as Copper(II) acetate, for example in a molar ratio ranging from 1 to 1.5 with respect to intermediate (VI-1). The solution may be stirred at room temperature, for example for between 2 hours and 8 hours. Then, the reaction mixture can be evaporated to dryness. The residue can then be extracted with an extracting solvent, for example chloroform, filtered and evaporated under reduced pressure. The residue can then be purified, for example by silica gel column chromatography (CHCl₃), then cooled and triturated in proper solvent(s), such as EtOH, filtered, for example on sintered glassware, washed with a minimum of proper solvent(s), such as EtOH, then EtO₂ and petroleum ether and dried under pressure to give intermediate (V-3).

STEP 2: Intermediate (IV-5) my be obtained from intermediate (V-3) under the conditions (A) as implemented in STEP 1A of GP1.

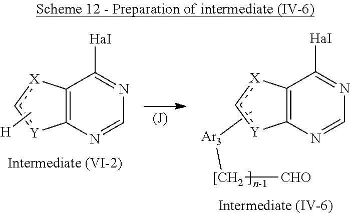

Intermediate (IV-6) my be obtained from intermediate (VI-2) under the conditions (J) as implemented in STEP 1 detailed in Scheme 11 here-above.

Intermediate compounds (IV-1), (III-2), (III-3), (IV-2), (III-6) and (III-7) wherein X or Y is —NR⁵— and R⁵ is a (C₁-C₆)alkyl group substituted with a (C₅-C₁₁)aryl group can be obtained from intermediate compounds (IV-1), (III-2), (III-3), (IV-2), (III-6) and (III-7) respectively wherein X or Y is —NR⁵— and R⁵ is hydrogen, for example under conditions (K) such a detailed here-below.

### Conditions (K)

Intermediate compound (IV-1), (III-2), (III-3), (IV-2), (III-6) or (III-7), wherein X or Y is —NR⁵— and R⁵ is hydrogen, may be placed in suspension in an appropriate solvent, for example acetonitrile, then treated with a base, such a Cs₂CO₃, in particular at a molar ratio of between 2.5 and 3.5 with respect to the Intermediate compound, and with a (C₅-C₁₁)aryl groups substituted by a (C₁-C₆)alkyl halide, in particular bromide, at a molar ratio of between 1 and 2 with respect to the Intermediate compound, and stirred at a temperature ranging from 50° C. to 90° C. for between 1 hour and 3 hours. Then the solvent can be evaporated to dryness under reduced pressure. After cooling, the residue can be triturated in water and extracted with a proper solvent, such as dichloromethane, for example in an amount of between 15 mL and 40 mL. The organic layer can be separated, dried, for example over sodium sulfate and activated charcoal, and evaporated to dryness. The residue can finally be purified, for example by silica gel column chromatography (CH₂Cl₂) to give the corresponding intermediate compound (IV-1), (III-2), (III-3), (IV-2), (III-6) or (III-7) wherein X or Y is —NR⁵— and R⁵ is a (C₁-C₆)alkyl group substituted with a (C₅-C₁₁)aryl group.

Intermediates compounds of formulae (V-1), (IV-4), (VI-1) and (VI-2) are commercially available or can be prepared according to methods known to the person skilled in the art.

Some intermediate compounds involved in the synthesis of compounds of formula (I) as defined here-above are novel.

Therefore, the present invention relates to intermediate compounds of following formula (III), or pharmaceutically acceptable salts thereof:

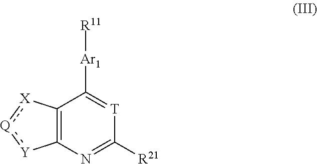

wherein R¹¹ is a hydrogen atom, a halogen atom, or a —(CH₂)ₙ₋₁—CHO,
R²¹ is a hydrogen atom, a halogen atom, or a (C₅-C₁₁)aryl or (C₅-C₁₁)heteroaryl group, optionally substituted by a halogen atom or —(CH₂)ₙ₋₁—CHO,
X is —CH—, —S— or —NR⁵¹— or —N—,
Y is —CH—, —NR⁵¹—, —S— or —NR⁶¹—CH₂—,
T is —CH— and Q is —CH— or T is —N— and Q is —CR¹⁰¹— or —N—,
Each [symbol] is independently a simple or double bond, and at most one of [symbol] is a double bond,
R⁵¹, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more halogen atoms, (C₁-C₆)alkoxy or —(CH₂)ₙ₋₁—CHO,
R⁶¹, if present, is a hydrogen atom, a (C₁-C₆)alkyl group, a group —COOR⁸¹ or a (C₅-C₁₁)aryl or (C₅-C₁₁)heteroaryl group, optionally substituted by one or more halogen atoms, (C₁-C₆)alkoxy or —(CH₂)ₙ₋₁—CHO,
Ar₁ and n are as defined here-above for formula (I),
R⁸¹ represents a hydrogen atom or a (C₁-C₆)alkyl group,
R¹⁰¹ is a hydrogen atom or a (C₅-C₁₁)aryl group or (C₅-C₁₁)heteroaryl,
provided that one or two of X, Q and Y comprises a heteroatom, and
with the proviso that, at least one of R¹¹, R²¹, and, if present, R⁵¹ or R⁶¹, contains a group —(CH₂)ₙ₋₁—CHO.

In particular, the intermediate compounds of formula (III) are of following formula (III-a), or pharmaceutically acceptable salts thereof:

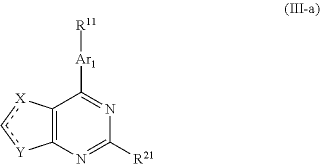

wherein R¹¹ is a hydrogen atom, a halogen atom, or a —(CH₂)ₙ₋₁—CHO,
R²¹ is a hydrogen atom, a halogen atom, or a (C₅-C₁₁)aryl or (C₅-C₁₁)heteroaryl group, optionally substituted by a halogen atom or —(CH₂)ₙ₋₁—CHO,
X is —CH—, —S— or —NR⁵¹—,
Y is —CH—, —NR⁵¹—, —S— or —NR⁶¹—CH₂—,
Each [symbol] is independently a simple or double bond, and at most one of [symbol] is a double bond,
R⁵¹, if present, is a hydrogen atom, a (C₁-C₆)alkyl group or a (C₅-C₁₁)aryl or (C₅-C₁₁)heteroaryl group, optionally substituted by one or more halogen atoms or —(CH₂)ₙ₋₁—CHO,
R⁶¹, if present, is a hydrogen atom, a (C₁-C₆)alkyl group, a group —COOR⁸¹ or a (C₅-C₁₁)aryl or (C₅-C₁)heteroaryl group, optionally substituted by one or more halogen atoms or —(CH₂)ₙ₋₁—CHO,
Ar₁ and n are as defined here-above for formula (I′),
R⁸¹ represents a hydrogen atom or a (C₁-C₆)alkyl group,
provided that one of X and Y comprises a heteroatom, and
with the proviso that, at least one of R¹¹, R²¹, and, if present, R⁵¹ or R⁶¹, contains a group —(CH₂)ₙ₋₁—CHO.

The present invention also relates to intermediate compounds of following formula (I), or pharmaceutically acceptable salts thereof:

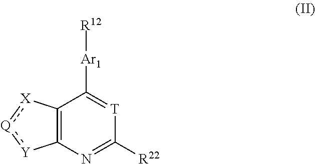

wherein R¹² is a hydrogen atom, a halogen atom, or a —(CH₂)ₙ₋₁CH═N-Alk-NR³R⁴,
R²² is a hydrogen atom, a halogen atom, or a (C₅-C₁₁)aryl or (C₅-C₁₁)heteroaryl group, optionally substituted by a halogen atom or —(CH₂)ₙ₋₁CH═N-Alk-NR³R⁴,
X is —CH—, —S—, —NR⁵²— or —N—,
Y is —CH—, —NR⁵²—, —S— or —NR⁶²—CH₂—,
T is —CH— and Q is —CH— or T is —N— and Q is —CR¹⁰²— or —N—,
Each [symbol] is independently a simple or double bond, and at most one of [symbol] is a double bond,
R⁵², if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more halogen atoms, (C₁-C₆)alkoxy or —(CH₂)ₙ₋₁CH═N-Alk-NR³R⁴,
R⁶², if present, is a hydrogen atom, a (C₁-C₆)alkyl group, a group —COOR⁸² or a (C₅-C₁₁)aryl group or (C₅-C₁₁)heteroaryl, optionally substituted by one or more halogen atoms, (C₁-C₆)alkoxy or —(CH₂)ₙ₋₁CH═N-Alk-NR³R⁴,
R¹⁰² is a hydrogen atom or a (C₅-C₁₁)aryl group or (C₅-C₁₁)heteroaryl,
Ar₁, Alk, R³, R⁴ and n are as defined here-above for formula (I),
provided that one or two of X, Q and Y comprises a heteroatom, and
with the proviso that, at least one of R¹², R²², and, if present, R⁵² or R⁶², contains a group —(CH₂)ₙ₋₁CH═N-Alk-NR³R⁴.

In particular, the intermediate compounds of formula (II) are of following formula (II-a), or pharmaceutically acceptable salts thereof:

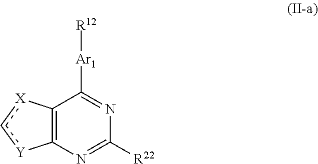

wherein R¹² is a hydrogen atom, a halogen atom, or a —(CH₂)ₙ₋₁CH═N-Alk-NR³R⁴,
R²² is a hydrogen atom, a halogen atom, or a (C₅-C₁₁)aryl or (C₅-C₁₁)heteroaryl group, optionally substituted by a halogen atom or —(CH₂)ₙ₋₁CH═N-Alk-NR³R⁴,
X is —CH—, —S— or —NR⁵²,
Y is —CH—, —NR⁵²—, —S— or —NR⁶²—CH₂—,
Each [symbol] is independently a simple or double bond, and at most one of [symbol] is a double bond,
R⁵², if present, is a hydrogen atom, a (C₁-C₆)alkyl group or a (C₅-C₁₁)aryl or (C₅-C₁₁)heteroaryl group, optionally substituted by one or more halogen atoms or —(CH₂)ₙ₋₁CH═N-Alk-NR³R⁴,
R⁶², if present, is a hydrogen atom, a (C₁-C₆)alkyl group, a group —COOR⁸² or a (C₅-C₁₁)aryl group or (C₅-C₁₁)heteroaryl, optionally substituted by one or more halogen atoms or —(CH₂)ₙ₋₁CH═N-Alk-NR³R⁴,
Ar₁, Alk, R³, R⁴ and n are as defined here-above for formula (I′),
provided that one of X and Y comprises a heteroatom, and
   with the proviso that, at least one of R¹², R²², and, if present, R⁵² or R⁶², contains a group —(CH₂)ₙ₋₁CH═N-Alk-NR³R⁴.

The process for obtaining compounds of formula (I) is based on the Suzuki coupling between halogen atom borne by compounds of formula (IV) as defined below, with at least one (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted by one or more —(CH₂)ₙ₋₁—CHO; followed by imine formation through reaction between the formyl group and a diamine, then by the reduction of these imine functions, and/or on direct amination of the halogen atom, if present, as R²³ of compound of formula (IV).

Accordingly, the present invention further relates to a synthesis process for manufacturing new compounds of formula (I), (Ia), (Ia-a), (Ia-b), (Ia-c), (Ia-d), (Ia-e), (Ib), (Ib-a), (Ic), (Ic-a), (Ic-b), (Ic-c), (Id), (Id-a), (Id-b), (Id-c), (Ie), (Ie-a), (Ie-b), (Ie-b′), (Ie-b″), (If), (If-a), (If-b), (Ig), (Ig-a) or any of the compounds (1) to (49) as defined above, comprising at least the steps of:
(a) Providing a compound having following formula (IV):

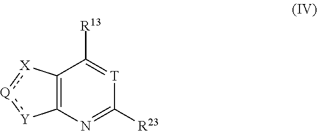

wherein R¹³ is a halogen atom, or a (C₅-C₁₁)arylene or (C₅-C₁₁)heteroarylene group optionally substituted by a halogen atom,
R²³ is a hydrogen atom, a halogen atom or a (C₅-C₁₁)aryl or (C₅-C₁₁)heteroaryl group, optionally substituted by a halogen atom,
X is —CH—, —S—, —NR⁵³— or —N—,
Y is —CH—, —NR⁵³—, —S— or —NR⁶³—CH₂—,
T is —CH— and Q is —CH— or T is —N— and Q is —CR¹⁰³— or —N—,
Each [symbol] is independently a simple or double bond, and at most one of [symbol] is a double bond,
R⁵³, if present, is a hydrogen atom, a (C₁-C₆)alkyl group optionally substituted by one or more (C₅-C₁₁)aryl or (C₅-C₁₁)heteroaryl group, a (C₅-C₁₁)aryl or (C₅-C₁₁)heteroaryl group optionally substituted by one or more halogen atoms or (C₁-C₆)alkoxy, or a protecting group,
R⁶³, if present, is a hydrogen atom, a (C₁-C₆)alkyl group, a group —COOR⁸³, a (C₅-C₁₁)aryl or (C₅-C₁₁)heteroaryl group optionally substituted by one or more halogen atoms or C₁-C₆)alkoxy, or a protecting group,
R⁸³ represents a hydrogen atom or a (C₁-C₆)alkyl group,
R¹⁰³ is a hydrogen atom or a (C₅-C₁₁)aryl group or (C₅-C₁₁)heteroaryl,
provided that one or two of X, Q and Y comprises a heteroatom, and
with the proviso that, at least one of R¹³ and R²³ is a halogen atom or, if present, R⁵³ or R⁶³ is a hydrogen atom,
(b) Optionally reacting the intermediate compound of formula (IV) with at least one (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted by one or more —(CH₂)ₙ₋₁—CHO under reactive conditions suitable for grafting a function (C₅-C₁₁)aryl-(CH₂)ₙ₋₁—CHO or (C₅-C₁₁)heteroaryl-(CH₂)ₙ₋₁—CHO, such reacting being eventually followed by deprotecting the function —NR⁵³— or —NR⁶³ if present and/or reacting the function —NR⁵³— or —NR⁶³ where R⁵³ and R⁶³ are hydrogen atoms, if present, under conditions suitable for grafting a (C₁-C₆)alkyl group substituted by one or more (C₅-C₁₁)aryl or (C₅-C₁₁)heteroaryl group, and thus obtaining a compound of formula (III) as defined here-above;
(c) Coupling the formaldehyde function(s) from compound of formula (III) obtained in step (b) with a diamine compound of formula H₂N-Alk-NR³R⁴ under reactive conditions suitable for the formation of imine functions leading to a compound of formula (II) as defined here-above, and eventually coupling the halogen atom, when present as R²¹ of compound (III), with the diamine compound of formula H₂N-Alk-NR³R⁴ to form an amine function —NH-Alk-NR³R⁴, or coupling the halogen atom, when present as R²³ of compound of formula (IV), with a diamine compound of formula H₂N-Alk-NR³R⁴ under reactive conditions suitable for the formation of an amine function —NH-Alk-NR³R⁴; and
(d) When necessary, coupling the imine function(s) from compound of formula (II) obtained in step (c) to suitable reactive conditions for reducing the imine function(s) to amine function(s) —NH-Alk-NR³R⁴.

In particular, the present invention further relates to a synthesis process for manufacturing new compounds of formula (I), (Ia), (Ia-a), (Ia-b), (Ia-c), (Ia-d), (Ia-e), (Ib), (Ib-a), (Ic), (Ic-a), (Ic-b), (Ic-c), (Id), (Id-a), (Id-b), (Id-c) or any of the compounds (1) to (32) as defined above, comprising at least the steps of:
(a) Providing a compound having following formula (IV-a):

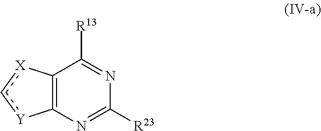

wherein R¹³ is a halogen atom, or a (C₅-C₁₁)arylene or (C₅-C₁₁)heteroarylene group optionally substituted by a halogen atom,
R²³ is a hydrogen atom, a halogen atom or a (C₅-C₁₁)aryl or (C₅-C₁₁)heteroaryl group, optionally substituted by a halogen atom,
X is —CH—, —S— or —NR⁵³—,
Y is —CH—, —NR⁵³—, —S— or —NR⁶³—CH₂—,
Each [symbol] is independently a simple or double bond, and at most one of [symbol] is a double bond,
R⁵³, if present, is a hydrogen atom, a (C₁-C₆)alkyl group or a (C₅-C₁₁)aryl or (C₅-C₁₁)heteroaryl group, optionally substituted by one or more halogen atoms,
R⁶³, if present, is a hydrogen atom, a (C₁-C₆)alkyl group, a group —COOR⁸³ or a (C₅-C₁₁)aryl or (C₅-C₁₁)heteroaryl group, optionally substituted by one or more halogen atoms,
R⁸³ represents a hydrogen atom or a (C₁-C₆)alkyl group,
provided that one of X and Y comprises a heteroatom, and
with the proviso that, at least one of R¹³ and R²³ is a halogen atom or, if present, R⁵³ or R⁶³ is a hydrogen atom,
(b) Optionally reacting the intermediate compound of formula (IV-a) with at least one (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted by one or more —(CH₂)ₙ₋₁—CHO under reactive conditions suitable for grafting a function (C₅-C₁₁)aryl-(CH₂)ₙ₋₁—CHO or (C₅-C₁₁)heteroaryl-(CH₂)ₙ₋₁—CHO and thus obtaining a compound of formula (III-a) as defined here-above;
(c) Coupling the formaldehyde function(s) from compound of formula (III-a) obtained in step (b) with a diamine compound of formula H₂N-Alk-NR³R⁴ under reactive conditions suitable for the formation of imine functions leading to a compound of formula (II-a) as defined hereabove, and eventually coupling the halogen atom, when present as R² of compound (III-a), with the diamine compound of formula H₂N-Alk-NR³R⁴ to form an amine function —NH-Alk-NR³R⁴, or coupling the halogen atom, when present as R²³ of compound of formula (IV-a), with a diamine compound of formula H₂N-Alk-NR³R⁴ under reactive conditions suitable for the formation of an amine function —NH-Alk-NR³R⁴; and
(d) When necessary, coupling the imine function(s) from compound of formula (II-a) obtained in step (c) to suitable reactive conditions for reducing the imine function(s) to amine function(s) —NH-Alk-NR³R⁴.

In particular, during step (b) the compound of formula (IV) or (IV-a) may be placed in solution into a proper solvent, such as 1,2-dimethoxyethane, dioxane, toluene, benzene, tetrahydrofuran or dimethylformamide, preferably 1,2-dimethoxyethane, in presence of the (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, for example with MIDA, or a (C₅-C₁₁)aryl or (C₅-C₁₁)heteroaryl trifluoro borate salt, in particular potassium salt, substituted by one or more —(CH₂)ₙ₋₁—CHO, in presence of a solution of palladium catalyst, such as tetrakis(triphenylphosphine) palladium. A previously degassed for between 5 and 20 minutes with an inert gas, such as nitrogen, aqueous solution of an inorganic base such as Na₂CO₃, K₂CO₃, NaHCO₃, NaOH or KOH, preferably Na₂CO₃, for example in a concentration of 2M, may then be added at room temperature.

Then, the mixture can be warmed to reflux and stirred, for example for between 12 hours and 36 hours, under an inert gas, for example nitrogen, and positive pressure.

The reaction mixture can be cooled down to room temperature and the solvent be evaporated under vacuum. The organic layer can be extracted with an appropriate extracting solvent, such as CH₂Cl₂ and the organic phase can be filtered, for example on filter paper, then washed with water, for example 2 or 3 times with between 10 mL and 20 mL of water, dried, for example over anhydrous sodium sulfate and activated charcoal, filtered and evaporated under vacuum.

Otherwise, the reaction mixture can be cooled down to room temperature, filtered, for example on filter paper, and washed successively with one or more appropriate solvents, such as 1,2-dimethoxyethane, water, ethanol and/or diethyl ether

The residue can then be cooled and triturated with a minimum of proper solvent(s), for example EtOH and EtO₂ and filtered, for example on sintered glassware, to give the crude product. The residue can then be purified, for example by silica gel column chromatography, then optionally further cooled and triturated again in a proper solvent, such as EtOH, filtered, for example on sintered glassware. The eventually purified residue can be washed with a minimum of proper solvent(s), for example EtOH, EtO₂ and/or petroleum ether and dried under pressure to give intermediate (III) or (III-a).

During step (c), the compound of formula (III) or (III-a) obtained in step (b) or alternatively the compound of formula (IV) or (IV-a) wherein R²³ is a halogen atom, may be added to a solution of the appropriate diamine in a proper solvent, such as ethanol, methanol, tetrahydrofuran, toluene or benzene, preferably ethanol. The reaction mixture may then be heated under reflux, for example for between 2 hours and 24 hours, and then evaporated to dryness under reduced pressure. After cooling, the residue can be extracted with an appropriate extracting solvent, for example dichloromethane, in an amount such as between 15 and 50 mL. The organic layer can then be dried, for example over sodium sulfate and activated charcoal and evaporated to dryness to obtain respectively compound of formula (II) or alternatively compound of formula (I) wherein R² is —NH-Alk-NR³R⁴.

During step (d), compound of formula (II) or (II-a) may be placed in solution into a proper alcoholic solvent, such as methanol. Then, a reducing agent, such a sodium borohydride can be added portion-wise at a temperature of between −5° C. to 5° C. The reaction mixture can then be stirred at a temperature ranging from 20° C. and 80° C., for example for between 1 hour and 3 hours. Then it can be evaporated to dryness under reduced pressure. After cooling, the residue can be triturated in water and extracted with an extracting solvent, such as dichloromethane, for example between 15 and 40 mL. The organic layer can be separated, dried, for example over sodium sulfate and activated charcoal and evaporated to dryness to give compounds (I) as defined above, except those for which R² is —NH-Alk-NR³R⁴.

Some compounds of the invention are described with their structure in the below Table 2, which is merely illustrative and does not limit the scope of the present invention.

TABLE 2
Structure of compounds (1) to (49). Formulas were generated
using ChemDraw Prime 16.0.

| Cpd | | Basic | | Sub- |
|---|---|---|---|---|
| No | Salt | formula | Formula (I) | group |
| 1A 1B | — Oxalate | C₃₆H₄₅N7 C₃₆H₄₅N7• 4(COOH)₂ | 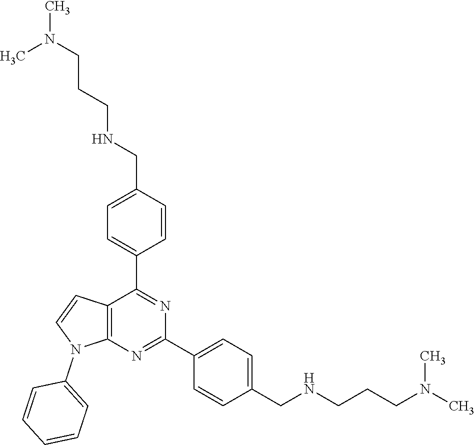 | (Ia-a) |
| 2A 2B | — Oxalate | C₄₂H₅₅N₉ C₄₂H₅₅N₉• 6(COOH)₂ | 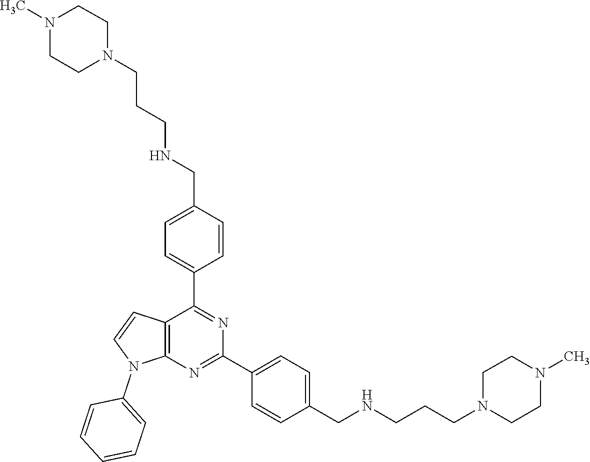 | (Ia-a) |
| 3A 3B | — Oxalate | C₃₈H₄₉N₇ C₃₈H₄₉N₇• 4(COOH)₂ | 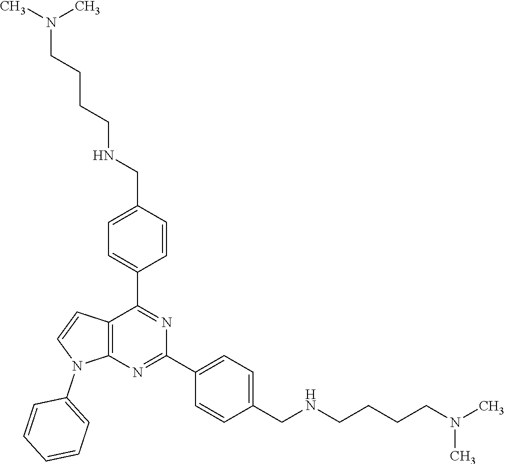 | (Ia-a) |
| 4A 4B | — Oxalate | C₄₄H₅₉N₉ C₄₄H₅₉N₉• 6(COOH)₂ | 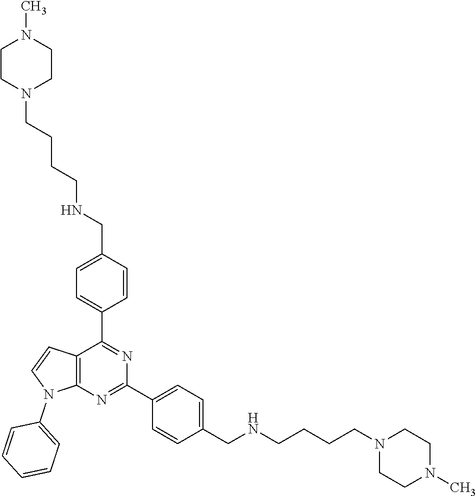 [SMILES: CN1CCN(CCCCNCc2ccc(-c3nc(-c4ccc(CNCCCCN5CCN(C)CC5)cc4)c4ccn(-c5ccccc5)c4n3)cc2)CC1] | (Ia-a) |
| 5A 5B | — Oxalate | C₄₀H₅₃N₇ C₄₀H₅₃N₇• 4(COOH)₂ | 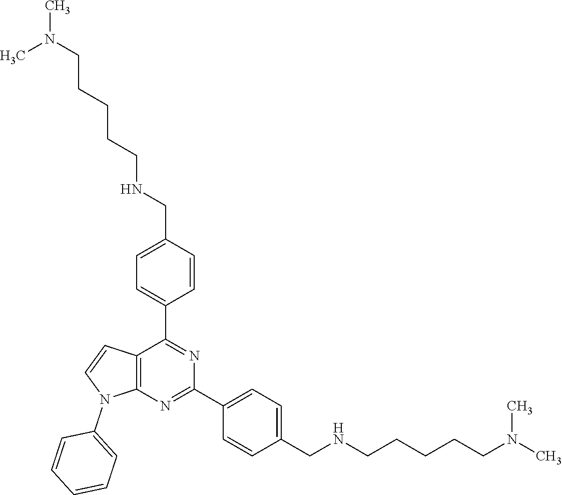 | (Ia-a) |
| 6A 6B | — Oxalate | C₃₄H₄₃N₇S C₃₄H₄₃N₇S• 4(COOH)₂ | 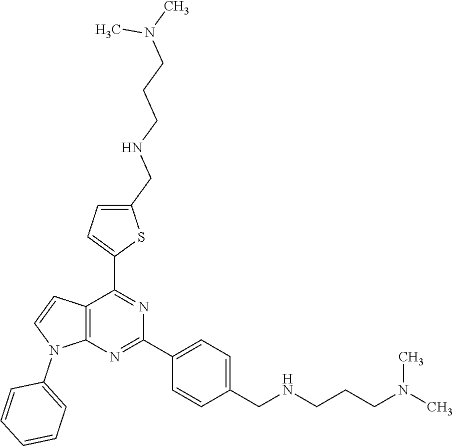 [SMILES: CN(C)CCCNCc1ccc(-c2nc(-c3ccc(CNCCCN(C)C)s3)c3ccn(-c4ccccc4)c3n2)cc1] | (Ia-a) |
| 7A 7B | — Oxalate | C₃₄H₄₃N₇S C₃₄H₄₃N₇S• 4(COOH)₂ | 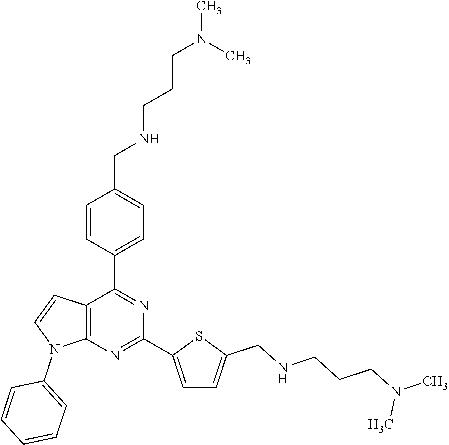 [SMILES: CN(C)CCCNCc1ccc(-c2nc(-c3ccc(CNCCCN(C)C)s3)nc3c2ccn3-c2ccccc2)cc1] | (Ia-a) |
| 8A 8B | — Oxalate | C₃₁H₄₃N₇ C₃₁H₄₃N₇• 4(COOH)₂ | 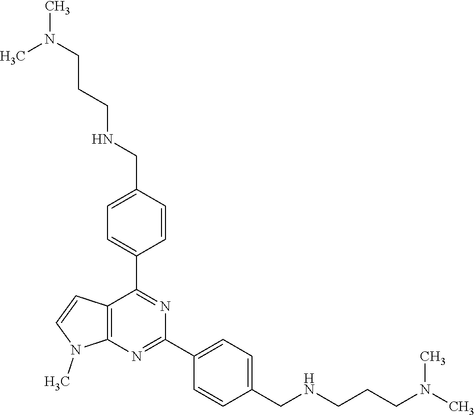 | (Ia-a) |
| 9A 9B | — Oxalate | C₃₇H₅₃N₉ C₃₇H₅₃N₉• 6(COOH)₂ | 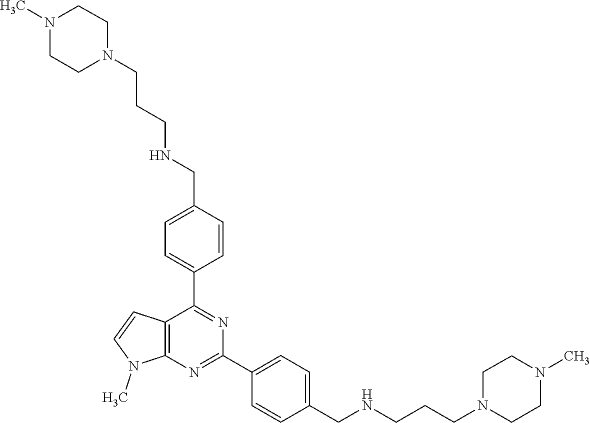 | (Ia-a) |
| 10A 10B | — Oxalate | C₄₅H₆₃N₇O₈ C₄₅H₆₃N₇O₈• 2(COOH)₂ | 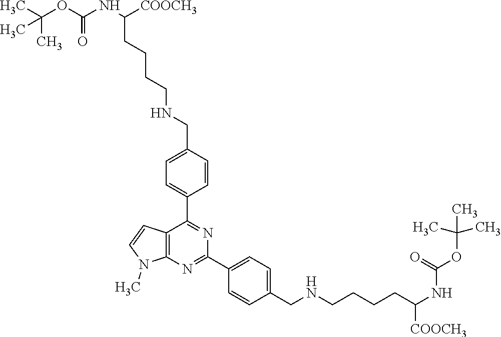 [SMILES: COC(=O)C(CCCCNCc1ccc(-c2nc(-c3ccc(CNCCCCC(NC(=O)OC(C)(C)C)C(=O)OC)cc3)c3ccn(C)c3n2)cc1)NC(=O)OC(C)(C)C] | (Ia-a) |
| 11A 11B | — Oxalate | C₃₅H₄₇N₇O₄ C₃₅H₄₇N₇O₄• 4(COOH)₂ | 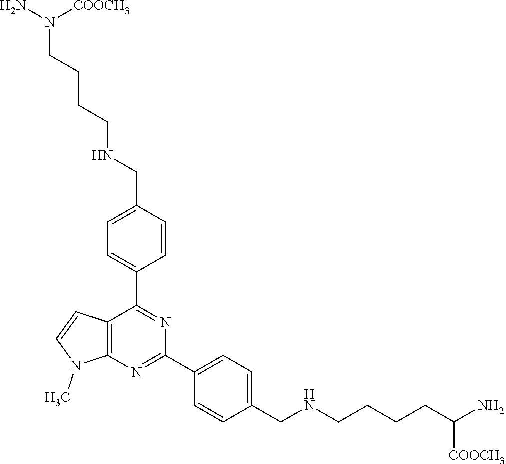 [SMILES: COC(=O)C(N)CCCCNCc1ccc(-c2nc(-c3ccc(CNCCCCN(N)C(=O)OC)cc3)c3ccn(C)c3n2)cc1] | (Ia-a) |
| 12A 12B | — Oxalate | C₃₀H₃₁N₅ C₃₀H₃₁N₅• 2(COOH)₂ | 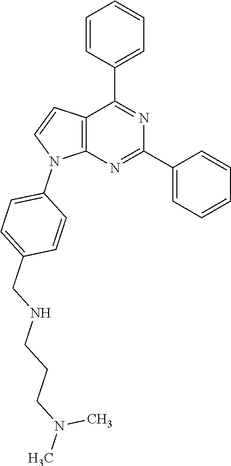 [SMILES: CN(C)CCCNCc1ccc(-n2ccc3c(-c4ccccc4)nc(-c4ccccc4)nc32)cc1] | (Ia-b) |
| 13A 13B | — Oxalate | C₄₂H₅₉N₉ C₄₂H₅₉N₉• 6(COOH)₂ | 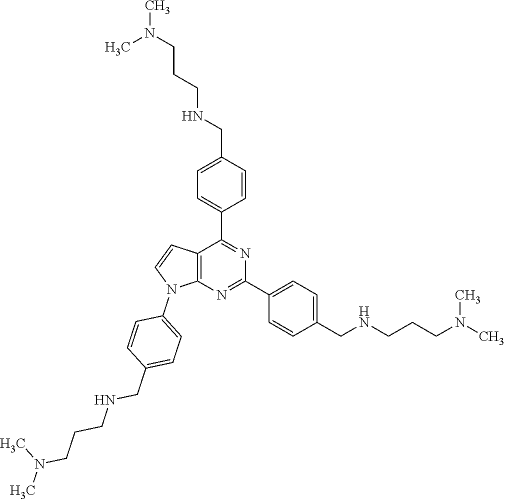 | (Ia-b) |
| 14A 14B | — Oxalate | C₂₉H₃₉N₇ C₂₉H₃₉N₇• 4(COOH)₂ | 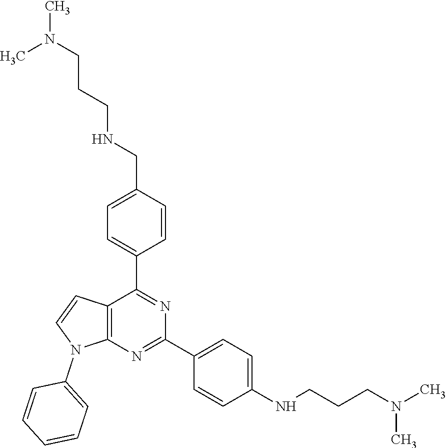 | (Ia-c) |
| 15A 15B | — Oxalate | C₁₈H₂₂ClN₅ C₁₈H₂₂ClN₅• 2(COOH)₂ | 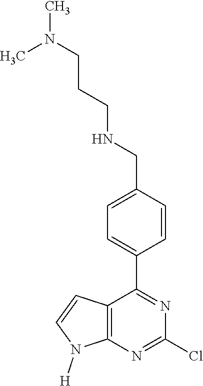 [SMILES: CN(C)CCCNCc1ccc(-c2nc(Cl)nc3[nH]ccc23)cc1] | (Ia-d) |
| 16A 16B | — Oxalate | C₂₁H₂₇ClN₆ C₂₁H₂₇ClN₆• 3(COOH)₂ | 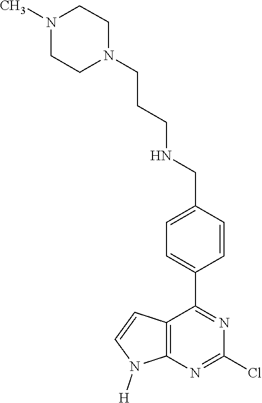 [SMILES: CN1CCN(CCCNCc2ccc(-c3nc(Cl)nc4[nH]ccc34)cc2)CC1] | (Ia-d) |
| 17A 17B | — Oxalate | C₂₄H₂₆ClN₅ C₂₄H₂₆ClN₅• 2(COOH)₂ | 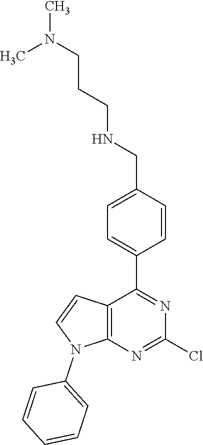 [SMILES: CN(C)CCCNCc1ccc(-c2nc(Cl)nc3c2ccn3-c2ccccc2)cc1] | (Ia-d) |
| 18A 18B | — Oxalate | C₂₇H₃₁ClN₆ C₂₇H₃₁ClN₆• 3(COOH)₂ | 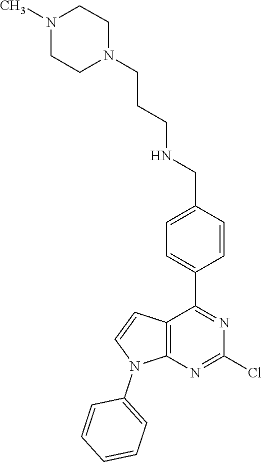 [SMILES: CN1CCN(CCCNCc2ccc(-c3nc(Cl)nc4c3ccn4-c3ccccc3)cc2)CC1] | (Ia-d) |
| 19A 19B | — Oxalate | C₂₂H₂₄ClN₅S C₂₂H₂₄ClN₅S• 2(COOH)₂ | 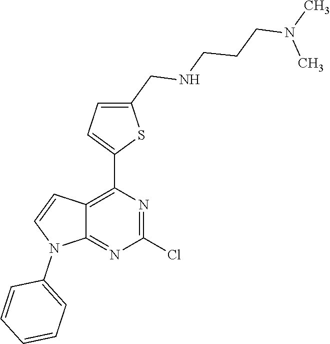 [SMILES: CN(C)CCCNCc1ccc(-c2nc(Cl)nc3c2ccn3-c2ccccc2)s1] | (Ia-d) |
| 20A 20B | — Oxalate | C₂₂H₂₄ClN₅S C₂₂H₂₄ClN₅S• 2(COOH)₂ | 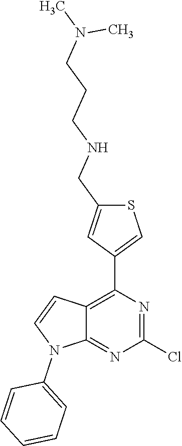 [SMILES: CN(C)CCCNCc1cc(-c2nc(Cl)nc3c2ccn3-c2ccccc2)cs1] | (Ia-d) |
| 21A 21B | — Oxalate | C₂₂H₂₄ClN₅S C₂₂H₂₄ClN₅S• 2(COOH)₂ | 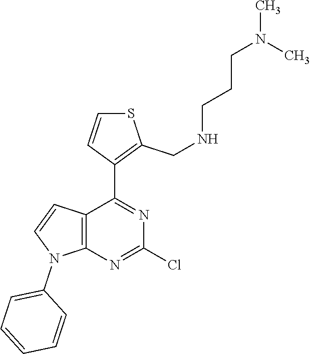 [SMILES: CN(C)CCCNCc1sccc1-c1nc(Cl)nc2c1ccn2-c1ccccc1] | (Ia-d) |
| 22A 22B | — Oxalate | C₂₄H₂₆ClN₅ C₂₄H₂₆ClN₅• 2(COOH)₂ | 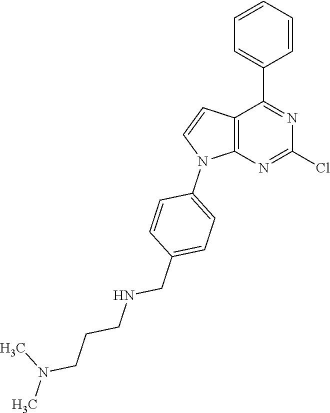 [SMILES: CN(C)CCCNCc1ccc(-n2ccc3c(-c4ccccc4)nc(Cl)nc32)cc1] | (Ia-e) |
| 23A 23B | — Oxalate | C₂₇H₃₁ClN₆ C₂₇H₃₁ClN₆• 3(COOH)₂ | 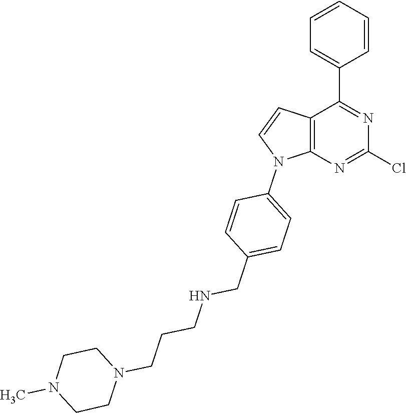 [SMILES: CN1CCN(CCCNCc2ccc(-n3ccc4c(-c5ccccc5)nc(Cl)nc43)cc2)CC1] | (Ia-e) |
| 24A 24B | — Oxalate | C₃₆H₅₃N₇O₂ C₃₆H₅₃N₇O₂• 4(COOH)₂ | 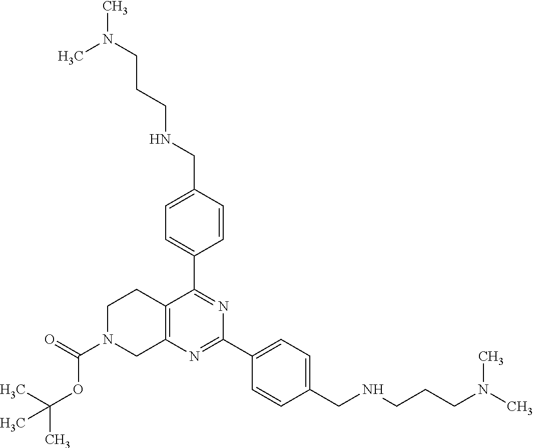 [SMILES: CN(C)CCCNCc1ccc(-c2nc3c(c(-c4ccc(CNCCCN(C)C)cc4)n2)CCN(C(=O)OC(C)(C)C)C3)cc1] | (Ib-a) |
| 25A 25B | — Oxalate | C₃₁H₄₅N₇ C₃₁H₄₅N₇• 5(COOH)₂ | 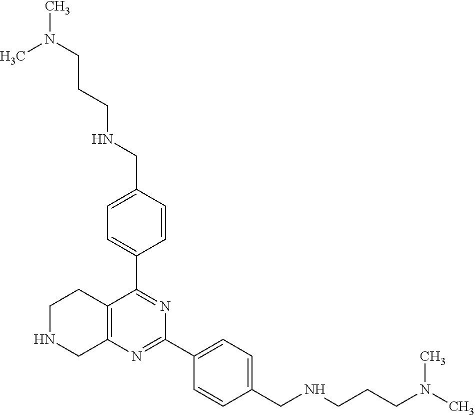 | (Ib-a) |
| 26A 26B | — Oxalate | C₃₀H₄₀N₆S C₃₀H₄₀N₆S• 4(COOH)₂ | 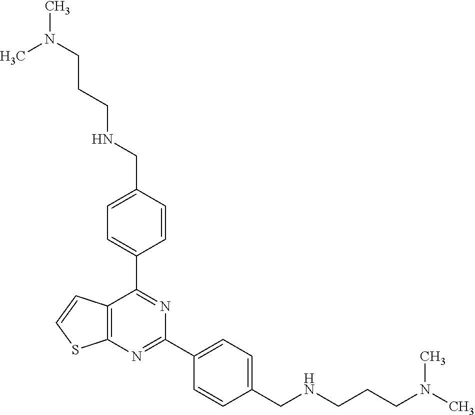 | (Ic-a) |
| 27A 27B | — Oxalate | C₃₆H₅₀N₈S C₃₆H₅₀N₈S• 6(COOH)₂ | 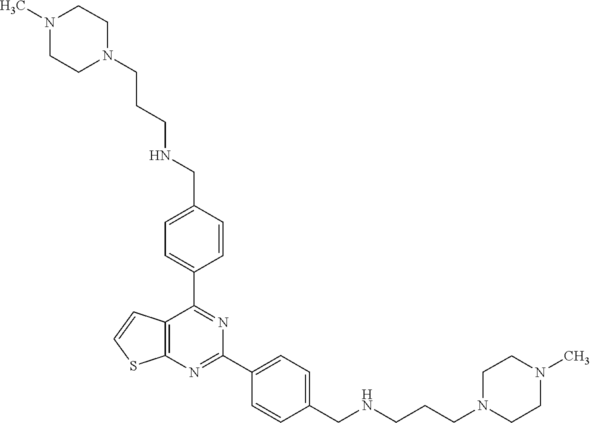 | (Ic-a) |
| 28A 28B | — Oxalate | C₂₃H₃₄N₆S C₂₃H₃₄N₆S• 4(COOH)₂ | 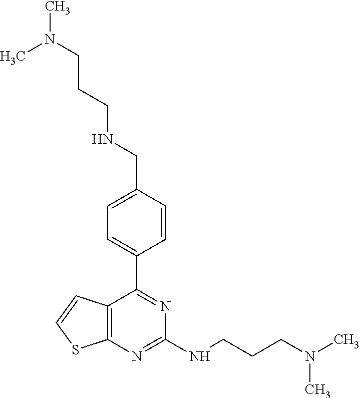 | (Ic-b) |
| 29A 29B | — Oxalate | C₃₀H₄₀N₆S C₃₀H₄₀N₆S• 4(COOH)₂ | 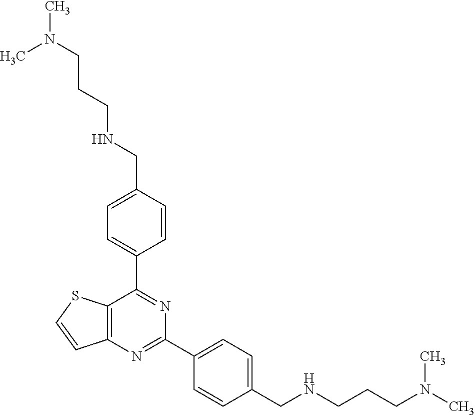 | (Id-a) |
| 30A 30B | — Oxalate | C₃₆H₅₀N₈S C₃₆H₅₀N₈S• 6(COOH)₂ | 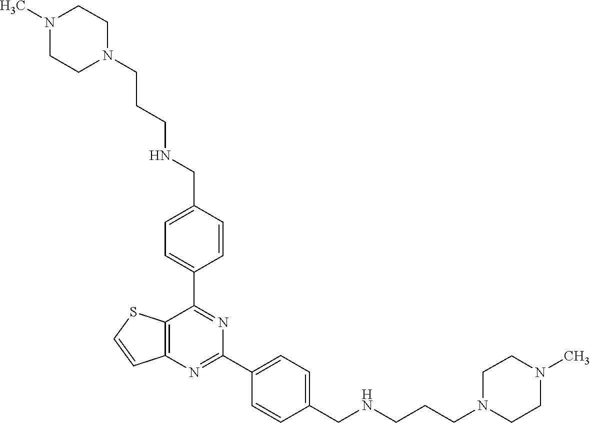 | (Id-a) |
| 31A 31B | — Oxalate | C₂₃H₃₄N₆S C₂₃H₃₄N₆S• 4(COOH)₂ | 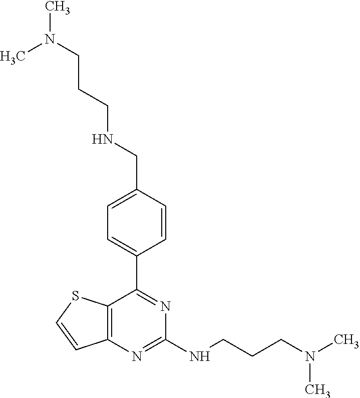 | (Id-b) |
| 32A 32B | — Oxalate | C₃₈H₄₉N₇ C₃₈H₄₉N₇• 4(COOH)₂ | 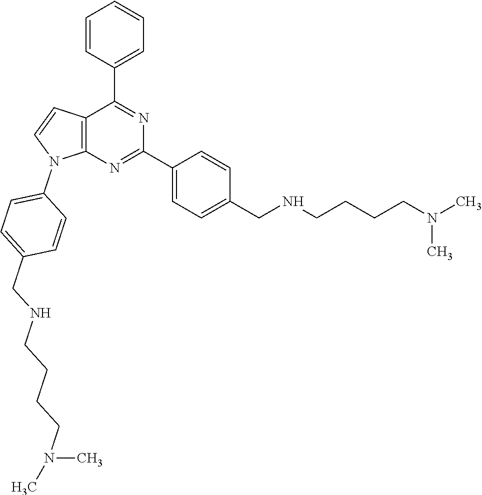 [SMILES: CN(C)CCCCNCc1ccc(-c2nc(-c3ccccc3)c3ccn(-c4ccc(CNCCCCN(C)C)cc4)c3n2)cc1] | (Ia-b) |
| 33A 33B | — Oxalate | C₄₂H₅₇N₇ C₄₂H₅₇N₇• 4(COOH)₂ | 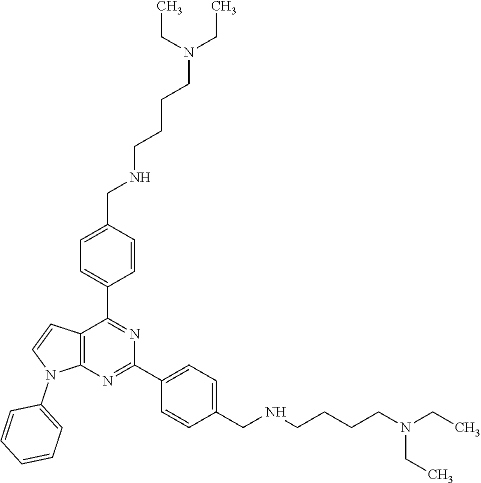 [SMILES: CCN(CC)CCCCNCc1ccc(-c2nc(-c3ccc(CNCCCCN(CC)CC)cc3)c3ccn(-c4ccccc4)c3n2)cc1] | (Ia-a) |
| 34A 34B | — Oxalate | C₄₈H₆₉N₇ C₄₈H₆₉N₇• 4(COOH)₂ | 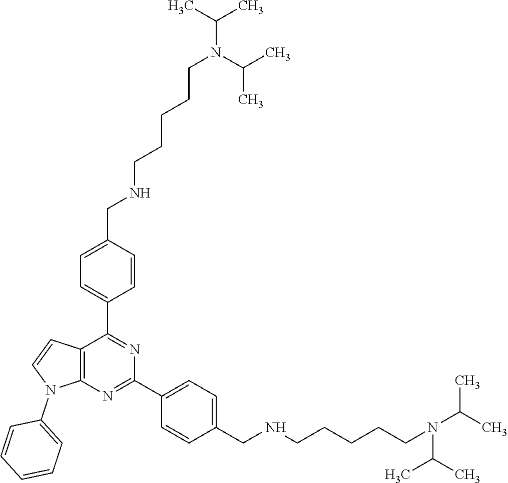 [SMILES: CC(C)N(CCCCCNCc1ccc(-c2nc(-c3ccc(CNCCCCCN(C(C)C)C(C)C)cc3)c3ccn(-c4ccccc4)c3n2)cc1)C(C)C] | (Ia-a) |
| 35A 35B | — Oxalate | C₄₀H₅₃N₇ C₄₀H₅₃N₇• 4(COOH)₂ | 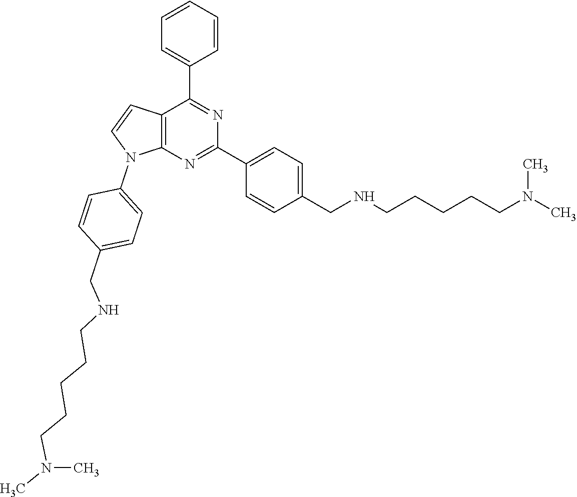 | (Ia-b) |
| 36A 36B | — Oxalate | C₃₅H₄₄N₈ C₃₅H₄₄N₈• 4(COOH)₂ | 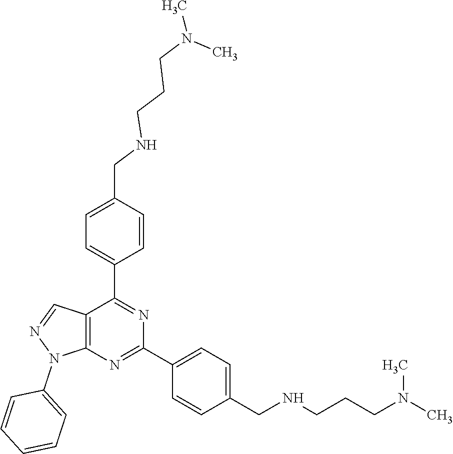 [SMILES: CN(C)CCCNCc1ccc(-c2nc(-c3ccc(CNCCCN(C)C)cc3)c3cnn(-c4ccccc4)c3n2)cc1] | (Ie-a) |
| 37A 37B | — Oxalate | C₃₇H₄₈N₈ C₃₇H₄₈N₈• 4(COOH)₂ | 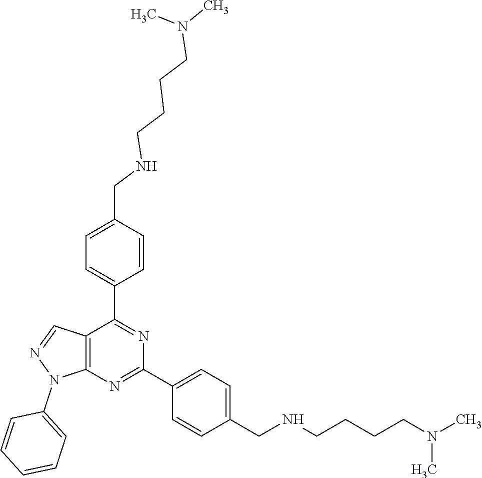 [SMILES: CN(C)CCCCNCc1ccc(-c2nc(-c3ccc(CNCCCCN(C)C)cc3)c3cnn(-c4ccccc4)c3n2)cc1] | (Ie-a) |
| 38A 38B | — Oxalate | C₃₉H₅₂N₈ C₃₉H₅₂N₈• 4(COOH)₂ | 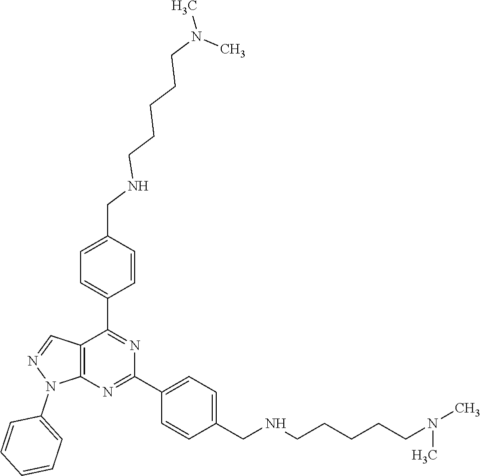 [SMILES: CN(C)CCCCCNCc1ccc(-c2nc(-c3ccc(CNCCCCCN(C)C)cc3)c3cnn(-c4ccccc4)c3n2)cc1] | (Ie-a) |
| 39A 39B | — Oxalate | C₄₃H₅₈N₁₀ C₄₃H₅₈N₁₀• 6(COOH)₂ | 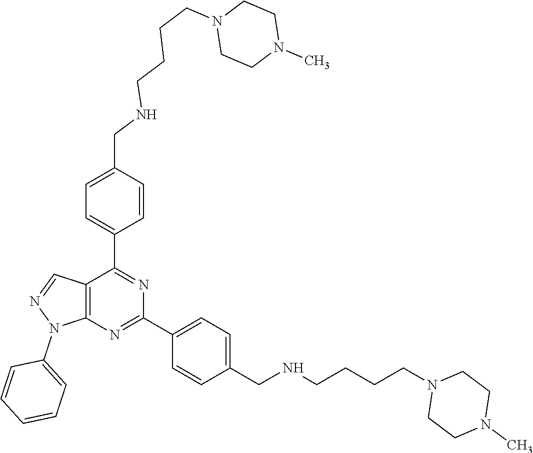 [SMILES: CN1CCN(CCCCNCc2ccc(-c3nc(-c4ccc(CNCCCCN5CCN(C)CC5)cc4)c4cnn(-c5ccccc5)c4n3)cc2)CC1] | (Ie-a) |
| 40A 40B | — Oxalate | C₃₇H₄₈N₈ C₃₇H₄₈N₈• 4(COOH)₂ | 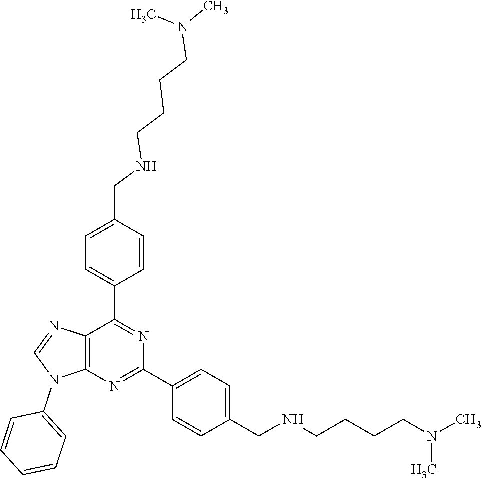 [SMILES: CN(C)CCCCNCc1ccc(-c2nc(-c3ccc(CNCCCCN(C)C)cc3)c3ncn(-c4ccccc4)c3n2)cc1] | (Ie-b) |
| 41A 41B | — Oxalate | C₃₉H₅₂N₈ C₃₉H₅₂N₈• 4(COOH)₂ | 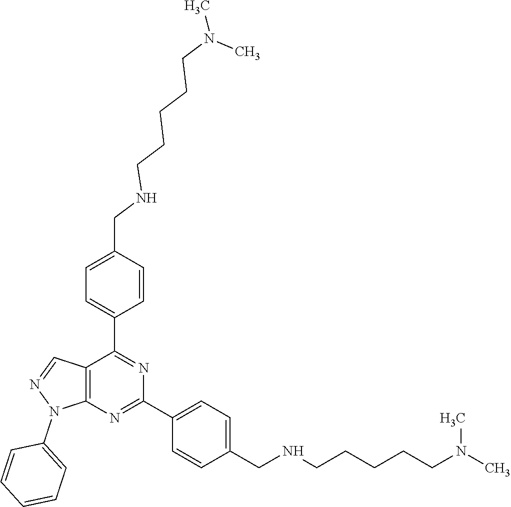 [SMILES: CN(C)CCCCCNCc1ccc(-c2nc(-c3ccc(CNCCCCCN(C)C)cc3)c3cnn(-c4ccccc4)c3n2)cc1] | (Ie-b) |
| 42A 42B | — Oxalate | C₄₃H₅₈N₁₀ C₄₃H₅₈N₁₀• 6(COOH)₂ | 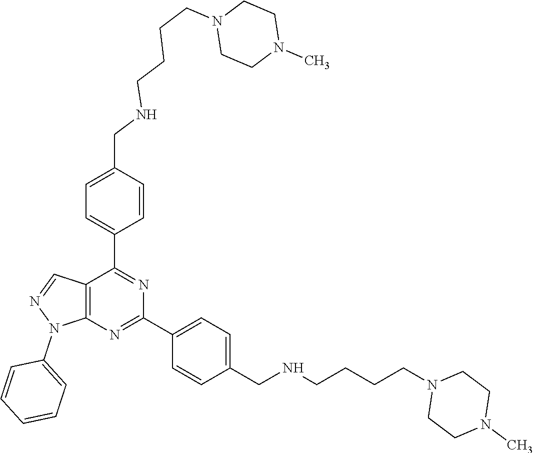 [SMILES: CN1CCN(CCCCNCc2ccc(-c3nc(-c4ccc(CNCCCCN5CCN(C)CC5)cc4)c4cnn(-c5ccccc5)c4n3)cc2)CC1] | (Ie-b) |
| 43A 43B | — Oxalate | C₂₃H₂₅ClN₆ C₂₃H₂₅ClN₆• 2(COOH)₂ | 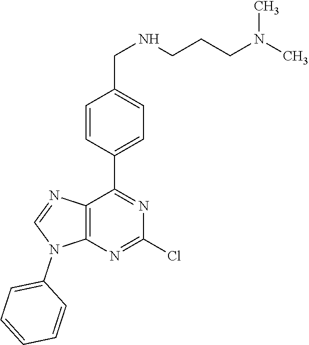 [SMILES: CN(C)CCCNCc1ccc(-c2nc(Cl)nc3c2ncn3-c2ccccc2)cc1] | (Ie-b) |
| 44A 44B | — Oxalate | C₃₃H₄₆N₆ C₃₃H₄₆N₆• 4(COOH)₂ | 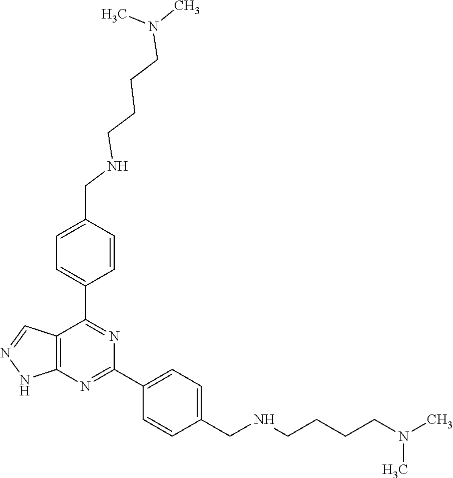 [SMILES: CN(C)CCCCNCc1ccc(-c2nc(-c3ccc(CNCCCCN(C)C)cc3)c3cn[nH]c3n2)cc1] | (If-a) |
| 45A 45B | — Oxalate | C₃₉H₅₆N₈ C₃₉H₅₆N₈• 6(COOH)₂ | 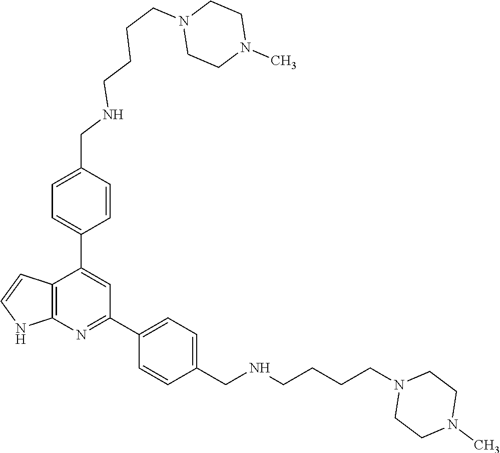 [SMILES: CN1CCN(CCCCNCc2ccc(-c3cc(-c4ccc(CNCCCCN5CCN(C)CC5)cc4)c4cc[nH]c4n3)cc2)CC1] | (If-a) |
| 46A 46B | — Oxalate | C₄₀H₅₂N₆ C₄₀H₅₂N₆• 4(COOH)₂ | 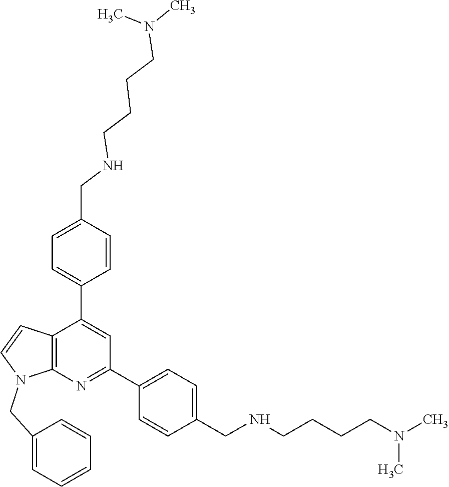 [SMILES: CN(C)CCCCNCc1ccc(-c2cc(-c3ccc(CNCCCCN(C)C)cc3)c3ccn(Cc4ccccc4)c3n2)cc1] | (If-a) |
| 47A 47B | — Oxalate | C₂₅H₂₇ClN₄ C₂₅H₂₇ClN₄• 2(COOH)₂ | 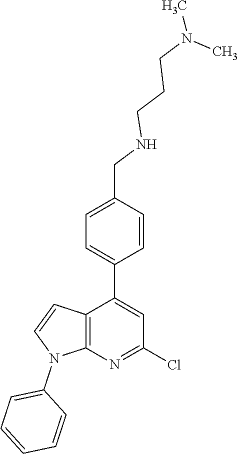 [SMILES: CN(C)CCCNCc1ccc(-c2cc(Cl)nc3c2ccn3-c2ccccc2)cc1] | (If-a) |
| 48A 48B | — Oxalate | C₃₉H₅₁N₇O C₃₉H₅₁N₇O• 4(COOH)₂ | 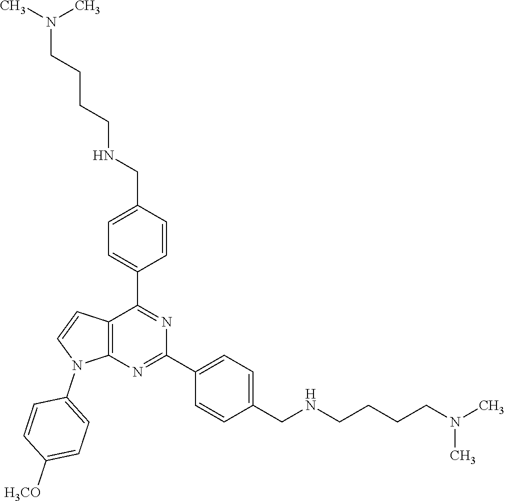 | (Ia-b) |
| 49A 49B | — Oxalate | C₃₈H₄₈N₆S C₃₈H₄₈N₆S• 4(COOH)₂ | 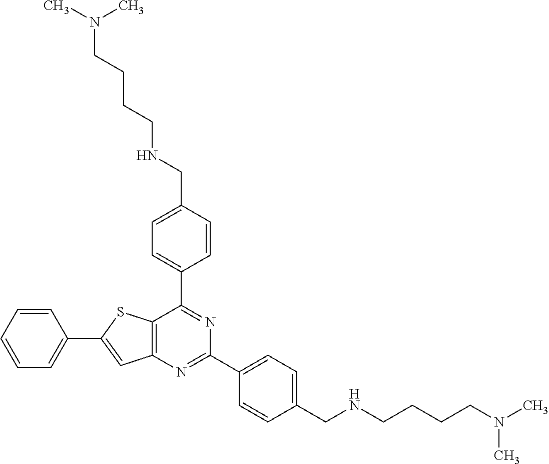 [SMILES: CN(C)CCCCNCc1ccc(-c2nc(-c3ccc(CNCCCCN(C)C)cc3)c3sc(-c4ccccc4)cc3n2)cc1] | (Ig-a) |

The below Table 3 describes the analytical and spectroscopic data of the compounds introduced in Table 1.

Melting points were determined with a SM-LUX-POL Leitz hot-stage microscope and are uncorrected. IR spectra were recorded on a NICOLET 380FT-IR spectrophotometer. ¹H NMR analyses (300 MHz) and ¹³C NMR spectra (75 MHz) were recorded with tetramethylsilane as an internal standard using a BRUKER AVANCE 300 spectrometer. Splitting patterns have been designated as follows: s=singlet; bs=broad singlet; d=doublet; t=triplet; q=quartette; qt=quintuplet, dd=double doublet; ddd=double doublet; and m=multiplet. Processing and analyses of the spectra were performed with MestReNova.

Analytical Thin-Layer Chromatography (TLC) was carried out on 0.25 precoated silica gel plates (POLYGRAM SIL G/UV254) and visualization of compounds after UV light irradiation. Silica gel 60 (70-230 mesh) was used for column chromatography. Microwave experiments were carried out at atmospheric pressure using a focused microwave reactor (CEM Discover). High-resolution mass spectra (electrospray in positive mode, ESIpor MALDI-TOF MS) were recorded on a Waters Q-TOF Ultima apparatus.

TABLE 3
Spectroscopic and analytical characterization of compounds (1) to (49)

| Cpd | Basic | | | |
|---|---|---|---|---|
| No | formula | Yield | Mp | Description |
| 1A | C₃₆H₄₅N₇ | 96% | | Pale-yellow oil. ¹H NMR δ (300 MHz, CDCl₃): 8.59 (d, 2H, J = 8.25 Hz, H-2′ and H-6′), 8.24 (d, |
| | | | | 2H, J = 8.25 Hz, H-2″ and H-6″), 7.89 (d, 2H, J = 8.10 Hz, H-2″′ and H-6″′), 7.59-7.52 (m, 5H, H-6, |
| | | | | H-3′, H-5′, H-3″ and H-5″), 7.43 (t, 2H, J = 8.10 Hz, H-3″′ and H-5″′), 7.38 (t, 1H, J = 8.10 Hz, H- |
| | | | | 4″′), 6.96 (d, 1H, J = 3.90 Hz, H-5), 3.91 (s, 2H, NCH₂), 3.86 (s, 2H, NCH₂), 2.73 (t, 2H, J = 7.20 |
| | | | | Hz, NCH₂), 2.69 (t, 2H, J = 7.20 Hz, NCH₂), 2.35 (t, 2H, J = 7.20 Hz, NCH₂), 2.32 (t, 2H, J = 7.20 |
| | | | | Hz, NCH₂), 2.24 (s, 6H, N(CH₃)₂), 2.22 (s, 6H, N(CH₃)₂), 1.77-1.64 (m, 4H, 2CH₂); ¹³C NMR δ (75 |
| | | | | MHz, CDCl₃): 159.6 (C-2), 158.9 (C-4), 154.0 (C-7a), 144.1 (C-4″′), 143.5 (C-4′), 139.2 (C-1″′), |
| | | | | 139.0 (C-1′), 138.7 (C-1″), 130.7 (C-2″ and C-6″), 130.5 (C-2′ and C-6′), 129.8 (C-3″′, C-5″′ and C- |
| | | | | 4″′), 129.6 (C-3′ and C-5′), 129.5 (C-3″ and C-5″), 127.9 (C-6), 125.1 (C-2″′ and C-6″′), 116.2 (C-4a), |
| | | | | 103.6 (C-5), 59.5 (NCH₂), 55.2 (NCH₂), 49.3 (NCH₂), 49.2 (NCH₂), 47.0 (N(CH₃)₂), 29.4 (CH₂) |
| | | | | MALDI-TOF MS m/z [M + H]⁺ Calculed: 576.3814, Found: 576.3778 |
| 1B | C₃₆H₄₅N₇•4(COOH)₂ | 70% | 179° | Beige crystals |
| | | | C. | |
| 2A | C₄₂H₅₅N₉ | 84% | | Yellow oil. ¹H NMR δ (300 MHz, CDCl₃): 8.55 (d, 2H, J = 8.40 Hz, H-2′ and H-6′), 8.19 (d, 2H, J = |
| | | | | 8.40 Hz, H-2″ and H-6″), 7.83 (d, 2H, J = 8.50 Hz, H-2″′ and H-6″′), 7.53-7.46 (m, 5H, H-3′, H-5′, |
| | | | | H-3″, H-5″ and H-6), 7.38 (d, 2H, J = 8.40 Hz, H-3″′ and H-5″′), 7.29 (t, 1H, J = 8.50 Hz, H-4″′), 6.89 |
| | | | | (d, 1H, J = 3.90 Hz, H-5), 3.84 (s, 2H, NCH₂), 3.80 (s, 2H, NCH₂), 2.67 (t, 2H, J = 6.90 Hz, NCH₂), |
| | | | | 2.64 (t, 2H, J = 6.90 Hz, NCH₂), 2.41-2.35 (m, 20H, 10NCH₂), 2.23 (s, 3H, NCH₃), 2.21 (s, 3H, |
| | | | | NCH₃), 1.71-1.64 (m, 4H, 2CH₂) ¹³C NMR δ (75 MHz, CDCl₃): 159.5 (C-2), 158.8 (C-4), 153.9 (C- |
| | | | | 7a), 144.0 (C-4″), 143.5 (C-4′), 139.2 (C-1″′), 138.9 (C-1′), 138.6 (C-1″), 130.6 (C-2″ and C-6″), 130.4 |
| | | | | (C-2′ and C-6′), 129.7 (C-3″′, C-5″′ and C-4″′), 129.6 (C-3′ and C-5′), 129.4 (C-3″ and C-5″), 127.9 |
| | | | | (C-6), 125.0 (C-2″′ and C-6″′), 116.1 (C-4a), 103.6 (C-5), 58.3 (NCH₂), 56.5 (NCH₂), 55.1 (NCH₂), |
| | | | | 54.6 (NCH₂), 49.5 (NCH₂), 49.4 (NCH₂), 47.4 (NCH₃), 28.3 (CH₂); MALDI-TOF MS m/z [M + H]⁺ |
| | | | | Calculed: 686.4658, Found: 686.4621 |
| 2B | C₄₂H₅₅N₉•6(COOH)₂ | 74% | 194° | White crystals |
| | | | C. | |
| 3A | C₃₈H₄₉N₇ | 67% | | Yellow oil. ¹H NMR δ (300 MHz, CDCl₃): 8.57 (d, 2H, J = 8.40 Hz, H-2′ and H-6′), 8.21 (d, 2H, J = |
| | | | | 8.40 Hz, H-2″ and H-6″), 7.84 (d, 2H, J = 7.80 Hz, H-2″′ and H-6″′), 7.56-7.49 (m, 5H, H-3′, H-5′, |
| | | | | H-3″, H-5″ and H-6), 7.40 (d, 2H, J = 7.80 Hz, H-3″′ and H-5″′), 7.32 (t, 1H, J = 7.80 Hz, H-4″′), 6.91 |
| | | | | (d, 1H, J = 3.60 Hz, H-5), 3.87 (s, 2H, NCH₂), 3.83 (s, 2H, NCH₂), 2.68-2.61 (m, 4H, 2NCH₂), 2.28 |
| | | | | (t, 2H, J = 7.20 Hz, NCH₂), 2.26 (t, 2H, J = 7.20 Hz, NCH₂), 2.20 (s, 6H, N(CH₃)₂), 2.17 (s, 6H, |
| | | | | N(CH₃)₂), 1.54-1.49 (m, 8H, 4CH₂); ¹³C NMR δ (75 MHz, CDCl₃): 159.5 (C-2), 158.8 (C-4), 153.9 |
| | | | | (C-7a), 143.9 (C-4″), 143.3 (C-4′), 139.2 (C-1″′), 139.0 (C-1′), 138.7 (C-1″), 130.6 (C-2″ and C-6″), |
| | | | | 130.4 (C-2′ and C-6′), 129.8 (C-3″′, C-5″′ and C-4″′), 129.6 (C-3′ and C-5′), 129.5 (C-3″ and C-5″), |
| | | | | 127.9 (C-6), 125.0 (C-2″′ and C-6″′), 116.1 (C-4a), 103.6 (C-5), 61.0 (NCH₂), 55.1 (NCH₂), 50.6 |
| | | | | (NCH₂), 50.5 (NCH₂), 46.8 (NCH₃), 29.3 (CH₂), 26.9 (CH₂); MALDI-TOF MS m/z [M + H]⁺ |
| | | | | Calculed: 604.4128, Found: 604.4089 |
| 3B | C₃₈H₄₉N₇•4(COOH)₂ | 89% | 146° | White crystals |
| | | | C. | |
| 4A | C₄₄H₅₉N₉ | 76% | | Yellow oil. ¹H NMR δ (300 MHz, CDCl₃): 8.59 (d, 2H, J = 8.10 Hz, H-2′ and H-6′), 8.25 (d, 2H, J = |
| | | | | 8.10 Hz, H-2″ and H-6″), 7.90 (d, 2H, J = 7.80 Hz, H-2″′ and H-6″′), 7.62-7.53 (m, 5H, H-3′, H-5′, |
| | | | | H-3″, H-5″ and H-6), 7.43 (t, 2H, J = 7.80 Hz, H-3″′ and H-5″′), 7.40 (t, 1H, J = 7.8 Hz, H-4″′), 6.99 |
| | | | | (d, 1H, J = 3.60 Hz, H-5), 3.92 (s, 2H, NCH₂), 3.87 (s, 2H, NCH₂), 2.72-2.66 (m, 4H, 2NCH₂), 2.56- |
| | | | | 2.30 (m, 20H, 10NCH₂), 2.28 (s, 3H, NCH₃), 2.27 (s, 3H, NCH₃), 1.58-1.52 (m, 8H, 4CH₂) MALDI- |
| | | | | TOF MS m/z [M + H]⁺ Calculed: 715.5050, Found: 715.5823 |
| 4B | C₄₄H₅₉N₉•6(COOH)₂ | 71% | 192° | Beige crystals |
| | | | C. | |
| 5A | C₄₀H₅₃N₇ | 98% | | Pale-yellow oil. ¹H NMR δ (300 MHz, CDCl₃): 8.56 (d, 2H, J = 8.10 Hz, H-2′ and H-6′), 8.19 (d, |
| | | | | 2H, J = 8.10 Hz, H-2″ and H-6″), 7.83 (d, 2H, J = 8.10 Hz, H-2″′ and H-6″′), 7.55-7.48 (m, 5H, H-3′, |
| | | | | H-5′, H-3″, H-5″ and H-6), 7.41-7.33 (m, 3H, H-3″′, H-4″′ and H-5″′), 6.90 (d, 1H, J = 3.60 Hz, H-5), |
| | | | | 3.86 (s, 2H, NCH₂), 3.82 (s, 2H, NCH₂), 2.67-2.58 (m, 4H, 2NCH₂), 2.18 (s, 6H, N(CH₂)₂), 2.17 (s, |
| | | | | 6H, N(CH₂)₂), 1.56-1.41 (m, 8H, 4CH₂), 1.36-1.28 (m, 4H, 2CH₂); ¹³C NMR δ (75 MHz, CDCl₃): |
| | | | | 159.5 (C-2), 158.8 (C-4), 153.9 (C-7a), 144.0 (C-4″), 143.5 (C-4′), 139.2 (C-1″′), 138.9 (C-1′), 138.6 |
| | | | | (C-1″), 130.6 (C-2″ and C-6″), 130.4 (C-2′ and C-6′), 129.8 (C-3″′, C-5″′ and C-4″′), 129.6 (C-3′ and |
| | | | | C-5′), 129.5 (C-3″ and C-5″), 127.9 (C-6), 125.0 (C-2″′ and C-6″′), 116.1 (C-4a), 103.6 (C-5), 61.1 |
| | | | | (NCH₂), 55.2 (NCH₂), 50.8 (NCH₂), 50.7 (NCH₂), 46.9 (N(CH₃)₂), 31.4 (CH₂), 29.0 (CH₂), 26.6 |
| | | | | (CH₂); MALDI-TOF MS m/z [M + H]⁺ Calculed: 632.4441, Found: 632.4437 |
| 5B | C₄₀H₅₃N₇•4(COOH)₂ | 76% | 201° | Pale-yellow crystals |
| | | | C. | |
| 6A | C₃₄H₄₃N₇S | 97% | | Pale-yellow oil. ¹H NMR δ (300 MHz, CDCl₃): 8.56 (d, 2H, J = 8.40 Hz, H-2′ and H-6′), 7.90 (d, |
| | | | | 1H, J = 3.60 Hz, H-6), 7.87 (d, 2H, J = 8.40 Hz, H-3′ and H-5′), 7.60-7.54 (m, 3H, H-2″, H-6″ and H- |
| | | | | thioph.), 7.44-7.36 (m, 3H, H-3″, H-5″ and H-thioph.), 7.06 (d, 1H, J = 3.60 Hz, H-thioph.), 7.00 (d, |
| | | | | 1H, J = 3.60 Hz, H-5), 4.08 (s, 2H, NCH₂), 3.87 (s, 2H, NCH₂), 2.78 (t, 2H, J = 6.90 Hz, NCH₂), 2.69 |
| | | | | (t, 2H, J = 6.90 Hz, NCH₂), 2.36 (t, 2H, J = 6.90 Hz, NCH₂), 2.33 (t, 2H, J = 6.90 Hz, NCH₂), 2.24 |
| | | | | (s, 6H, N(CH₃)₂), 2.23 (s, 6H, N(CH₃)₂), 1.79-1.68 (m, 4H, 2CH₂); ¹³C NMR δ (75 MHz, CDCl₃): |
| | | | | 159.3 (C-2), 154.1 (C-4), 152.8 (C-7a), 150.6 (C-1″), 143.6 (2 C-thioph.), 139.1 (C-4′), 138.6 (C-1′), |
| | | | | 130.7 (C-3″ and C-5″), 129.8 (C-thioph.), 129.6 (C-3′ and C-5′), 129.4 (C-2′, C-6′ and C-4″), 128.0 |
| | | | | (C-thioph.), 127.3 (C-6), 125.1 (C-2″ and C-6″), 113.7 (C-4a), 103.2 (C-5), 59.5 (NCH₂), 55.3 |
| | | | | (NCH₂), 50.4 (NCH₂), 49.2 (NCH₂), 47.0 (N(CH₃)₂), 29.4 (CH₂); MALDI-TOF MS m/z [M + H]⁺ |
| | | | | Calculed: 582.3379, Found: 582.3371 |
| 6B | C₃₄H₄₃N₇S•4(COOH)₂ | 72% | 221° | Pale-yellow crystals |
| | | | C. | |
| 7A | C₃₄H₄₃N₇S | 96% | | Yellow oil. ¹H NMR δ (300 MHz, CDCl₃): 8.22 (d, 2H, J = 8.10 Hz, H-2′ and H-6′), 7.93 (d, 1H, J = |
| | | | | 3.00 Hz, H-6), 7.89 (d, 2H, J = 8.10 Hz, H-3′ and H-5′), 7.61-7.52 (m, 5H, H-2″, H-6″, H-3″, H-5″ |
| | | | | and H-thioph.), 7.42 (t, 1H, J = 7.80 Hz, H-4″), 6.99-6.94 (m, 2H, H-5 and H-thioph.), 4.03 (s, 2H, |
| | | | | NCH₂), 3.93 (s, 2H, NCH₂), 2.75 (t, 4H, J = 7.20 Hz, 2 NCH₂), 2.37 (t, 2H, J = 7.20 Hz, NCH₂), 2.34 |
| | | | | (t, 2H, J = 7.20 Hz, NCH₂), 2.26 (s, 6H, N(CH₃)₂), 2.24 (s, 6H, N(CH₃)₂), 1.77-1.69 (m, 4H, 2CH₂); |
| | | | | ¹³C NMR δ (75 MHz, CDCl₃): 159.0 (C-2), 153.6 (C-4), 149.0 (C-7a), 148.1 (C-1″), 144.9 (C- |
| | | | | thioph.), 144.1 (C-thioph.), 139.1 (C-4′), 138.3 (C-1′), 130.7 (C-3″ and C-5″), 130.5 (C-3′ and C-5′), |
| | | | | 129.8 (C-2′ and C-6′), 129.5 (C-thioph.), 128.7 (C-thioph.), 128.0 (C-4″), 127.2 (C-6), 125.0 (C-2″ |
| | | | | and C-6″), 112.6 (C-4a), 103.9 (C-5), 59.5 (NCH₂), 55.2 (NCH₂), 50.4 (NCH₂), 49.3 (NCH₂), 49.0 |
| | | | | (NCH₂), 47.0 (N(CH₃)₂), 29.4 (CH₂); MALDI-TOF MS m/z [M + H]⁺ Calculated: 582.3379, Found: |
| | | | | 582.3367 |
| 7B | C₃₄H₄₃N₇S•4(COOH)₂ | 62% | 178° | Beige crystals |
| | | | C. | |
| 8A | C₃₁H₄₃N₇ | 98% | | Yellow oil. ¹H NMR δ (300 MHz, CDCl₃): 8.63 (d, 2H, J = 8.10 Hz, H-2′ and H-6′), 8.24 (d, 2H, J = |
| | | | | 8.10 Hz, H-2″ and H-6″), 7.51 (d, 2H, J = 8.10 Hz, H-3′, H-5′), 7.45 (d, 2H, J = 8.10 Hz, H-3″ and |
| | | | | H-5″), 7.19 (d, 1H, J = 3.60 Hz, H-6), 6.79 (d, 1H, J = 3.60 Hz, H-5), 3.94 (s, 2H, NCH₃), 3.90 (s, |
| | | | | 2H, NCH₂), 3.88 (s, 2H, NCH₂), 2.74-6.65 (m, 4H, 2NCH₂), 2.37-2.30 (m, 4H, 2NCH₂), 2.23 (s, 6H, |
| | | | | N(CH₃)₂), 2.22 (s, 6H, N(CH₃)₂), 1.80-1.69 (m, 4H, 2CH₂); ¹³C NMR δ (75 MHz, CDCl₃): 158.8 (C- |
| | | | | 2), 158.0 (C-4), 154.4 (C-7a), 143.7 (C-4″), 143.0 (C-4′), 139.1 (C-1′), 138.8 (C-1″), 131.2 (C-6), |
| | | | | 130.4 (C-2″ and C-6″), 129.7 (C-2′ and C-6′), 129.5 (C-3″, C-5″, C-3′ and C-5′), 115.1 (C-4a), 101.6 |
| | | | | (C-5), 59.4 (NCH₂), 55.1 (NCH₂), 49.2 (NCH₂), 46.9 (N(CH₃)₂), 32.4 (NH3), 29.3 (CH₂); MALDI- |
| | | | | TOF MS m/z [M + H]⁺ Calculed: 514.3658, Found: 514.3664 |
| 8B | C₃₁H₄₃N₇•4(COOH)₂ | 53% | 180° | Pale-yellow crystals |
| | | | C. | |
| 9A | C₃₇H₅₃N₉ | 95% | | Yellow oil. ¹H NMR δ (300 MHz, CDCl₃): 8.60 (d, 2H, J = 8.10 Hz, H-2′ and H-6′), 8.21 (d, 2H, J = |
| | | | | 8.10 Hz, H-2″ and H-6″), 7.48 (d, 2H, J = 8.10 Hz, H-3′ and H-5′), 7.41 (d, 2H, J = 8.10 Hz, H-3″, |
| | | | | H-5″), 7.16 (d, 1H, J = 3.60 Hz, H-6), 6.76 (d, 1H, J = 3.60 Hz, H-5), 3.91 (s, 3H, NCH₃), 3.86 (s, |
| | | | | 2H, NCH₂), 3.84 (s, 2H, NCH₂), 2.71-2.64 (m, 4H, 2NCH₂), 2.57-2.34 (m, 20H, 10NCH₂), 2.24 (s, |
| | | | | 6H, 2NCH₃), 1.75-1.68 (m, 4H, 2CH₂) ¹³C NMR δ (75 MHz, CDCl₃): 158.8 (C-2), 158.1 (C-4), |
| | | | | 154.4 (C-7a), 143.8 (C-4″), 143.2 (C-4′), 139.2 (C-1′), 138.8 (C-1″), 131.2 (C-6), 130.4 (C-2″ and C- |
| | | | | 6″), 129.7 (C-2′ and C-6′), 129.4 (C-3′, C-5′, C-3″ and C-5″), 115.1 (C-4a), 101.6 (C-5), 58.4 (NCH₂), |
| | | | | 56.5 (NCH₂ₚᵢₚ), 55.1 (NCH₂), 54.6 (NCH₂ₚᵢₚ), 49.4 (NCH₂), 47.4 (NCH₃), 32.4 (NCH₃), 28.3 (CH₂); |
| | | | | MALDI-TOF MS m/z [M + H]⁺ Calculed: 624.4502, Found: 624.4487 |
| 9B | C₃₇H₅₃N₉•6(COOH)₂ | 45% | 204° | Beige crystals |
| | | | C. | |
| 10A | C₄₅H₆₃N₇O₈ | 75% | | Yellow oil. ¹H NMR δ (300 MHz, CDCl₃): 8.62 (d, 2H, J = 8.40 Hz, H-2′ and H-6′), 8.23 (d, |
| | | | | 2H, J = 8.40 Hz, H-2″ and H-6″), 7.53 (d, 2H, J = 8.40 Hz, H-3′ and H-5′), 7.48 (d, 2H, J = 8.40 Hz, |
| | | | | H-3″ and H-5″), 7.20 (d, 1H, J = 3.60 Hz, H-6), 6.78 (d, 1H, J = 3.60 Hz, H-5), 5.14 (d, 2H, J = 7.80 |
| | | | | Hz, 2NH), 4.31-4.27 (m, 2H, 2CH), 3.94 (s, 3H, NCH₃), 3.90 (s, 4H, 2NCH₂), 3.74 (s, 3H, COOCH₃), |
| | | | | 3.73 (s, 3H, COOCH₃), 3.19 (bs, 2H, 2NH), 2.70 (t, 4H, J = 7.20 Hz, 2NCH₂), 1.87-1.43 (m, 12H, |
| | | | | 6CH₂). 1.40 (s, 18H, 2C(CH₃)₃). MALDI-TOF MS m/z [M + H]⁺ Calculed: 830.4816, Found: |
| | | | | 830.4827. |
| 10B | C₄₅H₆₃N₇O₈ | 72% | 173° | Pale-yellow crystals |
| | 2(COOH)₂ | | C. | |
| 11A | C₃₅H₄₇N₇O₄ | 82% | | Yellow oil. ¹H NMR δ (300 MHz, CDCl₃): 8.64 (d, 2H, J = 8.40 Hz, H-2′ and H-6′), 8.24 (d, 2H, J = |
| | | | | 8.40 Hz, H-2″ and H-6″), 7.52 (d, 2H, J = 8.40 Hz, H-3′ and H-5′), 7.46 (d, 2H, J = 8.40 Hz, H-3″ |
| | | | | and H-5″), 7.23 (d, 1H, J = 3.60 Hz, H-6), 6.83 (d, 1H, J = 3.60 Hz, H-5), 3.98 (s, 3H, NCH₃), 3.91 |
| | | | | (s, 2H, NCH₂), 3.89 (s, 2H, NCH₂), 3.73 (s, 3H, COOCH₃), 3.72 (s, 3H, COOCH₃), 3.50-3.44 (m, |
| | | | | 2H, 2CH), 2.73-2.65 (m, 4H, 2NCH₂), 1.83-1.43 (m, 12H, 6CH₂). MALDI-TOF MS m/z [M + H]⁺ |
| | | | | Calculed: 630.3768, Found: 630.3769. |
| 11B | C₃₅H₄₇N₇O₄ | 57% | 211° | Yellow crystals |
| | 4(COOH)₂ | | C. | |
| 12A | C₃₀H₃₁N₅ | 76% | | Pale-yellow oil. ¹H NMR δ (300 MHz, CDCl₃): 8.67 (dd, 2H, J = 8.10 Hz and J = 1.50 Hz, H-2′ and |
| | | | | H-6′), 8.30 (dd, 2H, J = 8.10 Hz and J = 1.50 Hz, H-2″ and H-6″), 7.87 (d, 2H, J = 8.40 Hz, H-2″′ and |
| | | | | H-6″′), 7.65-7.46 (m, 9H, H-3′, H-4′, H-5′, H-3″, H-4″, H-5″, H-3″′, H-5″′ and H-6) 7.00 (d, 1H, J = |
| | | | | 3.60 Hz, H-5), 3.91 (s, 2H, NCH₂), 2.76 (t, 2H, J = 7.20 Hz, NCH₂), 2.39 (t, 2H, J = 7.20 Hz, NCH₂), |
| | | | | 2.27 (s, 6H, N(CH₃)₂), 1.98 (bs, 1H, NH), 1.77 (qt, 2H, J = 7.20 Hz, CH₂). ¹³C NMR δ (75 MHz, |
| | | | | CDCl₃): 159.6 (C-2), 159.1 (C-4), 157.0 (C-7a), 140.4 (C-1″′), 140.2 (C-4″′), 139.8 (C-1″), 137.9 (C- |
| | | | | 1′), 131.4 (C-4″), 131.1 (C-4′), 130.4 (C-3″′, C-5″′, C-3″ and C-5″ and C-6), 130.1 (C-3′ and C-5′), |
| | | | | 129.7 (C-2″ and C-6″), 129.6 (C-2′ and C-6′), 125.1 (C-2″′ and C-6″′), 116.3 (C-4a), 103.5 (C-5), 59.5 |
| | | | | (NCH₂), 55.0 (NCH₂), 49.4 (NCH₂), 47.0 (N(CH₃)₂), 29.4 (CH₂); MALDI-TOF MS m/z [M + H]⁺ |
| | | | | Calculed: 462.2657, Found: 462.2637 |
| 12B | C₃₀H₃₁N₅ | 58% | 238° | White crystals |
| | 2(COOH)₂ | | C. | |
| 13A | C₄₂H₅₉N₉ | 86% | | Yellow oil. ¹H NMR δ (300 MHz, CDCl₃): 8.60 (d, 2H, J = 8.40 Hz, H-2′ and H-6′), 8.25 (d, 2H, J = |
| | | | | 8.40 Hz, H-2″ and H-6″), 7.87 (d, 2H, J = 8.40 Hz, H-2″′ and H-6″′), 7.60-7.52 (m, 5H, H-6, H-3′, |
| | | | | H-5′, H-3″ and H-5″), 7.44 (d, 2H, J = 8.40 Hz, H-3″′ and H-5″′), 6.99 (d, 1H, J = 3.60 Hz, H-5), 3.93 |
| | | | | (s, 2H, NCH₂), 3.91 (s, 2H, NCH₂), 3.88 (s, 2H, NCH₂), 2.79-2.68 (m, 6H, 3NCH₂), 2.40-2.28 (m, |
| | | | | 6H, 3NCH₂), 2.26 (s, 6H, N(CH₃)₂), 2.24 (s, 6H, N(CH₃)₂), 2.23 (s, 6H, N(CH₃)₂), 1.79-1.66 (m, 6H, |
| | | | | 3CH₂); ¹³C NMR δ (75 MHz, CDCl₃): 159.6 (C-2), 159.0 (C-4), 154.0 (C-7a), 144.0 (C-1″′), 143.5 |
| | | | | (C-4′), 1404.4 (C-4″) 139.0 (C-4″′), 138.7 (C-1″), 138.0 (C-1′), 131.2 (C-6), 130.5 (C-3″′ and C-5″′), |
| | | | | 130.4 (C-3′ and C-5′), 129.5 (C-3″ and C-5″), 129.6 (C-2′ and C-6′), 129.5 (C-2″ and C-6″), 125.1 (C- |
| | | | | 2′″ and C-6″′), 116.1 (C-4a), 103.5 (C-5), 59.5 (NCH₂), 55.2 (NCH₂), 55.0 (NCH₂), 54.8 (NCH₂), 49.3 |
| | | | | (NCH₂), 49.2 (NCH₂), 47.0 (N(CH₃)₂), 29.5 (CH₂); MALDI-TOF MS m/z [M + 2H]⁺ Calculed: |
| | | | | 691.5050, Found: 691.5035 |
| 13B | C₄₂H₅₉N₉•6(COOH)₂ | 42% | 145° | Pale-yellow crystals |
| | | | C. | |
| 14A | C₂₉H₃₉N₇ | 95% | | Yellow oil. ¹H NMR δ (300 MHz, CDCl₃): 8.00 (d, 2H, J = 8.10 Hz, H-2′ and H-6′), 7.78 (d, 1H, J = |
| | | | | 8.10 Hz, H-3′ and H-5′), 7.47 (t, 2H, J = 7.80 Hz, H-3″ and H-5″), 7.45 (d, 2H, J = 7.80 Hz, H-2″ |
| | | | | and H-6″), 7.28 (t, 1H, J = 7.80 Hz, H-4″), 7.17 (d, 1H, J = 3.75 Hz, H-6), 6.71 (d, 1H, J = 3.75 Hz, |
| | | | | H-5), 3.85 (s, 2H, NCH₂), 3.51 (q, 2H, J = 6.90 Hz, NCH₂), 2.68 (t, 2H, J = 6.90 Hz, NCH₂), 2.40- |
| | | | | 2.27 (m, 4H, 2 NCH₂), 2.21 (s, 6H, N(CH₃)₂), 2.20 (s, 6H, N(CH₃)₂), 1.84-1.77 (m, 2H, CH₂), 1.72- |
| | | | | 1.63 (m, 2H, CH₂); MALDI-TOF MS m/z [M + H]⁺ Calculed: 486.33, Found: 486.33 |
| 14B | C₂₉H₃₉N₇•4(COOH)₂ | 67% | 134° | Yellow crystals |
| | | | C. | |
| 15A | C₁₈H₂₂ClN₅ | 86% | | Yellow oil. ¹H NMR δ (300 MHz, CDCl₃):, 8.08 (d, 2H, J = 8.40 Hz, H-2′ and H-6′), 7.47 (d, 2H, J = |
| | | | | 8.40 Hz, H-3′ and H-5′), 7.37 (d, 1H, J = 3.60 Hz, H-6), 6.79 (d, 1H, J = 3.60 Hz, H-5), 3.89 (s, 2H, |
| | | | | NCH₂), 2.73 (t, 2H, J = 6.90 Hz, NCH₂), 2.38 (t, 2H, J = 6.90 Hz, NCH₂), 2.30 (s, 6H, N(CH₃)₂), 1.75 |
| | | | | (qt, 2H, J = 6.90 Hz, CH₂); ¹³C NMR δ (75 MHz, CDCl₃): 166.5 (C-2), 155.6 (C-4), 154.2 (C-7a), |
| | | | | 144.7 (C-4′), 137.0 (C-1′), 130.5 (C-2′ and C-6′), 129.8 (C-3′ and C-5′), 128.4 (C-6), 115.8 (C-4a), |
| | | | | 102.7 (C-5), 59.4 (NCH₂), 55.0 (NCH₂), 49.3 (NCH₂), 46.8 (N(CH₃)₂), 29.3 (CH₂); MALDI-TOF MS |
| | | | | m/z [M + H]⁺ Calculed: 344.1642, Found: 344.1631 |
| 15B | C₁₈H₂₂ClN₅•2(COOH)₂ | 92% | 192° | White crystals |
| | | | C. | |
| 16A | C₂₁H₂₇ClN₆ | 97% | | Pale-yellow oil. ¹H NMR δ (300 MHz, CDCl₃): 8.09 (d, 2H, J = 8.40 Hz, H-2′ and H-6′), 7.48 (d, |
| | | | | 2H, J = 8.40 Hz, H-3′ and H-5′), 7.39 (d, 1H, J = 3.60 Hz, H-6), 6.81 (d, 1H, J = 3.60 Hz, H-5), 3.88 |
| | | | | (s, 2H, NCH₂), 2.72 (t, 2H, J = 6.90 Hz, NCH₂), 2.54-2.44 (m, 11H, NH and 5NCH₂), 2.28 (s, 3H, |
| | | | | NCH₃), 1.75 (qt, 2H, J = 6.90 Hz, CH₂); ¹³C NMR δ (75 MHz, CDCl₃): 160.6 (C-2), 155.6 (C-4), |
| | | | | 154.3 (C-7a), 144.7 (C-4′), 137.1 (C-1′), 130.5 (C-2′ and C-6′), 129.8 (C-2′ and C-5′), 128.3 (C-6), |
| | | | | 115.8 (C-4a), 102.7 (C-5), 58.3 (NCH₂), 56.5 (NCH₂ₚᵢₚ), 55.0 (NCH₂), 54.5 (NCH₂ₚᵢₚ), 49.5 (NCH₂), |
| | | | | 47.3 (NCH₃), 28.2 (CH₂); MALDI-TOF MS m/z [M + H]⁺ Calculed: 399.2064, Found: 399.2052 |
| 16B | C₂₁H₂₇ClN₆•3(COOH)₂ | 57% | 215° | Pale-yellow crystals |
| | | | C. | |
| 17A | C₂₄H₂₆ClN₅ | 97% | | Colorless oil. ¹H NMR δ (300 MHz, CDCl₃): 8.07 (d, 2H, J = 8.10 Hz, H-2′ and H-6′), 7.68 (d, 2H, |
| | | | | J = 8.10 Hz, H-3′and H-5′), 7.54-7.48 (m, 5H, H-2″, H-6″, H-3″, H-5″ and H-6), 7.37 (t, 1H, J = 7.50 |
| | | | | Hz, H-4″), 6.93 (d, 1H, J = 3.60 Hz, H-5), 3.87 (s, 2H, NCH₂), 2.69 (t, 2H, J = 6.90 Hz, NCH₂), 2.33 |
| | | | | (t, 2H, J = 6.90 Hz, NCH₂), 2.23 (s, 6H, N(CH₃)₂), 2.08 (bs, 1H, NH), 1.70 (qt, 2H, J = 6.90 Hz, |
| | | | | 2CH₂); ¹³C NMR δ (75 MHz, CDCl₃): 161.2 (C-2), 155.7 (C-4), 154.0 (C-7a), 144.9 (C-4′), 138.3 |
| | | | | (C-1′), 136.9 (C-1″), 130.9 (C-2′ and C-6′), 130.8 (C-4″), 130.5 (C-2″ and C-5″), 129.9 (C-3′ and C- |
| | | | | 5′), 128.8 (C-6), 125.3 (C-3″ and C-5″), 116.6 (C-4a), 103.7 (C-5), 59.4 (NCH₂), 55.1 (NCH₂), 49.3 |
| | | | | (NCH₂), 46.9 (N(CH₃)₃), 29.4 (CH₂); MALDI-TOF MS m/z [M + H]⁺ Calculed: 420.1955, Found: |
| | | | | 420.1943 |
| 17B | C₂₄H₂₆ClN₅•2(COOH)₂ | 62% | 217° | White crystals |
| | | | C. | |
| 18A | C₂₇H₃₁ClN₆ | 93% | | Pale-yellow oil. ¹H NMR δ (300 MHz, CDCl₃): 8.09 (d, 2H, J = 8.40 Hz, H-2′ and H-6′), 7.71 (d, |
| | | | | 2H, J = 8.40 Hz, H-3′ and H-5′), 7.58-7.50 (m, 5H, H-2″, H-6″, H-3″, H-5″ and H-6), 7.40 (t, 1H, J = |
| | | | | 7.40 Hz, H-4″), 6.96 (d, 1H, J = 3.90 Hz, H-5), 3.89 (s, 2H, NCH₂), 2.71 (t, 2H, J = 6.90 Hz, NCH₂), |
| | | | | 2.58-2.41 (m, 10H, 5NCH₂), 2.28 (s, 3H, NCH₃), 2.05 (bs, 1H, NH), 1.78 (qt, 2H, J = 6.90 Hz, CH₂) |
| | | | | ¹³C NMR δ (75 MHz, CDCl₃): 161.2 (C-2), 154.0 (C-4), 153.6 (C-7a), 144.9 (C-4′), 138.3 (C-1′), |
| | | | | 136.9 (C-1″), 131.6 (C-2′ and C-6′), 131.3 (C-4″), 131.0 (C-2″ and C-6″), 130.7 (C-3′ and C-5′), 128.8 |
| | | | | (C-6), 125.4 (C-3″ and C-5″), 116.1 (C-4a), 103.7 (C-5), 58.4 (NCH₂), 56.5 (NCH₂ₚᵢₚ), 55.1 (NCH₂), |
| | | | | 54.6 (NCH₂ₚᵢₚ), 49.5 (NCH₂), 47.4 (NCH₃), 28.3 (CH₂); MALDI-TOF MS m/z [M + H]⁺ Calculed: |
| | | | | 475.2077, Found: 475.2367. |
| 18B | C₂₇H₃₁ClN₆•3(COOH)₂ | 76% | 249° | Pale yellow crystals |
| | | | C. | |
| 19A | C₂₂H₂₄ClN₅S | 95% | | Yellow oil. ¹H NMR δ (300 MHz, CDCl₃): 7.91 (d, 1H, J = 3.60 Hz, H-6), 7.69 (d, 2H, J = 8.40 Hz, |
| | | | | H-2′ and H-6′), 7.54 (t, 2H, J = 8.40 Hz, H-3′ and H-5′), 7.51 (d, 1H, J = 3.60 Hz, H-thioph), 7.38 (t, |
| | | | | 1H, J = 8.10 Hz, H-4′), 7.07 (d, 1H, J = 3.60 Hz, H-thioph), 7.02 (d, 1H, J = 3.60 Hz, H-5), 4.06 (s, |
| | | | | 2H, NH₂), 2.76 (t, 2H, J = 7.20 Hz, NCH₂), 2.36 (t, 2H, J = 7.20 Hz, NCH₂), 2.24 (s, 6H, N(CH₃)₂), |
| | | | | 1.85 (bs, 1H, NH), 1.72 (qt, 2H, J = 7.20 Hz, CH₂); ¹³C NMR δ (75 MHz, CDCl₃): 155.3 (C-2), 154.8 |
| | | | | (C-4), 154.1 (C-7a), 152.3 (C-1′), 141.9 (C-thioph), 138.2 (C-thioph), 131.3 (C-thioph), 130.9 (C-3′ |
| | | | | and C-5′), 130.6 (C-4′), 128.8 (C-thioph), 127.3 (C-6), 125.4 (C-2′ and C-6′), 114.2 (C-4a), 103.4 (C- |
| | | | | 5), 59.3 (NCH₂), 50.2 (NCH₂), 49.1 (NCH₂), 46.9 (N(CH₃)₂), 29.3 (CH₂); MALDI-TOF MS m/z |
| | | | | [M + H]⁺ Calculed: 426.1519, Found: 426.1513. |
| 19B | C₂₂H₂₄ClN₅S•2(COOH)₂ | 67% | 235° | Yellow crystals |
| | | | C. | |
| 20A | C₂₂H₂₄ClN₅S | 84% | | Yellow oil. ¹H NMR δ (300 MHz, CDCl₃): 8.09 (d, 1H, J = 1.20 Hz, H-thioph), 7.70 (dd, 1H, J = |
| | | | | 1.20 × 1.20 Hz, H-thioph), 7.68 (d, 2H, J = 7.50 Hz, H-2′ and H-6′), 7.53 (t, 2H, J = 7.50 Hz, H-3′ and |
| | | | | H-5′), 7.51 (d, 1H, J = 3.60 Hz, H-6), 7.38 (t, 1H, J = 7.50 Hz, H-4′), 6.94 (d, 1H, J = 3.60 Hz, H-5), |
| | | | | 4.05 (s, 2H, NH₂), 2.75 (t, 2H, J = 7.20 Hz, NCH₂), 2.34 (t, 2H, J = 7.20 Hz, NCH₂), 2.23 (s, 6H, |
| | | | | N(CH₃)₂), 1.91 (bs, 1H, NH), 1.71 (qt, 2H, J = 7.20 Hz, CH₂); ¹³C NMR δ (75 MHz, CDCl₃): 156.2 |
| | | | | (C-4), 155.6 (C-2), 154.1 (C-7a), 147.5 (C-thioph), 140.1 (C-thioph), 138.3 (C-1′), 130.9 (C-3′ and |
| | | | | C-5′), 130.6 (C-thioph), 129.2 (C-thioph), 128.8 (C-4′), 126.2 (C-6), 125.4 (C-2′ and C-6′), 115.6 (C- |
| | | | | 4a), 103.4 (C-5), 59.4 (NCH₂), 50.0 (NCH₂), 49.1 (NCH₂), 47.0 (N(CH₃)₂), 29.3 (CH₂); MALDI-TOF |
| | | | | MS m/z [M + H]⁺ Calculed: 426.1519, Found: 426.1619. |
| 20B | C₂₂H₂₄ClN₅S•2(COOH)₂ | 65% | 223° | White crystals |
| | | | C. | |
| 21A | C₂₂H₂₄ClN₅S | 97% | | Yellow oil. ¹H NMR δ (300 MHz, CDCl₃): 7.72 (d, 2H, J = 7.50 Hz, H-2′ and H-6′), 7.59 (d, 1H, J = |
| | | | | 5.40 Hz, H-thioph.), 7.57 (t, 2H, J = 7.50 Hz, H-3′ and H-5′), 7.52 (d, 1H, J = 3.70 Hz, H-6), 7.42 |
| | | | | (t, 1H, J = 7.50 Hz, H-4′), 7.34 (d, 1H, J = 5.40 Hz, H-thioph.), 6.85 (d, 1H, J = 3.70 Hz, H-5), 4.20 |
| | | | | (s, 2H, NH₂), 2.77 (t, 2H, J = 7.20 Hz, NCH₂), 2.40 (bs, 1H, NH), 2.36 (t, 2H, J = 7.20 Hz, NCH₂), |
| | | | | 2.23 (s, 6H, N(CH₃)₂), 1.76 (qt, 2H, J = 7.20 Hz, CH₂); ¹³C NMR δ (75 MHz, CDCl₃): 157.6 (C-4), |
| | | | | 154.9 (C-2), 153.9 (C-7a), 149.3 (C-1′), 138.3 (C-thioph.), 135.4 (C-thioph.), 131.0 (C-3′ and C-5′), |
| | | | | 130.4 (C-4′), 130.0 (C-thioph.), 128.9 (C-thioph.), 125.4 (C-2′ and C-6′), 125.2 (C-6), 117.0 (C-4a), |
| | | | | 103.6 (C-5), 59.4 (NCH₂), 49.1 (NCH₂), 49.0 (NCH₂), 46.9 (N(CH₃)₂), 29.6 (CH₂); MALDI-TOF MS |
| | | | | m/z [M + H]⁺ Calculed: 426.1519, Found: 426.1554 |
| 21B | C₂₂H₂₄ClN₅S•2(COOH)₂ | 71% | 175° | Pale-yellow crystals |
| | | | C. | |
| 22A | C₂₄H₂₆ClN₅ | 92% | | Pale-yellow crystals. ¹H NMR δ (300 MHz, CDCl₃): 8.16-8.09 (m, 2H, H-2′ and H-6′), 7.65 (d, 2H, |
| | | | | J = 8.40 Hz, H-2″ and H-6″), 7.56-7.47 (m, 6H, H-3′, H-4′, H-5′, H-3″, H-5″, and H-6), 6.94 (d, |
| | | | | 1H, J = 3.60 Hz, H-5), 3.85 (s, 2H, NCH₂), 2.71 (t, 2H, J = 6.90 Hz, NCH₂), 2.35 (t, 2H, J = 6.90 Hz, |
| | | | | NCH₂), 2.23 (s, 6H, N(CH₃)₂), 1.93 (bs, 1H, NH), 1.71 (qt, 2H, J = 6.90 Hz, CH₂). ¹³C NMR δ (75 |
| | | | | MHz, CDCl₃): 161.3 (C-2), 155.7 (C-4), 154.0 (C-7a), 141.4 (C-4″), 138.3 (C-1″), 136.9 (C-1′), 132.1 |
| | | | | (C-2″ and C-6″), 130.5 (C-4′), 130.3 (C-3″, C-5″, and C-6), 125.3 (C-3′ and C-5′), 116.7 (C-4a), 103.7 |
| | | | | (C-5), 59.4 (NCH₂), 54.9 (NCH₂), 49.3 (NCH₂), 47.0 (N(CH₃)₃), 29.4 (CH₂); MALDI-TOF MS m/z |
| | | | | [M + H]⁺ Calculed: 420.1955, Found: 420.1938. |
| 22B | C₂₄H₂₆ClN₅•2(COOH)₂ | 73% | 208° | White crystals |
| | | | C. | |
| 23A | C₂₇H₃₁ClN₆ | 81% | | Pale-yellow oil. ¹H NMR δ (300 MHz, CDCl₃): 8.14-8.08 (m, 2H, H-2′ and H-6′), 7.65 (d, 2H, J = |
| | | | | 8.40 Hz, H-2″, H-6″), 7.57-7.53 (m, 3H, H-3′, H-4′ and H-5′), 7.51 (d, 1H, J = 3.60 Hz, H-6), 7.48 (d, |
| | | | | 2H, J = 8.40 Hz, H-3″ and H-5″) 6.95 (d, 1H, J = 3.60 Hz, H-5), 3.85 (s, 2H, NCH₂), 2.71 (t, 2H, J = |
| | | | | 6.90 Hz, NCH₂), 2.61-2.43 (m, 10H, 5NCH₂), 2.28 (s, 3H, NCH₃), 1.74 (qt, 2H, J = 6.90 Hz, CH₂) |
| | | | | ¹³C NMR δ (75 MHz, CDCl₃): 161.4 (C-2), 155.7 (C-4), 154.0 (C-7a), 141.3 (C-4″), 138.3 (C-1″), |
| | | | | 137.0 (C-1′), 132.1 (C-4′), 130.9 (C-6), 131.0 (C-2″ and C-6″), 130.4 (C-2′ and C-6′), 130.3 (C-3″ and |
| | | | | C-5″), 125.3 (C-3′ and C-5′), 116.7 (C-4a), 103.6 (C-5), 58.3 (NCH₂), 57.0 (NCH₂ₚᵢₚ), 54.8 (NCH₂), |
| | | | | 54.6 (NCH₂ₚᵢₚ), 49.5 (NCH₂), 47.4 (NCH₃), 28.3 (CH₂); MALDI-TOF MS m/z [M + H]⁺ Calculed: |
| | | | | 475.2377, Found: 475.2354. |
| 23B | C₂₇H₃₁ClN₆•3(COOH)₂ | 63% | 227° | White crystals |
| | | | C. | |
| 24A | C₃₆H₅₃N₇O₂ | 67% | | Orange oil. ¹H NMR δ (300 MHz, CDCl₃): 8.44 (d, 2H, J = 8.10 Hz, H-2′ and H-6′), 7.69 (d, 2H, J = |
| | | | | 8.10 Hz, H-2″ and H-6″), 7.47 (d, 2H, J = 8.10 Hz, H-3′, H-5′), 7.43 (d, 2H, J = 8.10 Hz, H-3″ and |
| | | | | H-5″), 4.76 (s, 2H, NCH₂), 3.88 (s, 2H, NCH₂), 3.87 (s, 2H, NCH₂), 3.64 (t, 2H, J = 5.10 Hz, NCH₂), |
| | | | | 2.93 (t, 2H, J = 5.10 Hz, CH₂), 2.75-2.66 (m, 4H, 2NCH₂), 2.38-2.32 (m, 4H, 2NCH₂), 2.24 (s, 6H, |
| | | | | N(CH₃)₂), 2.22 (s, 6H, N(CH₃)₂), 1.77-1.65 (m, 4H, 2CH₂), 1.54 (s, 9H, C(CH₃)₃); MALDI-TOF MS |
| | | | | m/z [M + 2H]⁺ Calculed: 617.4417, Found: 617.4410 |
| 24B | C₃₆H₅₃N₇O₂•4(COOH)₂ | 64% | 156° | Yellow crystals |
| | | | C. | |
| 25A | C₃₁H₄₅N₇ | 91% | | Orange oil. ¹H NMR δ (300 MHz, CDCl₃): 8.44 (d, 2H, J = 8.40 Hz, H-2′ and H-6′), 7.68 (d, 2H, J = |
| | | | | 8.40 Hz, H-2″ and H-6″), 7.46 (d, 2H, J = 8.40 Hz, H-3′, H-5′), 7.42 (d, 2H, J = 8.40 Hz, H-3″ and |
| | | | | H-5″), 4.21 (s, 2H, NCH₂), 3.90 (s, 2H, NCH₂), 3.89 (s, 2H, NCH₂), 3.11 (t, 2H, J = 5.10 Hz, NCH₂), |
| | | | | 2.96 (bs, 2H, 2NH) 2.90 (t, 2H, J = 5.10 Hz, CH₂), 2.79-2.73 (m, 4H, 2NCH₂), 2.42-2.33 (m, 4H, |
| | | | | 2NCH₂), 2.26 (s, 6H, N(CH₃)₂), 2.24 (s, 6H, N(CH₃)₂), 1.77-1.69 (m, 4H, 2CH₂); MALDI-TOF MS |
| | | | | m/z [M + 2H]⁺ Calculed: 517.3893, Found: 517.3880. |
| 25B | C₃₁H₄₅N₇•5(COOH)₂ | 61% | 160° | Yellow crystals |
| | | | C. | |
| 26A | C₃₀H₄₀N₆S | 98% | | Yellow oil. ¹H NMR δ (300 MHz, CDCl₃): 8.58 (d, 2H, J = 8.40 Hz, H-2′ and H-6′), 8.01 (d, 2H, J = |
| | | | | 8.40 Hz, H-2″ and H-6″), 7.56 (d, 1H, J = 6.00 Hz, H-6), 7.52 (d, 2H, J = 8.40 Hz, H-3′ and H-5′), |
| | | | | 7.47 (d, 1H, J = 6.00 Hz, H-5), 7.44 (d, 2H, J = 8.40 Hz, H-3″ and H-5″), 3.89 (s, 2H, NCH₂), 3.86 |
| | | | | (s, 2H, NCH₂), 2.72 (t, 2H, J = 6.90 Hz, NCH₂), 2.69 (t, 2H, J = 6.90 Hz, NCH₂), 2.34 (t, 2H, J = |
| | | | | 6.90 Hz, NCH₂), 2.31 (t, 2H, J = 6.90 Hz, NCH₂), 2.22 (s, 6H, N(CH₃)₂), 2.21 (s, 6H, N(CH₃)₂), 2.09 |
| | | | | (bs, 1H, NH), 1.76-1.63 (m, 4H, 2CH₂); MALDI-TOF MS m/z [M + H]⁺ Calculed: 517.3113, Found: |
| | | | | 517.3102. |
| 26B | C₃₀H₄₀N₆S•4(COOH)₂ | 90% | 193° | White crystals |
| | | | C. | |
| 27A | C₃₆H₅₀N₈S | 97% | | Pale-yellow oil. ¹H NMR δ (300 MHz, CDCl₃): 8.55 (d, 2H, J = 8.10 Hz, H-2′ and H-6′), 7.98 (d, |
| | | | | 2H, J = 8.10 Hz, H-2″ and H-6″), 7.54 (d, 1H, J = 6.00 Hz, H-6), 7.52-7.40 (m, 5H, H-3′, H-5′, H-3″, |
| | | | | H-5″ and H-5), 3.86 (s, 2H, NCH₂), 3.83 (s, 2H, NCH₂), 2.69 (t, 2H, J = 6.60 Hz, NCH₂), 2.66 (t, 2H, |
| | | | | J = 6.60 Hz, NCH₂), 2.54-2.28 (m, 20H, 10 NCH₂), 2.23 (s, 6H, 2 NCH₃), 1.73-1.65 (m, 4H, 2 CH₂); |
| | | | | ¹³C NMR δ (75 MHz, CDCl₃): 172.2 (C-7a), 161.7 (C-2), 160.9 (C-4), 144.4 (C-4″), 144.2 (C-4′), |
| | | | | 138.2 (C-1′), 137.8 (C-1″), 130.7 (C-2″ and C-6″), 130.0 (C-2′ and C-6′), 129.9 (C-3″ and C-5″), 129.5 |
| | | | | (C-3′ and C-5′), 127.6 (C-5), 127.0 (C-4a), 122.3 (C-6), 58.4 (NCH₂), 56.5 (NCH₂pip.), 55.1 (NCH₂), |
| | | | | 54.6 (NCH₂pip.), 49.5 (NCH₂), 47.4 (NCH₃), 28.3 (CH₂); MALDI-TOF MS m/z [M + 2H]⁺ Calculed: |
| | | | | 628.4035, Found: 628.4024. |
| 27B | C₃₆H₅₀N₈S•6(COOH)₂ | 53% | 220° | Pale-yellow crystals |
| | | | C. | |
| 28A | C₂₃H₃₄N₆S | 52% | | Yellow oil. ¹H NMR δ (300 MHz, CDCl₃): 7.84 (d, 2H, J = 8.10 Hz, H-2′ and H-6′), 7.47 (d, 2H, J = |
| | | | | 8.10 Hz, H-3′ and H-5′), 7.30 (d, 1H, J = 6.30 Hz, H-6), 6.99 (d, 1H, J = 6.30 Hz, H-5), 5.76 (t, 1H, |
| | | | | J = 5.60 Hz, NH), 3.89 (s, 2H, NCH₂), 3.60 (q, 2H, J = 6.90 Hz, NCH₂), 2.71 (t, 2H, J = 6.90 Hz, |
| | | | | NCH₂), 2.43 (t, 2H, J = 6.90 Hz, NCH₂), 2.35 (t, 2H, J = 6.90 Hz, NCH₂), 2.26 (s, 6H, N(CH₃)₂), 2.24 |
| | | | | (s, 6H, N(CH₃)₂), 1.84 (qt, 2H, J = 6.90 Hz, CH₂), 1.72 (qt, 2H, J = 6.90 Hz, CH₂); ¹³C NMR δ (75 |
| | | | | MHz, CDCl₃): 164.9 (C-4), 163.0 (C-2), 161.1 (C-7a), 143.9 (C-4′), 138.1 (C-1′), 130.3 (C-2′ and C- |
| | | | | 6′), 129.7 (C-3′ and C-5′), 122.4 (C-5), 120.7 (C-6), 109.3 (C-4a), 59.5 (NCH₂), 55.1 (NCH₂), 54.8 |
| | | | | (NCH₂), 49.3 (NCH₂), 46.9 (N(CH₃)₂), 42.0 (NCH₂), 29.3 (CH₂), 28.7 (CH₂); MALDI-TOF MS m/z |
| | | | | [M + 2H]⁺ Calculed: 428.2809, Found: 428.2722. |
| 28B | C₂₃H₃₄N₆S•4(COOH)₂ | 57% | 146° | Yellow crystals |
| | | | C. | |
| 29A | C₃₀H₄₀N₆S | 96% | | Yellow oil. ¹H NMR δ (300 MHz, CDCl₃): 8.59 (d, 2H, J = 8.40 Hz, H-2′ and H-6′), 8.26 (d, 2H, J = |
| | | | | 8.40 Hz, H-2″ and H-6″), 7.97 (d, 1H, J = 5.70 Hz, H-6), 7.61 (d, 1H, J = 5.70 Hz, H-7), 7.53 (d, |
| | | | | 2H, J = 8.40 Hz, H-3′ and H-5′), 7.45 (d, 2H, J = 8.40 Hz, H-3″ and H-5″), 3.89 (s, 2H, NCH₂), 3.87 |
| | | | | (s, 2H, NCH₂), 2.71 (t, 2H, J = 7.20 Hz, NCH₂), 2.69 (t, 2H, J = 7.20 Hz, NCH₂), 2.33 (t, 2H, J = 7.2 |
| | | | | Hz, NCH₂), 2.31 (t, 2H, J = 7.20 Hz, NCH₂), 2.21 (s, 6H, N(CH₃)₂), 2.20 (s, 6H, N(CH₃)₂), 1.75-1.63 |
| | | | | (m, 4H, 2CH₂); ¹³C NMR δ (75 MHz, CDCl₃): 164.5 (C-4a), 162.5 (C-2), 160.8 (C-4), 145.2 (C-4″), |
| | | | | 144.1 (C-4′), 138.3 (C-1′), 137.6 (C-1″), 137.2 (C-7), 130.1 (C-2″ and C-6″), 129.9 (C-2′, C-6′, C-3″ |
| | | | | and C-5″), 129.6 (C-3′ and C-5′), 126.9 (C-7a), 126.5 (C-6), 59.5 (NCH₂), 55.2 (NCH₂), 49.3 (NCH₂), |
| | | | | 46.9 (2 N(CH₃)₂), 29.4 (CH₂); MALDI-TOF MS m/z [M + H]⁺ Calculed: 517.3113, Found: 517.3099. |
| 29B | C₃₀H₄₀N₆S•4(COOH)₂ | 75% | 196° | White crystals |
| | | | C. | |
| 30A | C₃₆H₅₀N₈S | 87% | | Yellow oil. ¹H NMR δ (300 MHz, CDCl₃): 8.56 (d, 2H, J = 8.10 Hz, H-2′ and H-6′), 8.23 (d, 2H, J = |
| | | | | 8.10 Hz, H-2″ and H-6″), 7.94 (d, 1H, J = 5.40 Hz, H-6), 7.58 (d, 1H, J = 5.40 Hz, H-7), 7.49 (d, |
| | | | | 2H, J = 8.10 Hz, H-3′ and H-5′), 7.42 (d, 2H, J = 8.10 Hz, H-3″ and H-5″), 3.85 (s, 2H, NCH₂), 3.83 |
| | | | | (s, 2H, NCH₂), 2.67 (t, 2H, J = 6.60 Hz, NCH₂), 2.65 (t, 2H, J = 6.60 Hz, NCH₂), 2.55-2.25 (m, 20H, |
| | | | | 10 NCH₂), 2.22 (s, 6H, 2 NCH₃), 1.72-1.64 (m, 4H, 2 CH₂); ¹³C NMR δ (75 MHz, CDCl₃): 164.5 |
| | | | | (C-4a), 162.5 (C-2), 160.7 (C-4), 145.1 (C-4″), 144.1 (C-4′), 138.2 (C-1′), 137.6 (C-1″), 137.2 (C-7), |
| | | | | 130.0 (C-2″ and C-6″), 129.8 (C-2′, C-6′, C-3″ and C-5″), 129.5 (C-3′ and C-5′), 126.9 (C-7a), 126.5 |
| | | | | (C-6), 58.4 (NCH₂), 56.5 (NCH₂pip.), 55.1 (NCH₂), 54.6 (NCH₂pip.), 49.5 (NCH₂), 47.4 (NCH₃), |
| | | | | 28.3 (CH₂); MALDI-TOF MS m/z [M + 2H]⁺ Calculed: 628.4035, Found: 628.4022. |
| 30B | C₃₆H₅₀N₈S•6(COOH)₂ | 76% | 212° | White crystals |
| | | | C. | |
| 31A | C₂₃H₃₄N₆S | 96% | | Yellow-orange oil. ¹H NMR δ (300 MHz, CDCl₃): 8.04 (d, 2H, J = 8.40 Hz, H-2′ and H-6′), 7.76 (d, |
| | | | | 1H, J = 5.40 Hz, H-6), 7.44 (d, 2H, J = 8.40 Hz, H-3′ and H-5′), 7.21 (d, 1H, J = 5.40 Hz, H-7), 5.69 |
| | | | | (t, 1H, J = 5.70 Hz, NH), 3.83 (s, 2H, NCH₂), 3.54 (q, 2H, J = 6.90 Hz, NCH₂), 2.65 (t, 2H, J = 6.90 |
| | | | | Hz, NCH₂), 2.38 (t, 2H, J = 6.90 Hz, NCH₂), 2.31 (t, 2H, J = 6.90 Hz, NCH₂), 2.20 (s, 6H, N(CH₃)₂), |
| | | | | 2.18 (s, 6H, N(CH₃)₂), 1.80 (qt, 2H, J = 6.90 Hz, CH₂), 1.63 (qt, 2H, J = 6.90 Hz, CH₂); ¹³C NMR δ |
| | | | | (75 MHz, CDCl₃): 165.2 (C-4), 162.7 (C-2), 161.8 (C-7a), 144.5 (C-4′), 137.8 (C-1′), 137.0 (C-7), |
| | | | | 129.7 (C-2′, C-6′, C-3′ and C-5′), 124.9 (C-6), 119.3 (C-4a), 59.3 (NCH₂), 59.1 (NCH₂), 55.0 (NCH₂), |
| | | | | 49.2 (NCH₂), 46.9 (N(CH₃)₂), 41.9 (NCH₂), 29.3 (CH₂), 28.7 (CH₂); MALDI-TOF MS m/z [M + H]⁺ |
| | | | | Calculed: 427.2644, Found: 427.2663. |
| 31B | C₂₃H₃₄N₆S•4(COOH)₂ | 55% | 179° | Yellow crystals |
| | | | C. | |
| 32A | C₃₈H₄₉N₇ | 82% | | Pale-yellow oil. ¹H NMR δ (300 MHz, CDCl₃): 8.61 (d, 2H, J = 8.40 Hz, H-2′ and H-6′), 8.28 (dd, |
| | | | | 2H, J = 7.90 and 1.70 Hz, H-2″ and H-6″), 7.86 (d, 2H, J = 8.40 Hz, H-2″′ and H-6″′), 7.63-7.52 (m, |
| | | | | 6H, H-6, H-3″, H-4″, H-5″, H-3″ and H-5″), 7.44 (d, 2H, J = 8.40 Hz, H-3′ and H-5′), 6.98 (d, 1H, J = |
| | | | | 3.60 Hz, H-5), 3.90 (s, 2H, NCH₂), 3.88 (s, 2H, NCH₂), 2.78-2.66 (m, 4H, 2NCH₂), 2.34-2.24 (m, |
| | | | | 4H, 2NCH₂), 2.23 (s, 6H, N(CH₃)₂), 2.21 (s, 6H, N(CH₃)₂), 1.60-1.48 (m, 8H, 4CH₂); ¹³C NMR δ |
| | | | | (75 MHz, CDCl₃): 159.5 (C-2), 159.0 (C-4), 153.9 (C-7a), 143.2 (C-1″′), 140.2 (C-4′), 140.0 (C-4″′), |
| | | | | 139.0 (C-1″), 138.0 (C-1′), 131.4 (C-4″), 130.4 (C-3″, C-5″, C-3″′ and C-5″′), 130.1 (C-3′ and C-5′), |
| | | | | 129.9 (C-6), 129.6 (C-2′, C-6′, C-2″ and C-6″), 125.0 (C-2″′ and C-6″′), 116.2 (C-4a), 103.5 (C-5), |
| | | | | 61.0 (NCH₂), 55.1 (NCH₂), 54.9 (NCH₂), 50.8 (NCH₂), 50.5 (NCH₂), 46.8 (NCH₃), 29.3 (CH₂), 26.9 |
| | | | | (CH₂); MALDI-TOF MS m/z [M + H]⁺ Calculed: 604.4128, Found: 604.4165. |
| 32B | C₃₈H₄₉N₇•4(COOH)₂ | 52% | 151° | Pale-yellow crystals |
| | | | C. | |
| 33A | C₄₂H₅₇N₇ | 78% | | Yellow oil. ¹H NMR δ (300 MHz, CDCl₃): 8.61 (d, 2H, J = 8.10 Hz, H-2′ and H-6′), 8.27 (d, 2H, J = |
| | | | | 8.10 Hz, H-2″ and H-6″), 7.93 (d, 2H, J = 7.80 Hz, H-2″′ and H-6″′), 7.64-7.56 (m, 5H, H-3′, H-5′, |
| | | | | H-3″, H-5″ and H-6), 7.46 (d, 2H, J = 7.80 Hz, H-3″′ and H-5″′), 7.43 (t, 1H, J = 7.80 Hz, H-4″′), 7.01 |
| | | | | (d, 1H, J = 3.60 Hz, H-5), 3.95 (s, 2H, NCH₂), 3.90 (s, 2H, NCH₂), 2.76-2.66 (m, 4H, 2NCH₂), 2.57 |
| | | | | (q, 4H, J = 7.20 Hz, 2NCH₂), 2.55 (q, 4H, J = 7.20 Hz, 2NCH₂), 2.50-2.45 (m, 4H, 2NCH₂), 1.60- |
| | | | | 1.52 (m, 8H, 4CH₂), 1.06 (t, 6H, J = 7.20 Hz, 2CH₃), 1.03 (t, 6H, J = 7.20 Hz, 2CH₃); ¹³C NMR □ |
| | | | | (75 MHz, CDCl₃): 159.6 (C-2), 158.9 (C-4), 154.0 (C-7a), 144.0 (C-4″), 143.4 (C-4′), 139.2 (C-1″′), |
| | | | | 139.0 (C-1′), 138.7 (C-1″), 130.7 (C-2″ and C-6″), 130.5 (C-2′ and C-6′), 129.8 (C-3″′, C-5″′ and C- |
| | | | | 4″′), 129.6 (C-3′ and C-5′), 129.5 (C-3″ and C-5″), 127.9 (C-6), 125.1 (C-2″′ and C-6″′), 116.2 (C-4a), |
| | | | | 103.6 (C-5), 55.2 (NCH₂), 54.2 (NCH₂), 50.7 (NCH₂), 50.6 (NCH₂), 48.2 (NCH₂), 29.6 (CH₂), 26.2 |
| | | | | (CH₂), 13.0 (CH₃); MALDI-TOF MS m/z [M + 2H]⁺ Calculed: 661.4831, Found: 661.4852. |
| 33B | C₄₂H₅₇N₇•4(COOH)₂ | 80% | 151° | White crystals |
| | | | C. | |
| 34A | C₄₈H₆₉N₇ | 75% | | Yellow oil. ¹H NMR δ (300 MHz, CDCl₃): 8.61 (d, 2H, J = 8.40 Hz, H-2′ and H-6′), 8.26 (d, 2H, J = |
| | | | | 8.40 Hz, H-2″ and H-6″), 7.92 (d, 2H, J = 8.10 Hz, H-2″′ and H-6″′), 7.63-7.54 (m, 5H, H-3′, H-5′, |
| | | | | H-3″, H-5″ and H-6), 7.46-7.38 (m, 3H, H-3″′, H-4″′ and H-5″′), 7.00 (d, 1H, J = 3.90 Hz, H-5), 3.94 |
| | | | | (s, 2H, NCH₂), 3.88 (s, 2H, NCH₂), 3.10-3.00 (m, 4H, 4NCH), 2.73-2.63 (m, 4H, 2NCH₂), 2.45-2.37 |
| | | | | (m, 4H, 2NCH₂), 1.61-1.53 (m, 4H, 2CH₂), 1.49-1.39 (m, 4H, 2CH₂), 1.37-1.28 (m, 4H, 2CH₂), 1.04 |
| | | | | (d, 12H, J = 6.50 Hz, 4CH₃), 1.03 (d, 12H, J = 6.50 Hz, 4CH₃); ¹³C NMR □ (75 MHz, CDCl₃): |
| | | | | 159.6 (C-2), 158.9 (C-4), 154.0 (C-7a), 144.1 (C-4″), 143.6 (C-4′), 139.2 (C-1″′), 139.0 (C-1′), 138.7 |
| | | | | (C-1″), 130.7 (C-2″ and C-6″), 130.5 (C-2′ and C-6′), 129.8 (C-3″′, C-5″′ and C-4″′), 129.6 (C-3′ and |
| | | | | C-5′), 129.5 (C-3″ and C-5″), 127.9 (C-6), 125.1 (C-2″′ and C-6″′), 116.1 (C-4a), 103.6 (C-5), 55.3 |
| | | | | (NCH₂), 50.1 (NCH₂), 50.8 (NCH₂), 50.0 (NCH), 46.8 (NCH₂), 32.8 (CH₂), 31.5 (CH₂), 26.6 (CH₂), |
| | | | | 22.0 CH₃); MALDI-TOF MS m/z [M + H]⁺ Calculed: 744.5693, Found: 744.5674. |
| 34B | C₄₈H₆₉N₇•4(COOH)₂ | 71% | 166° | Pale-yellow crystals |
| | | | C. | |
| 35A | C₄₀H₅₃N₇ | 91% | | Pale-yellow oil. ¹H NMR δ (300 MHz, CDCl₃): 8.61 (d, 2H, J = 8.10 Hz, H-2′ and H-6′), 8.29 (dd, |
| | | | | 2H, J = 8.10 and 1.60 Hz, H-2″ and H-6″), 7.86 (d, 2H, J = 8.10 Hz, H-2″′ and H-6″′), 7.64-7.53 (m, |
| | | | | 6H, H-6, H-3″, H-4″, H-5″, H-3″′ and H-5″′), 7.44 (d, 2H, J = 8.10 Hz, H-3′ and H-5′), 7.00 (d, 1H, J = |
| | | | | 3.90 Hz, H-5), 3.91 (s, 2H, NCH₂), 3.88 (s, 2H, NCH₂), 2.76-2.63 (m, 4H, 2NCH₂), 2.34-2.24 (m, |
| | | | | 4H, 2NCH₂), 2.25 (s, 6H, N(CH₃)₂), 2.24 (s, 6H, N(CH₃)₂), 1.63-1.33 (m, 12H, 6CH₂); ¹³C NMR □ |
| | | | | (75 MHz, CDCl₃): 159.6 (C-2), 159.0 (C-4), 154.0 (C-7a), 143.5 (C-1″′), 140.4 (C-4′), 140.0 (C-4″′), |
| | | | | 139.0 (C-1″), 137.9 (C-1′), 131.4 (C-4″), 130.4 (C-3″, C-5″, C-3″′ and C-5″′), 130.1 (C-3′ and C-5′), |
| | | | | 129.6 (C-6), 129.5 (C-2′, C-6′, C-2″ and C-6″), 125.0 (C-2″′ and C-6″′), 116.2 (C-4a), 103.5 (C-5), |
| | | | | 61.1 (NCH₂), 55.2 (NCH₂), 54.9 (NCH₂), 50.9 (NCH₂), 50.6 (NCH₂), 46.9 (NCH₃), 31.4 (CH₂), 29.0 |
| | | | | (CH₂), 26.6 (CH₂); MALDI-TOF MS m/z [M + H]⁺ Calculed: 632.4440, Found: 632.4421. |
| 35B | C₄₀H₅₃N₇•4(COOH)₂ | 62% | 138° | White crystals |
| | | | C. | |
| 36A | C₃₅H₄₄N₈ | 95% | | Yellow oil. ¹H NMR δ (300 MHz, CDCl₃): 8.69 (d, 2H, J = 8.10 Hz, H-2′ and H-6′), 8.52 (s, 1H, H- |
| | | | | 3), 8.42 (dd, 2H, J = 8.60 and 1.20 Hz, H-2″′ and H-6″′), 8.32 (d, 2H, J = 8.10 Hz, H-2″ and H-6″), |
| | | | | 7.65-7.59 (m, 4H, H-3′, H-5′, H-3″′ and H-5″′), 7.53 (d, 2H, J = 8.10 Hz, H-3″ and H-5″), 7.41 (t, 1H, |
| | | | | J = 8.60 Hz, H-4″′), 3.97 (s, 2H, NCH₂), 3.95 (s, 2H, NCH₂), 2.79 (t, 2H, J = 6.90 Hz, NCH₂), 2.78 |
| | | | | (t, 2H, J = 6.90 Hz, NCH₂), 2.41 (t, 2H, J = 6.90 Hz, NCH₂), 2.40 (t, 2H, J = 6.90 Hz, NCH₂), 2.28 |
| | | | | (s, 6H, N(CH₃)₂), 2.27 (s, 6H, N(CH₃)₂), 1.78 (qt, 4H, J = 6.90 Hz, 2CH₂); ¹³C NMR □ (75 MHz, |
| | | | | CDCl₃): 163.1 (C-4), 161.8 (C-6), 145.2 (C-7a), 143.6 (C-4″ and C-4′), 140.4 (C-1″′), 138.2 (C-1′), |
| | | | | 137.4 (C-1″), 135.8 (C-3), 130.6 (C-2″, C-6″, C-2′, C-6′, C-3″′ and C-5″′), 130.2 (C-3′ and C-5′), 129.8 |
| | | | | (C-3″ and C-5″), 127.9 (C-4″′), 122.7 (C-2″′ and C-6″′), 112.6 (C-3a), 59.3 (NCH₂), 54.9 (NCH₂), |
| | | | | 54.8 (NCH₂), 49.3 (NCH₂), 46.9 (N(CH₃)₂), 29.0 (CH₂), 28.5 (CH₂); MALDI-TOF MS m/z [M + H]⁺ |
| | | | | Calculed: 577.3767, Found: 577.3762. |
| 36B | C₃₅H₄₄N₈•4(COOH)₂ | 46% | 162° | Pale-yellow crystals |
| | | | C. | |
| 37A | C₃₇H₄₈N₈ | 75% | | Pale-yellow oil. ¹H NMR δ (300 MHz, CDCl₃): 8.69 (d, 2H, J = 8.10 Hz, H-2′ and H-6′), 8.53 (s, |
| | | | | 1H, H-3), 8.43 (dd, 2H, J = 8.40 and 1.20 Hz, H-2″′ and H-6″′), 8.33 (d, 2H, J = 8.10 Hz, H-2″ and |
| | | | | H-6″), 7.66-7.59 (m, 4H, H-3′, H-5′, H-3″′ and H-5″′), 7.51 (d, 2H, J = 8.10 Hz, H-3″ and H-5″), 7.41 |
| | | | | (t, 1H, J = 8.40 Hz, H-4″′), 3.97 (s, 2H, NCH₂), 3.93 (s, 2H, NCH₂), 2.74-2.70 (m, 4H, 2NCH₂), 2.35- |
| | | | | 2.27 (m, 4H, 2NCH₂), 2.24 (s, 6H, N(CH₃)₂), 2.22 (s, 6H, N(CH₃)₂), 1.60-1.52 (m, 8H, 4CH₂); ¹³C |
| | | | | NMR □ (75 MHz, CDCl₃): 163.1 (C-4), 161.7 (C-6), 145.7 (C-7a), 144.8 (C-4″ and C-4′), 140.4 (C- |
| | | | | 1″′), 137.9 (C-1′), 137.2 (C-1″), 135.7 (C-3), 130.5 (C-2″, C-6″, C-2′, C-6′, C-3″′ and C-5″′), 130.1 |
| | | | | (C-3′ and C-5′), 129.6 (C-3″ and C-5″), 127.8 (C-4″′), 122.6 (C-2″′ and C-6″′), 112.5 (C-3a), 61.1 |
| | | | | (NCH₂), 55.1 (NCH₂), 50.7 (NCH₂), 46.9 (N(CH₃)₂), 29.4 (CH₂), 26.9 (CH₂); MALDI-TOF MS m/z |
| | | | | [M + H]⁺ Calculed: 605.4080, Found: 605.4069. |
| 37B | C₃₇H₄₈N₈•4(COOH)₂ | 53% | 129° | Pale-yellow crystals |
| | | | C. | |
| 38A | C₃₉H₅₂N₈ | 92% | | Yellow oil. ¹H NMR δ (300 MHz, CDCl₃): 8.68 (d, 2H, J = 8.10 Hz, H-2′ and H-6′), 8.51 (s, 1H, H- |
| | | | | 3), 8.42 (dd, 2H, J = 8.40 and 1.20 Hz, H-2″′ and H-6″′), 8.31 (d, 2H, J = 8.10 Hz, H-2″ and H-6″), |
| | | | | 7.64-7.58 (m, 4H, H-3′, H-5′, H-3″′ and H-5″′), 7.50 (d, 2H, J = 8.10 Hz, H-3″ and H-5″), 7.40 (t, 1H, |
| | | | | J = 8.40 Hz, H-4″′), 3.94 (s, 2H, NCH₂), 3.91 (s, 2H, NCH₂), 2.71 (t, 2H, J = 6.90 Hz, NCH₂), 2.69 |
| | | | | (t, 2H, J = 6.90 Hz, NCH₂), 2.31-2.27 (m, 4H, 2NCH₂), 2.24 (s, 6H, N(CH₃)₂), 2.23 (s, 6H, N(CH₃)₂), |
| | | | | 1.62-1.34 (m, 12H, 6CH₂); ¹³C NMR □ (75 MHz, CDCl₃): 163.1 (C-4), 161.8 (C-6), 145.7 (C-7a), |
| | | | | 144.8 (C-4″ and C-4′), 140.4 (C-1″′), 137.9 (C-1′), 137.3 (C-1″), 135.7 (C-3), 130.5 (C-2″, C-6″, C-2′ |
| | | | | and C-6′), 130.4 (C-3″′ and C-5″′), 130.1 (C-3′ and C-5′), 129.6 (C-3″ and C-5″), 127.8 (C-4″), 122.6 |
| | | | | (C-2″′ and C-6″′), 112.5 (C-3a), 61.1 (NCH₂), 55.1 (NCH₂), 50.8 (NCH₂), 46.9 (N(CH₃)₂), 31.4 (CH₂), |
| | | | | 31.2 (CH₂), 29.0 (CH₂), 26.9 (CH₂), 26.3 (CH₂); MALDI-TOF MS m/z [M + H]⁺ Calculed: 633.4393, |
| | | | | Found: 633.4382. |
| 38B | C₃₉H₅₂N₈•4(COOH)₂ | 61% | 145° | Pale-yellow crystals |
| | | | C. | |
| 39A | C₄₃H₅₇N₁₀ | 70% | | Yellow oil. ¹H NMR δ (300 MHz, CDCl₃): 8.70 (d, 2H, J = 8.10 Hz, H-2′ and H-6′), 8.52 (s, 1H, H- |
| | | | | 3), 8.42 (dd, 2H, J = 7.80 and 1.20 Hz, H-2″′ and H-6″′), 8.32 (d, 2H, J = 8.10 Hz, H-2″ and H-6″), |
| | | | | 7.68-7.56 (m, 6H, H-3′, H-5′, H-3″′, H-5″′, H-3″ and H-5″), 7.41 (t, 1H, J = 7.80 Hz, H-4″′), 3.98 (s, |
| | | | | 2H, NCH₂), 3.96 (s, 2H, NCH₂), 2.75 (t, 4H, J = 6.60 Hz, 2NCH₂), 2.59-2.39 (m, 20H, 10NCH₂), |
| | | | | 2.29 (s, 3H, NCH₃), 2.27 (s, 3H, NCH₃), 1.69-1.59 (m, 8H, 4CH₂); ¹³C NMR □ (75 MHz, CDCl₃): |
| | | | | 163.1 (C-4), 161.8 (C-6), 145.6 (C-7a), 144.6 (C-4″ and C-4′), 140.4 (C-1″′), 138.0 (C-1′), 137.3 (C- |
| | | | | 1″), 135.7 (C-3), 130.5 (C-2″, C-6″, C-2′, C-6′, C-3″′ and C-5″′), 130.1 (C-3′ and C-5′), 129.6 (C-3″ |
| | | | | and C-5″), 127.8 (C-4″′), 122.7 (C-2″′ and C-6″′), 112.7 (C-3a), 59.9 (NCH₂), 56.5 (NCH₂), 55.1 |
| | | | | (NCH₂), 54.6 (NCH₂), 50.8 (NCH₂), 50.6 (NCH₂), 47.5 (N(CH₃)₂), 29.5 (CH₂), 26.1 (CH₂); MALDI- |
| | | | | TOF MS m/z [M + H]⁺ Calculed: 715.4924, Found: 715.4910 |
| 39B | C₄₃H₅₇N₁₀•6(COOH)₂ | 76% | 219° | White crystals |
| | | | C. | |
| 40A | C₃₇H₄₈N₈ | 78% | | Yellow oil. ¹H NMR δ (300 MHz, CDCl₃) 8.94 (d, 2H, J = 8.40 Hz, H-2′ and H-6′), 8.62 (d, 2H, J = |
| | | | | 8.40 Hz, H-2″ and H-6″), 8.40 (s, 1H, H-6), 7.91 (d, 2H, J = 7.80 Hz, H-2″′ and H-6″′), 7.66 (t, 2H, J = |
| | | | | 7.80 Hz, H-3″′ and H-5″′), 7.58 (d, 2H, J = 8.40 Hz, H-3′ and H-5′), 7.52 (t, 1H, J = 7.80 Hz, H-4″′), |
| | | | | 7.49 (d, 2H, J = 8.40 Hz, H-3″ and H-5″), 3.94 (s, 2H, NCH₂), 3.91 (s, 2H, NCH₂), 2.72 (t, 2H, J = |
| | | | | 6.60 Hz, NCH₂), 2.70 (t, 2H, J = 6.60 Hz, NCH₂), 2.36-2.28 (m, 4H, 2NCH₂), 2.25 (s, 6H, N(CH₃)₂), |
| | | | | 2.23 (s, 6H, N(CH₃)₂), 1.76-1.52 (m, 8H, 4CH₂); ¹³C NMR □ (75 MHz, CDCl₃): 160.5 (C-2), 156.1 |
| | | | | (C-4), 154.4 (C-7a), 144.8 (C-4′ and C-4″), 143.6 (C-1″′), 138.5 (C-4a), 136.2 (C-1′ and C-1″), 131.4 |
| | | | | (C-3″′ and C-5″′), 131.2 (C-3′ and C-5′), 129.9 (C-3″ and C-5″), 129.8 (C-2′ and C-6′), 129.7 (C-2″ |
| | | | | and C-6″), 129.5 (C-6), 128.2 (C-4″′), 124.8 (C-2″′ and C-6″′), 61.0 (NCH₂), 55.1 (NCH₂), 50.6 |
| | | | | (NCH₂), 46.8 (N(CH₃)₂), 29.4 (CH₂), 27.0 (CH₂); MALDI-TOF MS m/z [M + H]⁺ Calculed: 605.4080, |
| | | | | Found: 605.4065. |
| 40B | C₃₇H₄₈N₈•4(COOH)₂ | 73% | 160° | White crystals |
| | | | C. | |
| 41A | C₃₉H₅₂N₈ | 93% | | Yellow oil. %); ¹H NMR δ (300 MHz, CDCl₃) 8.93 (d, 2H, J = 8.40 Hz, H-2′ and H-6′), 8.61 (d, 2H, |
| | | | | J = 8.40 Hz, H-2″ and H-6″), 8.40 (s, 1H, H-6), 7.90 (d, 2H, J = 7.80 Hz, H-2″′ and H-6″′), 7.66 (t, |
| | | | | 2H, J = 7.80 Hz, H-3″′ and H-5″′), 7.57 (d, 2H, J = 8.40 Hz, H-3′ and H-5′), 7.52 (t, 1H, J = 7.80 Hz, |
| | | | | H-4″′), 7.47 (d, 2H, J = 8.40 Hz, H-3″ and H-5″), 3.93 (s, 2H, NCH₂), 3.89 (s, 2H, NCH₂), 2.70 (t, |
| | | | | 2H, J = 6.90 Hz, NCH₂), 2.67 (t, 2H, J = 6.90 Hz, NCH₂), 2.32-2.26 (m, 4H, 2NCH₂), 2.24 (s, 6H, |
| | | | | N(CH₃)₂), 2.23 (s, 6H, N(CH₃)₂), 1.62-1.46 (m, 8H, 4CH₂), 1.41-1.33 (m, 4H, 2CH₂); ¹³C NMR □ |
| | | | | (75 MHz, CDCl₃): 160.3 (C-2), 156.0 (C-4), 154.3 (C-7a), 145.0 (C-4′ and C-4″), 144.0 (C-1″′), |
| | | | | 138.3 (C-4a), 136.2 (C-1′ and C-1″), 131.3 (C-3″′ and C-5″′), 131.1 (C-3′ and C-5′), 129.8 (C-3″ and |
| | | | | C-5″), 129.7 (C-2′ and C-6′), 129.6 (C-2″ and C-6″), 129.4 (C-6), 128.2 (C-4″′), 124.6 (C-2″′ and C- |
| | | | | 6″′), 61.1 (NCH₂), 55.2 (NCH₂), 50.7 (NCH₂), 46.9 (N(CH₃)₂), 31.4 (CH₂), 29.0 (CH₂), 26.6 (CH₂); |
| | | | | MALDI-TOF MS m/z [M + H]⁺ Calculed: 633.4393, Found: 633.4379. |
| 41B | C₃₉H₅₂N₈•4(COOH)₂ | 76% | 215° | Pale-yellow crystals |
| | | | C. | |
| 42A | C₄₃H₅₈N₁₀ | 68% | | Pale-yellow oil. ¹H NMR δ (300 MHz, CDCl₃): 8.92 (d, 2H, J = 8.10 Hz, H-2′ and H-6′), 8.59 (d, |
| | | | | 2H, J = 8.10 Hz, H-2″ and H-6″), 8.38 (s, 1H, H-6), 7.87 (d, 2H, J = 7.80 Hz, H-2″′ and H-6″′), 7.63 |
| | | | | (t, 2H, J = 7.80 Hz, H-3″′ and H-5″′), 7.55 (d, 2H, J = 8.10 Hz, H-3′ and H-5′), 7.49 (t, 1H, J = 7.80 |
| | | | | Hz, H-4″′), 7.45 (d, 2H, J = 8.10 Hz, H-3″ and H-5″), 3.91 (s, 2H, NCH₂), 3.87 (s, 2H, NCH₂), 2.69 |
| | | | | (t, 2H, J = 6.90 Hz, NCH₂), 2.67 (t, 2H, J = 6.90 Hz, NCH₂), 2.62-2.34 (m, 20H, 10NCH₂), 2.27 (s, |
| | | | | 3H, NCH₃), 2.26 (s, 3H, NCH₃), 1.58-1.48 (m, 8H, 4CH₂), 1.65-1.51 (m, 4H, 2CH₂); ¹³C NMR □ (75 |
| | | | | MHz, CDCl₃): 160.5 (C-2), 156.1 (C-4), 154.4 (C-7a), 144.8 (C-4′ and C-4″), 143.7 (C-1″′), 138.5 |
| | | | | (C-4a), 136.2 (C-1′ and C-1″), 131.3 (C-3″′ and C-5″′), 131.2 (C-3′ and C-5′), 129.9 (C-3″ and C-5″), |
| | | | | 129.7 (C-2′ and C-6′), 129.6 (C-2″ and C-6″), 129.4 (C-6), 128.3 (C-4″′), 124.7 (C-2″′ and C-6″′), 59.9 |
| | | | | (NCH₂), 56.5 (NCH₂), 55.1 (NCH₂), 54.6 (NCH₂), 50.5 (NCH₂), 47.4 (NCH₃), 29.4 (CH₂), 26.1 |
| | | | | (CH₂); MALDI-TOF MS m/z [M + H]⁺ Calculed: 715.4924, Found: 715.4911. |
| 42B | C₄₃H₅₈N₁₀•6(COOH)₂ | 74% | 174° | White crystals |
| | | | C. | |
| 43A | C₂₃H₂₅ClN₆ | 86% | | Yellow oil. ¹H NMR δ (300 MHz, CDCl₃): 8.76 (d, 2H, J = 8.40 Hz, H-2′ and H-6′), 8.33 (s, 1H, H- |
| | | | | 6), 7.69 (d, 2H, J = 7.80 Hz, H-2″ and H-6″), 7.57 (t, 2H, J = 7.80 Hz, H-3″ and H-5″), 7.50 (d, 2H, |
| | | | | J = 8.40 Hz, H-3′ and H-5′), 7.46 (t, 1H, J = 7.80 Hz, H-4″), 3.88 (s, 2H, NCH₂), 2.70 (t, 2H, J = 6.90 |
| | | | | Hz, NCH₂), 2.33 (t, 2H, J = 6.90 Hz, NCH₂), 2.22 (s, 6H, N(CH₃)₂), 1.70 (qt, 2H, J = 6.90 Hz, CH₂); |
| | | | | ¹³C NMR □ (75 MHz, CDCl₃): 158.3 (C-2), 156.2 (C-4), 154.9 (C-7a), 146.0 (C-1″ and C-4′), 135.2 |
| | | | | (C-4a), 134.4 (C-1′), 131.6 (C-3″ and C-5″), 131.4 (C-3′ and C-5′), 130.1 (C-6 and C-4″), 129.7 (C-2′ |
| | | | | and C-6′), 125.0 (C-2″ and C-6″), 59.5 (NCH₂), 55.1 (NCH₂), 49.3 (NCH₂), 46.9 (N(CH₃)₂), 29.3 |
| | | | | (CH₂); MALDI-TOF MS m/z [M + H]⁺ Calculed: 421.1907, Found: 421.1895. |
| 43B | C₂₃H₂₅ClN₆•2(COOH)₂ | 81% | 235° | White crystals |
| | | | C. | |
| 44A | C₃₃H₄₆N₆ | 68% | | Yellow oil. ¹H NMR δ (300 MHz, CDCl₃): 11.72 (s, 1H, NH), 8.06 (d, 2H, J = 8.10 Hz, H-2′ and H- |
| | | | | 6′), 7.80 (d, 2H, J = 8.10 Hz, H-2″ and H-6″), 7.63 (s, 1H, H-5), 7.54-7.49 (m, 4H, H-3′, H-5′, H-3″ |
| | | | | and H-5″), 7.27 (d, 1H, J = 3.30 Hz, H-2), 6.69 (d, 1H, J = 3.30 Hz, H-3), 3.93 (s, 2H, NCH₂), 3.92 |
| | | | | (s, 2H, NCH₂), 2.74 (t, 4H, J = 6.60 Hz, 2NCH₂), 2.34-2.26 (m, 4H, 2NCH₂), 2.24 (s, 6H, N(CH₃)₂), |
| | | | | 2.23 (s, 6H, N(CH₃)₂), 1.63-1.51 (m, 8H, 4CH₂); ¹³C NMR □ (75 MHz, CDCl₃): 153.3 (C-6), 153.1 |
| | | | | (C-7a), 144.0 (C-4), 141.9 (C-4′), 141.7 (C-1′), 141.0 (C-4″), 139.3 (C-1″), 130.0 (C-2′, C-6′, C-3′, C- |
| | | | | 5′, C-3″ and C-5″), 128.9 (C-2″ and C-6″), 127.6 (C-2), 118.9 (C-3a), 114.4 (C-5), 101.5 (C-3), 61.0 |
| | | | | (NCH₂), 55.0 (NCH₂), 50.7 (NCH₂), 46.8 (N(CH₃)₂), 29.3 (CH₂), 27.0 (CH₂); MALDI-TOF MS m/z |
| | | | | [M + H]⁺ Calculed: 527.3862, Found: 527.3855 |
| 44B | C₃₃H₄₆N₆•4(COOH)₂ | 52% | 125° | Pale-yellow crystals |
| | | | C. | |
| 45A | C₃₉H₅₆N₈ | 65% | | Yellow-orange oil. ¹H NMR δ (300 MHz, CDCl₃): 12.28 (s, 1H, NH), 8.04 (d, 2H, J = 8.10 Hz, H- |
| | | | | 2′ and H-6′), 7.78 (d, 2H, J = 8.10 Hz, H-2″ and H-6″), 7.60 (s, 1H, H-5), 7.50-7.47 (m, 4H, H-3′, H- |
| | | | | 5′, H-3″ and H-5″), 7.24 (d, 1H, J = 3.30 Hz, H-2), 6.66 (d, 1H, J = 3.30 Hz, H-3), 3.89 (s, 2H, NCH₂), |
| | | | | 3.88 (s, 2H, NCH₂), 2.73-2.69 (m, 4H, 2NCH₂), 2.51-2.32 (m, 20H, 10NCH₂), 2.28 (s, 3H, NCH₃), |
| | | | | 2.27 (s, 3H, NCH₃), 1.62-1.55 (m, 8H, 4CH₂); ¹³C NMR □ (75 MHz, CDCl₃): 153.0 (C-6), 151.2 (C- |
| | | | | 7a), 143.8 (C-4), 142.1 (C-4′), 142.0 (C-1′), 141.0 (C-4″), 139.2 (C-1″), 130.0 (C-2′, C-6′, C-3′, C-5′, |
| | | | | C-3″ and C-5″), 128.8 (C-2″ and C-6″), 127.7 (C-2), 118.9 (C-3a), 114.4 (C-5), 101.4 (C-3), 59.9 |
| | | | | (NCH₂), 56.5 (NCH₂), 55.1 (NCH₂), 54.6 (NCH₂), 50.7 (NCH₂), 47.4 (NCH₃), 29.4 (CH₂), 26.1 |
| | | | | (CH₂); MALDI-TOF MS m/z [M + H]⁺ Calculed: 637.4706, Found: 637.4712. |
| 45B | C₃₉H₅₆N₈•6(COOH)₂ | 57% | 176° | Yellow crystals |
| | | | C. | |
| 46A | C₄₀H₅₂N₆ | 80% | | Yellow oil. ¹H NMR δ (300 MHz, CDCl₃): 8.16 (d, 2H, J = 8.10 Hz, H-2′ and H-6′), 7.78 (d, 2H, J = |
| | | | | 8.10 Hz, H-2″ and H-6″), 7.68 (s, 1H, H-5), 7.50 (d, 2H, J = 8.10 Hz, H-3′ and H-5′), 7.44 (d, 2H, |
| | | | | J = 8.10 Hz, H-3″ and H-5″), 7.38-7.28 (m, 5H, 5H-_{Phen.}), 7.24 (d, 1H, J = 3.60 Hz, H-2), 6.67 (d, 1H, |
| | | | | J = 3.60 Hz, H-3), 5.62 (s, 2H CH₂Ph), 3.90 (s, 2H, NCH₂), 3.88 (s, 2H, NCH₂), 2.73 (t, 2H, J = 6.60 |
| | | | | Hz, NCH₂), 2.70 (t, 2H, J = 6.60 Hz, NCH₂), 2.30-2.25 (m, 4H, 2NCH₂), 2.24 (s, 6H, N(CH₃)₂), 2.23 |
| | | | | (s, 6H, N(CH₃)₂), 1.59-1.50 (m, 8H, 4CH₂); ¹³C NMR □ (75 MHz, CDCl₃): 152.6 (C-6), 152.3 (C- |
| | | | | 7a), 143.7 (C-4), 142.0 (C-4′), 141.7 (C-1′), 139.7 (C-1″′), 139.5 (C-4″), 139.3 (C-1″), 130.0 (C-2′, C- |
| | | | | 6′, C-3′, C-5′, C-3″, C-5″, C-2″′ and C-6″′), 129.2 (C-2″ and C-6″), 129.0 (C-2), 128.4 (C-3″′ and C- |
| | | | | 5″′), 128.3 (C-4″′), 118.7 (C-3a), 113.8 (C-5), 101.6 (C-3), 61.1 (NCH₂), 55.1 (NCH₂), 50.8 (NCH₂), |
| | | | | 50.6 (NCH₂), 49.4 50.8 (NCH₂), 46.8 (N(CH₃)₂), 29.4 (CH₂), 26.9 (CH₂); MALDI-TOF MS m/z |
| | | | | [M + H]⁺ Calculed: 617.4332, Found: 617.4347. |
| 46B | C₄₀H₅₂N₆•4(COOH)₂ | 69% | 136° | Pale-yellow crystals |
| | | | C. | |
| 47A | C₂₅H₂₇ClN₄ | 78% | | Yellow oil. ¹H NMR δ (300 MHz, CDCl₃): 8.04 (d, 2H, J = 8.40 Hz, H-2′ and H-6′), 7.88 (d, 2H, J = |
| | | | | 7.80 Hz, H-2″ and H-6″), 7.68 (s, 1H, H-5), 7.58 (d, 1H, J = 3.60 Hz, H-2), 7.56 (t, 2H, J = 7.80 |
| | | | | Hz, H-3″ and H-5″), 7.43 (d, 2H, J = 8.40 Hz, H-3′ and H-5′), 7.38 (t, 1H, J = 7.80 Hz, H-4″), 6.75 |
| | | | | (d, 1H, J = 3.60 Hz, H-3), 3.87 (s, 2H, NCH₂), 2.72 (t, 2H, J = 6.90 Hz, NCH₂), 2.35 (t, 2H, J = 6.90 |
| | | | | Hz, NCH₂), 2.24 (s, 6H, N(CH₃)₂), 1.72 (qt, 2H, J = 6.90 Hz, CH₂); ¹³C NMR □ (75 MHz, CDCl₃): |
| | | | | 153.5 (C-6), 152.6 (C-4), 142.4 (C-7a and C-1″), 139.2 (C-4′), 137.6 (C-1′), 130.6 (C-3″ and C-5″), |
| | | | | 129.9 (C-3′ and C-5′), 129.7 (C-2), 128.4 (C-2′ and C-6′), 127.7 (C-4″), 125.1 (C-2″ and C-6″), 120.9 |
| | | | | (C-3a), 115.3 (C-5), 101.6 (C-3), 59.5 (NCH₂), 55.0 (NCH₂), 49.2 (NCH₂), 46.9 (N(CH₃)₂), 20.8 |
| | | | | (CH₂); MALDI-TOF MS m/z [M + H]⁺ Calculed: 419.2002, Found: 419.1991. |
| 47B | C₂₅H₂₇ClN₄•2(COOH)₂ | 53% | 229° | White crystals |
| | | | C. | |
| 48A | C₃₉H₅₁N₇O | 61% | | Yellow oil; ¹H NMR δ (300 MHz, CDCl₃): 8.60 (d, 2H, J = 8.10 Hz, H-2′ and H-6′), 8.26 (d, 2H, J = |
| | | | | 8.10 Hz, H-2″ and H-6″), 7.78 (d, 2H, J = 9.00 Hz, H-2″′ and H-6″′), 7.56 (d, 2H, J = 8.10 Hz, H- |
| | | | | 3′ and H-5′), 7.52 (d, J = 3.90 Hz, H-6), 7.44 (d, 2H, J = 8.10 Hz, H-3″ and H-5″), 7.12 (d, 2H, J = |
| | | | | 9.00 Hz, H-3″′ and H-5″′), 6.97 (d, 1H, J = 3.90 Hz, H-5), 3.94 (s, 2H, NCH₂), 3.92 (s, 3H, OCH₃), |
| | | | | 3.87 (s, 2H, NCH₂), 2.73-2.66 (m, 4H, 2NCH₂), 2.30 (t, 2H, J = 7.20 Hz, NCH₂), 2.27 (t, 2H, J = 7.20 |
| | | | | Hz, NCH₂), 2.22 (s, 6H, N(CH₃)₂), 2.21 (s, 6H, N(CH₃)₂), 1.58-1.47 (m, 8H, 4CH₂); ¹³C NMR δ (75 |
| | | | | MHz, CDCl₃): 159.5 (C-2), 159.4 (C-4), 158.7 (C-4″′), 153.9 (C-7a), 143.8 (C-4″), 143.0 (C-4′), |
| | | | | 139.1 (C-1″′), 138.8 (C-1′), 132.3 (C-1″), 130.5 (C-2″ and C-6″), 130.2 (C-6), 129.8 (C-2′ and C-6′), |
| | | | | 129.6 (C-3″ and C-5″), 129.5 (C-3′ and C-5′), 126.5 (C-2″′ and C-6″′), 115.8 (C-3″′ and C-5″′), 116.1 |
| | | | | (C-4a), 103.1 (C-5), 61.0 (NCH₂), 56.9 (OCH₃), 55.0 (NCH₂), 50.6 (NCH₂), 50.5 (NCH₂), 46.8 |
| | | | | (NCH₃), 29.3 (CH₂), 26.9 (CH₂); MALDI-TOF MS m/z [M + H]⁺ Calculed: 634.4233, Found: |
| | | | | 634.4245. |
| 48B | C₃₉H₅₁N₇O•4(COOH)₂ | 85% | 135° | Yellow crystals |
| | | | C. | |
| 49A | C₃₈H₄₈N₆S | 81% | | Yellow oil; ¹H NMR δ (300 MHz, CDCl₃): 8.57 (d, 2H, J = 8.40 Hz, H-2′ and H-6′), 8.26 (d, 2H, J = |
| | | | | 8.40 Hz, H-2″ and H-6″), 7.75-7.71 (m, 3H, H-7 and 2H-phenyl), 7.53 (d, 2H, J = 8.40 Hz, H-3′ |
| | | | | and H-5′), 7.45 (d, 2H, J = 8.40 Hz, H-3″ and H-5″), 7.42-7.38 (m, 3H-phenyl), 3.88 (s, 2H, NCH₂), |
| | | | | 3.86 (s, 2H, NCH₂), 2.67 (t, 4H, J = 6.60 Hz, 2NCH₂), 2.25 (t, 4H, J = 6.60 Hz, 2NCH₂), 2.20 (s, 6H, |
| | | | | N(CH₃)₂), 2.17 (s, 6H, N(CH₃)₂), 1.58-1.47 (m, 8H, 4CH₂); ¹³C NMR δ (75 MHz, CDCl₃): 165.2 (C- |
| | | | | 4), 162.5 (C-2), 159.9 (C-7a), 154.8 (C-6), 144.8 (C-4′), 143.5 (C-4″), 138.5 (C-1″′), 137.6 (C-1′), |
| | | | | 134.2 (C-1″), 131.3 (C-4″′), 130.6 (C-3″′ and C-5″), 130.0 (C-2″ and C-6″), 129.9 (C-2′ and C-6′), |
| | | | | 129.8 (C-3″ and C-5″), 129.7 (C-2″′ and C-6″′), 128.0 (C-3′ and C-5′), 127.0 (C-4a), 121.5 (C-7), 61.0 |
| | | | | (NCH₂), 55.0 (NCH₂), 50.7 (NCH₂), 46.8 (2N(CH₃)₂), 29.3 (CH₂), 26.9 (CH₂); MALDI-TOF MS m/z |
| | | | | [M + H]⁺ Calculed: 621.3739, Found: 621.3744. |
| 49B | C₃₈H₄₈N₆S•4(COOH)₂ | 77% | 165° | Pale-yellow crystals |
| | | | C. | |

### Pathologies

The compounds of formula (I) may be useful in the treatment and/or in the prevention of infectious diseases, in particular viral and/or parasitic infectious diseases.

Parasitic infectious diseases may be amoebiasis, giardiasis, trichomoniasis, African Sleeping Sickness, American Sleeping Sickness, trypanosomiasis, leishmaniasis (Kala-Azar), balantidiasis, toxoplasmosis, malaria, acanthamoeba keratitis, babesiosis and Helminth infections, such as schistosomiasis or ascariasis.

Viral infectious diseases may be acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, Covid-19, Respiratory Syncytial Virus (RSV) infection, and herpes zoster.

Unless instructed otherwise, all the disclosed compounds may specifically be considered herein for the treatment or prevention of Coronaviridae, which may thus refer indifferently to any member of the said Coronaviridae family in the sense of the Baltimore convention, although particular selections of viruses will be considered hereafter as preferred embodiments.

As used herein, the term “Coronaviridae” refers to the corresponding family of RNA viruses belonging to the group IV of the Baltimore classification, which is itself part of the Cornidovirineae suborder and of the Nidovirales Order. The Coronaviridae family includes both the Letovirinae and Orthocoronavirinae subfamilies.

As used herein, the term “Letovirinae” refers to the corresponding family of the Baltimore classification, which includes the Alphaletovirus genus, the Milecovirus subgenus, which includes (in a non-exhaustive manner) the Microhyla letovirus 1 species.

As used herein, the term “Orthocoronavirinae” refers to the corresponding family of the Baltimore classification, which includes the Alphacoronavirus, Betacoronavirus, Deltacoronavirus, and Gammacoronavirus genus.

As used herein, the term “Alphacoronavirus” refers to the corresponding family of the Baltimore classification, which includes the Colacovirus, Decacovirus, Duvinacovirus, Luchacovirus, Minacovirus, Minunacovirus, Myotacovirus, Myctacovirus, Pedacovirus, Rhinacovirus, Setracovirus, and Tegacovirus subgenus. In a non-exhaustive manner, this includes the following species: Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Alphacoronavirus 1.

As used herein, the term “Betacoronavirus” refers to the corresponding family of the Baltimore classification, which includes the Embecovirus, Hibecovirus, Merbecovirus, Nobecovirus, and Sarbecovirus subgenus. In a non-exhaustive manner, this includes the following species: Betacoronavirus 1, China Rattus coronavirus HKU24, Human coronavirus HKU1, Murine coronavirus, Bat Hp-betacoronavirus Zhejiang 2013, Hedgehog coronavirus 1, Middle East respiratory syndrome-related coronavirus, Pipistrellus bat coronavirus HKU5, Tylonycteris bat coronavirus HKU4, Hedgehog coronavirus 1, Middle East respiratory syndrome-related coronavirus, Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus.

As used herein, the term “Severe acute respiratory syndrome-related coronavirus”, or SARS virus, includes, in a non-exhaustive manner, the SARS-CoV, SARSr-CoV WIV1, SARSr-CoV HKU3, SARSr-CoV RP3, and SARS-CoV-2; including strains responsible for COVID-19 and their mutants.

As used herein, the term “Deltacoronavirus” refers to the corresponding family of the Baltimore classification, which includes the Andecovirus, Buldecovirus, Herdecovirus, and Moordecovirus subgenus. In a non-exhaustive manner, this includes the following species: Wigeon coronavirus HKU20, Bulbul coronavirus HKU11, Coronavirus HKU15, Munia coronavirus HKU13, White-eye coronavirus HKU16, Night heron coronavirus HKU19, Common moorhen coronavirus HKU21.

As used herein, the term “Gammacoronavirus” refers to the corresponding family of the Baltimore classification, which includes the Cegacovirus and Igacovirus subgenus. In a non-exhaustive manner, this includes the following species: Beluga whale coronavirus SW1 and Avian coronavirus.

In particular, the compounds of formula (I) are particularly useful in the prevention and/or treatment of infectious diseases induced by parasites and/or viruses, of which replication cycle key processes involve DNA or RNA G-quadruplexes from the parasite and/or virus and/or host cell genome or transcriptome.

According to a particular embodiment, the compounds of formula (I) of the present invention may be useful in the treatment and/or in the prevention of infectious diseases induced by parasites and/or viruses selected among P. falciparum, L. donovani, Trypanosoma brucei, Ascaris lumbricoides, S. mansoni, human immunodeficiency virus (HIV), herpes simplex virus (HSV), human papillomavirus (HPV), Epstein-Barr virus (EBV), Hepatitis C virus (HCV) and SARS-CoV-2.

Still according to this particular embodiment, the compounds of formula (I) of the present invention may be useful in the treatment and/or prevention of Severe acute respiratory syndrome (SARS) induced by SARS-CoV-2, acquired immunodeficiency syndrome, African Sleeping Sickness, American Sleeping Sickness, trypanosomiasis, leishmaniasis (Kala-Azar) or malaria.

The following examples are provided as illustrations and in no way limit the scope of this invention.

The following examples illustrate in detail the preparation of some compounds according to the invention. The structures of the products obtained have been confirmed by NMR analyses and mass spectroscopy.

### Example 1:—Synthesis of Some Starting Reagents (SR)

2,4-dichloro-7H-pyrrolo[2,3-d]pyrimidine (SR1) is available from Appolo Scientific.

5-amino-1-phenyl-1H-pyrazole-4-carboxamide (SR4) is available from Sigma-Aldrich.

2,4-dichloro-7H-imidazo[4,5-d]pyrimidine (SR6) is available from Sigma-Aldrich.

4,6-dichloro-1H-pyrrolo[2,3-b]pyridine (SR8) is available from Sigma-Aldrich.

### Example 1.1: Preparation of 2,4-dichloro-7-phenyl-7H-pyrrolo[2,3-d]pyrimidine (SR2)

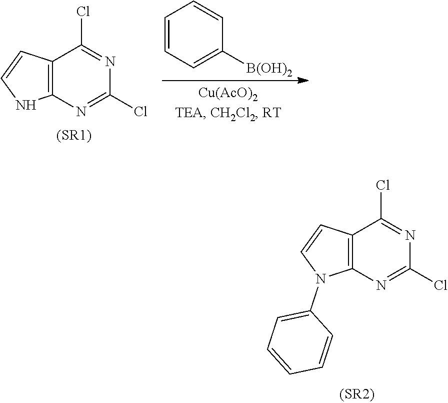 [SMILES: Clc1nc(Cl)c2cc[nH]c2n1 ; OB(O)c1ccccc1 ; Clc1nc(Cl)c2ccn(-c3ccccc3)c2n1]

2,4-Dichloro-7H-pyrrolo[2,3-d]pyrimidine (5 mmol), phenylboronic acid (6 mmol, 1.2 equiv.) and copper (II) acetate (5 mmol) in methanol (75 mL) was stirred at room temperature for 4 hours. Then, the reaction mixture was evaporated to dryness. The residue was extracted with chloroform, filtered and evaporated under reduced pressure. The residue was then purified by silica gel column chromatography (CHCl₃), then cooled and triturated in EtOH, filtered on sintered glassware, washed with a minimum of EtOH, then EtO₂ and petroleum ether and dried under pressure to give the solid product. White crystals (34%).

### Example 1.2: Preparation of 2,4-Dichloro-7-(4-formylphenyl)-7H-pyrrolo[2,3-d]pyrimidine (SR3)

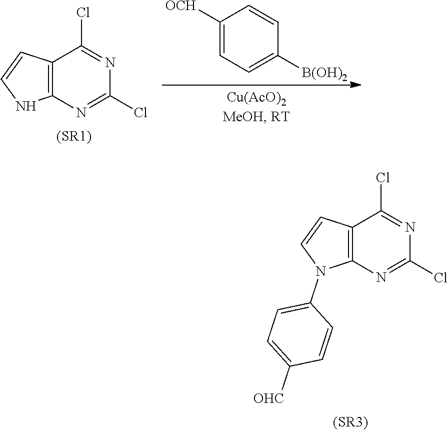

Compound (SR3) is prepared as detailed in Scheme 11 here-above.

2,4-Dichloro-7H-pyrrolo[2,3-d]pyrimidine (5 mmol), 4-formylphenylboronic acid (6 mmol, 1.2 equiv.) and copper (II) acetate (5 mmol) in methanol (75 mL) was stirred at room temperature for 4 hours. Then, the reaction mixture was evaporated to dryness. The residue was extracted with chloroform, filtered and evaporated under reduced pressure. The residue was then purified by silica gel column chromatography (CHCl₃), then cooled and triturated in EtOH, filtered on sintered glassware, washed with a minimum of EtOH, then EtO₂ and petroleum ether and dried under pressure to give the solid product.

White crystals (36%); Mp=224° C.; ¹H NMR δ (300 MHz, CDCl₃): 10.11 (s, 1H, CHO), 8.11 (d, 2H, J=8.40 Hz, H-2′ and H-6′), 7.97 (d, 2H, J=8.40 Hz, H-3′ and H-5′), 7.64 (d, 1H, J=3.90 Hz, H-6), 6.87 (d, 1H, J=3.90 Hz, H-5).

### Example 1.3: Preparation of 4,6-dichloro-1-phenyl-1H-pyrazolo[3,4-d]pyrimidine (SR5)

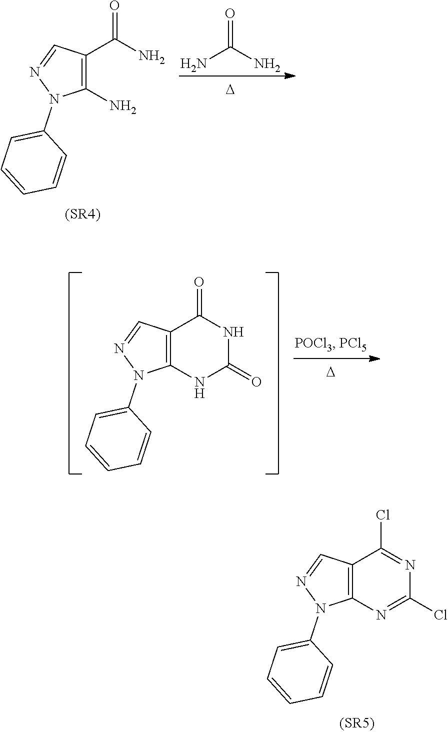

5-Amino-1-phenyl-pyrazole-4-carboxamide (5 mmol) was fused and stirred with urea (32.5 mmol, 6.5 equiv.) at approximately 200° C. for 3 hours. Then, the reaction mixture was cooled, and dissolved in dilute sodium hydroxide; the solution was treated with charcoal and filtered. The filtrate was acidified with glacial acetic acid and the white precipitate collected. The solid was washed with water and dried to give the 1-phenyl-1H-pyrazolo[3,4-d]pyrimidin-4,6-dione. The latter (1.6 mmol) was then refluxed with a mixture of 1.5 g of phosphorus pentachloride and 5 mL of phosphorus oxychloride. The solution was refluxed for 2 hours. The excess phosphorus oxychloride was removed under reduced pressure and the residue poured with vigorous stirring, onto crushed ice. The solution was extracted with chloroform (25 mL). The organic layer was washed with 20 mL of water, dried over Na₂SO₄, filtered and evaporated to dryness. The product was recrystallized from heptane to give the solid product.

White crystals (85%); Mp=127° C.; ¹H NMR δ (300 MHz, CDCl₃): 8.36 (s, 1H, H-3), 8.16 (d, 2H, J=8.70 Hz, H-2′ and H-6′), 7.59 (t, 2H, J=8.70 Hz, H-3′ and H-5′), 7.43 (t, 1H, J=8.70 Hz, H-4′).

### Example 1.4: Preparation of 2,4-dichloro-7-phenyl-7H-imidazo[4,5-d]pyrimidine (SR7)

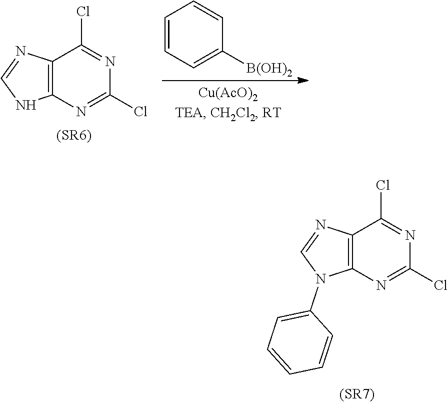 [SMILES: Clc1nc(Cl)c2nc[nH]c2n1 ; OB(O)c1ccccc1 ; Clc1nc(Cl)c2ncn(-c3ccccc3)c2n1]

2,4-dichloro-7H-imidazo[4,5-d]pyrimidine (2.7 mmol), phenylboronic acid (8.1 mmol, 3.0 equiv.), 1,10-phenantroline (5.4 mmol, 2.0 equiv.), copper (II) acetate (2.7 mmol) and 2 g of molecular sieves (4 Å) in dichloromethane (30 mL) was stirred at room temperature for 4 days. Then, 250 mL of methanol were added to the reaction mixture which was then filtered on Celite. The filtrate was evaporated to dryness. The residue was then purified by silica gel column chromatography (CH₂Cl₂) to give the solid product.

White crystals (51%); Mp=240° C.; ¹H NMR δ (300 MHz, CDCl₃): 8.40 (s, 1H, H-6), 7.72-7.68 (m, 2H, H-phenyl), 7.64-7.62 (m, 2H, H-phenyl), 7.58-7.52 (m, 1H, H-phenyl).

### Example 1.5: Preparation of 2,4-dichloro-7-phenyl-7H-imidazo[4,5-d]pyrimidine (SR9)

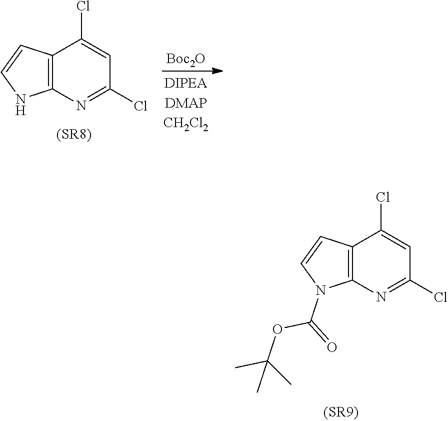 [SMILES: "Boc2O" ; Clc1cc(Cl)c2cc[nH]c2n1 ; CC(C)(C)OC(=O)n1ccc2c(Cl)cc(Cl)nc21]

To a solution of 4,6-Dichloro-1H-pyrrolo[2,3-b]pyridine (5.3 mmol) in anhydrous CH₂Cl₂ (20 mL), DIPEA (6.4 mmol, 1.2 eq.), DMAP (1.1 mmol, 0.2 eq.) and Boc₂O (8.0 mmol, 1.5 eq.) were added. The reaction mixture was stirred under reflux for 10 min. The solvent was evaporated under reduced pressure and the obtained solid was purified by column chromatography (eluent-CHCl₃) to give the 4,6-dichloro-1-tert-butoxycarbonyl-1H-pyrrolo[2,3-b]pyridine (SR9).

White crystals (90%); Mp=74° C.; ¹H NMR δ (300 MHz, CDCl₃): 7.46 (d, 1H, J=3.90 Hz, H-2), 6.96 (s, 1H, H-5), 6.30 (d, 1H, J=3.90 Hz, H-3), 1.51 (s, 9H C(CH₃)₃).

### Example 1.6: Preparation of 4,6-dichloro-1H-pyrrolo[2,3-b]pyridine (SR10)

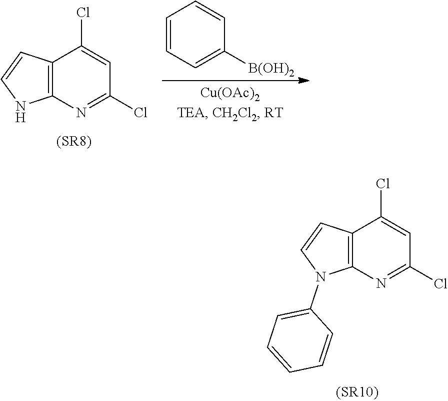 [SMILES: Clc1cc(Cl)c2cc[nH]c2n1 ; OB(O)c1ccccc1 ; Clc1cc(Cl)c2ccn(-c3ccccc3)c2n1]

To a solution of 4,6-Dichloro-1H-pyrrolo[2,3-b]pyridine (2.7 mmol) in dichloromethane (10 mL) were added phenylboronic acid (3 mmol, 1.1 equiv.), copper (II) acetate (5.4 mmol, 2.2 equiv.) and Et₃N (27 mmol, 10 equiv.). The mixture was stirred at room temperature for 24 hours. Water was added to quench the reaction, and the resulting mixture was extracted with EtOAc (two times). The combined organic phases were washed with saturated brine, dried over anhydrous Na₂SO₄, filtered, and the filtrate was concentrated in vacuum. The residue was then purified by silica gel column chromatography (CH₂Cl₂) to give the solid product (SR10).

White crystals (32%); Mp=56° C.; ¹H NMR δ (300 MHz, CDCl₃): 7.72 (dd, 2H, J=8.70 and 1.50 Hz, H-2′ and H-6′), 7.58-7.53 (m, 2H, H-3′ and H-5′), 7.52 (d, 1H, J=3.90 Hz, H-2), 7.39 (m, 1H, H-4′), 7.23 (s, 1H, H-5), 6.75 (d, 1H, J=3.90 Hz, H-3).

### Example 2—Preparation of Compounds (1) to (11), (24), (26), (27), (29), (30), (33) to (42) and (44) to (46)

Compounds (1) to (11), (24), (26), (27), (29), (30), (33) to (42) and (44) to (46) are prepared following general protocol 6 (GP6) as defined in Scheme 3 here-above.

### Example 2.1—Synthesis of Compounds (1) to (5), (33) and (34)

### Step 1A (Ar₁=Ar₂): Preparation of 2,4-Bis(4-formylphenyl)-7-phenyl-7H-pyrrolo[2,3-d]pyrimidine

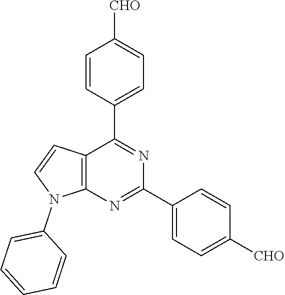 [SMILES: O=Cc1ccc(-c2nc(-c3ccc(C=O)cc3)c3ccn(-c4ccccc4)c3n2)cc1]

To a solution of 4.37 mmol of (SR2), 1.44 g of 4-formylphenyl boronic acid (9.63 mmol, 2.2 eq.) and 506 mg (0.437 mmol, 0.1 eq.) of tetrakis(triphenylphosphine) palladium in 45 mL of 1,2-dimethoxyethane, 5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, EtO₂ and petroleum ether and dried under pressure to give the solid product.

Pale-yellow crystals (76%); Mp=146° C.; IR νₘₐₓ (KBr)/cm⁻¹ 1690 and 1686 (C═O); ¹H NMR δ (300 MHz, CDCl₃): 10.17 (s, 1H, CHO), 10.09 (s, 1H, CHO), 8.70 (d, 2H, J=8.40 Hz, H-2′ and H-6′), 8.52 (d, 2H, J=8.40 Hz, H-2″ and H-6″), 8.25 (d, 1H, J=3.60 Hz, H-6), 8.16 (d, 2H, J=8.10 Hz, H-2′″ and H-6′″), 8.05 (d, 2H, J=8.40 Hz, H-3′ and H-5′), 7.98 (d, 2H, J=8.40 Hz, H-3″ and H-5″), 7.66 (t, 2H, J=8.10 Hz, H-3′″ and H-5″), 7.49 (t, 1H, J=8.10 Hz, H-4′″), 7.25 (d, 1H, J=3.60 Hz, H-5).

### Step 2 Preparation of 2,4-bis[(substituted-iminomethyl)]-7-phenyl-7H-pyrrolo[2,3-d]pyrimidines

To a solution of the appropriate diamines (0.126 mmol, 2.1 eq.) in ethanol (7 mL) was added the 2,4-bis(4-formylphenyl)-7-phenyl-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Preparation of 2,4-bis[(substituted-aminomethyl)]-7-phenyl-7H-pyrrolo[2,3-d]pyrimidine

To a solution of 2,4-bis[(substituted-iminomethyl)]-7-phenyl-7H-pyrrolo[2,3-d]pyrimidines (0.4 mmol) in methanol (10 mL) was added portion-wise at 0° C. sodium borohydride (3.2 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 2,4-bis[(substituted-aminomethyl)]-7-phenyl-7H-pyrrolo[2,3-d]pyrimidines.

### Compound (1)

STEP 1A is performed as disclosed here-above.

### Step 2: Synthesis of 2,4-bis{4-[(3-dimethylaminopropyl)iminomethyl]phenyl}-7-phenyl-7H-pyrrolo[2,3-d]pyrimidine

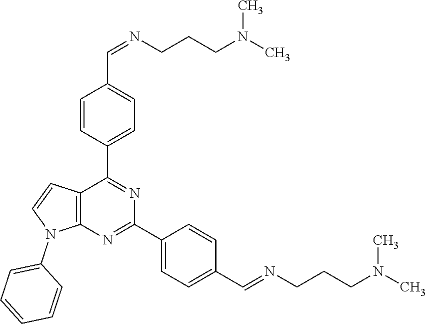 [SMILES: CN(C)CCC/N=C\c1ccc(-c2nc(-c3ccc(/C=N/CCCN(C)C)cc3)nc3c2ccn3-c2ccccc2)cc1]

Pale-yellow crystals (97%) Mp=141° C.; ¹H NMR δ (300 MHz, CDCl₃): 8.67 (d, 2H, J=8.40 Hz, H-2′ and H-6′), 8.39 (s, 1H, CH═N), 8.35 (s, 1H, CH═N), 8.30 (d, 2H, J=8.40 Hz, H-2″ and H-6″), 7.93 (d, 2H, J=8.40 Hz, H-3′ and H-5′), 7.88-7.82 (m, 4H, H-2′″, H-6′″, H-3″ and H-5″), 7.57 (t, 2H, J=7.80 Hz, H-3′″ and H-5″), 7.56 (d, 1H, J=3.60 Hz, H-6), 7.40 (t, 1H, J=7.80 Hz, H-4′″), 6.94 (d, 1H, J=3.60 Hz, H-5), 3.74-3.65 (m, 4H, 2NCH₂), 2.42-2.35 (m, 4H, 2NCH₂), 2.27 (s, 6H, N(CH₃)₂), 2.25 (s, 6H, N(CH₃)₂), 1.98-1.85 (m, 4H, 2CH₂).

STEP 3 leads to compound (1A) as detailed in Table 3.

The corresponding oxalate salt, compound (1B), has also been obtained as detailed in Table 3.

### Compound (2)

STEP 1A is performed as disclosed here above

### Step 2: Synthesis of 2,4-bis{4-[(3-(4-methylpiperazin-1-yl)propyl)iminomethyl]phenyl}-7-phenyl-7H-pyrrolo[2,3-d]pyrimidine

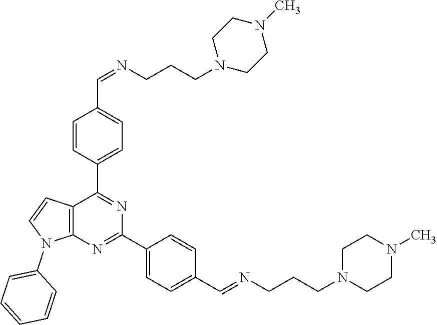 [SMILES: CN1CCN(CCC/N=C\c2ccc(-c3nc(-c4ccc(/C=N/CCCN5CCN(C)CC5)cc4)nc4c3ccn4-c3ccccc3)cc2)CC1]

Yellow oil (58%); ¹H NMR δ (300 MHz, CDCl₃): 8.66 (d, 2H, J=8.40 Hz, H-2′ and H-6′), 8.38 (s, 1H, CH═N), 8.33 (s, 1H, CH═N), 8.30 (d, 2H, J=8.40 Hz, H-2″ and H-6″), 7.92 (d, 2H, J=8.40 Hz, H-3′ and H-5′), 7.87 (dd, 2H, J=7.85 and 1.20 Hz, H-2′″, H-6′″), 7.82 (d, 2H, J=8.40 Hz, H-3″ and H-5″), 7.58 (d, 1H, J=3.60 Hz, H-6), 7.57 (t, 2H, J=7.85 Hz, H-3′″ and H-5″), 7.40 (t, 1H, J=7.85 Hz, H-4′″), 6.95 (d, 1H, J=3.60 Hz, H-5), 3.73-3.64 (m, 4H, 2NCH₂), 2.57-2.35 (m, 20H, 10NCH₂), 2.25 (s, 3H, NCH₃), 2.35 (s, 3H, NCH₃), 1.98-1.85 (m, 4H, 2CH₂).

STEP 3 leads to compound (2A) as detailed in Table 3.

The corresponding oxalate salt, compound (2B) has also been obtained as detailed in Table 3.

### Compound (3)

STEP 1A is performed as disclosed here-above

### Step 2: Synthesis of 2,4-bis{4-[(3-dimethylaminobutyl)iminomethyl]phenyl}-7-phenyl-7H-pyrrolo[2,3-d]pyrimidine

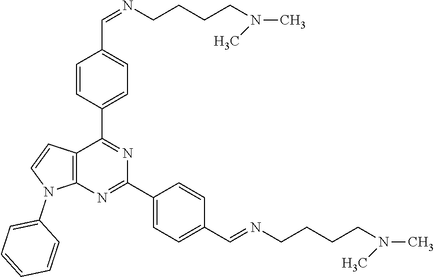 [SMILES: CN(C)CCCC/N=C\c1ccc(-c2nc(-c3ccc(/C=N/CCCCN(C)C)cc3)nc3c2ccn3-c2ccccc2)cc1]

Yellow oil (98%); ¹H NMR δ (300 MHz, CDCl₃): 8.67 (d, 2H, J=8.40 Hz, H-2′ and H-6′), 8.39 (s, 1H, CH═N), 8.35 (s, 1H, CH═N), 8.31 (d, 2H, J=8.40 Hz, H-2″ and H-6″), 7.94 (d, 2H, J=8.40 Hz, H-3′ and H-5′), 7.88 (dd, 2H, J=8.40 and 1.20 Hz H-2′″, H-6′″), 7.83 (d, 2H, J=8.40 Hz, H-3″ and H-5″), 7.59 (d, 1H, J=3.90 Hz, H-6), 7.58 (t, 2H, J=8.40 Hz, H-3′″ and H-5′″), 7.41 (t, 1H, J=7.80 Hz, H-4′″), 6.96 (d, 1H, J=3.90 Hz, H-5), 3.73-3.65 (m, 4H, 2NCH₂), 2.34 (t, 2H, J=7.20 Hz, NCH₂), 2.32 (t, 2H, J=7.20 Hz, NCH₂), 2.24 (s, 6H, N(CH₃)₂), 2.23 (s, 6H, N(CH₃)₂), 1.81-1.71 (m, 4H, 2CH₂), 1.65-1.54 (m, 4H, 2CH₂).

STEP 3 leads to compound (3A) as detailed in Table 3.

The corresponding oxalate salt, compound (3B), has also been obtained as detailed in Table 3.

### Compound (4)

STEP 1A is performed as disclosed here-above.

### Step 2: Synthesis of 2,4-bis{4-[(3-(4-methylpiperazin-1-yl)butyl)iminomethyl]phenyl}-7-phenyl-7H-pyrrolo[2,3-d]pyrimidine

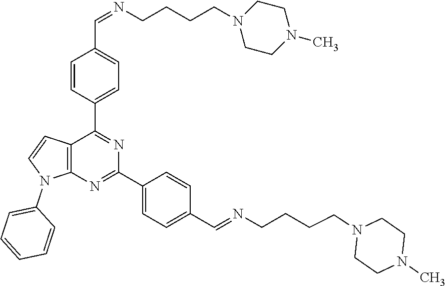 [SMILES: CN1CCN(CCCC/N=C\c2ccc(-c3nc(-c4ccc(/C=N/CCCCN5CCN(C)CC5)cc4)nc4c3ccn4-c3ccccc3)cc2)CC1]

Yellow oil (97%); ¹H NMR δ (300 MHz, CDCl₃): 8.65 (d, 2H, J=8.40 Hz, H-2′ and H-6′), 8.36 (s, 1H, CH═N), 8.31 (s, 1H, CH═N), 8.29 (d, 2H, J=8.40 Hz, H-2″ and H-6″), 7.91 (d, 2H, J=8.40 Hz, H-3′ and H-5′), 7.85 (d, 2H, J=8.10 Hz, H-2′″, H-6′″), 7.80 (d, 2H, J=8.40 Hz, H-3″ and H-5″), 7.57 (d, 1H, J=3.60 Hz, H-6), 7.55 (t, 2H, J=8.10 Hz, H-3′″ and H-5″), 7.38 (t, 1H, J=8.10 Hz, H-4′″), 6.94 (d, 1H, J=3.60 Hz, H-5), 3.67-3.63 (m, 4H, 2NCH₂), 2.54-2.36 (m, 20H, 10NCH₂), 2.26 (s, 3H, NCH₃), 2.25 (s, 3H, NCH₃), 1.82-1.66 (m, 4H, 2CH₂), 1.62-1.50 (m, 4H, 2CH₂).

STEP 3 leads to compound (4A).

The corresponding oxalate salt, compound (4B) has also been obtained.

### Compound (5)

STEP 1A is performed as disclosed here-above.

### Step 2: Synthesis of 2,4-bis{4-[(3-dimethylaminopentyl)iminomethyl]phenyl}-7-phenyl-7H-pyrrolo[2,3-d]pyrimidine

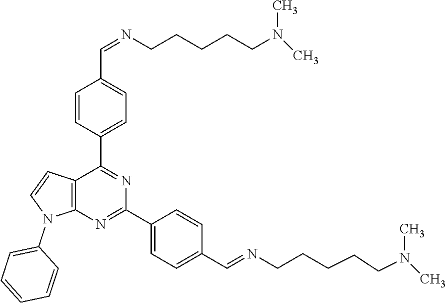 [SMILES: CN(C)CCCCC/N=C\c1ccc(-c2nc(-c3ccc(/C=N/CCCCCN(C)C)cc3)nc3c2ccn3-c2ccccc2)cc1]

Yellow oil (98%); ¹H NMR δ (300 MHz, CDCl₃): 8.63 (d, 2H, J=8.40 Hz, H-2′ and H-6′), 8.33 (s, 1H, CH═N), 8.29 (s, 1H, CH═N), 8.25 (d, 2H, J=8.40 Hz, H-2″ and H-6″), 7.88 (d, 2H, J=8.40 Hz, H-3′ and H-5′), 7.82-7.78 (m, 4H, H-2′″, H-6′″, H-3″ and H-5″), 7.52 (t, 2H, J=7.80 Hz, H-3′″ and H-5′″) 7.50 (d, 1H, J=3.60 Hz, H-6), 7.33 (t, 1H, J=7.80 Hz, H-4′″), 6.87 (d, 1H, J=3.60 Hz, H-5), 3.64 (t, 2H, J=7.20 Hz NCH₂), 3.62 (t, 2H, J=7.20 Hz NCH₂), 2.28-2.19 (m, 4H, 2NCH₂), 2.20 (s, 6H, N(CH₃)₂), 2.18 (s, 6H, N(CH₃)₂), 1.80-1.68 (m, 4H, 2CH₂), 1.55-1.47 (m, 4H, 2CH₂), 1.45-1.35 (m, 4H, 2CH₂).

STEP 3 leads to compound (5A) as detailed in Table 3.

The corresponding oxalate salt, compound (5B), has also been obtained as detailed in Table 3.

### Compound (33)

STEP 1A is performed as disclosed here-above

### Step 2: Synthesis of 2,4-bis{4-[(3-diethylaminobutyl)iminomethyl]phenyl}-7-phenyl-7H-pyrrolo[2,3-d]pyrimidine

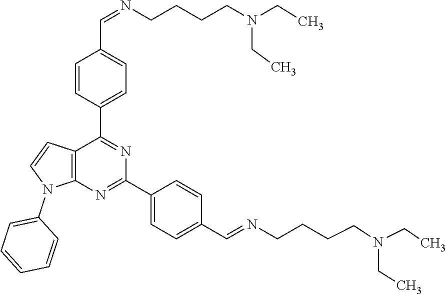 [SMILES: CCN(CC)CCCC/N=C\c1ccc(-c2nc(-c3ccc(/C=N/CCCCN(CC)CC)cc3)nc3c2ccn3-c2ccccc2)cc1]

Yellow oil (92%); ¹H NMR δ (300 MHz, CDCl₃): 8.68 (d, 2H, J=8.40 Hz, H-2′ and H-6′), 8.41 (s, 1H, CH═N), 8.36 (s, 1H, CH═N), 8.33 (d, 2H, J=8.40 Hz, H-2″ and H-6″), 7.96 (d, 2H, J=8.40 Hz, H-3′ and H-5′), 7.89 (dd, 2H, J=8.40 and 1.20 Hz H-2′″, H-6′″), 7.85 (d, 2H, J=8.40 Hz, H-3″ and H-5″), 7.63 (d, 1H, J=3.60 Hz, H-6), 7.61 (t, 2H, J=8.40 Hz, H-3′″ and H-5′″), 7.43 (t, 1H, J=7.80 Hz, H-4′″), 7.00 (d, 1H, J=3.60 Hz, H-5), 3.76-3.64 (m, 4H, 2NCH₂), 2.60-2.45 (m, 12H, 6NCH₂), 1.80-1.71 (m, 4H, 2CH₂), 1.63-1.52 (m, 4H, 2CH₂), 2.24 (t, 6H, J=7.20 Hz, 2CH₃), 2.23 (t, 6H, J=7.20 Hz, 2CH₃).

STEP 3 leads to compound (33A) as detailed in Table 3.

The corresponding oxalate salt, compound (33B), has also been obtained as detailed in Table 3.

### Compound (34)

STEP 1A is performed as disclosed here-above

### Step 2: Synthesis of 2,4-bis{4-[(3-diisopropylaminopentyl)iminomethyl]phenyl}-7-phenyl-7H-pyrrolo[2,3-d]pyrimidine

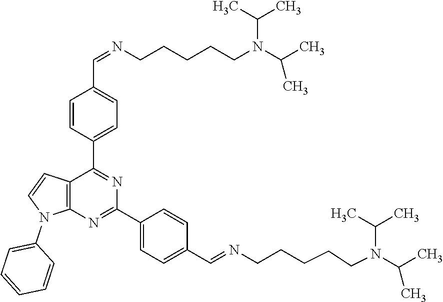 [SMILES: CC(C)N(CCCCC/N=C\c1ccc(-c2nc(-c3ccc(/C=N/CCCCCN(C(C)C)C(C)C)cc3)nc3c2ccn3-c2ccccc2)cc1)C(C)C]

Yellow oil (98%); ¹H NMR δ (300 MHz, CDCl₃): 8.70 (d, 2H, J=8.40 Hz, H-2′ and H-6′), 8.41 (s, 1H, CH═N), 8.36 (s, 1H, CH═N), 8.35 (d, 2H, J=8.40 Hz, H-2″ and H-6″), 7.97 (d, 2H, J=8.40 Hz, H-3′ and H-5′), 7.90 (d, 2H, J=7.80 Hz, H-2′″ and H-6′″), 7.86 (d, 2H, J=8.40 Hz, H-3″ and H-5″), 7.64 (d, 1H, J=3.60 Hz, H-6), 7.62 (t, 2H, J=7.80 Hz, H-3′″ and H-5″), 7.44 (t, 1H, J=7.80 Hz, H-4′″), 7.01 (d, 1H, J=3.60 Hz, H-5), 3.73-3.64 (m, 4H, 2NCH₂), 3.09-3.04 (m, 4H, 4NCH), 2.46-2.40 (m, 4H, 2NCH₂), 1.85-1.72 (m, 4H, 2CH₂), 1.56-1.50 (m, 4H, 2CH₂), 1.45-1.36 (m, 4H, 2CH₂), 1.06-1.02 (m, 24H, 8CH₃).

STEP 3 leads to compound (34A) as detailed in Table 3.

The corresponding oxalate salt, compound (34B), has also been obtained as detailed in Table 3.

### Example 2.2: Synthesis of Compound (6)

### Step 1A (Ar₁≠Ar₂): Preparation of 2-Chloro-4-(5-formylthien-2-yl)-7-phenyl-7H-pyrrolo[2,3-d]pyrimidine

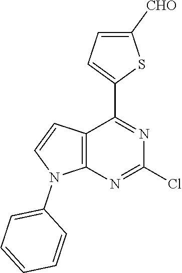 [SMILES: O=Cc1ccc(-c2nc(Cl)nc3c2ccn3-c2ccccc2)s1]

To a solution of 2.58 mmol of 2,4-dichloro-7-phenyl-7H-pyrrolo[2,3-d]pyrimidine, 0.89 g of 5-formyl-2-thiophene boronic acid (5.68 mmol, 2.2 eq.) and 300 mg (0.258 mmol, 0.1 eq.) of tetrakis(triphenylphosphine) palladium in 28 mL of 1,2-dimethoxyethane, 4 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, then EtO₂ and petroleum ether and dried under pressure to give the solid product. Yellow crystals (45%); Mp=198° C.; ¹H NMR δ (300 MHz, CDCl₃): 10.04 (s, 1H, CHO), 8.15 (d, 1H, J=3.90 Hz, H-6), 7.89 (d, 1H, J=4.00 Hz, H-thioph.), 7.71 (d, 2H, J=8.10 Hz, H-2′ and H-6′), 7.65 (d, 1H, J=4.00 Hz, H-thioph.), 7.58 (t, 2H, J=8.10 Hz, H-3′ and H-5′), 7.46 (t, 1H, J=8.10 Hz, H-4′), 7.10 (d, 1H, J=3.90 Hz, H-5).

### Step 1B: Preparation of 2-(4-Formylphenyl)-4-(5-formylthien-2-yl)-7-phenyl-7H-pyrrolo[2,3-d]pyrimidine

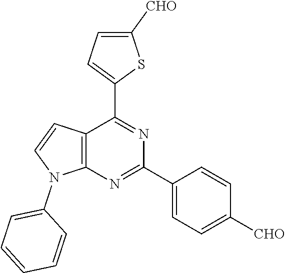 [SMILES: O=Cc1ccc(-c2nc(-c3ccc(C=O)s3)c3ccn(-c4ccccc4)c3n2)cc1]

To a solution of 0.53 mmol of 2-Chloro-4-(5-formylthien-2-yl)-7-phenyl-7H-pyrrolo[2,3-d]pyrimidine, 90 mg of 4-formylphenylboronic acid (0.58 mmol, 1.1 eq.) and 31 mg (0.0265 mmol, 0.05 eq.) of tetrakis(triphenylphosphine) palladium in 8 mL of 1,2-dimethoxyethane, 1 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (12 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, then EtO₂ and petroleum ether and dried under pressure to give the solid product. Yellow crystals (41%); Mp=234° C.; ¹H NMR δ (300 MHz, CDCl₃): 10.04 (s, 1H, CHO), 9.99 (s, 1H, CHO), 8.67 (d, 2H, J=8.40 Hz, H-2′ and H-6′), 8.17 (d, 1H, J=3.60 Hz, H-6), 7.98-7.96 (m, 3H, H-thioph., H-3′ and H-5′), 7.88-7.83 (m, 3H, H-thioph., H-2″ and H-6″), 7.60 (t, 2H, J=8.10 Hz, H-3″ and H-5″), 7.43 (t, 1H, J=8.10 Hz, H-4″), 7.14 (d, 1H, J=3.60 Hz, H-5).

### Step 2: Preparation of 2-{4-[(3-Dimethylaminopropyl)iminomethyl]phenyl}-4-{5-[(3-dimethylaminopropyl)iminomethyl]thien-2-yl}-7-phenyl-7H-pyrrolo[2,3-d]pyrimidine

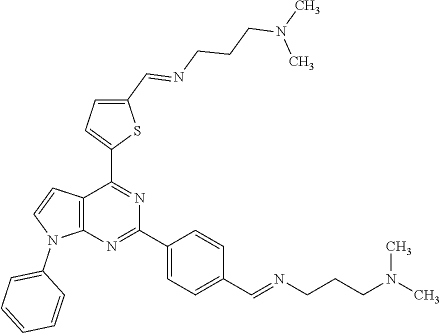 [SMILES: CN(C)CCC/N=C/c1ccc(-c2nc(-c3ccc(/C=N/CCCN(C)C)s3)c3ccn(-c4ccccc4)c3n2)cc1]

To a solution of 3-dimethylaminopropylamine (0.315 mmol, 2.15 eq.) in ethanol (5 mL) was added 2-(4-formylphenyl)-4-(5-formylthien-2-yl)-7-phenyl-7H-pyrrolo[2,3-d]pyrimidine (0.15 mmol). The reaction mixture was then heated under reflux for 15 h, and then evaporated to dryness under reduced pressure. After cooling, the residue was extracted with dichloromethane (15 mL). The organic layer was dried over sodium sulfate and activated charcoal and evaporated to dryness. Product was then used without further purification. Pale-yellow crystals (98%); Mp=152° C.; ¹H NMR δ (300 MHz, CDCl₃): 8.65 (d, 2H, J=8.10 Hz, H-2′ and H-6′), 8.42 (s, 1H, CH═N), 8.37 (s, 1H, CH═N), 7.99 (d, 1H, J=3.60 Hz, H-6), 7.84-7.80 (m, 4H, H-3′, H-5′, H-2″ and H-6″), 7.60-7.54 (m, 3H, H-3″, H-5″ and H-thioph.), 7.43-7.38 (m, 2H, H-4′ and H-thioph.), 7.01 (d, 1H, J=3.60 Hz, H-5), 3.70 (t, 4H, J=6.90 Hz, 2 NCH2), 2.42-2.36 (m, 4H, 2 NCH2), 2.27 (s, 12H, 2N(CH₃)₂), 1.95-1.92 (m, 4H, 2CH₂).

### Step 3: Preparation of Compound (6A) as Detailed in Table 3

To a solution of 2-{4-[(3-dimethylaminopropyl)iminomethyl]phenyl}-4-{5-[(3-dimethylaminopropyl)iminomethyl]thien-2-yl}-7-phenyl-7H-pyrrolo[2,3-d]pyrimidine (0.15 mmol) in methanol (5 mL) was added portion-wise at 0° C. sodium borohydride (1.2 mmol, 8 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the 2-{4-[(3-dimethylaminopropyl)aminomethyl]phenyl}-4-{5-[(3-dimethylaminopropyl)aminomethyl]thien-2-yl}-7-phenyl-7H-pyrrolo[2,3-d]pyrimidine.

The corresponding oxalate salt, compound (6B), has also been obtained as detailed in Table 3.

### Example 2.3: Synthesis of Compound (7)

### Step 1A (Ar₁≠Ar₂): Preparation of 2-Chloro-4-(4-formylphenyl)-7-phenyl-7H-pyrrolo[2,3-d]pyrimidine

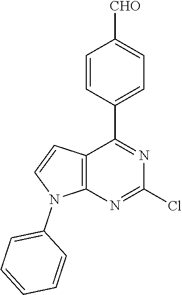 [SMILES: O=Cc1ccc(-c2nc(Cl)nc3c2ccn3-c2ccccc2)cc1]

To a solution of 2.0 mmol of 2,4-dichloro-7-phenyl-7H-pyrrolo[2,3-d]pyrimidine, 0.33 g of 4-formylphenylboronic acid (2.2 mmol, 1.1 eq.) and 116 mg (0.100 mmol, 0.05 eq.) of tetrakis(triphenylphosphine) palladium in 25 mL of 1,2-dimethoxyethane, 3 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, then EtO₂ and petroleum ether and dried under pressure to give the solid product.

Pale-yellow crystals (55%); Mp=182° C.; ¹H NMR δ (300 MHz, CDCl₃): 10.20 (s, 1H, CHO), 8.32 (d, 2H, J=8.40 Hz, H-2′ and H-6′), 8.10 (d, 2H, J=8.40 Hz, H-3′ and H-5′), 7.73 (d, 2H, J=7.40 Hz, H-2″ and H-6″), 7.63 (d, 1H, J=3.60 Hz, H-6), 7.59 (t, 2H, J=7.40 Hz, H-3″ and H-5″), 7.45 (t, 1H, J=7.40 Hz, H-4″), 7.00 (d, 1H, J=3.60 Hz, H-5).

### Step 1B: Synthesis of 4-(4-Formylphenyl)-2-(5-formylthien-2-yl)-7-phenyl-7H-pyrrolo[2,3-d]pyrimidine

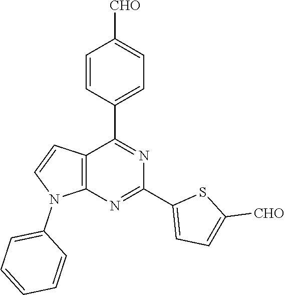 [SMILES: O=Cc1ccc(-c2nc(-c3ccc(C=O)s3)nc3c2ccn3-c2ccccc2)cc1]

To a solution of 0.84 mmol of 4-(4-formylphenyl)-7-phenyl-7H-pyrrolo[2,3-d]pyrimidine, 200 mg of 5-formyl-2-thiophene boronic acid (1.26 mmol, 1.5 eq.) and 48 mg (0.042 mmol, 0.05 eq.) of tetrakis(triphenylphosphine) palladium in 15 mL of 1,2-dimethoxyethane, 1.6 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (12 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, then EtO₂ and petroleum ether and dried under pressure to give the solid product. Pale-yellow crystals (23%); Mp=213° C.; ¹H NMR δ (300 MHz, CDCl₃): 10.15 (s, 1H, CHO), 9.98 (s, 1H, CHO), 8.43 (d, 2H, J=8.10 Hz, H-2′ and H-6′), 8.15-8.11 (m, 3H, H-thioph., H-2″ and H-6″), 7.87 (d, 2H, J=8.10 Hz, H-3′ and H-5′), 7.82 (d, 1H, J=3.30 Hz, H-thioph.), 7.72 (d, 1H, J=3.10 Hz, H-6), 7.63 (d, 2H, J=7.50 Hz, H-3″ and H-5″), 7.47 (t, 1H, J=7.50 Hz, H-4″), 7.05 (d, 1H, J=3.10 Hz, H-5).

### Step 2: Synthesis of 4-{4-[(3-Dimethylaminopropyl)iminomethyl]phenyl}-2-{5-[(3-dimethylaminopropyl)iminomethyl]thien-2-yl}-7-phenyl-7H-pyrrolo[2,3-d]pyrimidine

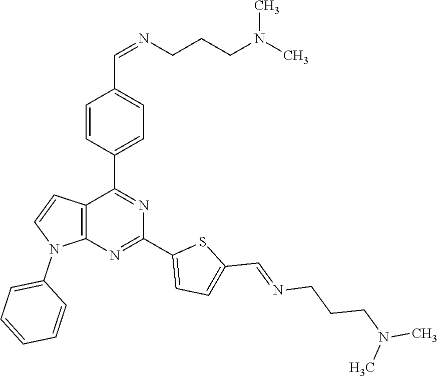 [SMILES: CN(C)CCC/N=C\c1ccc(-c2nc(-c3ccc(/C=N/CCCN(C)C)s3)nc3c2ccn3-c2ccccc2)cc1]

To a solution of 3-dimethylaminopropylamine (0.315 mmol, 2.15 eq.) in ethanol (5 mL) was added 4-(4-formylphenyl)-2-(5-formylthien-2-yl)-7-phenyl-7H-pyrrolo[2,3-d]pyrimidine (0.15 mmol). The reaction mixture was then heated under reflux for 5 h, and then evaporated to dryness under reduced pressure. After cooling, the residue was extracted with dichloromethane (15 mL). The organic layer was dried over sodium sulfate and activated charcoal and evaporated to dryness. Product was then used without further purification. Yellow oil (94%); ¹H NMR δ (300 MHz, CDCl₃): 8.41 (s, 1H, CH═N), 8.38 (s, 1H, CH═N), 8.31 (d, 2H, J=8.40 Hz, H-2′ and H-6′), 8.03 (d, 1H, J=3.60 Hz, H-6), 7.93 (d, 2H, J=8.40 Hz, H-3′ and H-5′), 7.85 (d, 2H, J=7.80 Hz, H-2″ and H-6″), 7.59 (d, 1H, J=3.60 Hz, H-thioph.), 7.58 (t, 2H, J=7.80 Hz, H-3″ and H-5″), 7.41 (t, 1H, J=7.80 Hz, H-4″), 7.31 (d, 1H, J=3.60 Hz, H-thioph.), 6.98 (d, 1H, J=3.60 Hz, H-5), 3.73 (t, 2H, J=6.90 Hz, NCH₂), 3.66 (t, 2H, J=6.90 Hz, NCH₂), 2.45-2.35 (m, 4H, 2 NCH₂), 2.29 (s, 6H, N(CH₃)₂), 2.26 (s, 6H, N(CH₃)₂), 1.98-1.89 (m, 4H, 2CH₂).

### Step 3: Synthesis of Compound (7A) as Detailed in Table 3

To a solution of 4-{4-[(3-dimethylaminopropyl)iminomethyl]phenyl}-2-{5-[(3-dimethylaminopropyl)iminomethyl]thien-2-yl}-7-phenyl-7H-pyrrolo[2,3-d]pyrimidine (0.14 mmol) in methanol (6 mL) was added portion-wise at 0° C. sodium borohydride (1.1 mmol, 8 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the 4-{4-[(3-dimethylaminopropyl)aminomethyl]phenyl}-2-{5-[(3-dimethylaminopropyl)aminomethyl]thien-2-yl}-7-phenyl-7H-pyrrolo[2,3-d]pyrimidine.

The corresponding oxalate salt, compound (7B) has also been obtained as detailed in Table 3.

### Example 2.4: Synthesis of Compounds (8) to (11)

### Step 1A (Ar₁=Ar₂): Synthesis of 2,4-Bis(4-formylphenyl)-7-methyl-7H-pyrrolo[2,3-d]pyrimidine

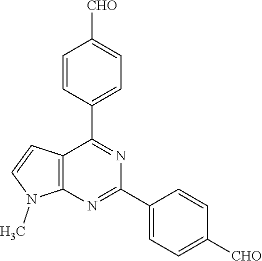 [SMILES: Cn1ccc2c(-c3ccc(C=O)cc3)nc(-c3ccc(C=O)cc3)nc21]

To a solution of 4.37 mmol of 2,4-dichloro-7-methyl-7H-pyrrolo[2,3-d]pyrimidine, 1.44 g of 4-formylphenyl boronic acid (9.63 mmol, 2.2 eq.) and 506 mg (0.437 mmol, 0.1 eq.) of tetrakis(triphenylphosphine) palladium in 45 mL of 1,2-dimethoxyethane, 5 mL of 2M Na₂CO₃ aqueous solution, previously degassed for 10 minutes with nitrogen, were added at room temperature. Then, the mixture was warmed to reflux and stirred for 24 hours under nitrogen positive pressure. The solvent was then evaporated under vacuum. The residue was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, EtO₂ and petroleum ether and dried under pressure to give the solid product.

### Step 2: Synthesis of 2,4-bis[(substituted-iminomethyl)]-7-methyl-7H-pyrrolo[2,3-d]pyrimidines

To a solution of the appropriate diamines (0.126 mmol, 2.1 eq.) in ethanol (7 mL) was added the 2,4-bis(4-formylphenyl)-7-methyl-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Synthesis of 2,4-bis[(substituted-aminomethyl)]-7-methyl-7H-pyrrolo[2,3-d]pyrimidines

To a solution of 2,4-bis[(substituted-iminomethyl)]-7-methyl-7H-pyrrolo[2,3-d]pyrimidines (0.4 mmol) in methanol (10 mL) was added portion-wise at 0° C. sodium borohydride (3.2 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 2,4-bis[(substituted-aminomethyl)-7-methyl-7H-pyrrolo[2,3-d]pyrimidines.

### Compound (8)

STEP 1A is performed as disclosed here-above.

### Step 2: Synthesis of 2,4-bis{4-[(3-dimethylaminopropyl)iminomethyl]phenyl}-7-methyl-7H-pyrrolo[2,3-d]pyrimidine

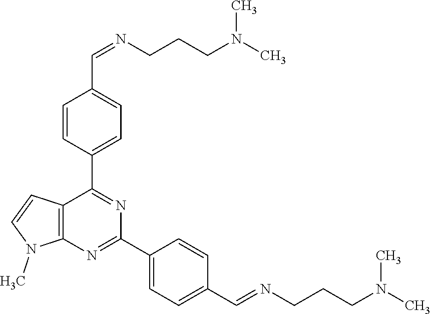 [SMILES: CN(C)CCC/N=C\c1ccc(-c2nc(-c3ccc(/C=N/CCCN(C)C)cc3)nc3c2ccn3C)cc1]

Yellow oil (98%); ¹H NMR δ (300 MHz, CDCl₃): 8.72 (d, 2H, J=8.40 Hz, H-2′ and H-6′), 8.38 (s, 1H, CH═N), 8.37 (s, 1H, CH═N), 8.31 (d, 2H, J=8.40 Hz, H-2″ and H-6″), 7.91 (d, 2H, J=8.40 Hz, H-3′ and H-5′), 7.85 (d, 2H, J=8.40 Hz, H-3″ and H-5″), 7.22 (d, 1H, J=3.45 Hz, H-6), 6.49 (d, 1H, J=3.45 Hz, H-5), 3.95 (s, 3H, NCH₃), 3.71-3.65 (m, 4H, 2NCH₂), 2.38 (t, 4H, J=6.90 Hz, 2NCH₂), 2.25 (s, 12H, N(CH₃)₂), 1.94-1.82 (m, 4H, 2CH₂).

STEP 3 leads to compound (8A) as detailed in Table 3.

The corresponding oxalate salt, compound (8B), has also been obtained as detailed in Table 3.

### Compound (9)

STEP 1A is performed as disclosed here-above.

### Step 2: Synthesis of 2,4-bis{4-[(3-(4-methylpiperazin-1-yl)propyl)iminomethyl]phenyl}-7-methyl-7H-pyrrolo[2,3-d]pyrimidine

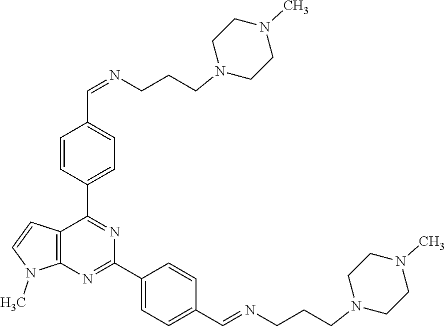 [SMILES: CN1CCN(CCC/N=C\c2ccc(-c3nc(-c4ccc(/C=N/CCCN5CCN(C)CC5)cc4)nc4c3ccn4C)cc2)CC1]

Orange-yellow oil (97%); ¹H NMR δ (300 MHz, CDCl₃): 8.62 (d, 2H, J=8.40 Hz, H-2′ and H-6′), 8.26 (s, 2H, 2CH═N), 8.20 (d, 2H, J=8.10 Hz, H-2″ and H-6″), 7.80 (d, 2H, J=8.10 Hz, H-3′ and H-5′), 7.75 (d, 2H, J=8.10 Hz, H-3″, H-5″), 7.08 (d, 1H, J=3.60 Hz, H-6), 6.65 (d, 1H, J=3.60 Hz, H-5), 3.8 (s, 3H, NCH₃), 3.60-3.52 (m, 4H, 2NCH₂), 2.41-2.20 (m, 20H, 10NCH₂), 2.18 (s, 6H, 2NCH₃), 1.89-1.78 (m, 4H, 2CH₂).

STEP 3 leads to compound (9A) as detailed in Table 3.

The corresponding oxalate salt, compound (B), has also been obtained as detailed in Table 3.

### Compound (10)

STEP 1A is performed as disclosed here-above.

### Step 2: Synthesis of 2,4-Bis{4-[(5-(tert-butoxycarbonylamino)-5-methoxycarbonyl)pentyl)iminomethyl]phenyl}-7-methyl-7H-pyrrolo[2,3-d]pyrimidine

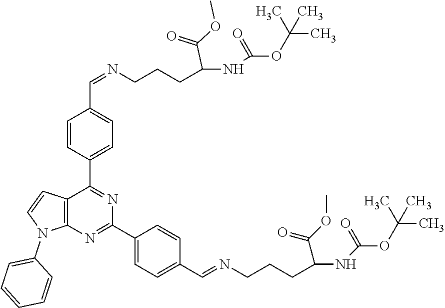 [SMILES: COC(=O)C(CCC/N=C\c1ccc(-c2nc(-c3ccc(/C=N/CCCC(NC(=O)OC(C)(C)C)C(=O)OC)cc3)nc3c2ccn3-c2ccccc2)cc1)NC(=O)OC(C)(C)C]

Yellow oil (88%); ¹H NMR δ (300 MHz, CDCl₃): 8.69 (d, 2H, J=8.40 Hz, H-2′ and H-6′), 8.31 (s, 1H, CH═N), 8.30 (s, 1H, CH═N), 8.27 (d, 2H, J=8.40 Hz, H-2″ and H-6″), 7.87 (d, 2H, J=8.40 Hz, H-3′ and H-5′), 7.80 (d, 2H, J=8.40 Hz, H-3″ and H-5″), 7.19 (d, 1H, J=3.60 Hz, H-6), 6.74 (d, 1H, J=3.60 Hz, H-5), 5.17 (d, 2H, J=8.00 Hz, 2NH), 4.29-4.27 (m, 2H, 2CH), 3.90 (s, 3H, NCH₃), 3.69 (s, 3H, COOCH₃), 3.68 (s, 3H, COOCH₃), 3.61-3.65 (m, 4H, 2NCH₂), 1.95-1.60 (m, 12H, 6CH₂). 1.39 (s, 18H, 2C(CH₃)₃).

STEP 3 leads to compound (10A)

The corresponding oxalate salt, compound (10B), has also been obtained as detailed in Table 3.

### Compound (11)

Compound (11A) is obtained from compound (10A) as detailed below.

To a solution of 2,4-bis{4-[(5-(tert-butoxycarbonylamino)-5-methoxycarbonyl)pentyl)aminomethyl]phenyl}-7-methyl-7H-pyrrolo[2,3-d]pyrimidine (0.08 mmol) in 6 mL of dichloromethane was added 1.0 mL of trifluoroacetic acid. The mixture was stirred at room temperature for 24 h, then neutralized with 65 mL of a saturated aqueous solution of potassium carbonate and extracted with 15 mL of dichloromethane. The organic layer was washed with water, then brine and dried with anhydrous sodium sulphate. The solvent was removed under reduced pressure to give the 2,4-bis{4-[(5-amino-5-(methoxycarbonyl)pentyl)aminomethyl]phenyl}-7-methyl-7H-pyrrolo[2,3-d]pyrimidine.

The corresponding oxalate salt, compound (11B), has also been obtained as detailed in Table 3.

### Example 2.5: Synthesis of Compound (24)

### Step 1A (Ar₁=Ar₂): Synthesis of Tert-butyl 2,4-bis(4-formylphenyl)-5,6-dihydropyrido[3,4-d]pyrimidine-7(8H)-carboxylate

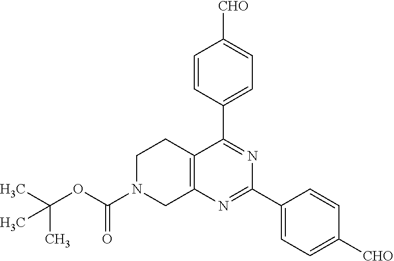 [SMILES: CC(C)(C)OC(=O)N1CCc2c(nc(-c3ccc(C=O)cc3)nc2-c2ccc(C=O)cc2)C1]

To a solution of 2.0 mmol of tert-butyl 2,4-dichloro-5,6-dihydropyrido[3,4-d]pyrimidine-7(8H)-carboxylate, 4.4 mmol of 4-formylphenyl boronic acid (2.2 eq.) and 230 mg (0.20 mmol, 0.1 eq.) of tetrakis(triphenylphosphine) palladium in 15 mL of 1,2-dimethoxyethane, 3 mL of 2M Na₂CO₃ aqueous solution, previously degassed for 10 minutes with nitrogen, were added at room temperature. Then, the mixture was warmed to reflux and stirred for 24 hours under nitrogen positive pressure. The solvent was then evaporated under vacuum. The residue was extracted with CH₂Cl₂ and the organic phase was filtered on filter paper, then washed with water (20 mL×2 times), dried over anhydrous sodium sulfate and activated charcoal, filtered and evaporated under vacuum. The residue was purified by silica gel column chromatography (CH₂Cl₂) to give the final product.

Yellow oil (23%); ¹H NMR δ (300 MHz, CDCl₃): 10.15 (s, 1H, CHO), 10.13 (s, 1H, CHO), 8.68 (d, 2H, J=8.40 Hz, H-2′ and H-6′), 8.06 (d, 2H, J=8.40 Hz, H-3″ and H-6″), 8.02 (d, 2H, J=8.40 Hz, H-3′ and H-5′), 7.89 (d, 2H, J=8.40 Hz, H-3″ and H-5″), 4.83 (s, 2H, NCH₂), 3.70 (t, 2H, J=5.40 Hz, NCH₂), 2.96 (t, 2H, J=5.40 Hz, CH₂), 1.56 (s, 9H, (CH₃)₃C).

### Step 2: Synthesis of Tert-butyl 2,4-bis{4-[(3-dimethylaminopropyl)iminomethyl]phenyl})-5,6-dihydropyrido[3,4-d]pyrimidine-7(8H)-carboxylate

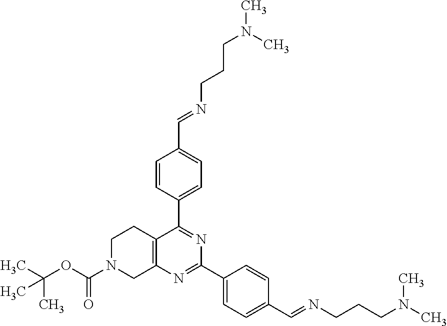 [SMILES: CN(C)CCC/N=C/c1ccc(-c2nc3c(c(-c4ccc(/C=N/CCCN(C)C)cc4)n2)CCN(C(=O)OC(C)(C)C)C3)cc1]

To a solution of 3-dimethylaminopropylamine (1.0 mmol, 2.15 eq.) in ethanol (8 mL) was added the tert-butyl 2,4-bis(4-formylphenyl)-5,6-dihydropyrido[3,4-d]pyrimidine-7(8H)-carboxylate (0.45 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Product was then used without further purification.

Orange oil (83%); ¹H NMR δ (300 MHz, CDCl₃): 8.54 (d, 2H, J=8.40 Hz, H-2′ and H-6′), 8.37 (s, 1H, CH═N), 8.35 (s, 1H, CH═N), 7.86 (d, 2H, J=8.40 Hz, H-2″ and H-6″), 7.81 (d, 2H, J=8.40 Hz, H-3′, H-5′), 7.75 (d, 2H, J=8.40 Hz, H-3″ and H-5″), 4.77 (s, 2H, NCH₂), 3.75-3.54 (m, 6H, 3NCH₂), 2.93 (t, 2H, J=5.40 Hz, CH₂), 2.41-2.24 (m, 4H, 2NCH₂), 2.23 (s, 6H, N(CH₃)₂), 2.22 (s, 6H, N(CH₃)₂), 1.94-1.87 (m, 4H, 2CH₂), 1.52 (m, 9H, C(CH₃)₃).

### Step 3: Synthesis of Compound (24A) as Detailed in Table 3

To a solution of tert-butyl 2,4-bis{4-[(3-dimethylaminopropyl)iminomethyl]phenyl})-5,6-dihydropyrido[3,4-d]pyrimidine-7(8H)-carboxylate (0.45 mmol) in methanol (10 mL) was added portion-wise at 0° C. sodium borohydride (3.6 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness.

The corresponding oxalate salt, compound (24B), has also been obtained as detailed in Table 3.

### Example 2.6: Synthesis of Compound (25)

STEPS 1 to 3 are processed as in Example 2.5 here-above.

STEP 4: Synthesis of compound (25A) as detailed in Table 3.

This Step is performed in accordance with Scheme 10 here-above.

To a solution of tert-butyl 2,4-bis{4-[(3-dimethylaminopropyl)aminomethyl]phenyl})-5,6-dihydropyrido[3,4-d]pyrimidine-7(8H)-carboxylate (0.08 mmol) in 5 mL of dichloromethane was added 0.5 mL of trifluoroacetic acid. The mixture was stirred at room temperature for 24 h, then neutralized with 65 mL of a saturated aqueous solution of potassium carbonate and extracted with 15 mL of dichloromethane. The organic layer was washed with water, then brine and dried with anhydrous sodium sulphate. The solvent was removed under reduced pressure.

The corresponding oxalate salt, compound (25B), has also been obtained as detailed in Table 3.

### Example 2.7. Synthesis of Compounds (26) and (27)

### Step 1A (Ar₁=Ar₂): Synthesis of 2,4-Bis(4-formylphenyl)thieno[2,3-d]pyrimidine

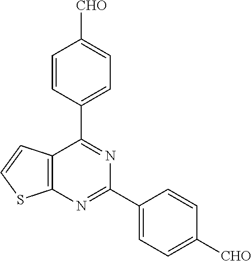 [SMILES: O=Cc1ccc(-c2nc(-c3ccc(C=O)cc3)c3ccsc3n2)cc1]

To a solution of 4.37 mmol of 2,4-dichlorothieno[2,3-d]pyrimidine, 1.44 g of 4-formylphenylboronic acid (9.63 mmol, 2.2 eq.) and 506 mg (0.437 mmol, 0.1 eq.) of tetrakis(triphenylphosphine) palladium in 45 mL of 1,2-dimethoxyethane, 5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, then EtO₂ and petroleum ether and dried under pressure to give the solid product.

White crystals (40%); Mp=216° C.; ¹H NMR δ (300 MHz, CDCl₃): 10.20 (s, 1H, CHO), 10.15 (s, 1H, CHO), 8.84 (d, 2H, J=8.40 Hz, H-2′ and H-6′), 8.26 (d, 2H, J=8.40 Hz, H-2″ and H-6″), 8.16 (d, 2H, J=8.40 Hz, H-3′ and H-5′), 8.06 (d, 2H, J=8.40 Hz, H-3″ and H-5″), 7.70 (d, 1H, J=6.15 Hz, H-6), 7.64 (d, 1H, J=6.15 Hz, H-5).

### Step 2: Synthesis of 2,4-bis[(substituted-iminomethyl)thieno[2,3-d]pyrimidines

To a solution of the appropriate diamines (0.126 mmol, 2.1 eq.) in ethanol (7 mL) was added (SR8) (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Synthesis of 2,4-bis[(substituted-aminomethyl)thieno[2,3-d]pyrimidines

To a solution of 2,4-bis[(substituted-iminomethyl)thieno[2,3-d]pyrimidines (0.4 mmol) in methanol (10 mL) was added portion-wise at 0° C. sodium borohydride (3.2 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 2,4-bis[(substituted-aminomethyl)thieno[2,3-d]pyrimidines.

### Compound (26)

STEP 1A is performed as disclosed here-above.

### Step 2: Synthesis of 2,4-Bis{4-[(4-dimethylaminopropyl)iminomethyl]phenyl}thieno[2,3-d]pyrimidine

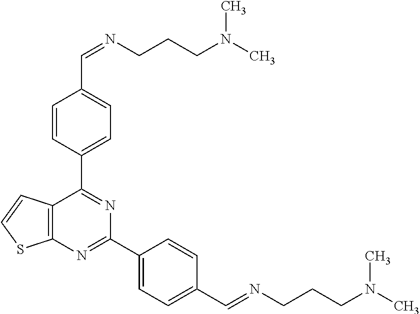 [SMILES: CN(C)CCC/N=C\c1ccc(-c2nc(-c3ccc(/C=N/CCCN(C)C)cc3)nc3sccc23)cc1]

Yellow oil (98%); ¹H NMR δ (300 MHz, CDCl₃): 8.69 (d, 2H, J=8.40 Hz, H-2′ and H-6′), 8.41 (s, 1H, CH═N), 8.38 (s, 1H, CH═N), 8.12 (d, 2H, J=8.40 Hz, H-2″ and H-6″), 7.94 (d, 2H, J=8.40 Hz, H-3′ and H-5′), 7.87 (d, 2H, J=8.40 Hz, H-3″ and H-5″), 7.59 (d, 1H, J=6.30 Hz, H-6), 7.55 (d, 1H, J=6.30 Hz, H-5), 3.75-3.67 (m, 4H, 2 NCH₂), 2.42-2.36 (m, 4H, 2 NCH₂), 2.27 (s, 6H, N(CH₃)₂), 2.26 (s, 6H, N(CH₃)₂), 1.98-1.87 (m, 4H, 2 CH₂)

STEP 3 leads to compound (26A) as detailed in Table 3.

The corresponding oxalate salt, compound (26B), has also been obtained as detailed in Table 3.

### Compound (27)

STEP 1 is performed as disclosed here-above

### Step 2: Synthesis of 2,4-Bis{4-[(3-(4-methylpiperazin-1-yl)propyl)iminomethyl]phenyl}thieno[2,3-d]pyrimidine

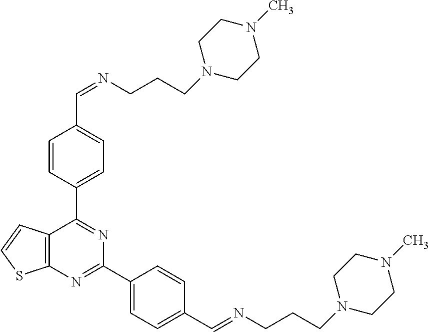 [SMILES: CN1CCN(CCC/N=C\c2ccc(-c3nc(-c4ccc(/C=N/CCCN5CCN(C)CC5)cc4)nc4sccc34)cc2)CC1]

Yellow oil (98%); ¹H NMR δ (300 MHz, CDCl₃): 8.59 (d, 2H, J=8.10 Hz, H-2′ and H-6′), 8.29 (s, 1H, CH═N), 8.27 (s, 1H, CH═N), 8.00 (d, 2H, J=8.10 Hz, H-2″ and H-6″), 7.83 (d, 2H, J=8.10 Hz, H-3′ and H-5′), 7.76 (d, 2H, J=8.10 Hz, H-3″ and H-5″), 7.47 (d, 1H, J=6.30 Hz, H-6), 7.45 (d, 1H, J=6.30 Hz, H-5), 3.65-3.56 (m, 4H, 2 NCH₂), 2.58-2.29 (m, 20H, 10 NCH₂), 2.21 (s, 3H, NCH₃), 2.20 (s, 3H, NCH₃), 1.88-1.82 (m, 4H, 2 CH₂).

STEP 3 leads to compound (27A) as detailed in Table 3.

The corresponding oxalate salt, compound (27B), has also been obtained as detailed in Table 3.

### Example 2.8. Synthesis of Compounds (29) and (30)

### Step 1A (Ar₁=Ar₂): Synthesis of 2,4-Bis(4-formylphenyl)thieno[3,2-d]pyrimidine

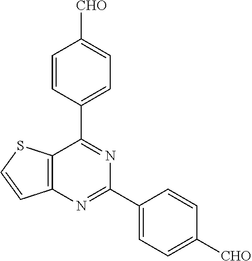 [SMILES: O=Cc1ccc(-c2nc(-c3ccc(C=O)cc3)c3sccc3n2)cc1]

To a solution of 4.37 mmol of 2,4-dichlorothieno[3,2-d]pyrimidine, 1.44 g of 4-formylphenylboronic acid (9.63 mmol, 2.2 eq.) and 506 mg (0.437 mmol, 0.1 eq.) of tetrakis(triphenylphosphine) palladium in 45 mL of 1,2-dimethoxyethane, 5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂/CH₃OH 95:5), then cooled and triturated again in EtOH, filtered on sintered glassware, washed with a minimum of EtOH, then EtO₂ and petroleum ether and dried under pressure to give the solid product. White crystals (45%); Mp=227° C.; ¹H NMR δ (300 MHz, CDCl₃): 10.20 (s, 1H, CHO), 10.16 (s, 1H, CHO), 8.86 (d, 2H, J=8.40 Hz, H-2′ and H-6′), 8.52 (d, 2H, J=8.40 Hz, H-2″ and H-6″), 8.17 (d, 2H, J=8.40 Hz, H-3′ and H-5′), 8.15 (d, 1H, J=5.70 Hz, H-6), 8.08 (d, 2H, J=8.40 Hz, H-3″ and H-5″), 7.76 (d, 1H, J=5.70 Hz, H-7).

### Step 2: Synthesis of 2,4-bis[(substituted-iminomethyl)thieno[3,2-d]pyrimidines

To a solution of the appropriate diamines (0.126 mmol, 2.1 eq.) in ethanol (7 mL) was added 2,4-Bis(4-formylphenyl)thieno[3,2-d]pyrimidine from STEP 1A (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Synthesis of 2,4-bis[(substituted-aminomethyl)thieno[3,2-d]pyrimidines

To a solution of 2,4-bis[(substituted-iminomethyl)thieno[3,2-d]pyrimidines (0.4 mmol) in methanol (10 mL) was added portion-wise at 0° C. sodium borohydride (3.2 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 2,4-bis[(substituted-aminomethyl)thieno[2,3-d]pyrimidines.

### Compound (29)

STEP 1A is performed as disclosed here-above.

### Step 2: Synthesis of 2,4-Bis{4-[(4-dimethylaminopropyl)iminomethyl]phenyl}thieno[3,2-d]pyrimidine

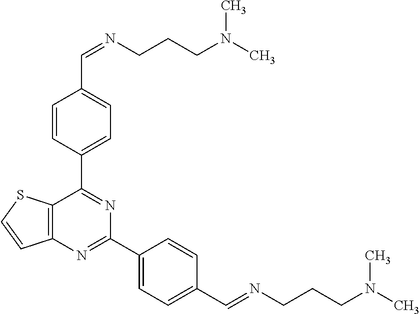 [SMILES: CN(C)CCC/N=C\c1ccc(-c2nc(-c3ccc(/C=N/CCCN(C)C)cc3)nc3ccsc23)cc1]

Yellow oil (98%); ¹H NMR δ (300 MHz, CDCl₃): 8.69 (d, 2H, J=8.40 Hz, H-2′ and H-6′), 8.40 (s, 1H, CH═N), 8.38 (s, 1H, CH═N), 8.35 (d, 2H, J=8.40 Hz, H-2″ and H-6″), 8.02 (d, 1H, J=5.70 Hz, H-6), 7.95 (d, 2H, J=8.40 Hz, H-3′ and H-5′), 7.88 (d, 2H, J=8.40 Hz, H-3″ and H-5″), 7.66 (d, 1H, J=6.30 Hz, H-7), 3.72 (t, 2H, J=6.90 Hz, NCH₂), 3.69 (t, 2H, J=6.90 Hz, NCH₂), 2.39 (t, 4H, J=6.90 Hz, 2 NCH₂), 1.97-1.86 (m, 4H, 2 CH₂).

STEP 3 leads to compound (29A) as detailed in Table 3.

The corresponding oxalate salt, compound (29B), has also been obtained as detailed in Table 3.

### Compound (30)

STEP 1A is performed as disclosed here-above.

### Step 2: Synthesis of 2,4-Bis{4-[(3-(4-methylpiperazin-1-yl)propyl)iminomethyl]phenyl}thieno[3,2-d]pyrimidine

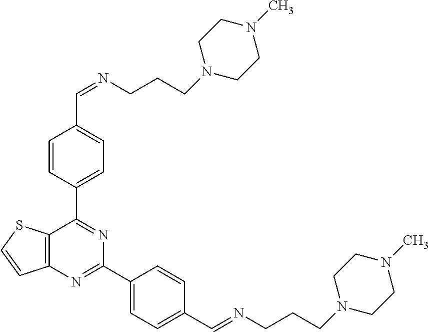 [SMILES: CN1CCN(CCC/N=C\c2ccc(-c3nc(-c4ccc(/C=N/CCCN5CCN(C)CC5)cc4)nc4ccsc34)cc2)CC1]

Yellow oil (85%); ¹H NMR δ (300 MHz, CDCl₃): 8.66 (d, 2H, J=8.40 Hz, H-2′ and H-6′), 8.35 (s, 1H, CH═N), 8.34 (s, 1H, CH═N), 8.32 (d, 2H, J=8.10 Hz, H-2″ and H-6″), 7.98 (d, 1H, J=5.70 Hz, H-6), 7.92 (d, 2H, J=8.40 Hz, H-3′ and H-5′), 7.84 (d, 2H, J=8.40 Hz, H-3″ and H-5″), 7.62 (d, 1H, J=5.70 Hz, H-7), 3.69-3.62 (m, 4H, 2 NCH₂), 2.62-2.36 (m, 20H, 10 NCH₂), 2.27 (s, 6H, 2 NCH₃), 1.94-1.82 (m, 4H, 2 CH₂).

STEP 3 leads to compound (30A).

The corresponding oxalate salt, compound (30B), has also been obtained.

### Example 2.9—Synthesis of Compounds (36) to (39)

### Step 1A (Ar₁=Ar₂): preparation of 4,6-Bis(4-formylphenyl)-1-phenyl-1H-pyrazolo[3,4-d]pyrimidine

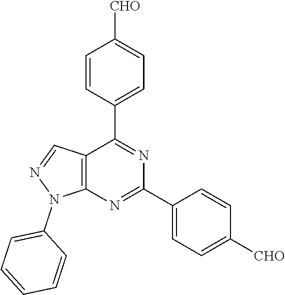 [SMILES: O=Cc1ccc(-c2nc(-c3ccc(C=O)cc3)c3cnn(-c4ccccc4)c3n2)cc1]

To a solution of 2.26 mmol of (SR5), 0.75 g of 4-formylphenyl boronic acid (5.0 mmol, 2.2 eq.) and 260 mg (0.226 mmol, 0.1 eq.) of tetrakis(triphenylphosphine)palladium in 25 mL of 1,2-dimethoxyethane, 3.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (12 mL×3 times), dried over anhydrous sodium sulfate and activated charcoal, filtered and evaporated under vacuum. The residue was cooled and triturated with a minimum of EtOH and Et₂O and filtered on sintered glassware to give the crude product. The residue was purified by silica gel column chromatography (CHCl₃/CH₃OH 95:5), then cooled and triturated again in EtOH, filtered on sintered glassware, washed with a minimum of EtOH, EtO₂ and petroleum ether and dried under pressure to give the solid product.

Pale-yellow crystals (48%); Mp=264° C.; ¹H NMR δ (300 MHz, CDCl₃): 10.22 (s, 1H, CHO), 10.17 (s, 1H, CHO), 8.91 (d, 2H, J=8.25 Hz, H-2′ and H-6′), 8.59 (s, 1H, H-3), 8.53 (d, 2H, J=8.25 Hz, H-2″ and H-6″), 8.39 (d, 2H, J=7.50 Hz, H-2′″ and H-6′″), 8.20 (d, 2H, J=8.25 Hz, H-3′ and H-5′), 8.09 (d, 2H, J=8.25 Hz, H-3″ and H-5″), 7.66 (t, 2H, J=7.50 Hz, H-3′″ and H-5′″), 7.47 (t, 1H, J=7.50 Hz, H-4′″).

### Step 2 Preparation of 4,6-bis[(substituted-iminomethyl)-1-phenyl-1H-pyrazolo[3,4-d]pyrimidines

To a solution of the appropriate diamines (0.126 mmol, 2.1 eq.) in ethanol (7 mL) was added the 4,6-bis(4-formylphenyl)-1-phenyl-1H-pyrazolo[3,4-d]pyrimidine (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Preparation of 4,6-bis[(substituted-aminomethyl)-1-phenyl-1H-pyrazolo[3,4-d]pyrimidines

To a solution of 4,6-bis[(substituted-iminomethyl)-1-phenyl-1H-pyrazolo[3,4-d]pyrimidines (0.4 mmol) in methanol (10 mL) was added portion-wise at 0° C. sodium borohydride (3.2 mmol, 8 eq.). The reaction mixture was then stirred at 60° C. for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 4,6-bis[(substituted-aminomethyl)-1-phenyl-1H-pyrazolo[3,4-d]pyrimidines.

### Compound (36)

STEP 1A is performed as disclosed here-above.

### Step 2: Synthesis of 4,6-Bis{4-[(3-dimethylaminopropyl)iminomethyl]phenyl}-1-phenyl-1H-pyrazolo[3,4-d]pyrimidine

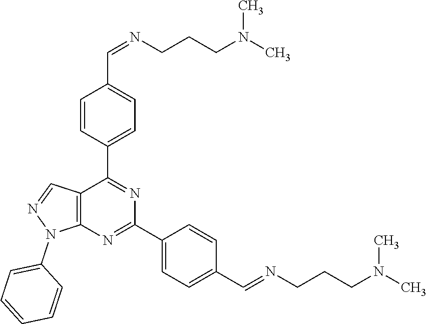 [SMILES: CN(C)CCC/N=C\c1ccc(-c2nc(-c3ccc(/C=N/CCCN(C)C)cc3)nc3c2cnn3-c2ccccc2)cc1]

Pale-yellow crystals (98%); Mp=145° C.; ¹H NMR δ (300 MHz, CDCl₃): 8.77 (d, 2H, J=8.10 Hz, H-2′ and H-6′), 8.52 (s, 1H, H-3), 8.44 (s, 1H, CH═N), 8.41-8.36 (m, 5H, CH═N, H-2″, H-6″, H-2′″ and H-6′″), 8.00 (d, 2H, J=8.10 Hz, H-3′ and H-5′), 7.91 (d, 2H, J=8.10 Hz, H-3″ and H-5″), 7.62 (t, 2H, J=7.65 Hz, H-3′″ and H-5″), 7.41 (t, 1H, J=7.65 Hz, H-4′″), 3.75 (t, 2H, J=7.20 Hz, NCH₂), 3.73 (t, 2H, J=7.20 Hz, NCH₂), 2.45 (t, 4H, J=7.20 Hz, 2NCH₂), 2.31 (s, 12H, 2N(CH₃)₂), 2.00-1.93 (m, 4H, 2CH₂).

STEP 3 leads to compound (36A) as detailed in Table 3.

The corresponding oxalate salt, compound (36B), has also been obtained as detailed in Table 3.

### Compound (37)

STEP 1A is performed as disclosed here-above.

### Step 2: Synthesis of 4,6-Bis{4-[(4-dimethylaminobutyl)iminomethyl]phenyl-1-phenyl}-1H-pyrazolo[3,4-d]pyrimidine

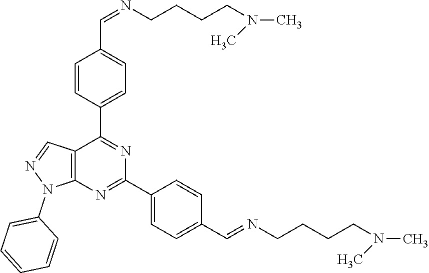 [SMILES: CN(C)CCCC/N=C\c1ccc(-c2nc(-c3ccc(/C=N/CCCCN(C)C)cc3)nc3c2cnn3-c2ccccc2)cc1]

Yellow oil (70%); ¹H NMR δ (300 MHz, CDCl₃): 8.76 (d, 2H, J=8.40 Hz, H-2′ and H-6′), 8.53 (s, 1H, H-3), 8.43-8.37 (m, 6H, 2CH═N, H-2″, H-6″, H-2′″ and H-6′″), 8.00 (d, 2H, J=8.40 Hz, H-3′ and H-5′), 7.90 (d, 2H, J=8.40 Hz, H-3″ and H-5″), 7.63 (t, 2H, J=7.80 Hz, H-3′″ and H-5″), 7.42 (t, 1H, J=7.80 Hz, H-4′″), 3.73 (t, 2H, J=6.90 Hz, NCH₂), 3.71 (t, 2H, J=6.90 Hz, NCH₂), 2.37 (t, 2H, J=6.90 Hz, NCH₂), 2.35 (t, 2H, J=6.90 Hz, NCH₂), 2.28 (s, 6H, N(CH₃)₂), 2.27 (s, 6H, N(CH₃)₂), 1.83-1.74 (m, 4H, 2CH₂), 1.66-1.56 (m, 4H, 2CH₂).

STEP 3 leads to compound (37A) as detailed in Table 3.

The corresponding oxalate salt, compound (37B), has also been obtained as detailed in Table 3.

### Compound (38)

STEP 1A is performed as disclosed here-above.

### Step 2: Synthesis of 4,6-Bis{4-[(5-dimethylaminopentyl)iminomethyl]phenyl}-1-phenyl-1H-pyrazolo[3,4-d]pyrimidine

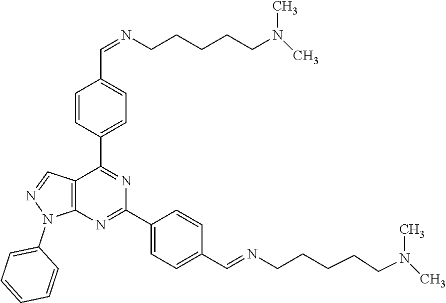 [SMILES: CN(C)CCCCC/N=C\c1ccc(-c2nc(-c3ccc(/C=N/CCCCCN(C)C)cc3)nc3c2cnn3-c2ccccc2)cc1]

Yellow crystals (93%); Mp=120° C.; ¹H NMR δ (300 MHz, CDCl₃): 8.74 (d, 2H, J=8.40 Hz, H-2′ and H-6′), 8.51 (s, 1H, H-3), 8.41-8.35 (m, 6H, 2CH═N, H-2″, H-6″, H-2′″ and H-6′″), 7.99 (d, 2H, J=8.40 Hz, H-3′ and H-5′), 7.89 (d, 2H, J=8.40 Hz, H-3″ and H-5″), 7.61 (t, 2H, J=7.50 Hz, H-3′″ and H-5″), 7.40 (t, 1H, J=7.50 Hz, H-4′″), 3.71 (t, 2H, J=6.90 Hz, NCH₂), 3.68 (t, 2H, J=6.90 Hz, NCH₂), 2.32 (t, 2H, J=6.90 Hz, NCH₂), 2.29 (t, 2H, J=6.90 Hz, NCH₂), 2.24 (s, 6H, N(CH₃)₂), 2.22 (s, 6H, N(CH₃)₂), 1.83-1.75 (m, 4H, 2CH₂), 1.59-1.40 (m, 8H, 4CH₂).

STEP 3 leads to compound (38A) as detailed in Table 3.

The corresponding oxalate salt, compound (38B), has also been obtained as detailed in Table 3.

### Compound (39)

STEP 1A is performed as disclosed here-above.

### Step 2: Synthesis of 4,6-Bis{4-[(4-(4-methylpiperazin-1-yl)butyl)iminomethyl]phenyl}-1-phenyl-1H-pyrazolo[3,4-d]pyrimidine

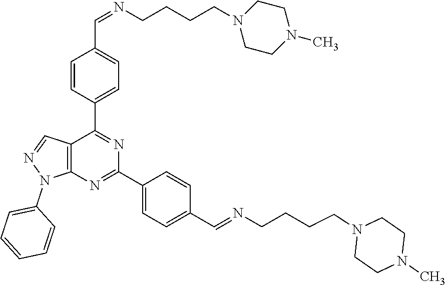 [SMILES: CN1CCN(CCCC/N=C\c2ccc(-c3nc(-c4ccc(/C=N/CCCCN5CCN(C)CC5)cc4)nc4c3cnn4-c3ccccc3)cc2)CC1]

Pale-yellow crystals (80%); Mp=132° C.; ¹H NMR δ (300 MHz, CDCl₃): 8.75 (d, 2H, J=8.10 Hz, H-2′ and H-6′), 8.52 (s, 1H, H-3), 8.41 (s, 1H, CH═N), 8.40-8.36 (m, 5H, CH═N, H-2″, H-6″, H-2′″ and H-6′″), 7.99 (d, 2H, J=8.10 Hz, H-3′ and H-5′), 7.88 (d, 2H, J=8.10 Hz, H-3″ and H-5″), 7.62 (t, 2H, J=7.50 Hz, H-3′″ and H-5″), 7.41 (t, 1H, J=7.50 Hz, H-4′″), 3.71 (t, 2H, J=6.90 Hz, NCH₂), 3.69 (t, 2H, J=6.90 Hz, NCH₂), 2.50-2.32 (m, 20H, 10NCH₂), 2.29 (s, 3H, NCH₃), 2.28 (s, 3H, NCH₃), 1.81-1.72 (m, 4H, 2CH₂), 1.67-1.45 (m, 4H, 2CH₂).

STEP 3 leads to compound (39A) as detailed in Table 3.

The corresponding oxalate salt, compound (39B), has also been obtained as detailed in Table 3.

### Example 2.10—Synthesis of Compounds (40) to (42)

### Step 1A (Ar₁=Ar₂): preparation 2,4-dichloro-7-phenyl-7H-imidazo[4,5-d]pyrimidine

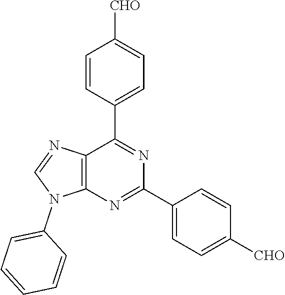 [SMILES: O=Cc1ccc(-c2nc(-c3ccc(C=O)cc3)c3ncn(-c4ccccc4)c3n2)cc1]

To a solution of 2.26 mmol of (SR7), 0.75 g of 4-formylphenyl boronic acid (5.0 mmol, 2.2 eq.) and 260 mg (0.226 mmol, 0.1 eq.) of tetrakis(triphenylphosphine)palladium in 25 mL of 1,2-dimethoxyethane, 3.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the precipitate was filtered, and washed successively with 1,2-dimethoxyethane, H₂O, EtOH, and Et₂O, and then dried under pressure to give the solid product.

White crystals (54%); Mp=256° C.; ¹H NMR δ (300 MHz, CDCl₃): 10.20 (s, 1H, CHO), 10.15 (s, 1H, CHO), 9.19 (d, 2H, J=8.10 Hz, H-2′ and H-6′), 8.84 (d, 2H, J=8.10 Hz, H-2″ and H-6″), 8.52 (s, 1H, H-6), 8.16 (d, 2H, J=8.10 Hz, H-3′ and H-5′), 8.06 (d, 2H, J=8.10 Hz, H-3″ and H-5″), 7.89 (d, 2H, J=7.50 Hz, H-2′″ and H-6′″), 7.71 (t, 2H, J=7.50 Hz, H-3′″ and H-5′″), 7.59 (t, 1H, J=7.50 Hz, H-4′″).

### Step 2 Preparation of 2,4-bis[(substituted-iminomethyl)-7-phenyl-7H-imidazo[4,5-d]pyrimidines

To a solution of the appropriate diamines (0.126 mmol, 2.1 eq.) in ethanol (7 mL) was added the 2,4-bis(4-formylphenyl)-7-phenyl-7H-imidazo[4,5-d]pyrimidine (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Preparation of 2,4-bis[(substituted-aminomethyl)-7-phenyl-7H-imidazo[4,5-d]pyrimidines

To a solution of 2,4-bis[(substituted-iminomethyl)-7-phenyl-7H-imidazo[4,5-d]pyrimidines (0.4 mmol) in methanol (10 mL) was added portion-wise at 0° C. sodium borohydride (3.2 mmol, 8 eq.). The reaction mixture was then stirred at 60° C. for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 2,4-bis[(substituted-aminomethyl)-7-phenyl-7H-imidazo[4,5-d]pyrimidines.

### Compound (40)

STEP 1A is performed as disclosed here-above.

### Step 2: Synthesis of 2,4-Bis{4-[(4-dimethylaminobutyl)iminomethyl]phenyl}-7-phenyl-7H-imidazo[4,5-d]pyrimidine

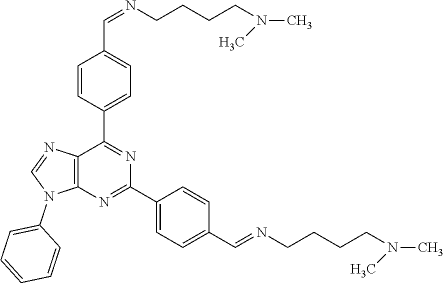 [SMILES: CN(C)CCCC/N=C\c1ccc(-c2nc(-c3ccc(/C=N/CCCCN(C)C)cc3)nc3c2ncn3-c2ccccc2)cc1]

Pale-yellow crystals (80%); Mp=140° C.; ¹H NMR δ (300 MHz, CDCl₃): 9.04 (d, 2H, J=8.40 Hz, H-2′ and H-6′), 8.68 (d, 2H, J=8.40 Hz, H-2″ and H-6″), 8.42 (s, 1H, H-6), 8.41 (s, 1H, CH═N), 8.37 (s, 1H, CH═N), 7.96 (d, 2H, J=8.40 Hz, H-3′ and H-5′), 7.90-7.84 (m, 4H, H-2′″, H-6′″, H-3″ and H-5″), 7.66 (t, 2H, J=7.60 Hz, H-3′″ and H-5″), 7.52 (t, 1H, J=7.60 Hz, H-4′″), 3.72 (t, 2H, J=6.90 Hz, NCH₂), 3.69 (t, 2H, J=6.90 Hz, NCH₂), 2.36 (t, 2H, J=6.90 Hz, NCH₂), 2.32 (t, 2H, J=6.90 Hz, NCH₂), 2.25 (s, 6H, N(CH₃)₂), 2.24 (s, 6H, N(CH₃)₂), 1.83-1.72 (m, 4H, 2CH₂), 1.66-1.56 (m, 4H, 2CH₂).

STEP 3 leads to compound (40A) as detailed in Table 3.

The corresponding oxalate salt, compound (40B), has also been obtained as detailed in Table 3.

### Compound (41)

STEP 1A is performed as disclosed here-above.

### Step 2: Synthesis of 2,4-Bis{4-[(5-dimethylaminopentyl)iminomethyl]phenyl}-7-phenyl-7H-imidazo[4,5-d]pyrimidine

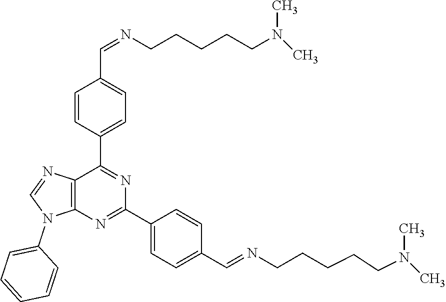 [SMILES: CN(C)CCCCC/N=C\c1ccc(-c2nc(-c3ccc(/C=N/CCCCCN(C)C)cc3)nc3c2ncn3-c2ccccc2)cc1]

Pale-yellow crystals (90%); Mp=115° C.; ¹H NMR δ (300 MHz, CDCl₃): 9.06 (d, 2H, J=8.40 Hz, H-2′ and H-6′), 8.70 (d, 2H, J=8.40 Hz, H-2″ and H-6″), 8.46 (s, 1H, H-6), 8.42 (s, 1H, CH═N), 8.39 (s, 1H, CH═N), 7.98 (d, 2H, J=8.40 Hz, H-3′ and H-5′), 7.92-7.86 (m, 4H, H-2′″, H-6′″, H-3″ and H-5″), 7.68 (t, 2H, J=7.50 Hz, H-3′″ and H-5″), 7.55 (t, 1H, J=7.50 Hz, H-4′″), 3.71 (t, 2H, J=6.90 Hz, NCH₂), 3.69 (t, 2H, J=6.90 Hz, NCH₂), 2.50 (t, 2H, J=6.90 Hz, NCH₂), 2.47 (t, 2H, J=6.90 Hz, NCH₂), 2.39 (s, 6H, N(CH₃)₂), 2.38 (s, 6H, N(CH₃)₂), 1.85-1.76 (m, 4H, 2CH₂), 1.70-1.63 (m, 4H, 2CH₂), 1.50-1.43 (m, 4H, 2CH₂).

STEP 3 leads to compound (41A) as detailed in Table 3.

The corresponding oxalate salt, compound (41B), has also been obtained as detailed in Table 3.

### Compound (42)

STEP 1A is performed as disclosed here-above.

### Step 2: Synthesis of 2,4-Bis{4-[(4-(4-methylpiperazin-1-yl)butyl)iminomethyl]phenyl}-7-phenyl-7H-imidazo[4,5-d]pyrimidine

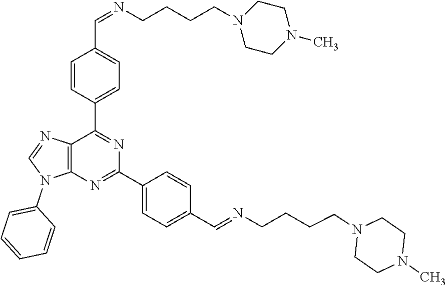 [SMILES: CN1CCN(CCCC/N=C\c2ccc(-c3nc(-c4ccc(/C=N/CCCCN5CCN(C)CC5)cc4)nc4c3ncn4-c3ccccc3)cc2)CC1]

White crystals (99%); Mp=135° C.; ¹H NMR δ (300 MHz, CDCl₃): 9.04 (d, 2H, J=8.40 Hz, H-2′ and H-6′), 8.66 (d, 2H, J=8.40 Hz, H-2″ and H-6″), 8.40 (s, 1H, H-6), 8.38 (s, 1H, CH═N), 8.34 (s, 1H, CH═N), 7.94 (d, 2H, J=8.40 Hz, H-3′ and H-5′), 7.88-7.81 (m, 4H, H-2′″, H-6′″, H-3″ and H-5″), 7.64 (t, 2H, J=7.80 Hz, H-3′″ and H-5″), 7.51 (t, 1H, J=7.80 Hz, H-4′″), 3.69 (t, 2H, J=6.90 Hz, NCH₂), 3.67 (t, 2H, J=6.90 Hz, NCH₂), 2.64-2.35 (m, 20H, 10NCH₂), 2.28 (s, 6H, 2NCH₃), 1.80-1.72 (m, 4H, 2CH₂), 1.65-1.51 (m, 4H, 2CH₂).

STEP 3 leads to compound (42A) as detailed in Table 3.

The corresponding oxalate salt, compound (42B), has also been obtained as detailed in Table 3.

### Example 2.11—Synthesis of Compounds (44) and (45)

### Step 1A (Ar₁=Ar₂): preparation 4,6-Bis(4-formylphenyl)-1-tert-butoxycarbonyl-1H-pyrrolo[2,3-b]pyridine

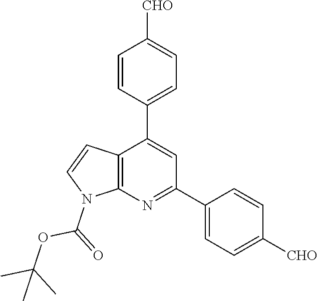 [SMILES: CC(C)(C)OC(=O)n1ccc2c(-c3ccc(C=O)cc3)cc(-c3ccc(C=O)cc3)nc21]

To a solution of 4.7 mmol of (SR9), 1.55 g of 4-formylphenyl boronic acid (10.3 mmol, 2.2 eq.) and 544 mg (0.47 mmol, 0.1 eq.) of tetrakis(triphenylphosphine)palladium in 45 mL of 1,2-dimethoxyethane, 5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×2 times), dried over anhydrous sodium sulfate and activated charcoal, filtered and evaporated under vacuum. The residue was cooled and triturated with a minimum of EtOH and Et₂O and filtered on sintered glassware to give the crude product. The residue was purified by silica gel column chromatography (CH₂Cl₂/CH₃OH 90:10) to give the solid product.

Yellow crystals (51%); Mp=170° C.; ¹H NMR δ (300 MHz, CDCl₃): 10.16 (s, 1H, CHO), 10.09 (s, 1H, CHO), 8.40 (d, 2H, J=8.10 Hz, H-2′ and H-6′), 8.07 (d, 2H, J=8.10 Hz, H-2″ and H-6″), 8.00 (d, 2H, J=8.10 Hz, H-3′ and H-5′), 7.89 (d, 2H, J=8.10 Hz, H-3″ and H-5″), 7.87 (s, 1H, H-5), 7.86 (d, 1H, J=3.90 Hz, H-2), 6.71 (d, 1H, J=3.90 Hz, H-3), 1.77 (s, 9H C(CH₃)₃).

### Intermediate Step: Preparation of 4,6-Bis(4-formylphenyl)-1H-pyrrolo[2,3-b]pyridine

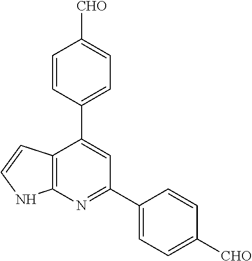 [SMILES: O=Cc1ccc(-c2cc(-c3ccc(C=O)cc3)c3cc[nH]c3n2)cc1]

To a solution of 4,6-bis(4-formylphenyl)-1-tert-butoxycarbonyl-1H-pyrrolo[2,3-b]pyridine (0.83 mmol) in anhydrous dichloromethane (20 mL) was added trifluoroacetic acid (TFA) (10 mL). The reaction mixture was stirred at room temperature for 5 min. Then dichloromethane and TFA were evaporated under reduced pressure to dryness, and after cooling the obtained residue was triturated in Et₂O, filtered, washed with Et₂O and dried under reduced pressure.

Pale-yellow crystals (98%); Mp=264° C.; ¹H NMR δ (300 MHz, CDCl₃): 13.44 (s, 1H, NH), 10.19 (s, 1H, CHO), 10.16 (s, 1H, CHO), 8.22 (d, 2H, J=8.40 Hz, H-2′ and H-6′), 8.18 (d, 2H, J=8.40 Hz, H-2″ and H-6″), 8.13 (d, 2H, J=8.40 Hz, H-3′ and H-5′), 8.01 (d, 2H, J=8.40 Hz, H-3″ and H-5″), 7.77 (s, 1H, H-5), 7.65 (dd, 1H, J=3.45 and 2.10 Hz, H-2), 6.85 (d, 1H, J=3.45 and 2.10 Hz, H-3).

### Step 2 Preparation of 4,6-bis[(substituted-iminomethyl)-1H-pyrrolo[2,3-b]pyridines

To a solution of the appropriate diamines (0.8 mmol, 2.15 eq.) in ethanol (12 mL) was added the 4,6-bis(4-formylphenyl)-1H-pyrrolo[2,3-b]pyridine (0.37 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Preparation of 4,6-bis[(substituted-aminomethyl)-1H-pyrrolo[2,3-b]pyridines

To a solution of 4,6-bis[(substituted-iminomethyl]-1H-pyrrolo[2,3-b]pyridine (0.4 mmol) in methanol (10 mL) was added portion-wise at 0° C. sodium borohydride (3.2 mmol, 8 eq.). The reaction mixture was then stirred at 60° C. for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the 4,6-bis[(substituted-aminomethyl)-1H-pyrrolo[2,3-b]pyridines.

### Compound (44)

STEP 1A is performed as disclosed here-above.

INTERMEDIATE STEP is performed as disclosed here-above.

### Step 2: Synthesis of 4,6-Bis{4-[(4-dimethylaminobutyl)iminomethyl]phenyl}-1H-pyrrolo[2,3-b]pyridine

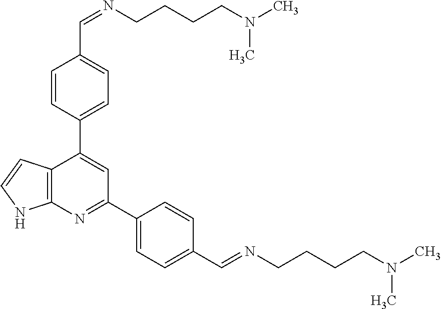 [SMILES: CN(C)CCCC/N=C\c1ccc(-c2cc(-c3ccc(/C=N/CCCCN(C)C)cc3)nc3[nH]ccc23)cc1]

Yellow oil (88%); ¹H NMR δ (300 MHz, CDCl₃): 12.04 (s, 1H, NH), 8.25 (s, 1H, CH═N), 8.24 (s, 1H, CH═N), 8.15 (d, 2H, J=8.10 Hz, H-2′ and H-6′), 7.85-7.71 (m, 6H, H-2″, H-6″, H-3′, H-5′, H-3″ and H-5″), 7.70 (s, 1H, H-5), 7.64 (d, 1H, J=3.60 Hz, H-2), 6.59 (d, 1H, J=3.60 Hz, H-3), 3.65-3.57 (m, 4H, 2NCH₂), 2.30-2.20 (m, 4H, 2NCH₂), 2.30 (s, 12H, 2N(CH₃)₂), 1.71-1.62 (m, 4H, 2CH₂), 1.55-1.43 (m, 4H, 2CH₂).

STEP 3 leads to compound (44A) as detailed in Table 3.

The corresponding oxalate salt, compound (44B), has also been obtained as detailed in Table 3.

### Compound (45)

STEP 1A is performed as disclosed here-above.

INTERMEDIATE STEP is performed as disclosed here-above.

### Step 2: Synthesis of 4,6-Bis{4-[(4-(4-methylpiperazin-1-yl)butyl)iminomethyl]phenyl}-1H-pyrrolo[2,3-b]pyridine

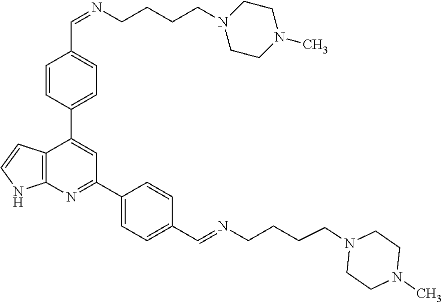 [SMILES: CN1CCN(CCCC/N=C\c2ccc(-c3cc(-c4ccc(/C=N/CCCCN5CCN(C)CC5)cc4)nc4[nH]ccc34)cc2)CC1]

Yellow-orange oil (97%); ¹H NMR δ (300 MHz, CDCl₃): 12.24 (s, 1H, NH), 8.34 (s, 2H, 2CH═N), 8.11 (d, 2H, J=8.10 Hz, H-2′ and H-6′), 7.90-7.82 (m, 6H, H-2″, H-6″, H-3′, H-5′, H-3″ and H-5″), 7.64 (s, 1H, H-5), 7.30 (d, 1H, J=3.60 Hz, H-2), 6.65 (d, 1H, J=3.60 Hz, H-3), 3.68-3.63 (m, 4H, 2NCH₂), 2.63-2.35 (m, 20H, 10NCH₂), 2.30 (s, 3H, NCH₃), 2.23 (s, 3H, NCH₃), 1.80-1.70 (m, 4H, 2CH₂), 1.65-1.52 (m, 4H, 2CH₂).

STEP 3 leads to compound (45A) as detailed in Table 3.

The corresponding oxalate salt, compound (45B), has also been obtained as detailed in Table 3.

### Example 2.12—Synthesis of Compound (46)

### Step 1A (Ar₁=Ar₂): Preparation 4,6-Bis(4-formylphenyl)-1-tert-butoxycarbonyl-1H-pyrrolo[2,3-b]pyridine is Performed as in Example 2.11 Here-Above

### INTERMEDIATE STEP1: Preparation of 4,6-Bis(4-formylphenyl)-1H-pyrrolo[2,3-b]pyridine is Performed as in Example 2.11 Here-Above

### Intermediate Step 2: Preparation of 4,6-Bis(4-formylphenyl)-1-benzyl-1H-pyrrolo[2,3-b]pyridine

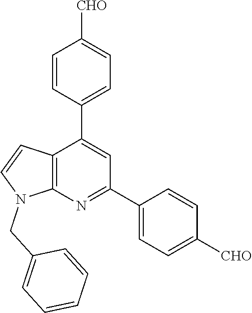 [SMILES: O=Cc1ccc(-c2cc(-c3ccc(C=O)cc3)c3ccn(Cc4ccccc4)c3n2)cc1]

A suspension of 4,6-bis(4-formylphenyl)-1H-pyrrolo[2,3-b]pyridine (1.0 mmol) in acetonitrile (10 mL) was treated with Cs₂CO₃ (3.0 mmol, 3 eq.) and benzyl bromide (1.5 mmol, 1.5 eq.), and stirred at 70° C. for 2 h. Then it was evaporated to dryness under reduced pressure. After cooling, the residue was triturated in water and extracted with dichloromethane (30 mL). The organic layer was separated, dried over sodium sulfate and activated charcoal and evaporated to dryness. The residue was purified by silica gel column chromatography (CH₂Cl₂) to give the solid product.

Yellow-orange crystals (51%); Mp=63° C.; ¹H NMR δ (300 MHz, CDCl₃) 10.14 (s, 1H, CHO), 10.10 (s, 1H, CHO), 8.38 (d, 2H, J=8.40 Hz, H-2′ and H-6′), 8.08 (d, 2H, J=8.40 Hz, H-2″ and H-6″), 8.01 (d, 2H, J=8.40 Hz, H-3′ and H-5′), 7.97 (d, 2H, J=8.40 Hz, H-3″ and H-5″), 7.78 (s, 1H, H-5), 7.40-7.32 (m, 6H, H-2 and 5H-_{Phen.}), 6.68 (d, 1H, J=3.60 Hz, H-3), 5.66 (s, 2H CH₂Ph).

### Step 2 Preparation of 4,6-Bis{4-[(4-dimethylaminobutyl)iminomethyl]phenyl}-1-benzyl-1H-pyrrolo[2,3-b]pyridine

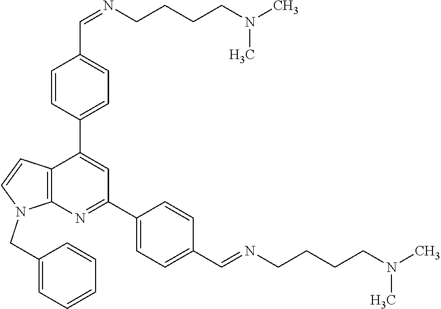 [SMILES: CN(C)CCCC/N=C\c1ccc(-c2cc(-c3ccc(/C=N/CCCCN(C)C)cc3)nc3c2ccn3Cc2ccccc2)cc1]

To a solution of the appropriate diamines (0.8 mmol, 2.15 eq.) in ethanol (12 mL) was added the 4,6-bis(4-formylphenyl)-1-benzyl-1H-pyrrolo[2,3-b]pyridine (0.37 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Preparation of 4,6-Bis{4-[(4-dimethylaminobutyl)aminomethyl]phenyl}-1-benzyl-1H-pyrrolo[2,3-b]pyridine

To a solution of 4,6-bis{4-[(4-dimethylaminobutyl)iminomethyl]phenyl}-1-benzyl-1H-pyrrolo[2,3-b]pyridine (0.4 mmol) in methanol (10 mL) was added portion-wise at 0° C. sodium borohydride (3.2 mmol, 8 eq.). The reaction mixture was then stirred at 60° C. for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness to give the compound (46A) as detailed in Table 3.

The corresponding oxalate salt, compound (46B), has also been obtained as detailed in Table 3.

### Example 3—Preparation of Compound (12)

Compound (12) is prepared following general protocol 11 (GP11) as defined here-above.

### Step 1A (Ar₁=Ar₂): Synthesis of 7-(4-Formylphenyl)-2,4-diphenyl-7H-pyrrolo[2,3-d]pyrimidine

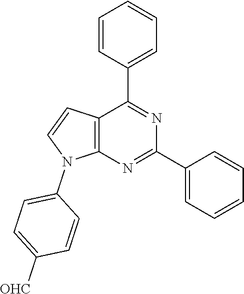 [SMILES: O=Cc1ccc(-n2ccc3c(-c4ccccc4)nc(-c4ccccc4)nc32)cc1]

To a solution of 4.0 mmol of (SR3), 8.8 mmol of phenylboronic acid (2.2 eq.) and 0.80 mmol (0.1 eq.) of tetrakis(triphenylphosphine) palladium in 15 mL of 1,2-dimethoxyethane, 3.0 mL of 2M Na₂CO₃ aqueous solution, previously degassed for 10 minutes with nitrogen, were added at room temperature. Then, the mixture was warmed to reflux and stirred for 24 hours under nitrogen positive pressure. The solvent was then evaporated under vacuum. The residue was extracted with CH₂Cl₂ and the organic phase was filtered on filter paper, then washed with water (15 mL×2 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, EtO₂ and petroleum ether and dried under pressure to give the solid product.

White crystals (61%) Mp=138° C.; ¹H NMR δ (300 MHz, CDCl₃): 10.11 (s, 1H, CHO), 8.66 (dd, 2H, J=7.80 Hz and J=1.50 Hz H-2′ and H-6′), 8.28 (dd, 2H, J=7.80 Hz and J=1.50 Hz, H-2″ and H6″), 8.20 (d, 2H, J=8.40 Hz, H-3″ and H5″), 7.64 (d, 1H, J=3.90 Hz, H-6), 7.62-7.49 (m, 6H, H-3′, H-4′, H-5, H-3″, H-4″ and H-5″), 7.05 (d, J=3.90 Hz, H-5).

### Step 2: Synthesis of 7-(4-[(3-Dimethylaminopropyl)iminomethyl]phenyl)-2,4-diphenyl-7H-pyrrolo[2,3-d]pyrimidine

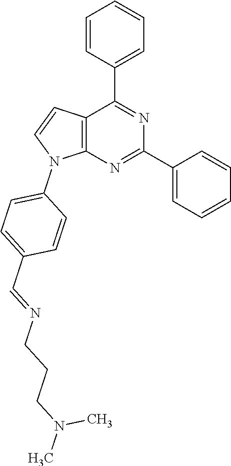 [SMILES: CN(C)CCC/N=C/c1ccc(-n2ccc3c(-c4ccccc4)nc(-c4ccccc4)nc32)cc1]

To a solution of 3-dimethylaminopropylamine (0.66 mmol, 1.1 eq.) in ethanol (7 mL) was added the 7-(4-formylphenyl)-2,4-diphenyl-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (20 mL). The organic layer was dried over sodium sulfate and activated charcoal and evaporated to dryness. Products were then used without further purification.

White crystals (88%), Mp=122° C.; ¹H NMR δ (300 MHz, CDCl₃): 8.67 (dd, 2H, J=7.80 Hz and J=1.20 Hz, H-2′ and H-6′), 8.40 (s, 1H, CH═N), 8.29 (dd, 2H, J=7.80 Hz and J=1.20 Hz, H-2″ and H-6″), 8.02 (d, 2H, J=8.40 Hz, H-3′″ and H-5′″), 7.97 (d, 2H, J=8.40 Hz, H-2′″ and H-6′″), 7.63-7.46 (m, 7H, H-3′, H-4′, H-5′, H-3″, H-4″, H-5″ and H-6), 7.02 (d, 1H, J=3.90 Hz, H-5), 3.73 (t, 2H, J=6.90 Hz, NCH₂), 2.46 (t, 2H, J=6.90 Hz, NCH₂), 2.32 (s, 6H, N(CH₃)₂), 1.97 (qt, 2H, J=6.90 Hz, CH₂).

### Step 3: Synthesis of Compound (12A)

To a solution of 7-(4-[(3-dimethylaminopropyl)iminomethyl]phenyl)-2,4-diphenyl-7H-pyrrolo[2,3-d]pyrimidine (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give compound (12A) as detailed in Table 3.

The corresponding oxalate salt, compound (12B) has also been obtained as detailed in Table 3.

### Example 4—Preparation of Compound (13)

Compound (13) is prepared following general protocol 15 (GP15) as defined here-above.

### Step 1A (Ar₁=Ar₂): Synthesis of 2,4,7-Tri(4-formylphenyl)-7H-pyrrolo[2,3-d]pyrimidine

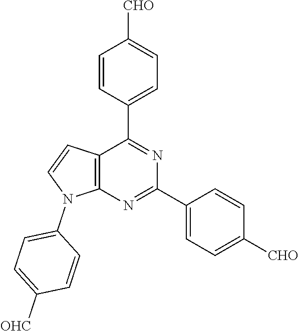 [SMILES: O=Cc1ccc(-c2nc(-c3ccc(C=O)cc3)c3ccn(-c4ccc(C=O)cc4)c3n2)cc1]

To a solution of 2 mmol of (SR3), 4.4 mmol of 4-formylphenyl boronic acid (2.2 eq.) and 240 mg (0.2 mmol, 0.1 eq.) of tetrakis(triphenylphosphine) palladium in 25 mL of 1,2-dimethoxyethane, 3.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CHCl₃ and the organic phase was filtered on filter paper, then washed with water (20 mL×3 times), dried over anhydrous sodium sulfate and activated charcoal, filtered and evaporated under vacuum. The residue was purified by silica gel column chromatography (CHCl₃), then cooled and triturated again in EtOH, filtered on sintered glassware, washed with a minimum of EtOH and EtO₂ and dried under pressure to give the solid product.

Pale-yellow crystals (22%) Mp >240° C.; ¹H NMR δ (300 MHz, CDCl₃): 10.17 (s, 1H, CHO), 10.11 (s, 1H, CHO), 10.10 (s, 1H, CHO), 8.75 (d, 2H, J=8.40 Hz, H-2′ and H-6′), 8.51 (d, 2H, J=8.40 Hz, H-2″ and H-6″), 8.35 (d, 1H, J=3.90 Hz, H-6), 8.33 (d, 2H, J=8.40 Hz, H-2′″, H-6′″), 8.18 (d, 2H, J=8.40 Hz, H-3′ and H-5′), 8.15 (d, 2H, J=8.40 Hz, H-3″, H-5″), 8.05 (d, 2H, J=8.40 Hz, H-3′″, H-5′″), 7.29 (d, 1H, J=3.90 Hz, H-5).

### Step 2: Synthesis of 2,4,7-Tri{4-[(3-dimethylaminopropyl)aminomethyl]phenyl}-7H-pyrrolo[2,3-d]pyrimidine

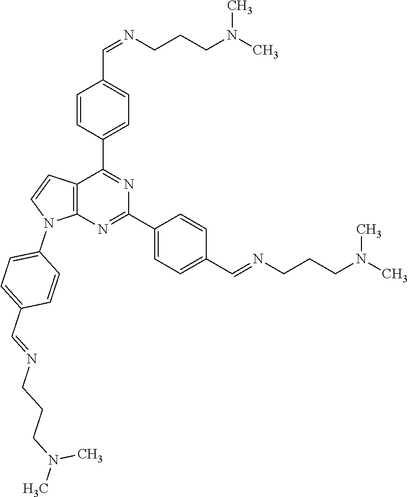 [SMILES: CN(C)CCC/N=C\c1ccc(-c2nc(-c3ccc(/C=N/CCCN(C)C)cc3)nc3c2ccn3-c2ccc(/C=N/CCCN(C)C)cc2)cc1]

To a solution of 3-dimethylaminopropylamine (0.726 mmol, 3.3 eq.) in ethanol (7 mL) was added the 2,4,7-tri(4-formylphenyl)-7H-pyrrolo[2,3-d]pyrimidine (0.22 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

Orange oil (98%); ¹H NMR δ (300 MHz, CDCl₃): 8.66 (d, 2H, J=8.40 Hz, H-2′ and H-6′), 8.39 (s, 1H, CH═N), 8.37 (s, 1H, CH═N), 8.36 (s, 1H, CH═N), 8.29 (d, 2H, J=8.40 Hz, H-2″ and H-6″), 7.99-7.92 (m, 6H, H-2′″, H-6′″, H-3′, H-5′, H-3″ and H-5″), 7.83 (d, 2H, J=8.40 Hz, H-3′″ and H-5′″), 7.62 (d, 1H, J=3.60 Hz, H-6), 6.98 (d, 1H, J=3.60 Hz, H-5), 3.73-3.66 (m, 6H, 3NCH₂), 2.41-2.34 (m, 6H, 3NCH₂), 2.26 (s, 6H, N(CH₃)₂), 2.24 (s, 12H, 2N(CH₃)₂), 1.94-1.87 (m, 6H, 3CH₂).

### Step 3: Synthesis of Compound (13A) as Detailed in Table 3

To a solution of 2,4,7-tri{4-[(3substituted-aminoalkyl)iminomethyl]phenyl}-7H-pyrrolo[2,3-d]pyrimidine (0.22 mmol) in methanol (7 mL) was added portion-wise at 0° C. sodium borohydride (1.32 mmol, 6 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the 2,4,7-tri{4-[(substituted-aminoalkyl)aminomethyl]aryl}-7H-pyrrolo[2,3-d]pyrimidine.

The corresponding oxalate salt, compound (13B), has also been obtained as detailed in Table 3.

### Example 5—Preparation of Compounds (14), (28) and (31)

Compounds (14), (28) and (31) are prepared following general protocol 5 (GP5) as defined here-above.

### Example 5.1: Synthesis of Compound (14)

### Step 1A: Synthesis of 2-Chloro-4-(4-formylphenyl)-7-phenyl-7H-pyrrolo[2,3-d]pyrimidine

This step is performed as STEP 1A of example 2.3.

### Step 2: Synthesis of 2-(3-dimethylaminopropylamino)-4-{4-[(4-dimethylaminopropyl)iminomethyl]phenyl}-7-phenyl-7H-pyrrolo[2,3-d]pyrimidine

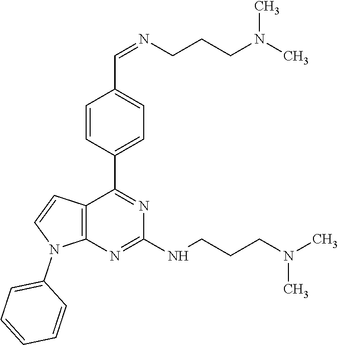 [SMILES: CN(C)CCC/N=C\c1ccc(-c2nc(NCCCN(C)C)nc3c2ccn3-c2ccccc2)cc1]

To a solution of 3-dimethylaminopropylamine (1.64 mmol, 3.0 eq.) in ethanol (7 mL) was added 2-Chloro-4-(4-formylphenyl)-7-phenyl-7H-pyrrolo[2,3-d]pyrimidine (0.55 mmol). The reaction mixture was then heated under reflux for 24 h, and then evaporated to dryness under reduced pressure. After cooling, the residue was extracted with dichloromethane (20 mL). The organic layer was dried over sodium sulfate and activated charcoal and evaporated to dryness. The crude product was then used without further purification.

### Step 3: Synthesis of Compound (14A) as Detailed in Table 3

To a solution of 2-(3-dimethylaminopropylamino)-4-{4-[(4-dimethylaminopropyl)iminomethyl]phenyl}-7-phenyl-7H-pyrrolo[2,3-d]pyrimidine (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the final product.

The corresponding oxalate salt, compound (14B), has also been obtained as detailed in Table 3.

### Example 5.2: Synthesis of Compound (28)

### Step 1A: Synthesis of 2-Chloro-4-(4-formylphenyl)thieno[2,3-d]pyrimidine

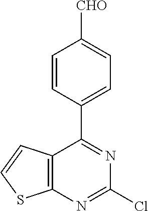 [SMILES: O=Cc1ccc(-c2nc(Cl)nc3sccc23)cc1]

To a solution of 3.0 mmol of 2,4-dichlorothieno[2,3-d]pyrimidine, 0.49 g of 4-formylphenyl boronic acid (3.3 mmol, 1.1 eq.) and 170 mg (0.15 mmol, 0.05 eq.) of tetrakis(triphenylphosphine) palladium in 25 mL of 1,2-dimethoxyethane, 4 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, then EtO₂ and petroleum ether and dried under pressure to give the solid product.

White crystals (33%); Mp=123° C.; ¹H NMR δ (300 MHz, CDCl₃): 10.17 (s, 1H, CHO), 8.15 (d, 2H, J=8.40 Hz, H-2′ and H-6′), 8.10 (d, 2H, J=8.40 Hz, H-3′ and H-5′), 7.67 (d, 1H, J=6.00 Hz, H-6), 7.58 (d, 1H, J=6.00 Hz, H-5).

### Step 2: Synthesis 2-(3-Dimethylaminopropylamino)-4-{4-[(4-dimethylaminopropyl)iminomethyl]phenyl}thieno[2,3-d]pyrimidine

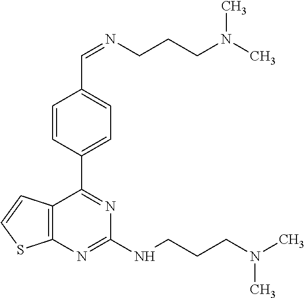 [SMILES: CN(C)CCC/N=C\c1ccc(-c2nc(NCCCN(C)C)nc3sccc23)cc1]

To a solution of the 3-dimethylaminopropylamine (1.64 mmol, 3.0 eq.) in ethanol (7 mL) was added 2-Chloro-4-(4-formylphenyl)thieno[2,3-d]pyrimidine (0.55 mmol). The reaction mixture was then heated under reflux for 24 h, and then evaporated to dryness under reduced pressure. After cooling, the residue was extracted with dichloromethane (20 mL). The organic layer was dried over sodium sulfate and activated charcoal and evaporated to dryness. The crude product was then used without further purification.

Orange oil (82%); ¹H NMR δ (300 MHz, CDCl₃): 8.23 (s, 1H, CH═N), 7.79 (d, 2H, J=8.10 Hz, H-2′ and H-6′), 7.73 (d, 2H, J=8.10 Hz, H-3′ and H-5′), 7.13 (d, 1H, J=6.00 Hz, H-6), 6.87 (d, 1H, J=6.00 Hz, H-5), 5.95 (t, 1H, J=5.70 Hz, NH), 3.56-3.48 (m, 2H, NCH₂), 2.84 (t, 2H, J=6.30 Hz, NCH₂), 2.30-2.25 (m, 2H, NCH₂), 2.12 (s, 6H, N(CH₃)₂), 2.11 (s, 6H, N(CH₃)₂), 1.70-1.61 (m, 4H, 2 CH₂).

### Step 3: Synthesis of Compound (28A) as Detailed in Table 3

To a solution of 2-(3-dimethylaminopropylamino)-4-{4-[(4-dimethylaminopropyl)iminomethyl]phenyl}thieno[2,3-d]pyrimidine (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the final product.

The corresponding oxalate salt, compound (28B), has also been obtained as detailed in Table 3.

### Example 5.3: Synthesis of Compound (31)

### Step 1A: Synthesis of 2-Chloro-4-(4-formylphenyl)thieno[3,2-d]pyrimidine

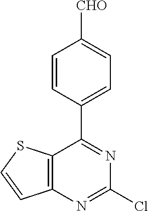 [SMILES: O=Cc1ccc(-c2nc(Cl)nc3ccsc23)cc1]

To a solution of 3.0 mmol of 2,4-dichlorothieno[3,2-d]pyrimidine, 0.49 g of 4-formylphenyl boronic acid (3.3 mmol, 1.1 eq.) and 170 mg (0.15 mmol, 0.05 eq.) of tetrakis(triphenylphosphine) palladium in 25 mL of 1,2-dimethoxyethane, 4 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (20 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, then EtO₂ and petroleum ether and dried under pressure to give the solid product. White crystals (37%); Mp=216° C.; ¹H NMR δ (300 MHz, CDCl₃): 10.17 (s, 1H, CHO), 8.39 (d, 2H, J=8.40 Hz, H-2′ and H-6′), 8.18 (d, 1H, J=5.70 Hz, H-6), 8.13 (d, 2H, J=8.40 Hz, H-3′ and H-5′), 7.64 (d, 1H, J=5.70 Hz, H-7).

### Step 2: Synthesis of 2-(3-Dimethylaminopropylamino)-4-{4-[(4-dimethylaminopropyl)iminomethyl]phenyl}thieno[3,2-d]pyrimidine

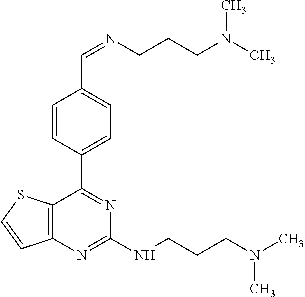 [SMILES: CN(C)CCC/N=C\c1ccc(-c2nc(NCCCN(C)C)nc3ccsc23)cc1]

To a solution of 3-dimethylaminopropylamine (1.64 mmol, 3.0 eq.) in ethanol (7 mL) was added 2-chloro-4-(4-formylphenyl)thieno[3,2-d]pyrimidine (0.55 mmol). The reaction mixture was then heated under reflux for 24 h, and then evaporated to dryness under reduced pressure. After cooling, the residue was extracted with dichloromethane (20 mL). The organic layer was dried over sodium sulfate and activated charcoal and evaporated to dryness. The crude product was then used without further purification.

Orange oil (97%); ¹H NMR δ (300 MHz, CDCl₃): 8.30 (s, 1H, CH═N), 8.10 (d, 2H, J=8.40 Hz, H-2′ and H-6′), 7.82 (d, 2H, J=8.40 Hz, H-3′ and H-5′), 7.76 (d, 1H, J=5.70 Hz, H-6), 7.20 (d, 1H, J=5.70 Hz, H-7), 5.72 (t, 1H, J=5.70 Hz, NH), 3.63 (t, 2H, J=6.30 Hz, NCH₂), 3.53 (t, 2H, J=6.30 Hz, NCH₂), 2.87 (t, 2H, J=6.30 Hz, NCH₂), 2.34 (t, 2H, J=6.30 Hz, NCH₂), 2.19 (s, 6H, N(CH₃)₂), 2.18 (s, 6H, N(CH₃)₂), 1.85-1.76 (m, 2H, CH₂), 1.71-1.67 (m, 2H, CH₂).

### Step 3: Synthesis of Compound (31A) as Detailed in Table 3

To a solution of 2-(3-dimethylaminopropylamino)-4-{4-[(4-dimethylaminopropyl)iminomethyl]phenyl}thieno[3,2-d]pyrimidine (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the final product.

The corresponding oxalate salt, compound (31B) has also been obtained as detailed in Table 3.

### Example 6—Preparation of Compounds (15) to (21)

Compounds (15) to (21) are prepared following general protocol 4 (GP4) as defined here-above.

### Step 1A: Synthesis of 2-chloro-4-(formylaryl)-7H-pyrrolo[2,3-d]pyrimidines

To a solution of 4.0 mmol of 2,4-dichloro-7H-pyrrolo[2,3-d]pyrimidine or 2,4-dichloro-7-R⁵-7H-pyrrolo[2,3-d]pyrimidine, 4.4 mmol of formylaryl boronic acid (1.1 eq.) and 0.40 mmol (0.05 eq.) of tetrakis(triphenylphosphine) palladium in 15 mL of 1,2-dimethoxyethane, 2.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The solvent was then evaporated under vacuum. The residue was extracted with CH₂Cl₂ and the organic phase was filtered on filter paper, then washed with water (15 mL×2 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, EtO₂ and petroleum ether and dried under pressure to give the solid product.

### Step 2: Synthesis of 2-chloro-4-[(substituted-iminomethyl)aryl]-7H-pyrrolo[2,3-d]pyrimidines

To a solution of the appropriate diamine (0.66 mmol, 1.1 eq.) in ethanol (7 mL) was added the 2-chloro-4-(formylaryl)-7-R-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (20 mL). The organic layer was dried over sodium sulfate and activated charcoal and evaporated to dryness. Products were then used without further purification.

### Step 3: Synthesis of 2-chloro-4-[(substituted-aminomethyl)aryl]-7H-pyrrolo[2,3-d]pyrimidines

To a solution of 2-chloro-4-[(substituted-iminomethyl)-7-R1-7H-pyrrolo[2,3-d]pyrimidine (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the final product.

### Example 6.1—Synthesis of Compounds (15), (16) and (17)

### Step 1A: Synthesis of 2-Chloro-4-(4-formylphenyl)-7H-pyrrolo[2,3-d]pyrimidine

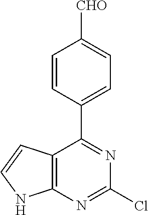 [SMILES: O=Cc1ccc(-c2nc(Cl)nc3[nH]ccc23)cc1]

Pale-yellow crystals (62%) Mp=221° C.; ¹H NMR δ (300 MHz, CDCl₃): 12.62 (bs, 1H, NH), 10.14 (s, 1H, CHO), 8.37 (d, 2H, J=8.40 Hz, H-2′ and H-6′), 8.12 (d, 2H, J=8.40 Hz, H-3′ and H-5′), 7.79 (d, 1H, J=3.60 Hz, H-6), 7.01 (d, 1H, J=3.60 Hz, H-5).

### Compound (15)

### Step 2: Synthesis of 2-Chloro-4-{4-[(3-dimethylaminopropyl)iminomethyl]phenyl}-7H-pyrrolo[2,3-d]pyrimidine

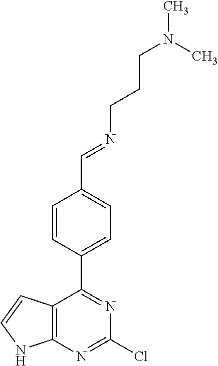 [SMILES: CN(C)CCC/N=C/c1ccc(-c2nc(Cl)nc3[nH]ccc23)cc1]

Yellow crystals (76%), Mp=145° C.; ¹H NMR δ (300 MHz, CDCl₃): 8.39 (s, 1H, CH═N), 8.21 (d, 2H, J=8.40 Hz, H-2′ and H-6′), 7.91 (d, 2H, J=8.40 Hz, H-3′ and H-5′), 7.48 (d, 1H, J=3.60 Hz, H-6), 6.86 (d, 1H, J=3.60 Hz, H-5), 3.73 (t, 2H, J=7.20 Hz, CH₂), 2.43 (s, 6H, NCH₂), 2.30 (s, 6H, N(CH₃)₂), 1.95 (qt, 2H, J=7.20 Hz, CH₂).

STEP 3 leads to compound (15A) as detailed in Table 3.

The corresponding oxalate salt, compound (15B) has also been obtained as detailed in Table 3.

### Compound (16)

### Step 2: Synthesis of 2-Chloro-4-{4-[(3-(4-methylpiperazin-1-yl)propyl)iminomethyl]phenyl}-7H-pyrrolo[2,3-d]pyrimidine

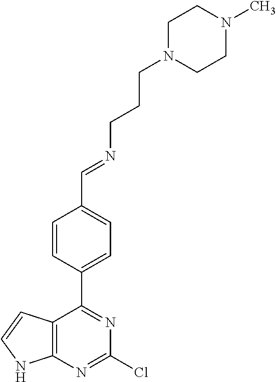 [SMILES: CN1CCN(CCC/N=C/c2ccc(-c3nc(Cl)nc4[nH]ccc34)cc2)CC1]

Yellow crystals (98%), Mp=97° C.; ¹H NMR δ (300 MHz, CDCl₃): 8.36 (s, 1H, CH═N), 8.18 (d, 2H, J=8.40 Hz, H-2′ and H-6′), 7.87 (d, 2H, J=8.40 Hz, H-3′ and H-5′), 3.70 (t, 2H, J=6.90 Hz, NCH₂), 2.60-2.47 (m, 10H, 5NCH₂), 2.33 (s, 3H, NCH₃), 1.93 (qt, 2H, J=6.90 Hz, CH₂).

STEP 3 leads to compound (16A) as detailed in Table 3.

The corresponding oxalate salt, compound (16B), has also been obtained as detailed in Table 3.

### Compound (17)

### Step 2: Synthesis of 2-Chloro-4-{4-[(3-dimethylaminopropyl)iminomethyl]phenyl}-7-phenyl-7H-pyrrolo[2,3-d]pyrimidine

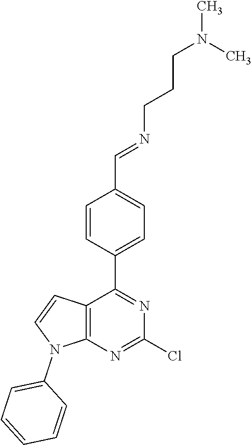 [SMILES: CN(C)CCC/N=C/c1ccc(-c2nc(Cl)nc3c2ccn3-c2ccccc2)cc1]

Yellow crystals (97%) Mp=42° C.; ¹H NMR δ (300 MHz, CDCl₃): 8.36 (s, 1H, CH═N), 8.15 (d, 2H, J=8.40 Hz, H-2′ and H-6′), 7.88 (d, 2H, J=8.40 Hz, H-3′ and H-5′), 7.66 (d, 2H, J=8.10 Hz, H-2″ and H-6″), 7.52 (d, 1H, J=3.60 Hz, H-6), 7.50 (t, 2H, J=8.10 Hz, H-3″ and H-5″), 7.36 (t, 1H, J=8.10 Hz, H-4″), 6.92 (d, 1H, J=3.60 Hz, H-5), 3.68 (t, 2H, J=6.90 Hz, NCH₂), 2.37 (t, 2H, J=6.90 Hz, NCH₂), 2.24 (s, 6H, N(CH₃)₂), 1.90 (qt, 2H, J=6.90 Hz, CH₂).

STEP 3 leads to compound (17A) as detailed in Table 3.

The corresponding oxalate salt, compound (17B), has also been obtained) as detailed in Table 3.

### Compound (18)

### Step 2: Synthesis of 2-Chloro-4-{4-[(3-(4-methylpiperazin-1-yl)propyl)iminomethyl]phenyl}-7-phenyl-7H-pyrrolo[2,3-d]pyrimidine

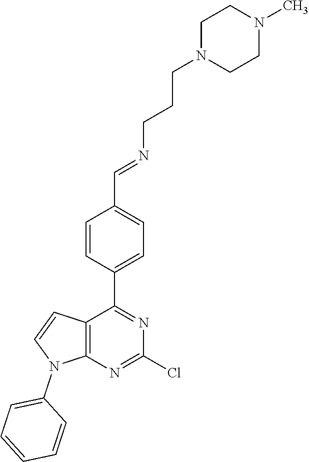 [SMILES: CN1CCN(CCC/N=C/c2ccc(-c3nc(Cl)nc4c3ccn4-c3ccccc3)cc2)CC1]

Yellow oil (98%); 1H NMR δ (300 MHz, CDCl3): 8.38 (s, 1H, CH═N), 8.18 (d, 2H, J=8.40 Hz, H-2′ and H-6′), 7.91 (d, 2H, J=8.40 Hz, H-3′ and H-5′), 7.71 (d, 2H, J=7.60 Hz, H-2″ and H-6″), 7.57 (d, 1H, J=3.90 Hz, H-6), 7.55 (t, 2H, J=7.60 Hz, H-3″ and H-5″), 7.41 (t, 1H, J=7.60 Hz, H-4″) 6.97 (d, 1H, J=3.90 Hz, H-5), 3.71 (t, 2H, J=6.90 Hz, NCH2), 2.62-2.33 (m, 10H, 5NCH2), 2.30 (s, 3H, NCH3), 1.94 (qt, 2H, J=6.90 Hz, CH2).

STEP 3 leads to compound (18A) as detailed in Table 3.

The corresponding oxalate salt, compound (18B) has also been obtained as detailed in Table 3.

### Example 6.2—Synthesis of Compounds (19), (20) and (21)

### Compound (19)

### Step 1A: Synthesis of 2-Chloro-4-(5-formylthien-2-yl)-7-phenyl-7H-pyrrolo[2,3-d]pyrimidine is Performed as STEP 1A of Example 2.2 Here-Above

### Step 2: Synthesis of 2-chloro-4-{5-[(3-dimethylaminopropyl)iminomethyl]thien-2-yl}-7-phenyl-7H-pyrrolo[2,3-d]pyrimidine

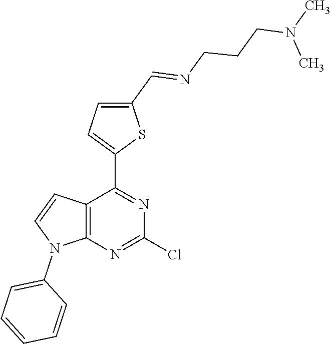 [SMILES: CN(C)CCC/N=C/c1ccc(-c2nc(Cl)nc3c2ccn3-c2ccccc2)s1]

Yellow oil (97%); ¹H NMR δ (300 MHz, CDCl₃): 8.43 (s, 1H, CH═N), 8.06 (d, 1H, J=3.60 Hz, H-6), 7.70 (d, 2H, J=8.40 Hz, H-2′ and H-6′), 7.57 (d, 1H, J=3.90 Hz, H-thioph), 7.55 (d, 2H, J=8.40 Hz, H-3′ and H-5′), 7.41 (t, 1H, J=8.10 Hz, H-4′), 7.39 (d, 1H, J=3.90 Hz, H-thioph), 7.10 (d, 1H, J=3.60 Hz, H-5), 3.69 (t, 2H, J=6.90 Hz, NCH₂), 2.39 (t, 2H, J=6.90 Hz, NCH₂), 2.27 (s, 6H, N(CH₃)₂), 1.90 (qt, 2H, J=6.90 Hz, CH₂).

STEP 3 leads to compound (19A) as detailed in Table 3.

The corresponding oxalate salt, compound (19B), has also been obtained as detailed in Table 3.

### Compound (20)

### Step 1A: Synthesis of 2-Chloro-4-{2-formylthien-4-yl}-7-phenyl-7H-pyrrolo[2,3-d]pyrimidine

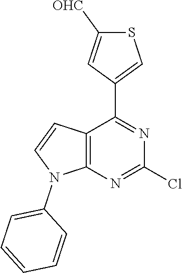 [SMILES: O=Cc1cc(-c2nc(Cl)nc3c2ccn3-c2ccccc2)cs1]

Pale-yellow crystals (27%), Mp=143° C. ¹H NMR δ (300 MHz, CDCl₃): 10.08 (s, 1H, CHO), 8.58 (dd, 1H, J=1.20 and 1.20 Hz, H-thioph.), 8.58 (d, 1H, J=1.20 Hz, H-thioph.), 7.73 (d, 2H, J=7.50 Hz, H-2′ and H-6′), 7.63 (d, 1H, J=3.60 Hz, H-6), 7.59 (t, 2H, J=7.50 Hz, H-3′ and H-5′), 7.45 (t, 1H, J=7.50 Hz, H-4′), 7.00 (d, 1H, J=3.60 Hz, H-5); ¹³C NMR δ (75 MHz, CDCl₃): 184.3 (CHO), 155.6 (C-4), 154.4 (C-2), 146.0 (C-7a), 144.3 (C-1′.), 141.7 (C-thioph.), 137.8 (C-thioph.), 137.4 (C-3′ and C-5′), 131.6 (C-4′), 131.0 (C-2′ and C-6′), 129.3 (C-6), 126.3 (C-thioph.), 125.4 (C-thioph.), 115.8 (C-4a), 102.7 (C-5).

### Step 2: Synthesis of 2-Chloro-4-{2-[(3-dimethylaminopropyl)iminomethyl]thien-4-yl}-7-phenyl-7H-pyrrolo[2,3-d]pyrimidine

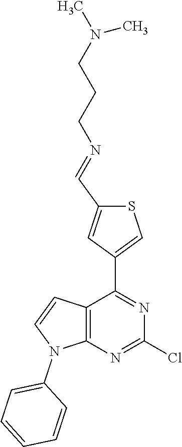 [SMILES: CN(C)CCC/N=C/c1cc(-c2nc(Cl)nc3c2ccn3-c2ccccc2)cs1]

Yellow oil (85%); ¹H NMR δ (300 MHz, CDCl₃): 8.44 (s, 1H, CH═N), 8.23 (dd, 1H, J=1.20×1.20 Hz, H-thioph), 8.04 (d, 1H, J=1.20 Hz, H-thioph), 7.68 (d, 2H, J=7.50 Hz, H-2′ and H-6′), 7.55 (d, 1H, J=3.60 Hz, H-6), 7.53 (t, 2H, J=7.50 Hz, H-3′ and H-5′), 7.39 (t, 1H, J=7.50 Hz, H-4′), 6.93 (d, 1H, J=3.60 Hz, H-5), 3.66 (t, 2H, J=7.20 Hz, NCH₂), 2.36 (t, 2H, J=7.20 Hz, NCH₂), 2.25 (s, 6H, N(CH₃)₂), 1.89 (qt, 2H, J=7.20 Hz, CH₂).

STEP 3 leads to compound (20A) as detailed in Table 3.

The corresponding oxalate salt, compound (20B), has also been obtained as detailed in Table 3.

### Compound (21)

### Step 1A: Synthesis of 2-Chloro-4-{2-formylthien-3-yl}-7-phenyl-7H-pyrrolo[2,3-d]pyrimidine

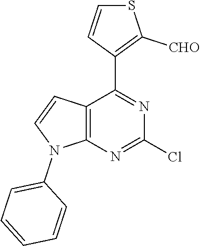 [SMILES: O=Cc1sccc1-c1nc(Cl)nc2c1ccn2-c1ccccc1]

Pale-yellow crystals (62%), Mp >260° C.; ¹H NMR δ (300 MHz, CDCl₃): 10.48 (d, 1H, J=1.00 Hz, CHO), 7.87 (dd, 1H, J=5.40 and 1.00 Hz, H-thioph.), 7.73 (d, 2H, J=7.80 Hz, H-2′ and H-6′), 7.70 (d, 1H, J=5.10 Hz, H-thioph.), 7.63 (d, 1H, J=3.75 Hz, H-6), 7.60 (t, 2H, J=7.80 Hz, H-3′ and H-5′), 7.47 (t, 1H, J=7.80 Hz, H-4′), 6.85 (d, 1H, J=3.75 Hz, H-5).

### Step 2: Synthesis of 2-Chloro-4-{2-[(3-dimethylaminopropyl)iminomethyl]thien-3-yl}-7-phenyl-7H-pyrrolo[2,3-d]pyrimidine

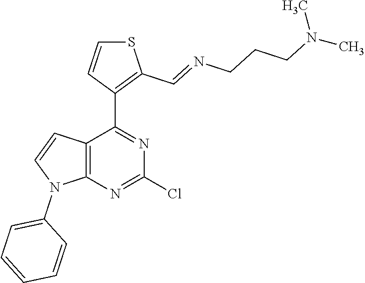 [SMILES: CN(C)CCC/N=C/c1sccc1-c1nc(Cl)nc2c1ccn2-c1ccccc1]

Yellow oil (44%); ¹H NMR δ (300 MHz, CDCl₃): 8.90 (s, 1H, CH═N), 7.73 (d, 2H, J=7.50 Hz, H-2′ and H-6′), 7.58 (t, 2H, J=7.50 Hz, H-3′ and H-5′), 7.55 (d, 1H, J=5.40 Hz, H-thioph.), 7.54 (d, 1H, J=3.60 Hz, H-6), 7.50 (d, 1H, J=5.40 Hz, H-thioph.), 7.44 (t, 1H, J=7.50 Hz, H-4′), 6.79 (d, 1H, J=3.60 Hz, H-5), 3.66 (t, 2H, J=6.90 Hz, NCH₂), 2.38 (t, 2H, J=6.90 Hz, NCH₂), 2.25 (s, 6H, N(CH₃)₂), 1.90 (qt, 2H, J=6.90 Hz, CH₂).

STEP 3 leads to compound (21A) as detailed in Table 3.

The corresponding oxalate salt, compound (21B), has also been obtained as detailed in Table 3.

### Example 7—Preparation of Compounds (22) and (23)

Compounds (22) and (23) are prepared following general protocol 9 (GP9) as defined here-above.

### Step 1A: Synthesis of 2-Chloro-7-(4-formylphenyl)-4-phenyl-7H-pyrrolo[2,3-d]pyrimidine

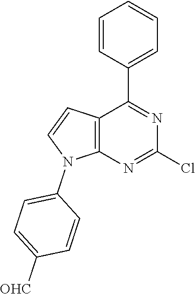 [SMILES: O=Cc1ccc(-n2ccc3c(-c4ccccc4)nc(Cl)nc32)cc1]

To a solution of 4.0 mmol of 2,4-dichloro-7-4-formylphenyl)-7H-pyrrolo[2,3-d]pyrimidine, 4.4 mmol of phenylboronic acid (1.1 eq.) and 0.40 mmol (0.05 eq.) of tetrakis(triphenylphosphine) palladium in 15 mL of 1,2-dimethoxyethane, 2.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The solvent was then evaporated under vacuum. The residue was extracted with CH₂Cl₂ and the organic phase was filtered on filter paper, then washed with water (15 mL×2 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂), then cooled and triturated again in EtOH, filtered on sintered glassware, washed with a minimum of EtOH, EtO₂ and petroleum ether and dried under pressure to give the solid product.

### Step 2: Synthesis of 2-chloro-4-phenyl-7-[(substituted-iminomethyl)-7H-pyrrolo[2,3-d]pyrimidines

To a solution of the appropriate diamine (0.66 mmol, 1.1 eq.) in ethanol (7 mL) was added the 2-chloro-4-phenyl-7-(4-formylphenyl)-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (20 mL). The organic layer was dried over sodium sulfate and activated charcoal and evaporated to dryness. Products were then used without further purification.

### Step 3: Synthesis of 2-chloro-4-phenyl-7-[(substituted-aminomethyl)-7H-pyrrolo[2,3-d]pyrimidines

To a solution of 2-chloro-4-phenyl-7-[(substituted-iminomethyl)-7H-pyrrolo[2,3-d]pyrimidines (0.55 mmol) in methanol (8 mL) was added portion-wise at 0° C. sodium borohydride (2.2 mmol, 4 eq.). The reaction mixture was then stirred at room temperature for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give the final product.

### Example 7.1: Synthesis of Compound (22)

### Step 2: Synthesis of 2-Chloro-7-(4-[(3-dimethylaminopropyl)iminomethyl]phenyl)-4-phenyl-7H-pyrrolo[2,3-d]pyrimidine

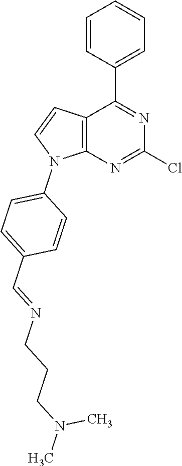 [SMILES: CN(C)CCC/N=C/c1ccc(-n2ccc3c(-c4ccccc4)nc(Cl)nc32)cc1]

Pale-yellow crystals (93%), Mp=97° C.; ¹H NMR δ (300 MHz, CDCl₃): 8.37 (s, 1H, CH═N), 8.16-8.12 (m, 2H, H-2′ and H-6′), 7.93 (d, 2H, J=8.50 Hz, H-2″ and H-6″), 7.83 (d, 2H, J=8.50 Hz, H-3″ and H-5″), 7.61-7.56 (m, 4H, H-3′, H-4′, H-5′ and H-6), 7.02 (d, 1H, J=3.60 Hz, H-5), 3.71 (t, 2H, J=7.20 Hz, NCH₂), 2.39 (t, 2H, J=7.20 Hz, NCH₂), 2.28 (s, 6H, N(CH₃)₂), 1.92 (qt, 2H, J=7.20 Hz, CH₂).

STEP 3 leads to compound (22A) as detailed in Table 3.

The corresponding oxalate salt, compound (22B), has also been obtained as detailed in Table 3.

### Example 7.2: Synthesis of Compound (23)

### Step 2: Synthesis of 2-Chloro-7-(4-[(3-(4-methylpiperazin-1-yl)propyl)iminomethyl]phenyl)-4-phenyl-7H-pyrrolo[2,3-d]pyrimidine

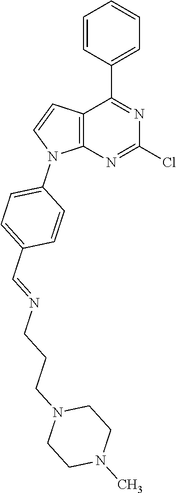 [SMILES: CN1CCN(CCC/N=C/c2ccc(-n3ccc4c(-c5ccccc5)nc(Cl)nc43)cc2)CC1]

Yellow oil (95%); ¹H NMR δ (300 MHz, CDCl₃): 8.34 (s, 1H, CH═N), 8.13-8.10 (m, 2H, H-2′ and H-6′), 7.89 (d, 2H, J=8.40 Hz, H-2″ and H-6″), 7.81 (d, 2H, J=8.40 Hz, H-3″ and H-5″), 7.60-7.55 (m, 4H, H-3′, H-4′, H-5′ and H-6), 6.99 (d, 1H, J=3.60 Hz, H-5), 3.69 (t, 2H, J=6.90 Hz, NCH₂), 2.61-2.42 (m, 10H, 5 NCH₂), 2.30 (s, 3H, NCH₃), 1.93 (qt, 2H, J=6.90 Hz, CH₂).

STEP 3: leads to compound (23A) as detailed in Table 3.

The corresponding oxalate salt, compound (23B), has also been obtained as detailed in Table 3.

### Example 8—Preparation of Compounds (32) and (35)

Compounds (32) and (35) are prepared following general protocol 8 (GP8) as defined here-above.

### Step 1A: Synthesis of 2-Chloro-7-(4-formylphenyl)-4-phenyl-7H-pyrrolo[2,3-d]pyrimidine

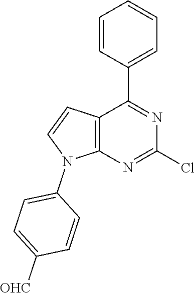 [SMILES: O=Cc1ccc(-n2ccc3c(-c4ccccc4)nc(Cl)nc32)cc1]

2-Chloro-7-(4-formylphenyl)-4-phenyl-7H-pyrrolo[2,3-d]pyrimidine is synthesized as detailed in STEP 1A of Example 7 here-above.

### Step 1B: Synthesis of 2,7-Bis(4-formylphenyl)-4-phenyl-7H-pyrrolo[2,3-d]pyrimidine

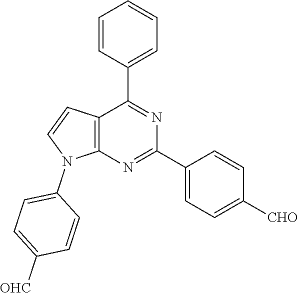 [SMILES: O=Cc1ccc(-c2nc(-c3ccccc3)c3ccn(-c4ccc(C=O)cc4)c3n2)cc1]

To a solution of 8.0 mmol of 2-chloro-4-phenyl-7-(4-formylphenyl)-7H-pyrrolo[2,3-d]pyrimidine, 143 mg of 4-formylphenyl boronic acid (0.96 mmol, 1.2 eq.) and 46 mg (0.04 mmol, 0.05 eq.) of tetrakis(triphenylphosphine) palladium in 10 mL of 1,2-dimethoxyethane, 1.5 mL of 2M Na₂CO₃ aqueous solution, previously degassed for 10 minutes with nitrogen, were added at room temperature. Then, the mixture was warmed to reflux and stirred for 24 hours under nitrogen positive pressure. The reaction mixture was cooled down to room temperature and the solvent was evaporated under vacuum. The organic layer was extracted with CH₂Cl₂ and the organic phase was filtered on filter paper, then washed with water (12 mL×3 times), dried over anhydrous sodium sulfate and activated charcoal, filtered and evaporated under vacuum. The residue was cooled and triturated with a minimum of EtOH and EtO₂ and filtered on sintered glassware to give the crude product. The residue was purified by silica gel column chromatography (CH₂Cl₂) to give the solid product.

White crystals (63%); Mp >260° C.; ¹H NMR δ (300 MHz, CDCl₃): 10.14 (s, 1H, CHO), 10.13 (s, 1H, CHO), 8.83 (d, 2H, J=8.40 Hz, H-2′ and H-6′), 8.29 (dd, 2H, J=7.80 and 1.65 Hz, H-2″ and H-6″), 8.21 (d, 2H, J=8.85 Hz, H-2′″ and H-6′″), 8.16 (d, 2H, J=8.85 Hz, H-3′″ and H-5′″), 8.04 (d, 2H, J=8.40 Hz, H-3′ and H-5′), 7.74 (d, 1H, J=3.90 Hz, H-6), 7.66-7.61 (m, 3H, H-3″, H-4″ and H-5″), 7.13 (d, 1H, J=3.90 Hz, H-5).

### Step 2: Synthesis of 2,7-bis[(substituted-iminomethyl)phenyl]-4-phenyl-7H-pyrrolo[2,3-d]pyrimidines

To a solution of the appropriate diamines (0.126 mmol, 2.1 eq. or 2.2 eq.) in ethanol (7 mL) was added the 2,7-bis(4-formylphenyl)-4-phenyl-7H-pyrrolo[2,3-d]pyrimidine (0.6 mmol). The reaction mixture was then heated under reflux for 5 h, and then evaporated to dryness under reduced pressure. After cooling, the residue was extracted with dichloromethane (40 mL). The organic layer was dried over sodium sulfate and activated charcoal and evaporated to dryness. Products were then used without further purification.

### Step 3: Synthesis of 2,7-bis[(substituted-aminomethyl)phenyl]-4-phenyl-7H-pyrrolo[2,3-d]pyrimidines

To a solution of 2,7-bis[(substituted-iminomethyl)phenyl]-4-phenyl-7H-pyrrolo[2,3-d]pyrimidines (0.4 mmol) in methanol (10 mL) was added portion-wise at 0° C. sodium borohydride (3.2 mmol, 8 eq.). The reaction mixture was then stirred at room temperature for 2 hours. Then it was evaporated to dryness under reduced pressure. After cooling, the residue was triturated in water and extracted with dichloromethane (40 mL). The organic layer was separated, dried over sodium sulfate and activated charcoal and evaporated to dryness.

### Example 8.1: Synthesis of Compound (32)

### Step 2: Synthesis of 2,7-bis{4-[(3-dimethylaminobutyl)iminomethyl]phenyl}-4-phenyl-7H-pyrrolo[2,3-d]pyrimidine

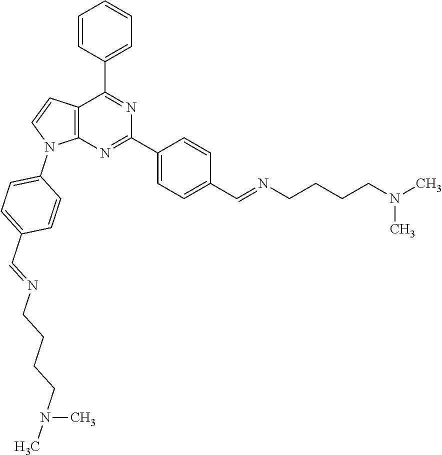 [SMILES: CN(C)CCCC/N=C/c1ccc(-c2nc(-c3ccccc3)c3ccn(-c4ccc(/C=N/CCCCN(C)C)cc4)c3n2)cc1]

Yellow oil (98%); ¹H NMR δ (300 MHz, CDCl₃): 8.66 (d, 2H, J=8.40 Hz, H-2′ and H-6′), 8.34 (s, 1H, CH═N), 8.33 (s, 1H, CH═N), 8.23 (dd, 2H, J=7.80 and 1.70 Hz, H-2″ and H-6″), 7.94 (d, 2H, J=8.70 Hz, H-2′″ and H-6′″), 7.88 (d, 2H, J=8.70 Hz, H-3′″ and H-5′″), 7.82 (d, 2H, J=8.40 Hz, H-3′ and H-5′), 7.59-7.50 (m, 4H, H-6, H-3″, H-4″ and H-5″), 6.93 (d, 1H, J=3.60 Hz, H-5), 3.67 (t, 4H, J=6.90 Hz, 2NCH₂), 2.36-2.30 (m, 4H, 2NCH₂), 2.24 (s, 6H, N(CH₃)₂), 2.23 (s, 6H, N(CH₃)₂), 1.79-1.72 (m, 4H, 2CH₂), 1.61-1.55 (m, 4H, 2CH₂).

Step 3 leads to compound (32A) as detailed in Table 3.

The corresponding oxalate salt, compound (32B) has also been obtained as detailed in Table 3.

### Example 8.2: Synthesis of Compound (35)

### Step 2: Synthesis of 2,7-bis{4-[(3-dimethylaminopentyl)iminomethyl]phenyl}-4-phenyl-7H-pyrrolo[2,3-d]pyrimidine

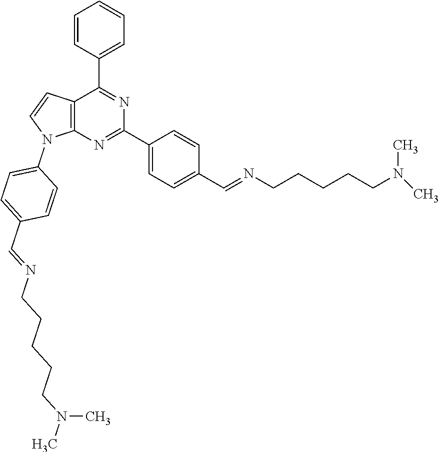 [SMILES: CN(C)CCCCC/N=C/c1ccc(-c2nc(-c3ccccc3)c3ccn(-c4ccc(/C=N/CCCCCN(C)C)cc4)c3n2)cc1]

Yellow oil (95%); ¹H NMR δ (300 MHz, CDCl₃): 8.68 (d, 2H, J=8.40 Hz, H-2′ and H-6′), 8.36 (s, 1H, CH═N), 8.35 (s, 1H, CH═N), 8.26 (dd, 2H, J=7.80 and 1.70 Hz, H-2″ and H-6″), 7.99 (d, 2H, J=8.80 Hz, H-2′″ and H-6′″), 7.94 (d, 2H, J=8.80 Hz, H-3′″ and H-5″), 7.84 (d, 2H, J=8.40 Hz, H-3′ and H-5′), 7.64-7.55 (m, 4H, H-6, H-3″, H-4″ and H-5″), 7.00 (d, 1H, J=3.60 Hz, H-5), 3.68 (t, 2H, J=6.90 Hz, NCH₂), 3.66 (t, 2H, J=6.90 Hz, NCH₂), 2.33-2.26 (m, 4H, 2NCH₂), 2.24 (s, 6H, N(CH₃)₂), 2.23 (s, 6H, N(CH₃)₂), 1.82-1.75 (m, 4H, 2CH₂), 1.60-1.49 (m, 4H, 2CH₂), 1.47-1.38 (m, 4H, 2CH₂).

STEP 3 leads to compound (35A) as detailed in Table 3.

The corresponding oxalate salt, compound (35B) has also been obtained as detailed in Table 3.

### Example 9—Preparation of Compounds (43) and (47)

Compounds (43) and (47) are prepared following general protocol 4 (GP4) as defined here-above.

### Example 9.1: Preparation of Compound (43)

### Step 1A: Preparation of 2-Chloro-4-(4-formylphenyl)-7-phenyl-7H-imidazo[4,5-d]pyrimidine

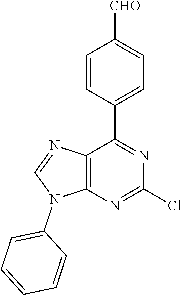 [SMILES: O=Cc1ccc(-c2nc(Cl)nc3c2ncn3-c2ccccc2)cc1]

To a solution of (SR7) (1.0 mmol), 4-formylphenylboronic acid (1.1 mmol, 1.1 eq.) and tetrakis(triphenylphosphine)palladium (0.05 mmol, 0.05 equiv.) in 15 mL of 1,2-dimethoxyethane, 2.5 mL of 2M Na₂CO₃ aqueous solution, previously degassed for 10 minutes with nitrogen, were added at room temperature. Then, the mixture was warmed to reflux and stirred for 24 hours under nitrogen pressure. The reaction mixture was cooled down to room temperature and the solvent was evaporated under vacuum. The organic layer was extracted with CH₂Cl₂ and the organic phase was filtered on filter paper, then washed with water (10 mL×3 times), dried over anhydrous sodium sulfate and activated charcoal, filtered and evaporated under vacuum. The residue was then purified by silica gel column chromatography (CH₂Cl₂) to give the solid product.

White crystals (25%); Mp=185° C.; ¹H NMR δ (300 MHz, CDCl₃): 10.17 (s, 1H, CHO), 9.05 (d, 2H, J=8.40 Hz, H-2′ and H-6′), 8.45 (s, 1H, H-6), 8.11 (d, 2H, J=8.40 Hz, H-3′ and H-5′), 7.75 (d, 2H, J=7.80 Hz, H-2″ and H-6″), 7.66 (t, 2H, J=7.80 Hz, H-3″ and H-5″), 7.54 (t, 1H, J=7.80 Hz, H-4″).

### Step 2: Preparation of 2-Chloro-4-{4-[(3-dimethylaminopropyl)iminomethyl]phenyl}-7-phenyl-7H-imidazo[4,5-d]pyrimidine

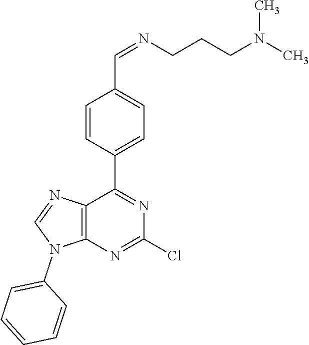 [SMILES: CN(C)CCC/N=C\c1ccc(-c2nc(Cl)nc3c2ncn3-c2ccccc2)cc1]

To a solution of the appropriate diamine (0.24 mmol, 1.1 eq.) in ethanol (5 mL) was added the 2-chloro-4-(4-formylphenyl)-7-phenyl-7H-imidazo[4,5-d]pyrimidine (0.22 mmol). The reaction mixture was then heated under reflux for 5 h, and then evaporated to dryness under reduced pressure. After cooling, the residue was extracted with dichloromethane (20 mL). The organic layer was dried over sodium sulfate and activated charcoal and evaporated to dryness. Products were then used without further purification.

Pale-yellow crystals (85%); Mp=133° C.; ¹H NMR δ (300 MHz, CDCl₃): 8.91 (d, 2H, J=8.40 Hz, H-2′ and H-6′), 8.41 (s, 1H, CH═N), 8.40 (s, 1H, H-6), 7.93 (d, 2H, J=8.40 Hz, H-3′ and H-5′), 7.73 (d, 2H, J=7.60 Hz, H-2′ and H-6′), 7.63 (t, 2H, J=7.60 Hz, H-3″ and H-5″), 7.52 (t, 1H, J=7.60 Hz, H-4″), 3.73 (t, 2H, J=6.90 Hz, NCH₂), 2.46 (t, 2H, J=6.90 Hz, NCH₂), 2.32 (t, 2H, J=6.90 Hz, NCH₂), 2.32 (s, 6H, N(CH₃)₂), 1.96 (m, 4H, 2CH₂), 1.66-1.56 (qt, 2H, J=6.90 Hz, CH₂).

### Step 3: Preparation of 2-Chloro-4-{4-[(3-dimethylaminopropyl)aminomethyl]phenyl}-7-phenyl-7H-imidazo[4,5-d]pyrimidine

To a solution of 2-Chloro-4-{4-[(3-dimethylaminopropyl)iminomethyl]phenyl}-7-phenyl-7H-imidazo[4,5-d]pyrimidine (0.2 mmol) in methanol (4 mL) was added portion-wise at 0° C. sodium borohydride (0.6 mmol, 3 eq.). The reaction mixture was then stirred at 60° C. for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give compound (43A) as detailed in Table 3.

The corresponding oxalate salt, compound (43B) has also been obtained as detailed in Table 3.

### Example 9.2: Preparation of Compound (47)

### Step 1A: Preparation of 6-Chloro-4-(4-formylphenyl)-1-phenyl-1H-pyrrolo[2,3-b]pyridine

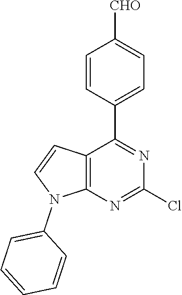 [SMILES: O=Cc1ccc(-c2nc(Cl)nc3c2ccn3-c2ccccc2)cc1]

To a solution of (SR10) (0.6 mmol), 4-formylphenylboronic acid (0.72 mmol, 1.2 eq.) and tetrakis(triphenylphosphine)palladium (0.03 mmol, 0.05 equiv.) in 8 mL of 1,2-dimethoxyethane, 1.2 mL of 2M Na₂CO₃ aqueous solution, previously degassed for 10 minutes with nitrogen, were added at room temperature. Then, the mixture was warmed to reflux and stirred for 24 hours under nitrogen pressure. The reaction mixture was cooled down to room temperature and the solvent was evaporated under vacuum. The organic layer was extracted with CH₂Cl₂ and the organic phase was filtered on filter paper, then washed with water (10 mL×3 times), dried over anhydrous sodium sulfate and activated charcoal, filtered and evaporated under vacuum. The residue was cooled and triturated with a minimum of EtOH, filtered on sintered glassware, and then washed with petroleum ether and dried under pressure to give the crude product.

Pale-yellow crystals (52%); Mp=140° C.; ¹H NMR δ (300 MHz, CDCl₃): 10.09 (s, 1H, CHO), 8.25 (d, 2H, J=8.40 Hz, H-2′ and H-6′), 7.98 (d, 2H, J=8.40 Hz, H-3′ and H-5′), 7.86 (d, 2H, J=7.50 Hz, H-2″ and H-6″), 7.76 (s, 1H, H-5), 7.65 (d, 1H, J=3.90 Hz, H-2), 7.61 (t, 2H, J=7.50 Hz, H-3″ and H-5″), 7.43 (t, 1H, J=7.50 Hz, H-4″), 6.80 (d, 1H, J=3.90 Hz, H-3).

### Step 2: Preparation of 6-Chloro-4-{4-[(3-dimethylaminopropyl)iminomethyl]phenyl}-1-phenyl-1H-pyrrolo[2,3-b]pyridine

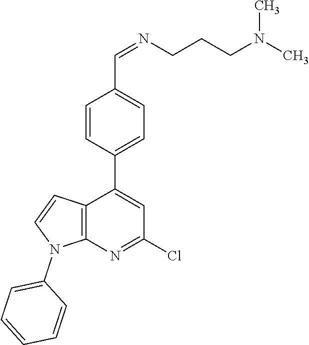 [SMILES: CN(C)CCC/N=C\c1ccc(-c2cc(Cl)nc3c2ccn3-c2ccccc2)cc1]

To a solution of the appropriate diamine (0.66 mmol, 1.1 eq.) in ethanol (7 mL) was added the 6-chloro-4-(4-formylphenyl)-1-phenyl-1H-pyrrolo[2,3-b]pyridine (0.6 mmol). The reaction mixture was then heated under reflux for 5 h, and then evaporated to dryness under reduced pressure. After cooling, the residue was extracted with dichloromethane (20 mL). The organic layer was dried over sodium sulfate and activated charcoal and evaporated to dryness. Products were then used without further purification.

Yellow oil (98%); ¹H NMR δ (300 MHz, CDCl₃): 8.35 (s, 1H, CH═N), 8.13 (d, 2H, J=8.40 Hz, H-2′ and H-6′), 7.87 (d, 2H, J=7.80 Hz, H-2″ and H-6″), 7.82 (d, 2H, J=8.40 Hz, H-3′ and H-5′), 7.72 (s, 1H, H-5), 7.61 (d, 1H, J=3.60 Hz, H-2), 7.57 (t, 2H, J=7.80 Hz, H-3″ and H-5″), 7.39 (t, 1H, J=7.80 Hz, H-4″), 6.77 (d, 1H, J=3.60 Hz, H-3), 3.70 (t, 2H, J=6.90 Hz, NCH₂), 2.46 (t, 2H, J=6.90 Hz, NCH₂), 2.32 (s, 6H, N(CH₃)₂), 1.95 (qt, 2H, J=6.90 Hz, CH₂).

### Step 3: Preparation of 6-Chloro-4-{4-[(3-dimethylaminopropyl)aminomethyl]phenyl}-1-phenyl-1H-pyrrolo[2,3-b]pyridine

To a solution of 6-Chloro-4-{4-[(3-dimethylaminopropyl)iminomethyl]phenyl}-1-phenyl-1H-pyrrolo[2,3-b]pyridine (0.2 mmol) in methanol (4 mL) was added portion-wise at 0° C. sodium borohydride (0.6 mmol, 3 eq.). The reaction mixture was then stirred at 60° C. for 2 hours. Then it was evaporated to dryness under reduced pressure. After cooling, the residue was triturated in water and extracted with dichloromethane (15 mL). The organic layer was separated, dried over sodium sulfate and activated charcoal and evaporated to dryness to give compound (47A) as detailed in Table 3.

The corresponding oxalate salt, compound (47B) has also been obtained as detailed in Table 3.

### Example 10: Biological Activity

### Material and Methods

In vitro Antiplasmodial Activity

The in vitro antiplasmodial activities were tested over concentrations ranging from 39 to 40 μm against culture-adapted Plasmodium falciparum: reference strains 3D7 and W2. The former strain is susceptible to chloroquine (CQ) but displays a decreased susceptibility to mefloquine (MQ); the latter is considered resistant to CQ. The parasites were cultivated in RPMI medium (Sigma-Aldrich, Lyon, France) supplemented with 0.5% Albumax I (Life Technologies Corporation, Paisley, UK), hypoxanthine (Sigma-Aldrich), and gentamicin (Sigma-Aldrich) with human erythrocytes and were incubated at 37° C. in a candle jar, as described in Desjardins R E et al., Antimicrob Agents Chemother, 1979; 16:710-18.

The P. falciparum drug susceptibility test was carried out in 96-well flat bottom sterile plates in a final volume of 250 μL. After a 48-hour incubation with the drugs, quantities of DNA in treated and control cultures of parasites in human erythrocytes were quantified using the SYBR Green I (Sigma-Aldrich) fluorescence-based method as described in Bennett T N et al, Antimicrob Agents Chemother, 2004; 48:1807-10. Briefly, after incubation, plates were frozen at −20° C. until use. Plates were then thawed for 2 hours at room temperature, and 100 μL of each homogenized culture was transferred to a well of a 96-well flat bottom sterile black plate (Nunc, Inc., Rochester, NY) that contained 100 μL of the SYBR Green I lysis buffer (2×SYBR Green, 20 mM Tris base pH 7.5, 5 mM EDTA, 0.008% w/v saponin, 0.08% w/v Triton X-100). Negative controls treated with solvent (typically DMSO or H₂O), and positive controls (CQ and MQ) were added to each set of experiments. Plates were incubated for 1 hour at room temperature and then read on a fluorescence plate reader (Tecan, Grödig, Austria) using excitation and emission wavelengths of 485 and 535 nm, respectively. Concentrations inhibiting 50% of the parasite's growth (half maximal inhibitory concentration or IC₅₀ values) were then calculated from the obtained experimental results by non-linear regression analysis of data from dose-response curves, using TableCurve 2D version 5.0 software (Systat Software, San Jose, CA). IC₅₀ values are reported as means calculated from three independent experiments, as described in H. Kaddouri et al., Antimicrob. Agents Chemother, 2006, 50, 3343.

### In Vitro Antitrypanosomal Activity

The effects of the tested compounds on the growth of T. brucei brucei were assessed using an Alamar Blue® assay described in B. Raz et al., Acta Trop. 1997, 68, 139. T. brucei brucei AnTat 1.9 (IMTA, Antwerpen, Belgium) was cultured in MEM with Earle's salts, supplemented according to the protocol disclosed in T. Baltz et al., EMBO J. 1985, 4, 1273, with the following modifications: 0.5 mM mercaptoethanol (Sigma-Aldrich), 1.5 mM 1-cysteine (Sigma-Aldrich), 0.05 mM bathocuproine sulfate (Sigma-Aldrich), and 20% heat-inactivated horse serum (Gibco, France) at 37° C. and 5% CO₂. Samples were incubated at an average density of 2000 parasites/well in sterile 96-well plates (Fisher, France) with various concentrations of compounds dissolved in 0.9% NaCl. All doses were tested in duplicate. Appropriate controls treated with solvents 0.9% NaCl or DMSO or with suramin, pentamidine, eflornithine, and fexinidazole (reference drugs purchased from Sigma-Aldrich and Fluorochem, UK) were added to each set of experiments. After a 69-hour incubation at 37° C., 10 μl of the viability marker Alamar Blue (Fisher) was added to each well, and the plates were incubated for 5 hour. The plates were read in a PerkinElmer ENSPIRE (Germany) microplate reader using an excitation wavelength of 530 nm and an emission wavelength of 590 nm. The IC₅₀ was defined as the concentration of drug necessary to inhibit by 50% the activity of T. brucei brucei compared to the control. IC₅₀ values were calculated using a non-linear regression analysis of dose-response curves performed using GraphPad Prism software (USA). IC₅₀ values were calculated from three independent experiments.

### Cytotoxicity Evaluation

The aim of this assay was to evaluate the impact of metabolic activation of the tested compounds on cell viability.

A cytotoxicity evaluation was performed using the method reported in Emami S A et al., Iran J Basic Med Sci 2012; 15:807-11 to determine the cytotoxic concentrations 50% (CC₅₀) and using doxorubicin as a cytotoxic reference compound. These assays were performed in human HepG2 cells. HepG2 cells were purchased from ATCC (ref HB-8065). These cells are a commonly used human hepatocarcinoma-derived cell line that has characteristics similar to those of primary hepatocytes. These cells express many hepatocyte-specific metabolic enzymes, thus enabling the cytotoxicity of tested product metabolites to be evaluated. Briefly, cells in 100 μl of complete RPMI medium [RPMI supplemented with 10% FCS, 1% 1-glutamine (200 mM), penicillin (100 U/ml), and streptomycin (100 μg/ml)] were inoculated into each well of 96-well plates and incubated at 37° C. in a humidified chamber in 6% CO₂. After 24 hours, 100 μl of medium with test compound at various concentrations dissolved in DMSO (final concentration less than 0.5% v/v) was added, and the plates were incubated for 72 hours at 37° C. Duplicate assays were performed for each sample. Each well was microscopically examined for precipitate formation before the medium was aspirated from the wells. After aspiration, 100 μl of MTT solution (0.5 mg/ml in medium without FCS) were then added to each well. Cells were incubated for 2 hours at 37° C. MTT solution was removed, and DMSO (100 μl) was added to dissolve the resulting blue formazan crystals. Plates were shaken vigorously (300 rpm) for 5 min. The absorbance was measured at 570 nm with 630 nm as reference wavelength in a BIO-TEK ELx808 Absorbance Microplate Reader. DMSO was used as blank and doxorubicin (Sigma-Aldrich) as positive control. Cell viability was calculated as percentage of control (cells incubated without compound). The CC₅₀ was determined from the dose-response curve using the TableCurve 2D version 5.0 software (Systat Software, San Jose, CA).

### FRET-Melting Experiments

Bioactive compounds have been selected for the subsequent FRET melting experiments. These were performed with dual-labeled oligonucleotides mimicking the Plasmodium telomeric sequences FPf1T [FAM-^{5′}(GGGTTTA)₃-GGG^{3′}-TAMRA](SEQ ID NO: 1) and FPf8T [FAM-^{5′}(GGGTTCA)₃GGG^{3′}-TAMRA](SEQ ID NO: 2), the Trypanosoma 9 and 11 chromosomic sequence FTrypBT (also named FEBR1T) [FAM-^{5′}GGGCAGGGGGTGATGGGGAGGAGCCAGGG^{3′}-TAMRA](SEQ ID NO: 3), the human telomeric sequence F21T [FAM-(GGGTTA)₃-GGG^{3′}-TAMRA](SEQ ID NO: 4) and the human duplex sequence FdxT [FAM^{5′}-TATAGCTATA-hexaethyleneglycol-TATAGCTATA^{3′}-TAMRA](SEQ ID NO: 5) (see De Cian et al, ChemBioChem, 2008, vol 9, pages 2730-9, and De Rache et al, Biochimie 2015, vol 115, pages 194-202). The oligonucleotides were pre-folded in 10 mM lithium cacodylate buffer (pH 7.2), with 10 mM KCl and 90 mM LiCl (K⁺ condition). The FAM emissions were recorded at 516 nm using a 492-nm excitation wavelength in the absence and presence of a single compound as a function of temperature (25 to 95° C.) in 96-well microplates by using a Stratagene MX3000P real-time PCR device at a rate of 1° C.·min−1. Data were normalized between 0 and 1, and the required temperature for half-denaturation of oligonucleotides corresponding to the emission value of 0.5 was taken as the Tm. Each experiment was performed in duplicate with 0.2 μM of labeled oligonucleotide and 2 μM of compound under K⁺ condition. For each compound, three independent experiments were carried out.

G4 ligands, typically tested at 1, 2 or 5 μM, lead to an increase in Tm, leading to ΔTm >0° C.

### RTqPCR SRAS-Cov-2

The anti-SARS-CoV-2 activity of the tested molecules was quantified using an infection assay developed by the Pasteur Institute. Shortly, A549 lung cancer cells stably expressing the human ACE2 receptor are infected by a SARS-CoV-2 strain. Cells are incubated with different concentrations of drug before and after viral inoculation. 72 hours after infection, viruses present in the supernatant of infected cells are quantified by quantitative RT-PCR of the N-gene. In parallel, cell viability is assessed using the CellTiter Glo kit (Promega). These two assays allow to evaluate the IC₅₀ values, which correspond to the concentrations of drug needed to inhibit 50% of viral replication or cytotoxic response.

TABLE 4

| | | | | | IC₅₀ | | | | | |
|---|---|---|---|---|---|---|---|---|---|---|
| | | | IC₅₀ | IC₅₀ T. brucei | RTqPCR | FRET-melting values in K⁺ conditions at 2 μM | | | | |
| Results | IC₅₀ Plasmodium falciparum | | HepG2 | brucei | SARS-CoV-2 | ΔTₘ (° C.) | ΔTₘ (° C.) | ΔTₘ (° C.) | ΔTₘ (° C.) | ΔTₘ (° C.) |
| Cpd No | 3D7 (μM) | W2 (μM) | (μM) | (μM) | (μM) | FPf1T | FPf8T | FtryBT | F21T | FdxT |
| 1B | 2.22 ± 0.12 | 2.71 ± 0.14 | 1.645 | 0.55 ± 0.07 | 0.33 | 23.6 | 23.5 | 20.5 | 22.8 | 1.2 |
| 2B | | 1.01 ± 0.16 | 1.08 | 0.88 ± 0.07 | | 19.1 | 20.0 | 17.3 | 19.6 | 0.7 |
| 3B | | 1.94 ± 0.16 | 3.54 | 0.38 ± 0.02 | 0.34 | 25.1 | 25.1 | 21.9 | 23.7 | 1.5 |
| 4B | | 1.58 ± 0.10 | 1.35 | 0.69 ± 0.04 | 0.69 | 19.8 | 21.0 | 17.4 | 20.4 | 0.6 |
| 5B | 0.53 ± 0.14 | 1.25 ± 0.08 | | 0.72 ± 0.03 | 0.12 | 30.8 | 31.6 | 28.3 | 30.9 | 5.2 |
| 6B | 1.21 ± 0.18 | 0.75 ± 0.16 | 1.74 | 0.14 ± 0.02 | | 26.3 | 26.6 | 23.1 | 25.5 | 1.7 |
| 7B | 0.88 ± 0.24 | 2.39 ± 0.08 | 1.91 | 0.63 ± 0.07 | | 11.5 | 12.8 | 10.5 | 13.8 | −0.3 |
| 8B | 1.95 ± 0.69 | 4.46 ± 0.60 | 16.42 | 0.55 ± 0.05 | | 10.3 | 10.6 | 9.4 | 10.9 | −0.2 |
| 9B | 17.83 ± 3.49 | 0.13 ± 0.05 | 39.52 | 1.00 ± 0.10 | | 10.1 | 10.1 | 8.4 | 10.4 | 0.0 |
| 10B | 0.61 ± 0.24 | 4.36 ± 1.70 | | 7.05 ± 0.66 | | 10.1 | 11.0 | 8.9 | 11.8 | −1.0 |
| 11B | >40 | 0.07 ± 0.01 | | 5.04 ± 0.73 | | | | | | — |
| 12B | 0.38 ± 0.25 | 9.14 | | | | | | | | |
| 13B | 2.44 ± 1.27 | 10.63 ± 0.69 | 26.7 | 0.86 ± 0.07 | 0.17 | 33.1 | 34.6 | 30.7 | 29.1 | 5.4 |
| 14B | 1.48 ± 0.29 | 0.29 ± 0.10 | 13.46 | 0.89 ± 0.09 | | | | | | −0.7 |
| 15B | 10.32 ± 0.22 | 15.22 ± 2.08 | 23.73 | 0.51 ± 0.07 | | | | | 0.3 | 0.1 |
| 16B | 10.51 ± 0.23 | 25.92 ± 4.63 | 63.54 | 1.34 ± 0.25 | | | | | 0.8 | 0.1 |
| 17B | 1.01 ± 0.41 | 0.29 ± 0.04 | 2.87 | 0.02 ± 0.00 | | | | | | −0.2 |
| 18B | 0.68 ± 0.13 | 6.96 ± 0.65 | | 0.06 | | | | | | |
| 19B | 1.97 ± 0.36 | 4.56 ± 0.56 | 1.88 | 0.05 ± 0.01 | | | | | 6.2 | −1.8 |
| 20B | 0.59 ± 0.17 | 13.59 ± 0.43 | | 0.08 | | | | | | |
| 22B | 0.80 ± 0.41 | 0.73 ± 0.22 | | 0.03 | | | | | | |
| 23B | 0.38 ± 0.25 | 9.14 | | | | | | | | |
| 24B | 1.63 ± 0.25 | 0.18 ± 0.04 | | 0.78 ± 0.06 | | 9.1 | 11.6 | 7.6 | 10.3 | −0.7 |
| 25B | 0.79 ± 0.12 | 2.23 ± 0.23 | | 2.43 ± 0.10 | | 10.7 | 11.1 | 8.0 | 12.3 | −0.9 |
| 26B | 4.46 ± 0.35 | 9.95 ± 1.47 | 2.79 | 0.46 ± 0.04 | | 11.2 | 13.2 | 8.6 | 14.1 | 0.3 |
| 27B | 1.95 ± 0.68 | 4.18 ± 0.03 | 11.15 | 0.89 ± 0.10 | | 9.6 | 9.0 | 7.9 | 9.72 | −0.10 |
| 28B | 0.03 ± 0.00 | 11.47 ± 2.47 | 35.2 | 1.10 ± 0.26 | | | | | 2.41 | −0.77 |
| 29B | 1.37 ± 0.24 | 2.17 ± 0.41 | 3.19 | 0.14 ± 0.02 | | 20.1 | 20.9 | 17.1 | 19.0 | 1.8 |
| 30B | 10.79 ± 2.95 | 0.03 ± 0.00 | 18.88 | 0.75 ± 0.10 | | 14.9 | 15.2 | 12.8 | 14.5 | 0.5 |
| 31B | 0.04 ± 0.01 | 5.69 ± 1.21 | >60 | 2.58 ± 0.21 | | 1.8 | 2.9 | 1.8 | 2.0 | −0.7 |
| 32B | 2.44 ± 0.56 | 2.12 ± 0.14 | | | 0.92 | | | | | |
| 33B | 2.47 ± 0.19 | 0.70 ± 0.15 | | | 0.55 | | | | | |
| 34B | 0.93 ± 0.15 | 0.77 ± 0.07 | | | 0.17 | | | | | |
| 35B | 6.76 ± 0.47 | 4.74 ± 0.90 | | | 0.56 | 25.4 | 33.76 | 21.55 | 27.23 | 3.49 |
| 36B | 2.51 ± 0.25 | 0.93 ± 0.15 | | | | | | | | |
| 37B | | 1.18 ± 0.12 | | | | | | | | |
| 38B | 0.35 ± 0.09 | 4.73 ± 0.28 | | | | | | | | |
| 39B | 1.83 ± 0.47 | 1.53 ± 0.09 | | | | | | | | |
| 40B | 4.74 ± 0.90 | 1.77 ± 0.09 | | | | 35.1 | 33.9 | 30.3 | 31.68 | 8.51 |
| 41B | 1.99 ± 0.90 | 1.99 ± 0.12 | | | | 36.4 | 34.9 | 33.0 | 34.0 | 9.7 |
| 42B | 0.70 ± 0.15 | 2.37 ± 0.13 | | | | 33.2 | 32.1 | 30. | 30.2 | 6.8 |
| 43B | 1.53 ± 0.39 | 0.83 ± 0.17 | | | | 2.4 | 3.3 | 2.7 | 1.7 | 0.04 |
| 47B | 0.15 ± 0.02 | 0.72 ± 0.30 | | | | 3.3 | 4.0 | 4.3 | 2.8 | −0.2 |

### Results

As shown in Table 4, most compounds of the present invention present an IC₅₀ activity against both W2 and 3D7 P. falciparum strains of less than 20 μM, in particular less than 17 μM.

Furthermore, most compounds of the present invention exhibit IC₅₀ activity against T. brucei brucei of less than 5 μM, in particular less than 3 μM.

Compounds are considered positive for G4 recognition if ΔTₘ >5° C. at 5 μM (or lower) ligand concentration.

Most of the tested compounds show G4 ΔTₘ of more than 5° C., in particular more than 10° C., some of them even exhibiting G4 ΔTₘ of more than 15° C.

Finally, some compounds of formula (I) present an IC₅₀ value against SARS-CoV-2 of less than 1 μM, even less than 0.7 μM.

### CONCLUSION

Based on the previous results, it can be concluded that the compounds of formula (I) are suitable chemical compounds in the prevention and/or treatment specifically of infectious diseases caused by P. falciparum or T. brucei parasites but also induced by SARS-CoV-2 virus, without cytotoxicity towards human cells.

More generally, compounds of formula (I) have proven to be particularly efficient for treating and/or preventing infectious diseases induced by parasites and/or viruses, of which replication cycle key processes involve DNA or RNA G-quadruplexes from the parasite and/or virus and/or host cell genome or transcriptome.

The present invention further relates to a pharmaceutical composition comprising at least one compound of formula (I) as defined above or any of its pharmaceutically acceptable salts or at least any of compounds (1) to (49), in particular (1) to (32), as defined above, and also at least one pharmaceutically acceptable excipient.

Pharmaceutical compositions of the invention can contain one or more compound(s) of the invention in any form described herein.

Still a further object of the present invention consists of the use of at least one compound of formula (I) as defined above or one of its pharmaceutically acceptable salts, or compounds (1) to (49), in particular (1) to (32), as defined above for preparing a drug for preventing and/or treating infectious diseases induced by parasites and/or viruses of which replication cycle key processes involve DNA or RNA G-quadruplexes from the parasite and/or virus and/or host cell genome or transcriptome, in particular induced by parasites and/or viruses selected among P. falciparum, L donovani or T. brucei, human immunodeficiency virus (HIV), herpes simplex virus (HSV), human papillomavirus (HPV), Epstein-Barr virus (EBV), Hepatitis C virus (HCV) and SARS-CoV-2.

Still a further object of the present invention consists of the use of at least one compound of formula (I) as defined above, or one of their pharmaceutically acceptable salts or any of compounds (1) to (49), in particular (1) to (32), as defined above for treating and/or preventing infectious diseases induced by parasites and/or viruses of which replication cycle key processes involve DNA or RNA G-quadruplexes from the parasite and/or virus and/or host cell genome or transcriptome, in particular induced by parasites and/or viruses selected among P. falciparum, L. donovani or T. brucei, human immunodeficiency virus (HIV), herpes simplex virus (HSV), human papillomavirus (HPV), Epstein-Barr virus (EBV), Hepatitis C virus (HCV) and SARS-CoV-2.

Another object of the invention relates to a therapeutic method for the treatment and/or for the prevention of infectious diseases induced by parasites and/or viruses of which replication cycle key processes involve DNA or RNA G-quadruplexes from the parasite and/or virus and/or host cell genome or transcriptome, in particular induced by parasites and/or viruses selected among P. falciparum, L. donovani or T. brucei, human immunodeficiency virus (HIV), herpes simplex virus (HSV), human papillomavirus (HPV), Epstein-Barr virus (EBV), Hepatitis C virus (HCV) and SARS-CoV-2, comprising at least a step of administering a therapeutically effective amount of a compound of formula (I) or of any of the compounds (1) to (49), in particular (1) to (32), as defined above, or one of their acceptable salts.

According to a particular embodiment, the treatment is continuous or non-continuous.

A “continuous treatment” means a long-term treatment which can be implemented with various administration frequencies, such as once every day, every three days, once a week, or once every two weeks or once every month.

According to one embodiment, the compound of formula (I), or any of its pharmaceutically acceptable salts, is administered at a dose varying from 0.1 to 1000 mg. in particular varying from 1 to 500 mg, or for example varying from 5 to 100 mg.

In a specific embodiment, the invention provides a use of a compound of formula (I) according to the invention or a pharmaceutically acceptable salt thereof or a method according to the invention wherein the compound of formula (I) is to be administered in combination with a co-agent useful in anyone of the hereabove mentioned diseases.

The inventive composition can further include one or more additives such as diluents, Excipients, stabilizers and preservatives. Such additives are well known to those skilled in the art and are described notably in “Ullmann's Encyclopedia of Industrial Chemistry, 6^{th} Ed.” (various editors, 1989-1998, Marcel Dekker) and in “Pharmaceutical Dosage Forms and Drug Delivery Systems” (ANSEL et al, 1994, WILLIAMS & WILKINS).

The aforementioned excipients are selected according to the dosage form and the desired mode of administration.

Compounds and compositions of this invention may be administered in any manner, including, but not limited to, orally, parenterally, sublingually, transdermally, vaginally, rectally, transmucosally, topically, intranasally via inhalation, via buccal or intranasal administration, or combinations thereof. Parenteral administration includes, but is not limited to, intravenous, intra-arterial, intra-peritoneal, subcutaneous, intramuscular, intra-thecal, and intra-articular. The compositions of this invention may also be administered in the form of an implant, which allows slow release of the compositions as well as a slow controlled i.v. infusion.

For example, a compound of formula (I) can be present in any pharmaceutical form which is suitable for enteral or parenteral administration, in association with appropriate excipients, for example in the form of plain or coated tablets, hard gelatin, soft shell capsules and other capsules, suppositories, or drinkable, such as suspensions, syrups, or injectable solutions or suspensions.

In a particular embodiment, a compound of formula (I) according to the invention is administered orally or intravenously.

1. A compound of formula (I), or a pharmaceutically acceptable salt thereof: 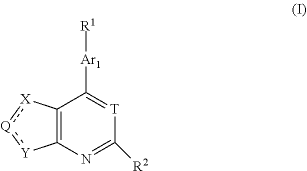
wherein
Ar₁ is a (C₅-C₁₁)arylene or (C₅-C₁₁)heteroarylene group,
R¹ is a hydrogen atom, a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group optionally substituted by a halogen atom or —(CH₂)ₙ—NH-Alk-NR³R⁴,
X is —CH—, —S—, —NR⁵— or —N—,
Y is —CH—, —NR⁵—, —S— or —NR⁶—CH₂—,
T is —CH— and Q is —CH— or T is —N— and Q is —CR¹⁰— or —N—,
each [symbol] is independently a single or double bond, and at most one of [symbol] is a double bond,
Alk is a (C₁-C₆)alkanediyl group, optionally substituted by one or more groups chosen from —COOR⁷ or —CONR³R⁴,
R⁵, if present, is a hydrogen atom, a (C₁-C₆)alkyl group optionally substituted by one or more (C₅-C₁₁)aryl or (C₅-C₁₁)heteroaryl group, a —(CH₂)ₙ—NH-Alk-NR³R⁴ or a (C₅-C₁₁)aryl or (C₅-C₁₁)heteroaryl group optionally substituted by one or more halogen atoms, (C₁-C₆)alkoxy or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl or (C₅-C₁₁)heteroaryl group optionally substituted by one or more halogen atoms, (C₁-C₆)alkoxy or —(CH₂)ₙ—NH-Alk-NR³R⁴,
each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more (C₁-C₅)alkyl groups,
n represents an integer chosen from 1, 2, 3, 4 and 5,
each R⁷, R⁸ and R⁹ independently represents a hydrogen atom or a (C₁-C₆)alkyl group,
R¹⁰ is a hydrogen atom or a (C₅-C₁₁)aryl group or (C₅-C₁₁)heteroaryl,
provided that at one or two of X, Q and Y comprises a heteroatom, and
with the proviso that, at least one of R¹, R², and, if present, R⁵ or R⁶, contains a group —NH-Alk-NR³R⁴.

2. A compound of formula (I) according to claim 1, or a pharmaceutically acceptable salt thereof, wherein:
Ar₁ is a (C₅-C₁₁)arylene, and R¹ is a hydrogen atom, a halogen atom, or a —(CH₂)ₙ—NH-Alk-NR³R⁴; or Ar₁ is a (C₅-C₁₁)heteroarylene group, and R¹ is a halogen atom or a —(CH₂)ₙ—NH-Alk-NR³R⁴, and
X is —CH—, —S—, —NR⁵—, or —N—,
Y is —CH—, —NR⁵—, —S— or —NR⁶—CH₂—, and
T is —N— and Q is —CH—,
or
Ar₁ is a (C₅-C₁₁)arylene or (C₅-C₁₁)heteroarylene group,
R¹ is a hydrogen atom, a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁)heteroaryl group optionally substituted by a halogen atom or —(CH₂)ₙ—NH-Alk-NR³R⁴,
X is —CH—, —S—, —NR⁵—, or —N—,
Y is —CH—, —NR⁵—, —S— or —NR⁶—CH₂—, and
T is —CH— and Q is —CH— or T is —N— and Q is —N—,
and [symbol]
, Alk, R⁵, R⁶, R³, R⁴ and n are as defined in claim 1,
provided that one or two of X, Q and Y comprises a heteroatom, and with the proviso that, at least one of R¹, R², and, if present, R⁵ or R⁶, contains a group —NH-Alk-NR³R⁴.

3. A compound of formula (I) as defined in claim 1, having formula (I′), or a pharmaceutically acceptable salt thereof: 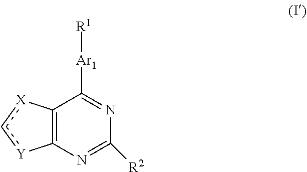
wherein:
Ar₁ is a (C₅-C₁₁)arylene, and R¹ is a hydrogen atom, a halogen atom, or a —(CH₂)ₙ—NH-Alk-NR³R⁴; or
Ar₁ is a (C₅-C₁₁)heteroarylene group, and R¹ is a halogen atom, or a —(CH₂)ₙ—NH-Alk-NR³R⁴,
R² is a hydrogen atom, a halogen atom, a —NH-Alk-NR³R⁴ or a (C₅-C₁₁)aryl or (C₅-C₁₁) heteroaryl group, optionally substituted by a halogen atom or —(CH₂)ₙ—NH-Alk-NR³R⁴,
X is —CH—, —S— or —NR⁵—,
Y is —CH—, —NR⁵—, —S— or —NR⁶—CH₂—,
each [symbol] is independently a simple or double bond, and at most one of [symbol] is a double bond,
Alk is a (C₁-C₆)alkanediyl group, optionally substituted by one or more groups chosen from —COOR⁷ or —CONR³R⁴,
R⁵, if present, is a hydrogen atom, a (C₁-C₆)alkyl group, a —(CH₂)ₙ—NH-Alk-NR³R⁴ or a (C₅-C₁₁)aryl or (C₅-C₁₁)heteroaryl group, optionally substituted by one or more halogen atoms or —(CH₂)ₙ—NH-Alk-NR³R⁴,
R⁶, if present, is a hydrogen atom, a (C₁-C₆)alkyl group, a group —COOR⁸, a —(CH₂)ₙ—NH-Alk-NR³R⁴ or a (C₅-C₁₁)aryl or (C₅-C₁₁)heteroaryl group, optionally substituted by one or more halogen atoms or —(CH₂)ₙ—NH-Alk-NR³R⁴,
each R³ and R⁴ independently represents a hydrogen atom, a (C₁-C₆)alkyl group, or a —COOR⁹ group, or alternatively R³ and R⁴ form together with the nitrogen atom bearing them a (C₃-C₈)heterocycloalkyl group, optionally substituted by one or more (C₁-C₅)alkyl groups,
n represents an integer chosen from 1, 2, 3, 4 and 5,
each R⁷, R⁸ and R⁹ independently represents a hydrogen atom or a (C₁-C₆)alkyl group,
provided that one of X and Y comprises a heteroatom, and
with the proviso that, at least one of R¹, R², and, if present, R⁵ or R⁶, contains a group —NH-Alk-NR³R⁴.

4. A compound of formula (I) according to claim 1, having formula (Ia-a), or a pharmaceutically acceptable salt thereof: 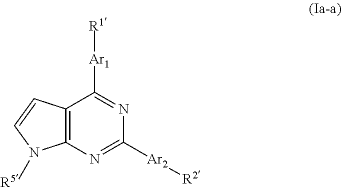
wherein:
each R^{1′} and R^{2′} is a —(CH₂)ₙ—NH-Alk-NR³R⁴,
R^{5″} is a (C₁-C₆)alkyl group or a phenyl group,
each Ar₁ and Ar₂ is independently a phenylene or a thiophenylene, and
each Alk, R³, R⁴ and n is as defined in claim 1.

5. A compound of formula (I) according to claim 1, having formula (Ia-b), or a pharmaceutically acceptable salt thereof: 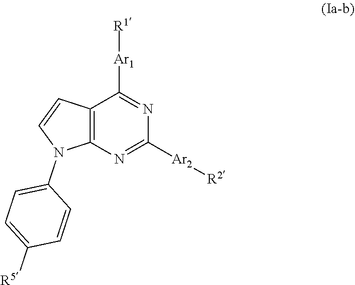
wherein:
Ar₁ is a phenylene and R^{1′} is a hydrogen atom or a —(CH₂)ₙ—NH-Alk-NR³R⁴, or
Ar₁ is a thiophenylene and R^{1′} is a —(CH₂)ₙ—NH-Alk-NR³R⁴,
R^{2′} is a hydrogen atom or a —(CH₂)ₙ—NH-Alk-NR³R⁴,
R^{5′} is a (C₁-C₆)alkoxy or —(CH₂)ₙ—NH-Alk-NR³R⁴,
Ar₂ is a phenylene or a thiophenylene, and
each Alk, R³, R⁴ and n is as defined in claim 1.

6. A compound of formula (I) according to claim 1, having formula (Ia-c), or a pharmaceutically acceptable salt thereof: 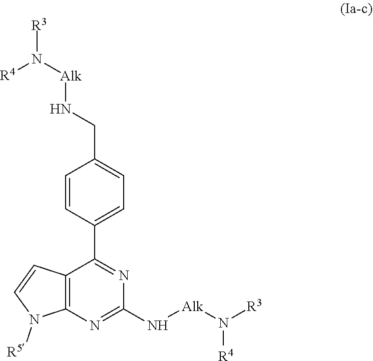
wherein:
R^{5″} is a hydrogen atom or a phenyl group, and
each Alk, R³, R⁴ and n is as defined in claim 1.

7. A compound of formula (I) claim 1, having formula (Ia-d), or a pharmaceutically acceptable salt thereof: 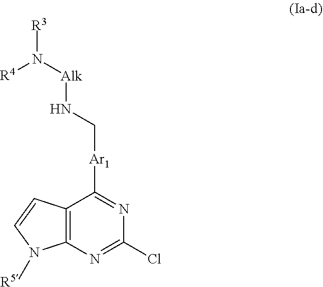
wherein Ar₁, Alk, R³, R⁴ are as defined in claim 1 and R^{5″} is a (C₁-C₆)alkyl group or a phenyl group.

8. A compound of formula (I) according to claim 1, having formula (Ia-e), or a pharmaceutically acceptable salt thereof: 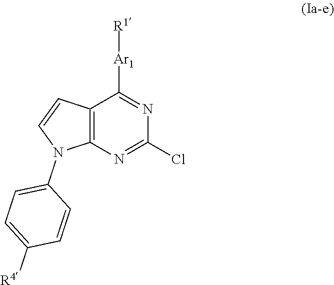
wherein Ar₁ is as defined in claim 1 and R^{1′} and R^{5′} are each a C₁-C₆)alkoxy or —(CH₂)ₙ—NH-Alk-NR³R⁴.

9. A compound of formula (I) according to claim 1, having formula (Ib-a), or a pharmaceutically acceptable salt thereof: 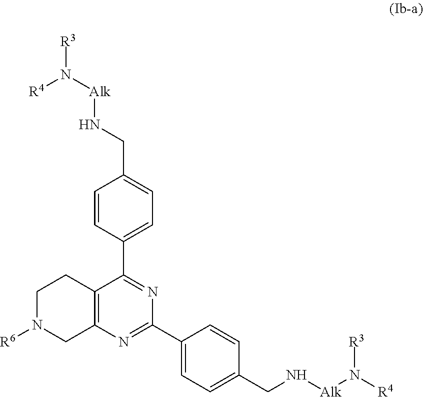
wherein R⁶, Alk, R³ and R⁴ are as defined in claim 1.

10. A compound of formula (I) according to claim 1 having formula (Ic-a) or a pharmaceutically acceptable salt thereof: 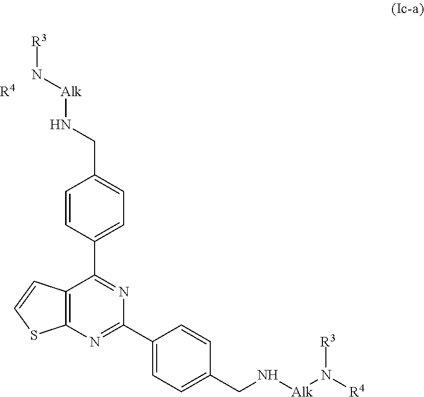
wherein Alk, R³ and R⁴ are as defined in claim 1.

11. A compound of formula (I) according to claim 1, having formula (Ic-b) or a pharmaceutically acceptable salt thereof: 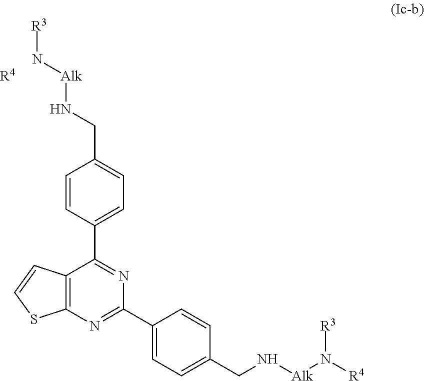
wherein each Alk, R³ and R⁴ is as defined in claim 1.

12. A compound of formula (I) according to claim 1, having formula (Id-a) or a pharmaceutically acceptable salt thereof: 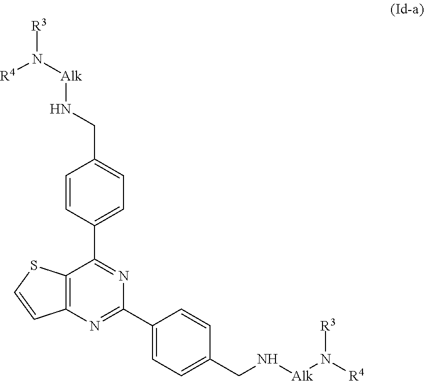
wherein Alk, R³ and R⁴ are as defined in claim 1.

13. Compound of formula (I) according to claim 1, having formula (Id-b) or a pharmaceutically acceptable salt thereof: 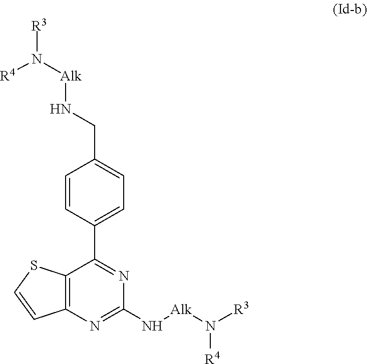
wherein Alk, R³ and R⁴ are as defined in claim 1.

14. Compound of formula (I) according to claim 1, having formula (Ie-a′) or a pharmaceutically acceptable salt thereof: 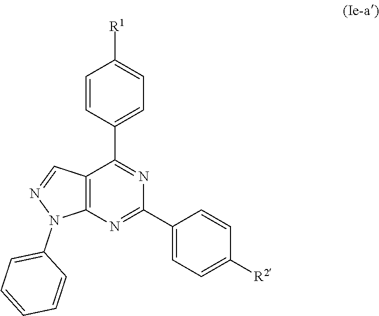
wherein R^{2′} is a hydrogen atom or a —(CH₂)ₙ—NH-Alk-NR³R⁴, and R¹, Alk, R³, R⁴ and n are as defined in claim 1, with the proviso that at least one of R¹ and R^{2′} contains a —NH-Alk-NR³R⁴.

15. Compound of formula (I) according to claim 1, having formula (Ie-b′) or a pharmaceutically acceptable salt thereof: 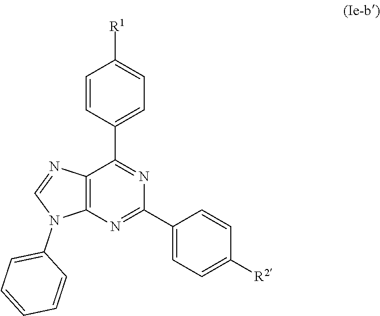
wherein R^{2′} is a hydrogen atom or a —(CH₂)ₙ—NH-Alk-NR³R⁴, and
R¹, Alk, R³, R⁴ and n are as defined in claim 1,
with the proviso that at least one of R¹ and R^{2′} contains a —NH-Alk-NR³R⁴.

16. Compound of formula (I) according to claim 1, having formula (Ie-b″) or a pharmaceutically acceptable salt thereof: 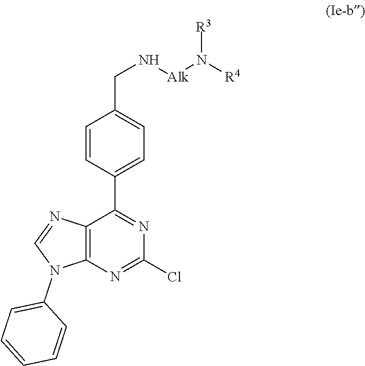
wherein Alk, R³, R⁴ and n are as defined in claim 1.

17. Compound of formula (I) according to claim 1, having formula (If-a′) or a pharmaceutically acceptable salt thereof: 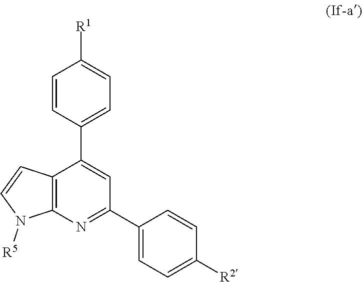
wherein R^{2′} is a hydrogen atom or a —(CH₂)ₙ—NH-Alk-NR³R⁴, and
R¹, Alk, R³, R⁴, R⁵ and n are as defined in claim 1,
with the proviso that at least one of R¹ and R^{2′} contains a —NH-Alk-NR³R⁴ and each R³ and R⁴ independently represent a (C₁-C₆)alkyl group.

18. Compound of formula (I) according to claim 1, having formula (If-a″) or a pharmaceutically acceptable salt thereof: 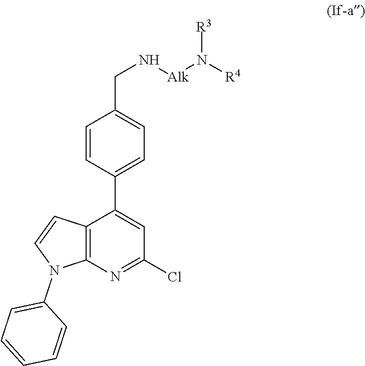
wherein Alk, R³, R⁴ and n are as defined in claim 1,
and
each R³ and R⁴ independently represent a (C₁-C₆)alkyl group.

19. Compound of formula (I) according to claim 1, having formula (Ig-a) or a pharmaceutically acceptable salt thereof: 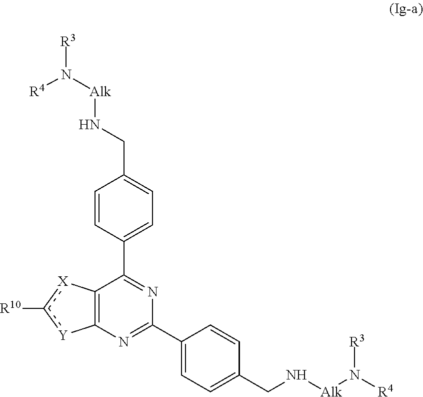
wherein Alk, R³ and R⁴ are as defined in claim 1, R¹⁰ is a (C₅-C₁₁)aryl group or (C₅-C₁₁)heteroaryl each R³ and R⁴ is a methyl and R¹⁰ is a phenyl.

20. A compound of formula (I) according to claim 1 selected from
(1A) 2,4-bis{4-[(3-dimethylaminopropyl)aminomethyl]phenyl}-7-phenyl-7H-pyrrolo[2,3-d]pyrimidine,
(1B) 2,4-bis{4-[(3-dimethylaminopropyl)aminomethyl]phenyl}-7-phenyl-7H-pyrrolo[2,3-d]pyrimidine oxalate,
(2A) 2,4-bis{4-[(3-(4-methylpiperazin-1-yl)propyl)aminomethyl]phenyl}-7-phenyl-7H-pyrrolo[2,3-d]pyrimidine,
(2B) 2,4-bis{4-[(3-(4-methylpiperazin-1-yl)propyl)aminomethyl]phenyl}-7-phenyl-7H-pyrrolo[2,3-d]pyrimidine oxalate,
(3A) 2,4-bis{4-[(3-dimethylaminobutyl)aminomethyl]phenyl}-7-phenyl-7H-pyrrolo[2,3-d]pyrimidine,
(3B) 2,4-bis{4-[(3-dimethylaminobutyl)aminomethyl]phenyl}-7-phenyl-7H-pyrrolo[2,3-d]pyrimidine oxalate,
(4A) 2,4-bis{4-[(3-(4-methylpiperazin-1-yl)butyl)aminomethyl]phenyl}-7-phenyl-7H-pyrrolo[2,3-d]pyrimidine,
(4B) 2,4-bis{4-[(3-(4-methylpiperazin-1-yl)butyl)aminomethyl]phenyl}-7-phenyl-7H-pyrrolo[2,3-d]pyrimidine oxalate,
(5A) 2,4-bis{4-[(3-dimethylaminopentyl)aminomethyl]phenyl}-7-phenyl-7H-pyrrolo[2,3-d]pyrimidine,
(5B) 2,4-bis{4-[(3-dimethylaminopentyl)aminomethyl]phenyl}-7-phenyl-7H-pyrrolo[2,3-d]pyrimidine oxalate,
(6A) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine,
(6B) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine oxalate,
(7A) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine,
(7B) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine oxalate,
(8A) 2,4-bis{4-[(3-dimethylaminopropyl)aminomethyl]phenyl}-7-methyl-7H-pyrrolo[2,3-d]pyrimidine,
(8B) 2,4-bis{4-[(3-dimethylaminopropyl)aminomethyl]phenyl}-7-methyl-7H-pyrrolo[2,3-d]pyrimidine oxalate,
(9A) 2,4-bis{4-[(3-(4-methylpiperazin-1-yl)propyl)aminomethyl]phenyl}-7-methyl-7H-pyrrolo[2,3-d]pyrimidine,
(9B) 2,4-bis{4-[(3-(4-methylpiperazin-1-yl)propyl)aminomethyl]phenyl}-7-methyl-7H-pyrrolo[2,3-d]pyrimidine oxalate,
(10A) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine,
(10B) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine oxalate,
(11A) 2,4-Bis{4-[(5-amino-5-(methoxycarbonyl)pentyl)aminomethyl]phenyl}-7-methyl-7H-pyrrolo[2,3-d]pyrimidine,
(11B) 2,4-Bis{4-[(5-amino-5-(methoxycarbonyl)pentyl)aminomethyl]phenyl}-7-methyl-7H-pyrrolo[2,3-d]pyrimidine oxalate,
(12A) 7-(4-[(3-Dimethylaminopropyl)aminomethyl]phenyl)-2,4-diphenyl-7H-pyrrolo[2,3-d]pyrimidine,
(12B) 7-(4-[(3-Dimethylaminopropyl)aminomethyl]phenyl)-2,4-diphenyl-7H-pyrrolo[2,3-d]pyrimidine oxalate,
(13A) 2,4,7-Tri{4-[(3-dimethylaminopropyl)aminomethyl]phenyl}-7H-pyrrolo[2,3-d]pyrimidine,
(13B) 2,4,7-Tri{4-[(3-dimethylaminopropyl)aminomethyl]phenyl}-7H-pyrrolo[2,3-d]pyrimidine oxalate,
(14A) 2-(3-Dimethylaminopropylamino)-4-{4-[(4-dimethylaminopropyl)aminomethyl]phenyl}-7-phenyl-7H-pyrrolo[2,3-d]pyrimidine,
(14B) 2-(3-Dimethylaminopropylamino)-4-{4-[(4-dimethylaminopropyl)aminomethyl]phenyl}-7-phenyl-7H-pyrrolo[2,3-d]pyrimidine oxalate,
(15A) 2-Chloro-4-{4-[(3-dimethylaminopropyl)aminomethyl]phenyl}-7H-pyrrolo[2,3-d]pyrimidine,
(15B) 2-Chloro-4-{4-[(3-dimethylaminopropyl)aminomethyl]phenyl}-7H-pyrrolo[2,3-d]pyrimidine oxalate,
(16A) 2-Chloro-4-{4-[(3-(4-methylpiperazin-1-yl)propyl)aminomethyl]phenyl}-7H-pyrrolo[2,3-d]pyrimidine,
(16B) 2-Chloro-4-{4-[(3-(4-methylpiperazin-1-yl)propyl)aminomethyl]phenyl}-7H-pyrrolo[2,3-d]pyrimidine oxalate,
(17A) 2-Chloro-4-{4-[(3-dimethylaminopropyl)aminomethyl]phenyl}-7-phenyl-7H-pyrrolo[2,3-d]pyrimidine,
(17B) 2-Chloro-4-{4-[(3-dimethylaminopropyl)aminomethyl]phenyl}-7-phenyl-7H-pyrrolo[2,3-d]pyrimidine oxalate,
(18A) 2-chloro-4-{4-[(3-(4-methylpiperazin-1-yl)propyl)aminomethyl]phenyl}-7-phenyl-7H-pyrrolo[2,3-d]pyrimidine,
(18B) 2-chloro-4-{4-[(3-(4-methylpiperazin-1-yl)propyl)aminomethyl]phenyl}-7-phenyl-7H-pyrrolo[2,3-d]pyrimidine oxalate,
(19A) 2-Chloro-4-{5-[(3-dimethylaminopropyl)aminomethyl]thien-2-yl}-7-phenyl-7H-pyrrolo[2,3-d]pyrimidine,
(19B) 2-Chloro-4-{5-[(3-dimethylaminopropyl)aminomethyl]thien-2-yl}-7-phenyl-7H-pyrrolo[2,3-d]pyrimidine oxalate,
(20A) 2-Chloro-4-{2-[(3-dimethylaminopropyl)aminomethyl]thien-4-yl}-7-phenyl-7H-pyrrolo[2,3-d]pyrimidine,
(20B) 2-Chloro-4-{2-[(3-dimethylaminopropyl)aminomethyl]thien-4-yl}-7-phenyl-7H-pyrrolo[2,3-d]pyrimidine oxalate,
(21A) 2-Chloro-4-{2-[(3-dimethylaminopropyl)aminomethyl]thien-3-yl}-7-phenyl-7H-pyrrolo[2,3-d]pyrimidine,
(21B) 2-Chloro-4-{2-[(3-dimethylaminopropyl)aminomethyl]thien-3-yl}-7-phenyl-7H-pyrrolo[2,3-d]pyrimidine oxalate,
(22A) 2-Chloro-7-(4-[(3-dimethylaminopropyl)aminomethyl]phenyl)-4-phenyl-7H-pyrrolo[2,3-d]pyrimidine,
(22B) 2-Chloro-7-(4-[(3-dimethylaminopropyl)aminomethyl]phenyl)-4-phenyl-7H-pyrrolo[2,3-d]pyrimidine oxalate,
(23A) 2-Chloro-7-(4-[(3-(4-methylpiperazin-1-yl)propyl)aminomethyl]phenyl)-4-phenyl-7H-pyrrolo[2,3-d]pyrimidine,
(23B) 2-Chloro-7-(4-[(3-(4-methylpiperazin-1-yl)propyl)aminomethyl]phenyl)-4-phenyl-7H-pyrrolo[2,3-d]pyrimidine oxalate,
(24A) Tert-butyl 2,4-bis{4-[(3-dimethylaminopropyl)aminomethyl]phenyl})-5,6-dihydropyrido[3,4-d]pyrimidine-7(8H)-carboxylate,
(24B) Tert-butyl 2,4-bis{4-[(3-dimethylaminopropyl)aminomethyl]phenyl})-5,6-dihydropyrido[3,4-d]pyrimidine-7(8H)-carboxylate oxalate,
(25A) 2,4-Bis{4-[(3-dimethylaminopropyl)aminomethyl]phenyl})-5,6,7,8-dihydropyrido[3,4-d]pyrimidine,
(25B) 2,4-Bis{4-[(3-dimethylaminopropyl)aminomethyl]phenyl})-5,6,7,8-dihydropyrido[3,4-d]pyrimidine oxalate,
(26A) 2,4-Bis{4-[(4-dimethylaminopropyl)aminomethyl]phenyl}thieno[2,3-d]pyrimidine,
(26B) 2,4-Bis{4-[(4-dimethylaminopropyl)aminomethyl]phenyl}thieno[2,3-d]pyrimidine oxalate,
(27A) 2,4-Bis{4-[(3-(4-methylpiperazin-1-yl)propyl)aminomethyl]phenyl}thieno[2,3-d]pyrimidine,
(27B) 2,4-Bis{4-[(3-(4-methylpiperazin-1-yl)propyl)aminomethyl]phenyl}thieno[2,3-d]pyrimidine oxalate,
(28A) 2-(3-Dimethylaminopropylamino)-4-{4-[(4-dimethylaminopropyl)aminomethyl]phenyl}thieno[2,3-d]pyrimidine,
(28B) 2-(3-Dimethylaminopropylamino)-4-{4-[(4-dimethylaminopropyl)aminomethyl]phenyl}thieno[2,3-d]pyrimidine oxalate,
(29A) 2,4-Bis{4-[(4-dimethylaminopropyl)aminomethyl]phenyl}thieno[3,2-d]pyrimidine,
(29B) 2,4-Bis{4-[(4-dimethylaminopropyl)aminomethyl]phenyl}thieno[3,2-d]pyrimidine oxalate,
(30A) 2,4-Bis{4-[(3-(4-methylpiperazin-1-yl)propyl)aminomethyl]phenyl}thieno[3,2-d]pyrimidine,
(30B) 2,4-Bis{4-[(3-(4-methylpiperazin-1-yl)propyl)aminomethyl]phenyl}thieno[3,2-d]pyrimidine oxalate,
(31A) 2-(3-Dimethylaminopropylamino)-4-{4-[(4-dimethylaminopropyl)aminomethyl]phenyl}thieno[3,2-d]pyrimidine,
(31B) 2-(3-Dimethylaminopropylamino)-4-{4-[(4-dimethylaminopropyl)aminomethyl]phenyl}thieno[3,2-d]pyrimidine oxalate,
(32A) 2,7-bis{4-[(3-dimethylaminobutyl)aminomethyl]phenyl}-4-phenyl-7H-pyrrolo[2,3-d]pyrimidine,
(32B) 2,7-bis{4-[(3-dimethylaminobutyl)aminomethyl]phenyl}-4-phenyl-7H-pyrrolo[2,3-d]pyrimidine oxalate,
(33A) 2,4-bis{4-[(3-diethylaminobutyl)aminomethyl]phenyl}-7-phenyl-7H-pyrrolo[2,3-d]pyrimidine,
(33B) 2,4-bis{4-[(3-diethylaminobutyl)aminomethyl]phenyl}-7-phenyl-7H-pyrrolo[2,3-d]pyrimidine oxalate,
(34A) 2,4-bis{4-[(3-diisopropylaminopentyl)aminomethyl]phenyl}-7-phenyl-7H-pyrrolo[2,3-d]pyrimidine,
(34B) 2,4-bis{4-[(3-diisopropylaminopentyl)aminomethyl]phenyl}-7-phenyl-7H-pyrrolo[2,3-d]pyrimidine oxalate,
(35A) 2,7-bis{4-[(3-dimethylaminopentyl)aminomethyl]phenyl}-4-phenyl-7H-pyrrolo[2,3-d]pyrimidine,
(35B) 2,7-bis{4-[(3-dimethylaminopentyl)aminomethyl]phenyl}-4-phenyl-7H-pyrrolo[2,3-d]pyrimidine oxalate,
(36A) 4,6-Bis{4-[(3-dimethylaminopropyl)aminomethyl]phenyl}-1-phenyl-1H-pyrazolo[3,4-d]pyrimidine,
(36B) 4,6-Bis{4-[(3-dimethylaminopropyl)aminomethyl]phenyl}-1-phenyl-1H-pyrazolo[3,4-d]pyrimidine oxalate,
(37A) 4,6-Bis{4-[(4-dimethylaminobutyl)aminomethyl]phenyl}-1-phenyl-1H-pyrazolo[3,4-d]pyrimidine,
(37B) 4,6-Bis{4-[(4-dimethylaminobutyl)aminomethyl]phenyl}-1-phenyl-1H-pyrazolo[3,4-d]pyrimidine,
(38A) 4,6-Bis{4-[(5-dimethylaminopentyl)aminomethyl]phenyl}-1-phenyl-1H-pyrazolo[3,4-d]pyrimidine,
(38B) 4,6-Bis{4-[(5-dimethylaminopentyl)aminomethyl]phenyl}-1-phenyl-1H-pyrazolo[3,4-d]pyrimidine oxalate,
(39A) 4,6-Bis{4-[(4-(4-methylpiperazin-1-yl)butyl)aminomethyl]phenyl}-1-phenyl-1H-pyrazolo[3,4-d]pyrimidine,
(39B) 4,6-Bis{4-[(4-(4-methylpiperazin-1-yl)butyl)aminomethyl]phenyl}-1-phenyl-1H-pyrazolo[3,4-d]pyrimidine oxalate,
(40A) 2,4-Bis{4-[(4-dimethylaminobutyl)aminomethyl]phenyl}-7-phenyl-7H-imidazo[4,5-d]pyrimidine,
(40B) 2,4-Bis{4-[(4-dimethylaminobutyl)aminomethyl]phenyl}-7-phenyl-7H-imidazo[4,5-d]pyrimidine oxalate,
(41A) 2,4-Bis{4-[(5-dimethylaminopentyl)aminomethyl]phenyl}-7-phenyl-7H-imidazo[4,5-d]pyrimidine,
(41B) 2,4-Bis{4-[(5-dimethylaminopentyl)aminomethyl]phenyl}-7-phenyl-7H-imidazo[4,5-d]pyrimidine oxalate,
(42A) 2,4-Bis{4-[(4-(4-methylpiperazin-1-yl)butyl)aminomethyl]phenyl}-7-phenyl-7H-imidazo[4,5-d]pyrimidine,
(42B) 2,4-Bis{4-[(4-(4-methylpiperazin-1-yl)butyl)aminomethyl]phenyl}-7-phenyl-7H-imidazo[4,5-d]pyrimidine oxalate,
(43A) 2-Chloro-4-{4-[(3-dimethylaminopropyl)aminomethyl]phenyl}-7-phenyl-7H-imidazo[4,5-d]pyrimidine,
(43B) 2-Chloro-4-{4-[(3-dimethylaminopropyl)aminomethyl]phenyl}-7-phenyl-7H-imidazo[4,5-d]pyrimidine oxalate,
(44A) 4,6-Bis{4-[(4-dimethylaminobutyl)aminomethyl]phenyl}-1H-pyrrolo[2,3-b]pyridine,
(44B) 4,6-Bis{4-[(4-dimethylaminobutyl)aminomethyl]phenyl}-1H-pyrrolo[2,3-b]pyridine oxalate,
(45A) 4,6-Bis{4-[(4-(4-methylpiperazin-1-yl)butyl)aminomethyl]phenyl}-1H-pyrrolo[2,3-b]pyridine,
(45B) 4,6-Bis{4-[(4-(4-methylpiperazin-1-yl)butyl)aminomethyl]phenyl}-1H-pyrrolo[2,3-b]pyridine oxalate,
(46A) 4,6-Bis{4-[(4-dimethylaminobutyl)aminomethyl]phenyl}-1-benzyl-1H-pyrrolo[2,3-b]pyridine,
(46B) 4,6-Bis{4-[(4-dimethylaminobutyl)aminomethyl]phenyl}-1-benzyl-1H-pyrrolo[2,3-b]pyridine oxalate,
(47A) 6-Chloro-4-{4-[(3-dimethylaminopropyl)aminomethyl]phenyl}-1-phenyl-1H-pyrrolo[2,3-b]pyridine,
(47B) 6-Chloro-4-{4-[(3-dimethylaminopropyl)aminomethyl]phenyl}-1-phenyl-1H-pyrrolo[2,3-b]pyridine oxalate,
(48A) 2,4-bis{4-[(3-dimethylaminobutyl)aminomethyl]phenyl}-7-(4-methoxyphenyl)-7H-pyrrolo[2,3-d]pyrimidine,
(48B) 2,4-bis{4-[(3-dimethylaminobutyl)aminomethyl]phenyl}-7-(4-methoxyphenyl)-7H-pyrrolo[2,3-d]pyrimidine oxalate,
(49A) 2,4-Bis{4-[(4-dimethylaminobutyl)aminomethyl]phenyl}-6-phenyl-thieno[3,2-d]pyrimidine, and
(49B) 2,4-Bis{4-[(4-dimethylaminobutyl)aminomethyl]phenyl}-6-phenyl-thieno[3,2-d]pyrimidine oxalate.

21. A pharmaceutical composition comprising at least one compound as defined in claim 1 or pharmaceutically acceptable salt thereof.

22. An intermediate compound of formula (III), or a pharmaceutically acceptable salt thereof: 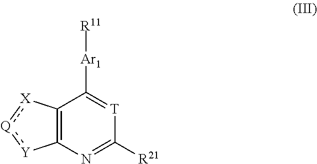
wherein:
R¹¹ is a hydrogen atom, a halogen atom, or a —(CH₂)ₙ₋₁—CHO,
R²¹ is a hydrogen atom, a halogen atom, or a (C₅-C₁₁)aryl or (C₅-C₁₁)heteroaryl group, optionally substituted by a halogen atom or —(CH₂)ₙ₋₁—CHO,
X is —CH—, —S—, —NR⁵¹— or —N—,
Y is —CH—, —NR⁵¹—, —S— or —NR⁶¹—CH₂—,
T is —CH— and Q is —CH— or T is —N— and Q is —CR¹⁰¹— or —N—,
each [symbol] is independently a simple or double bond, and at most one of [symbol] is a double bond,
R⁵¹, if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more halogen atoms, (C₁-C₆)alkoxy or —(CH₂)ₙ₋₁—CHO,
R⁶¹, if present, is a hydrogen atom, a (C₁-C₆)alkyl group, a group —COOR⁸¹ or a (C₅-C₁₁)aryl or (C₅-C₁₁)heteroaryl group, optionally substituted by one or more halogen atoms, (C₁-C₆)alkoxy or —(CH₂)ₙ₋₁—CHO,
Ar₁ and n are as defined in claim 1,
R⁸¹ represents a hydrogen atom or a (C₁-C₆)alkyl group,
R¹⁰¹ is a hydrogen atom or a (C₅-C₁₁)aryl group or (C₅-C₁₁)heteroaryl,
provided that one or two of X, Q and Y comprises a heteroatom, and
with the proviso that, at least one of R¹¹, R²¹, and, if present, R⁵¹ or R⁶¹, contains a group —(CH₂)ₙ₋₁—CHO.

23. An intermediate compound of formula (II), or a pharmaceutically acceptable salt thereof: 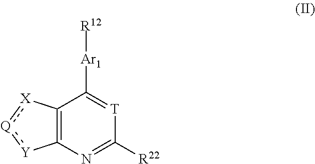
wherein:
R¹² is a hydrogen atom, a halogen atom, or a —(CH₂)ₙ₋₁CH═N-Alk-NR³R⁴,
R²² is a hydrogen atom, a halogen atom, or a (C₅-C₁₁)aryl or (C₅-C₁₁)heteroaryl group, optionally substituted by a halogen atom or —(CH₂)ₙ₋₁CH═N-Alk-NR³R⁴,
X is —CH—, —S—, —NR⁵²— or —N—,
Y is —CH—, —NR⁵²—, —S— or —NR⁶²—CH₂—,
T is —CH— and Q is —CH— or T is —N— and Q is —CR¹⁰²— or —N—,
each [symbol] is independently a simple or double bond, and at most one of [symbol] is a double bond,
R⁵², if present, is a hydrogen atom, a (C₁-C₆)alkyl group optionally substituted by one or more (C₅-C₁₁)aryl or (C₅-C₁₁)heteroaryl group, or a (C₅-C₁₁)aryl or (C₅-C₁₁)heteroaryl group optionally substituted by one or more halogen atoms, (C₁-C₆)alkoxy or —(CH₂)ₙ₋₁CH═N-Alk-NR³R⁴,
R⁶², if present, is a hydrogen atom, a (C₁-C₆)alkyl group, a group —COOR⁸² or a (C₅-C₁₁)aryl or (C₅-C₁₁)heteroaryl group, optionally substituted by one or more halogen atoms, C₁-C₆)alkoxy or —(CH₂)ₙ₋₁CH═N-Alk-NR³R⁴,
R¹⁰² is a hydrogen atom or a (C₅-C₁₁)aryl group or (C₅-C₁₁)heteroaryl,
Ar₁, Alk, R³, R⁴ and n are as defined in claim 1,
provided that one or two of X, Q and Y comprises a heteroatom, and
with the proviso that, at least one of R¹², R²², and, if present, R⁵² or R⁶², contains a group —(CH₂)ₙ₋₁CH═N-Alk-NR³R⁴.

24. Synthesis process for manufacturing a compound of formula (I) as defined in claim 1 or a pharmaceutically acceptable salt thereof, comprising at least the steps of:
(a) providing a compound having following formula (IV): 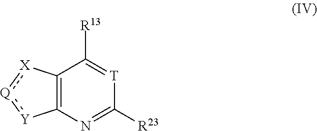
wherein
R¹³ is a halogen atom, or a (C₅-C₁₁)arylene or (C₅-C₁₁)heteroarylene group optionally substituted by a halogen atom,
R²³ is a hydrogen atom, a halogen atom or a (C₅-C₁₁) aryl or (C₅-C₁₁) heteroaryl group, optionally substituted by a halogen atom,
X is —CH—, —S— or —NR⁵³—,
Y is —CH—, —NR⁵³—, —S— or —NR⁶³—CH₂—,
T is —CH— and Q is —CH— or T is —N— and Q is —CR¹⁰³— or —N—,
each [symbol] is independently a simple or double bond, and at most one of [symbol] is a double bond,
R⁵³, if present, is a hydrogen atom, a (C₁-C₆)alkyl group optionally substituted by one or more (C₅-C₁₁)aryl or (C₅-C₁₁)heteroaryl group, a (C₅-C₁₁)aryl or (C₅-C₁₁)heteroaryl group optionally substituted by one or more halogen atoms or (C₁-C₆)alkoxy, or a protecting group,
R⁶³, if present, is a hydrogen atom, a (C₁-C₆)alkyl group, a group —COOR⁸³, a (C₅-C₁₁)aryl or (C₅-C₁₁)heteroaryl group, optionally substituted by one or more halogen atoms or C₁-C₆)alkoxy, or a protecting group,
R⁸³ represents a hydrogen atom or a (C₁-C₆)alkyl group,
R¹⁰³ is a hydrogen atom or a (C₅-C₁₁)aryl group or (C₅-C₁₁)heteroaryl
provided that one or two of X, Q and Y comprises a heteroatom, and
with the proviso that, at least one of R¹³ and R²³ is a halogen atom or, if present, R⁵³ or R⁶³ is a hydrogen atom,
(b) optionally reacting the intermediate compound of formula (IV) with at least one (C₅-C₁₁)aryl or (C₅-C₁₁)heteroaryl boronic acid, a (C₅-C₁₁)aryl or (C₅-C₁₁)heteroaryl boronate optionally substituted by one or more (C₁-C₄)alkyl and optionally protected, or a (C₅-C₁₁)aryl or (C₅-C₁₁)heteroaryl trifluoro borate salt, substituted by one or more —(CH₂)ₙ₋₁—CHO under reactive conditions suitable for grafting a function (C₅-C₁₁)aryl-(CH₂)ₙ₁—CHO or (C₅-C₁₁)heteroaryl-(CH₂)ₙ₋₁—CHO, such reacting being eventually followed by deprotecting the function —NR⁵³— or —NR⁶³ if present and/or reacting the function —NR⁵³— or —NR⁶³ where R⁵³ and R⁶³ are hydrogen atoms, if present, under conditions suitable for grafting a (C₁-C₆)alkyl group substituted by one or more (C₅-C₁₁)aryl or (C₅-C₁₁)heteroaryl group, and thus obtaining a compound of formula (III) as defined in claim 22;
(c) coupling the formaldehyde function(s) from compound of formula (III) obtained in step (b) with a diamine compound of formula H₂N-Alk-NR³R⁴ under reactive conditions suitable for the formation of imine functions leading to a compound of formula (II) and coupling the halogen atom, when present as R²¹ of compound (III), with the diamine compound of formula H₂N-Alk-NR³R⁴ to form an amine function —NH-Alk-NR³R⁴, or coupling the halogen atom, when present as R²³ of compound of formula (IV), with a diamine compound of formula H₂N-Alk-NR³R⁴ under reactive conditions suitable for the formation of an amine function —NH-Alk-NR³R⁴; and
(d) when necessary, coupling the imine function(s) from the compound of formula (II) obtained in step (c) under suitable reactive conditions for reducing the imine function(s) to amine function(s) —NH-Alk-NR³R⁴.

26. A method of treating an infectious disease comprising administering to a patient in need thereof a compound of formula (I) according to claim 1 or a pharmaceutically acceptable salt thereof.

25. (canceled)

26. A method of treating an infectious disease comprising administering to a patient in need thereof a compound of formula (I) according to claim 1 or pharmaceutically acceptable salt thereof.

27. The method of claim 26, wherein the infectious disease is induced by parasites and/or viruses of which replication cycle key processes involve DNA or RNA G-quadruplexes from the parasite and/or virus and/or host cell genome or transcriptome and is selected from the group consisting of P. falciparum, L. donovani, Trypanosoma brucei, Ascaris lumbricoides, S. mansoni, human immunodeficiency virus, herpes simplex virus, human papillomavirus, Epstein-Barr virus, Hepatitis C virus and SARS-CoV-2.

28. The method of claim 26, wherein the compound is selected from
(1A) 2,4-bis{4-[(3-dimethylaminopropyl)aminomethyl]phenyl}-7-phenyl-7H-pyrrolo[2,3-d]pyrimidine,
(1B) 2,4-bis{4-[(3-dimethylaminopropyl)aminomethyl]phenyl}-7-phenyl-7H-pyrrolo[2,3-d]pyrimidine oxalate,
(2A) 2,4-bis{4-[(3-(4-methylpiperazin-1-yl)propyl)aminomethyl]phenyl}-7-phenyl-7H-pyrrolo[2,3-d]pyrimidine,
(2B) 2,4-bis{4-[(3-(4-methylpiperazin-1-yl)propyl)aminomethyl]phenyl}-7-phenyl-7H-pyrrolo[2,3-d]pyrimidine oxalate,
(3A) 2,4-bis{4-[(3-dimethylaminobutyl)aminomethyl]phenyl}-7-phenyl-7H-pyrrolo[2,3-d]pyrimidine,
(3B) 2,4-bis{4-[(3-dimethylaminobutyl)aminomethyl]phenyl}-7-phenyl-7H-pyrrolo[2,3-d]pyrimidine oxalate,
(4A) 2,4-bis{4-[(3-(4-methylpiperazin-1-yl)butyl)aminomethyl]phenyl}-7-phenyl-7H-pyrrolo[2,3-d]pyrimidine,
(4B) 2,4-bis{4-[(3-(4-methylpiperazin-1-yl)butyl)aminomethyl]phenyl}-7-phenyl-7H-pyrrolo[2,3-d]pyrimidine oxalate,
(5A) 2,4-bis{4-[(3-dimethylaminopentyl)aminomethyl]phenyl}-7-phenyl-7H-pyrrolo[2,3-d]pyrimidine,
(5B) 2,4-bis{4-[(3-dimethylaminopentyl)aminomethyl]phenyl}-7-phenyl-7H-pyrrolo[2,3-d]pyrimidine oxalate,
(6A) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine,
(6B) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine oxalate,
(7A) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine,
(7B) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine oxalate,
(8A) 2,4-bis{4-[(3-dimethylaminopropyl)aminomethyl]phenyl}-7-methyl-7H-pyrrolo[2,3-d]pyrimidine,
(8B) 2,4-bis{4-[(3-dimethylaminopropyl)aminomethyl]phenyl}-7-methyl-7H-pyrrolo[2,3-d]pyrimidine oxalate,
(9A) 2,4-bis{4-[(3-(4-methylpiperazin-1-yl)propyl)aminomethyl]phenyl}-7-methyl-7H-pyrrolo[2,3-d]pyrimidine,
(9B) 2,4-bis{4-[(3-(4-methylpiperazin-1-yl)propyl)aminomethyl]phenyl}-7-methyl-7H-pyrrolo[2,3-d]pyrimidine oxalate,
(10A) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine,
(10B) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine oxalate,
(11A) 2,4-Bis{4-[(5-amino-5-(methoxycarbonyl)pentyl)aminomethyl]phenyl}-7-methyl-7H-pyrrolo[2,3-d]pyrimidine,
(11B) 2,4-Bis{4-[(5-amino-5-(methoxycarbonyl)pentyl)aminomethyl]phenyl}-7-methyl-7H-pyrrolo[2,3-d]pyrimidine oxalate,
(12A) 7-(4-[(3-Dimethylaminopropyl)aminomethyl]phenyl)-2,4-diphenyl-7H-pyrrolo[2,3-d]pyrimidine,
(12B) 7-(4-[(3-Dimethylaminopropyl)aminomethyl]phenyl)-2,4-diphenyl-7H-pyrrolo[2,3-d]pyrimidine oxalate,
(13A) 2,4,7-Tri{4-[(3-dimethylaminopropyl)aminomethyl]phenyl}-7H-pyrrolo[2,3-d]pyrimidine,
(13B) 2,4,7-Tri{4-[(3-dimethylaminopropyl)aminomethyl]phenyl}-7H-pyrrolo[2,3-d]pyrimidine oxalate,
(14A) 2-(3-Dimethylaminopropylamino)-4-{4-[(4-dimethylaminopropyl)aminomethyl]phenyl}-7-phenyl-7H-pyrrolo[2,3-d]pyrimidine,
(14B) 2-(3-Dimethylaminopropylamino)-4-{4-[(4-dimethylaminopropyl)aminomethyl]phenyl}-7-phenyl-7H-pyrrolo[2,3-d]pyrimidine oxalate,
(15A) 2-Chloro-4-{4-[(3-dimethylaminopropyl)aminomethyl]phenyl}-7H-pyrrolo[2,3-d]pyrimidine,
(15B) 2-Chloro-4-{4-[(3-dimethylaminopropyl)aminomethyl]phenyl}-7H-pyrrolo[2,3-d]pyrimidine oxalate,
(16A) 2-Chloro-4-{4-[(3-(4-methylpiperazin-1-yl)propyl)aminomethyl]phenyl}-7H-pyrrolo[2,3-d]pyrimidine,
(16B) 2-Chloro-4-{4-[(3-(4-methylpiperazin-1-yl)propyl)aminomethyl]phenyl}-7H-pyrrolo[2,3-d]pyrimidine oxalate,
(17A) 2-Chloro-4-{4-[(3-dimethylaminopropyl)aminomethyl]phenyl}-7-phenyl-7H-pyrrolo[2,3-d]pyrimidine,
(17B) 2-Chloro-4-{4-[(3-dimethylaminopropyl)aminomethyl]phenyl}-7-phenyl-7H-pyrrolo[2,3-d]pyrimidine oxalate,
(18A) 2-chloro-4-{4-[(3-(4-methylpiperazin-1-yl)propyl)aminomethyl]phenyl}-7-phenyl-7H-pyrrolo[2,3-d]pyrimidine,
(18B) 2-chloro-4-{4-[(3-(4-methylpiperazin-1-yl)propyl)aminomethyl]phenyl}-7-phenyl-7H-pyrrolo[2,3-d]pyrimidine oxalate,
(19A) 2-Chloro-4-{5-[(3-dimethylaminopropyl)aminomethyl]thien-2-yl}-7-phenyl-7H-pyrrolo[2,3-d]pyrimidine,
(19B) 2-Chloro-4-{5-[(3-dimethylaminopropyl)aminomethyl]thien-2-yl}-7-phenyl-7H-pyrrolo[2,3-d]pyrimidine oxalate,
(20A) 2-Chloro-4-{2-[(3-dimethylaminopropyl)aminomethyl]thien-4-yl}-7-phenyl-7H-pyrrolo[2,3-d]pyrimidine,
(20B) 2-Chloro-4-{2-[(3-dimethylaminopropyl)aminomethyl]thien-4-yl}-7-phenyl-7H-pyrrolo[2,3-d]pyrimidine oxalate,
(21A) 2-Chloro-4-{2-[(3-dimethylaminopropyl)aminomethyl]thien-3-yl}-7-phenyl-7H-pyrrolo[2,3-d]pyrimidine,
(21B) 2-Chloro-4-{2-[(3-dimethylaminopropyl)aminomethyl]thien-3-yl}-7-phenyl-7H-pyrrolo[2,3-d]pyrimidine oxalate,
(22A) 2-Chloro-7-(4-[(3-dimethylaminopropyl)aminomethyl]phenyl)-4-phenyl-7H-pyrrolo[2,3-d]pyrimidine,
(22B) 2-Chloro-7-(4-[(3-dimethylaminopropyl)aminomethyl]phenyl)-4-phenyl-7H-pyrrolo[2,3-d]pyrimidine oxalate,
(23A) 2-Chloro-7-(4-[(3-(4-methylpiperazin-1-yl)propyl)aminomethyl]phenyl)-4-phenyl-7H-pyrrolo[2,3-d]pyrimidine,
(23B) 2-Chloro-7-(4-[(3-(4-methylpiperazin-1-yl)propyl)aminomethyl]phenyl)-4-phenyl-7H-pyrrolo[2,3-d]pyrimidine oxalate,
(24A) Tert-butyl 2,4-bis{4-[(3-dimethylaminopropyl)aminomethyl]phenyl})-5,6-dihydropyrido[3,4-d]pyrimidine-7(8H)-carboxylate,
(24B) Tert-butyl 2,4-bis{4-[(3-dimethylaminopropyl)aminomethyl]phenyl})-5,6-dihydropyrido[3,4-d]pyrimidine-7(8H)-carboxylate oxalate,
(25A) 2,4-Bis{4-[(3-dimethylaminopropyl)aminomethyl]phenyl})-5,6,7,8-dihydropyrido[3,4-d]pyrimidine,
(25B) 2,4-Bis{4-[(3-dimethylaminopropyl)aminomethyl]phenyl})-5,6,7,8-dihydropyrido[3,4-d]pyrimidine oxalate,
(26A) 2,4-Bis{4-[(4-dimethylaminopropyl)aminomethyl]phenyl}thieno[2,3-d]pyrimidine,
(26B) 2,4-Bis{4-[(4-dimethylaminopropyl)aminomethyl]phenyl}thieno[2,3-d]pyrimidine oxalate,
(27A) 2,4-Bis{4-[(3-(4-methylpiperazin-1-yl)propyl)aminomethyl]phenyl}thieno[2,3-d]pyrimidine,
(27B) 2,4-Bis{4-[(3-(4-methylpiperazin-1-yl)propyl)aminomethyl]phenyl}thieno[2,3-d]pyrimidine oxalate,
(28A) 2-(3-Dimethylaminopropylamino)-4-{4-[(4-dimethylaminopropyl)aminomethyl]phenyl}thieno[2,3-d]pyrimidine,
(28B) 2-(3-Dimethylaminopropylamino)-4-{4-[(4-dimethylaminopropyl)aminomethyl]phenyl}thieno[2,3-d]pyrimidine oxalate,
(29A) 2,4-Bis{4-[(4-dimethylaminopropyl)aminomethyl]phenyl}thieno[3,2-d]pyrimidine,
(29B) 2,4-Bis{4-[(4-dimethylaminopropyl)aminomethyl]phenyl}thieno[3,2-d]pyrimidine oxalate,
(30A) 2,4-Bis{4-[(3-(4-methylpiperazin-1-yl)propyl)aminomethyl]phenyl}thieno[3,2-d]pyrimidine,
(30B) 2,4-Bis{4-[(3-(4-methylpiperazin-1-yl)propyl)aminomethyl]phenyl}thieno[3,2-d]pyrimidine oxalate,
(31A) 2-(3-Dimethylaminopropylamino)-4-{4-[(4-dimethylaminopropyl)aminomethyl]phenyl}thieno[3,2-d]pyrimidine,
(31B) 2-(3-Dimethylaminopropylamino)-4-{4-[(4-dimethylaminopropyl)aminomethyl]phenyl}thieno[3,2-d]pyrimidine oxalate,
(32A) 2,7-bis{4-[(3-dimethylaminobutyl)aminomethyl]phenyl}-4-phenyl-7H-pyrrolo[2,3-d]pyrimidine,
(32B) 2,7-bis{4-[(3-dimethylaminobutyl)aminomethyl]phenyl}-4-phenyl-7H-pyrrolo[2,3-d]pyrimidine oxalate,
(33A) 2,4-bis{4-[(3-diethylaminobutyl)aminomethyl]phenyl}-7-phenyl-7H-pyrrolo[2,3-d]pyrimidine,
(33B) 2,4-bis{4-[(3-diethylaminobutyl)aminomethyl]phenyl}-7-phenyl-7H-pyrrolo[2,3-d]pyrimidine oxalate,
(34A) 2,4-bis{4-[(3-diisopropylaminopentyl)aminomethyl]phenyl}-7-phenyl-7H-pyrrolo[2,3-d]pyrimidine,
(34B) 2,4-bis{4-[(3-diisopropylaminopentyl)aminomethyl]phenyl}-7-phenyl-7H-pyrrolo[2,3-d]pyrimidine oxalate,
(35A) 2,7-bis{4-[(3-dimethylaminopentyl)aminomethyl]phenyl}-4-phenyl-7H-pyrrolo[2,3-d]pyrimidine,
(35B) 2,7-bis{4-[(3-dimethylaminopentyl)aminomethyl]phenyl}-4-phenyl-7H-pyrrolo[2,3-d]pyrimidine oxalate,
(36A) 4,6-Bis{4-[(3-dimethylaminopropyl)aminomethyl]phenyl}-1-phenyl-1H-pyrazolo[3,4-d]pyrimidine,
(36B) 4,6-Bis{4-[(3-dimethylaminopropyl)aminomethyl]phenyl}-1-phenyl-1H-pyrazolo[3,4-d]pyrimidine oxalate,
(37A) 4,6-Bis{4-[(4-dimethylaminobutyl)aminomethyl]phenyl}-1-phenyl-1H-pyrazolo[3,4-d]pyrimidine,
(37B) 4,6-Bis{4-[(4-dimethylaminobutyl)aminomethyl]phenyl}-1-phenyl-1H-pyrazolo[3,4-d]pyrimidine,
(38A) 4,6-Bis{4-[(5-dimethylaminopentyl)aminomethyl]phenyl}-1-phenyl-1H-pyrazolo[3,4-d]pyrimidine,
(38B) 4,6-Bis{4-[(5-dimethylaminopentyl)aminomethyl]phenyl}-1-phenyl-1H-pyrazolo[3,4-d]pyrimidine oxalate,
(39A) 4,6-Bis{4-[(4-(4-methylpiperazin-1-yl)butyl)aminomethyl]phenyl}-1-phenyl-1H-pyrazolo[3,4-d]pyrimidine,
(39B) 4,6-Bis{4-[(4-(4-methylpiperazin-1-yl)butyl)aminomethyl]phenyl}-1-phenyl-1H-pyrazolo[3,4-d]pyrimidine oxalate,
(40A) 2,4-Bis{4-[(4-dimethylaminobutyl)aminomethyl]phenyl}-7-phenyl-7H-imidazo[4,5-d]pyrimidine,
(40B) 2,4-Bis{4-[(4-dimethylaminobutyl)aminomethyl]phenyl}-7-phenyl-7H-imidazo[4,5-d]pyrimidine oxalate,
(41A) 2,4-Bis{4-[(5-dimethylaminopentyl)aminomethyl]phenyl}-7-phenyl-7H-imidazo[4,5-d]pyrimidine,
(41B) 2,4-Bis{4-[(5-dimethylaminopentyl)aminomethyl]phenyl}-7-phenyl-7H-imidazo[4,5-d]pyrimidine oxalate,
(42A) 2,4-Bis{4-[(4-(4-methylpiperazin-1-yl)butyl)aminomethyl]phenyl}-7-phenyl-7H-imidazo[4,5-d]pyrimidine,
(42B) 2,4-Bis{4-[(4-(4-methylpiperazin-1-yl)butyl)aminomethyl]phenyl}-7-phenyl-7H-imidazo[4,5-d]pyrimidine oxalate,
(43A) 2-Chloro-4-{4-[(3-dimethylaminopropyl)aminomethyl]phenyl}-7-phenyl-7H-imidazo[4,5-d]pyrimidine,
(43B) 2-Chloro-4-{4-[(3-dimethylaminopropyl)aminomethyl]phenyl}-7-phenyl-7H-imidazo[4,5-d]pyrimidine oxalate,
(44A) 4,6-Bis{4-[(4-dimethylaminobutyl)aminomethyl]phenyl}-1H-pyrrolo[2,3-b]pyridine,
(44B) 4,6-Bis{4-[(4-dimethylaminobutyl)aminomethyl]phenyl}-1H-pyrrolo[2,3-b]pyridine oxalate,
(45A) 4,6-Bis{4-[(4-(4-methylpiperazin-1-yl)butyl)aminomethyl]phenyl}-1H-pyrrolo[2,3-b]pyridine,
(45B) 4,6-Bis{4-[(4-(4-methylpiperazin-1-yl)butyl)aminomethyl]phenyl}-1H-pyrrolo[2,3-b]pyridine oxalate,
(46A) 4,6-Bis{4-[(4-dimethylaminobutyl)aminomethyl]phenyl}-1-benzyl-1H-pyrrolo[2,3-b]pyridine,
(46B) 4,6-Bis{4-[(4-dimethylaminobutyl)aminomethyl]phenyl}-1-benzyl-1H-pyrrolo[2,3-b]pyridine oxalate,
(47A) 6-Chloro-4-{4-[(3-dimethylaminopropyl)aminomethyl]phenyl}-1-phenyl-1H-pyrrolo[2,3-b]pyridine,
(47B) 6-Chloro-4-{4-[(3-dimethylaminopropyl)aminomethyl]phenyl}-1-phenyl-1H-pyrrolo[2,3-b]pyridine oxalate,
(48A) 2,4-bis{4-[(3-dimethylaminobutyl)aminomethyl]phenyl}-7-(4-methoxyphenyl)-7H-pyrrolo[2,3-d]pyrimidine,
(48B) 2,4-bis{4-[(3-dimethylaminobutyl)aminomethyl]phenyl}-7-(4-methoxyphenyl)-7H-pyrrolo[2,3-d]pyrimidine oxalate,
(49A) 2,4-Bis{4-[(4-dimethylaminobutyl)aminomethyl]phenyl}-6-phenyl-thieno[3,2-d]pyrimidine, and
(49B) 2,4-Bis{4-[(4-dimethylaminobutyl)aminomethyl]phenyl}-6-phenyl-thieno[3,2-d]pyrimidine oxalate.

28. The pharmaceutical composition of claim 21, wherein the at least one compound is selected from
(1A) 2,4-bis{4-[(3-dimethylaminopropyl)aminomethyl]phenyl}-7-phenyl-7H-pyrrolo[2,3-d]pyrimidine,
(1B) 2,4-bis{4-[(3-dimethylaminopropyl)aminomethyl]phenyl}-7-phenyl-7H-pyrrolo[2,3-d]pyrimidine oxalate,
(2A) 2,4-bis{4-[(3-(4-methylpiperazin-1-yl)propyl)aminomethyl]phenyl}-7-phenyl-7H-pyrrolo[2,3-d]pyrimidine,
(2B) 2,4-bis{4-[(3-(4-methylpiperazin-1-yl)propyl)aminomethyl]phenyl}-7-phenyl-7H-pyrrolo[2,3-d]pyrimidine oxalate,
(3A) 2,4-bis{4-[(3-dimethylaminobutyl)aminomethyl]phenyl}-7-phenyl-7H-pyrrolo[2,3-d]pyrimidine,
(3B) 2,4-bis{4-[(3-dimethylaminobutyl)aminomethyl]phenyl}-7-phenyl-7H-pyrrolo[2,3-d]pyrimidine oxalate,
(4A) 2,4-bis{4-[(3-(4-methylpiperazin-1-yl)butyl)aminomethyl]phenyl}-7-phenyl-7H-pyrrolo[2,3-d]pyrimidine,
(4B) 2,4-bis{4-[(3-(4-methylpiperazin-1-yl)butyl)aminomethyl]phenyl}-7-phenyl-7H-pyrrolo[2,3-d]pyrimidine oxalate,
(5A) 2,4-bis{4-[(3-dimethylaminopentyl)aminomethyl]phenyl}-7-phenyl-7H-pyrrolo[2,3-d]pyrimidine,
(5B) 2,4-bis{4-[(3-dimethylaminopentyl)aminomethyl]phenyl}-7-phenyl-7H-pyrrolo[2,3-d]pyrimidine oxalate,
(6A) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine,
(6B) 2-{4-[(3-Dimethylaminopropyl)aminomethyl]phenyl}-4-{5-[(3-dimethylaminopropyl)aminomethyl]thien-2-yl}-7-phenyl-7H-pyrrolo[2,3-d]pyrimidine oxalate,
(7A) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine,
(7B) 4-{4-[(3-Dimethylaminopropyl)aminomethyl]phenyl}-2-{5-[(3-dimethylaminopropyl)aminomethyl]thien-2-yl}-7-phenyl-7H-pyrrolo[2,3-d]pyrimidine oxalate,
(8A) 2,4-bis{4-[(3-dimethylaminopropyl)aminomethyl]phenyl}-7-methyl-7H-pyrrolo[2,3-d]pyrimidine,
(8B) 2,4-bis{4-[(3-dimethylaminopropyl)aminomethyl]phenyl}-7-methyl-7H-pyrrolo[2,3-d]pyrimidine oxalate,
(9A) 2,4-bis{4-[(3-(4-methylpiperazin-1-yl)propyl)aminomethyl]phenyl}-7-methyl-7H-pyrrolo[2,3-d]pyrimidine,
(9B) 2,4-bis{4-[(3-(4-methylpiperazin-1-yl)propyl)aminomethyl]phenyl}-7-methyl-7H-pyrrolo[2,3-d]pyrimidine oxalate,
(10A) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine,
(10B) 2,4-Bis{4-[(5-(tert-butoxycarbonylamino)-5-methoxycarbonyl)pentyl)aminomethyl]phenyl}-7-methyl-7H-pyrrolo[2,3-d]pyrimidine oxalate,
(11A) 2,4-Bis{4-[(5-amino-5-(methoxycarbonyl)pentyl)aminomethyl]phenyl}-7-methyl-7H-pyrrolo[2,3-d]pyrimidine,
(11B) 2,4-Bis{4-[(5-amino-5-(methoxycarbonyl)pentyl)aminomethyl]phenyl}-7-methyl-7H-pyrrolo[2,3-d]pyrimidine oxalate,
(12A) 7-(4-[(3-Dimethylaminopropyl)aminomethyl]phenyl)-2,4-diphenyl-7H-pyrrolo[2,3-d]pyrimidine,
(12B) 7-(4-[(3-Dimethylaminopropyl)aminomethyl]phenyl)-2,4-diphenyl-7H-pyrrolo[2,3-d]pyrimidine oxalate,
(13A) 2,4,7-Tri{4-[(3-dimethylaminopropyl)aminomethyl]phenyl}-7H-pyrrolo[2,3-d]pyrimidine,
(13B) 2,4,7-Tri{4-[(3-dimethylaminopropyl)aminomethyl]phenyl}-7H-pyrrolo[2,3-d]pyrimidine oxalate,
(14A) 2-(3-Dimethylaminopropylamino)-4-{4-[(4-dimethylaminopropyl)aminomethyl]phenyl}-7-phenyl-7H-pyrrolo[2,3-d]pyrimidine,
(14B) 2-(3-Dimethylaminopropylamino)-4-{4-[(4-dimethylaminopropyl)aminomethyl]phenyl}-7-phenyl-7H-pyrrolo[2,3-d]pyrimidine oxalate,
(15A) 2-Chloro-4-{4-[(3-dimethylaminopropyl)aminomethyl]phenyl}-7H-pyrrolo[2,3-d]pyrimidine,
(15B) 2-Chloro-4-{4-[(3-dimethylaminopropyl)aminomethyl]phenyl}-7H-pyrrolo[2,3-d]pyrimidine oxalate,
(16A) 2-Chloro-4-{4-[(3-(4-methylpiperazin-1-yl)propyl)aminomethyl]phenyl}-7H-pyrrolo[2,3-d]pyrimidine,
(16B) 2-Chloro-4-{4-[(3-(4-methylpiperazin-1-yl)propyl)aminomethyl]phenyl}-7H-pyrrolo[2,3-d]pyrimidine oxalate,
(17A) 2-Chloro-4-{4-[(3-dimethylaminopropyl)aminomethyl]phenyl}-7-phenyl-7H-pyrrolo[2,3-d]pyrimidine,
(17B) 2-Chloro-4-{4-[(3-dimethylaminopropyl)aminomethyl]phenyl}-7-phenyl-7H-pyrrolo[2,3-d]pyrimidine oxalate,
(18A) 2-chloro-4-{4-[(3-(4-methylpiperazin-1-yl)propyl)aminomethyl]phenyl}-7-phenyl-7H-pyrrolo[2,3-d]pyrimidine,
(18B) 2-chloro-4-{4-[(3-(4-methylpiperazin-1-yl)propyl)aminomethyl]phenyl}-7-phenyl-7H-pyrrolo[2,3-d]pyrimidine oxalate,
(19A) 2-Chloro-4-{5-[(3-dimethylaminopropyl)aminomethyl]thien-2-yl}-7-phenyl-7H-pyrrolo[2,3-d]pyrimidine,
(19B) 2-Chloro-4-{5-[(3-dimethylaminopropyl)aminomethyl]thien-2-yl}-7-phenyl-7H-pyrrolo[2,3-d]pyrimidine oxalate,
(20A) 2-Chloro-4-{2-[(3-dimethylaminopropyl)aminomethyl]thien-4-yl}-7-phenyl-7H-pyrrolo[2,3-d]pyrimidine,
(20B) 2-Chloro-4-{2-[(3-dimethylaminopropyl)aminomethyl]thien-4-yl}-7-phenyl-7H-pyrrolo[2,3-d]pyrimidine oxalate,
(21A) 2-Chloro-4-{2-[(3-dimethylaminopropyl)aminomethyl]thien-3-yl}-7-phenyl-7H-pyrrolo[2,3-d]pyrimidine,
(21B) 2-Chloro-4-{2-[(3-dimethylaminopropyl)aminomethyl]thien-3-yl}-7-phenyl-7H-pyrrolo[2,3-d]pyrimidine oxalate,
(22A) 2-Chloro-7-(4-[(3-dimethylaminopropyl)aminomethyl]phenyl)-4-phenyl-7H-pyrrolo[2,3-d]pyrimidine,
(22B) 2-Chloro-7-(4-[(3-dimethylaminopropyl)aminomethyl]phenyl)-4-phenyl-7H-pyrrolo[2,3-d]pyrimidine oxalate,
(23A) 2-Chloro-7-(4-[(3-(4-methylpiperazin-1-yl)propyl)aminomethyl]phenyl)-4-phenyl-7H-pyrrolo[2,3-d]pyrimidine,
(23B) 2-Chloro-7-(4-[(3-(4-methylpiperazin-1-yl)propyl)aminomethyl]phenyl)-4-phenyl-7H-pyrrolo[2,3-d]pyrimidine oxalate,
(24A) Tert-butyl 2,4-bis{4-[(3-dimethylaminopropyl)aminomethyl]phenyl})-5,6-dihydropyrido[3,4-d]pyrimidine-7(8H)-carboxylate,
(24B) Tert-butyl 2,4-bis{4-[(3-dimethylaminopropyl)aminomethyl]phenyl})-5,6-dihydropyrido[3,4-d]pyrimidine-7(8H)-carboxylate oxalate,
(25A) 2,4-Bis{4-[(3-dimethylaminopropyl)aminomethyl]phenyl})-5,6,7,8-dihydropyrido[3,4-d]pyrimidine,
(25B) 2,4-Bis{4-[(3-dimethylaminopropyl)aminomethyl]phenyl})-5,6,7,8-dihydropyrido[3,4-d]pyrimidine oxalate,
(26A) 2,4-Bis{4-[(4-dimethylaminopropyl)aminomethyl]phenyl}thieno[2,3-d]pyrimidine,
(26B) 2,4-Bis{4-[(4-dimethylaminopropyl)aminomethyl]phenyl}thieno[2,3-d]pyrimidine oxalate,
(27A) 2,4-Bis{4-[(3-(4-methylpiperazin-1-yl)propyl)aminomethyl]phenyl}thieno[2,3-d]pyrimidine,
(27B) 2,4-Bis{4-[(3-(4-methylpiperazin-1-yl)propyl)aminomethyl]phenyl}thieno[2,3-d]pyrimidine oxalate,
(28A) 2-(3-Dimethylaminopropylamino)-4-{4-[(4-dimethylaminopropyl)aminomethyl]phenyl}thieno[2,3-d]pyrimidine,
(28B) 2-(3-Dimethylaminopropylamino)-4-{4-[(4-dimethylaminopropyl)aminomethyl]phenyl}thieno[2,3-d]pyrimidine oxalate,
(29A) 2,4-Bis{4-[(4-dimethylaminopropyl)aminomethyl]phenyl}thieno[3,2-d]pyrimidine,
(29B) 2,4-Bis{4-[(4-dimethylaminopropyl)aminomethyl]phenyl}thieno[3,2-d]pyrimidine oxalate,
(30A) 2,4-Bis{4-[(3-(4-methylpiperazin-1-yl)propyl)aminomethyl]phenyl}thieno[3,2-d]pyrimidine,
(30B) 2,4-Bis{4-[(3-(4-methylpiperazin-1-yl)propyl)aminomethyl]phenyl}thieno[3,2-d]pyrimidine oxalate,
(31A) 2-(3-Dimethylaminopropylamino)-4-{4-[(4-dimethylaminopropyl)aminomethyl]phenyl}thieno[3,2-d]pyrimidine,
(31B) 2-(3-Dimethylaminopropylamino)-4-{4-[(4-dimethylaminopropyl)aminomethyl]phenyl}thieno[3,2-d]pyrimidine oxalate,
(32A) 2,7-bis{4-[(3-dimethylaminobutyl)aminomethyl]phenyl}-4-phenyl-7H-pyrrolo[2,3-d]pyrimidine,
(32B) 2,7-bis{4-[(3-dimethylaminobutyl)aminomethyl]phenyl}-4-phenyl-7H-pyrrolo[2,3-d]pyrimidine oxalate,
(33A) 2,4-bis{4-[(3-diethylaminobutyl)aminomethyl]phenyl}-7-phenyl-7H-pyrrolo[2,3-d]pyrimidine,
(33B) 2,4-bis{4-[(3-diethylaminobutyl)aminomethyl]phenyl}-7-phenyl-7H-pyrrolo[2,3-d]pyrimidine oxalate,
(34A) 2,4-bis{4-[(3-diisopropylaminopentyl)aminomethyl]phenyl}-7-phenyl-7H-pyrrolo[2,3-d]pyrimidine,
(34B) 2,4-bis{4-[(3-diisopropylaminopentyl)aminomethyl]phenyl}-7-phenyl-7H-pyrrolo[2,3-d]pyrimidine oxalate,
(35A) 2,7-bis{4-[(3-dimethylaminopentyl)aminomethyl]phenyl}-4-phenyl-7H-pyrrolo[2,3-d]pyrimidine,
(35B) 2,7-bis{4-[(3-dimethylaminopentyl)aminomethyl]phenyl}-4-phenyl-7H-pyrrolo[2,3-d]pyrimidine oxalate,
(36A) 4,6-Bis{4-[(3-dimethylaminopropyl)aminomethyl]phenyl}-1-phenyl-1H-pyrazolo[3,4-d]pyrimidine,
(36B) 4,6-Bis{4-[(3-dimethylaminopropyl)aminomethyl]phenyl}-1-phenyl-1H-pyrazolo[3,4-d]pyrimidine oxalate,
(37A) 4,6-Bis{4-[(4-dimethylaminobutyl)aminomethyl]phenyl}-1-phenyl-1H-pyrazolo[3,4-d]pyrimidine,
(37B) 4,6-Bis{4-[(4-dimethylaminobutyl)aminomethyl]phenyl}-1-phenyl-1H-pyrazolo[3,4-d]pyrimidine,
(38A) 4,6-Bis{4-[(5-dimethylaminopentyl)aminomethyl]phenyl}-1-phenyl-1H-pyrazolo[3,4-d]pyrimidine,
(38B) 4,6-Bis{4-[(5-dimethylaminopentyl)aminomethyl]phenyl}-1-phenyl-1H-pyrazolo[3,4-d]pyrimidine oxalate,
(39A) 4,6-Bis{4-[(4-(4-methylpiperazin-1-yl)butyl)aminomethyl]phenyl}-1-phenyl-1H-pyrazolo[3,4-d]pyrimidine,
(39B) 4,6-Bis{4-[(4-(4-methylpiperazin-1-yl)butyl)aminomethyl]phenyl}-1-phenyl-1H-pyrazolo[3,4-d]pyrimidine oxalate,
(40A) 2,4-Bis{4-[(4-dimethylaminobutyl)aminomethyl]phenyl}-7-phenyl-7H-imidazo[4,5-d]pyrimidine,
(40B) 2,4-Bis{4-[(4-dimethylaminobutyl)aminomethyl]phenyl}-7-phenyl-7H-imidazo[4,5-d]pyrimidine oxalate,
(41A) 2,4-Bis{4-[(5-dimethylaminopentyl)aminomethyl]phenyl}-7-phenyl-7H-imidazo[4,5-d]pyrimidine,
(41B) 2,4-Bis{4-[(5-dimethylaminopentyl)aminomethyl]phenyl}-7-phenyl-7H-imidazo[4,5-d]pyrimidine oxalate,
(42A) 2,4-Bis{4-[(4-(4-methylpiperazin-1-yl)butyl)aminomethyl]phenyl}-7-phenyl-7H-imidazo[4,5-d]pyrimidine,
(42B) 2,4-Bis{4-[(4-(4-methylpiperazin-1-yl)butyl)aminomethyl]phenyl}-7-phenyl-7H-imidazo[4,5-d]pyrimidine oxalate,
(43A) 2-Chloro-4-{4-[(3-dimethylaminopropyl)aminomethyl]phenyl}-7-phenyl-7H-imidazo[4,5-d]pyrimidine,
(43B) 2-Chloro-4-{4-[(3-dimethylaminopropyl)aminomethyl]phenyl}-7-phenyl-7H-imidazo[4,5-d]pyrimidine oxalate,
(44A) 4,6-Bis{4-[(4-dimethylaminobutyl)aminomethyl]phenyl}-1H-pyrrolo[2,3-b]pyridine,
(44B) 4,6-Bis{4-[(4-dimethylaminobutyl)aminomethyl]phenyl}-1H-pyrrolo[2,3-b]pyridine oxalate,
(45A) 4,6-Bis{4-[(4-(4-methylpiperazin-1-yl)butyl)aminomethyl]phenyl}-1H-pyrrolo[2,3-b]pyridine,
(45B) 4,6-Bis{4-[(4-(4-methylpiperazin-1-yl)butyl)aminomethyl]phenyl}-1H-pyrrolo[2,3-b]pyridine oxalate,
(46A) 4,6-Bis{4-[(4-dimethylaminobutyl)aminomethyl]phenyl}-1-benzyl-1H-pyrrolo[2,3-b]pyridine,
(46B) 4,6-Bis{4-[(4-dimethylaminobutyl)aminomethyl]phenyl}-1-benzyl-1H-pyrrolo[2,3-b]pyridine oxalate,
(47A) 6-Chloro-4-{4-[(3-dimethylaminopropyl)aminomethyl]phenyl}-1-phenyl-1H-pyrrolo[2,3-b]pyridine,
(47B) 6-Chloro-4-{4-[(3-dimethylaminopropyl)aminomethyl]phenyl}-1-phenyl-1H-pyrrolo[2,3-b]pyridine oxalate,
(48A) 2,4-bis{4-[(3-dimethylaminobutyl)aminomethyl]phenyl}-7-(4-methoxyphenyl)-7H-pyrrolo[2,3-d]pyrimidine,
(48B) 2,4-bis{4-[(3-dimethylaminobutyl)aminomethyl]phenyl}-7-(4-methoxyphenyl)-7H-pyrrolo[2,3-d]pyrimidine oxalate,
(49A) 2,4-Bis{4-[(4-dimethylaminobutyl)aminomethyl]phenyl}-6-phenyl-thieno[3,2-d]pyrimidine, and
(49B) 2,4-Bis{4-[(4-dimethylaminobutyl)aminomethyl]phenyl}-6-phenyl-thieno[3,2-d]pyrimidine oxalate.


SEQUENCE LISTING (5 sequences)

SEQ ID NO: 1 - DNA, 24 bp, Artificial Sequence
  dual-labeled oligonucleotide mimicking the Plasmodium telomeric sequences FPf1T
gggtttagggtttagggtttaggg

SEQ ID NO: 2 - DNA, 24 bp, Artificial Sequence
  dual-labeled oligonucleotide mimicking the Plasmodium telomeric sequence FPf8T
gggttcagggttcagggttcaggg

SEQ ID NO: 3 - DNA, 29 bp, Artificial Sequence
  dual-labeled oligonucleotide mimicking the Trypanosoma 9 and 11 chromosomic sequence FTrypBT
gggcagggggtgatggggaggagccaggg

SEQ ID NO: 4 - DNA, 21 bp, Artificial Sequence
  dual-labeled oligonucleotide mimicking the human telomeric sequence F21T
gggttagggttagggttaggg

SEQ ID NO: 5 - DNA, 21 bp, Artificial Sequence
  dual-labeled oligonucleotide mimicking the human duplex sequence FdxT
  misc_feature (10)..(12)
  misc_feature (11)..(11): n is a, c, g, or t
tatagctatantatagctata